(19) United States
(12) Patent Application Publication
(10) Pub. No.: US 2006/0241173 A1
(43) Pub. Date: Oct. 26, 2006

(54) Compound

(75) Inventors: Michael John Reed, Slough (GB); Barry Victor Lloyd Potter, Slough (GB)

Correspondence Address: FROMMER LAWRENCE & HAUG, 745 FIFTH AVENUE- 10TH FL., NEW YORK, NY, 10151, US

(73) Assignee: Sterix Ltd.

(21) Appl. No.: 11/400,791

(22) Filed: Apr. 7, 2006

Related U.S. Application Data
(63) Continuation-in-part of application No. 10/327,500, filed on Dec. 20, 2002.
Application No. 10/327,500 is a division of application No. 09/638,315, filed on Aug. 14, 2000, now Pat. No. 6,506,792.
Application No. 09/638,315 is a continuation-in-part of application No. 09/238,345, filed on Jan. 27, 1999, now Pat. No. 6,187,766.
Application No. 09/238,345 is a division of application No. 09/111,927, filed on Jul. 8, 1998, now Pat. No. 6,011,024.
Application No. 09/111,927 is a continuation-in-part of application No. PCT/GB97/00600, filed on Mar. 4, 1997.
(63) Continuation-in-part of application No. 10/991,137, filed on Nov. 17, 2004.
Application No. 10/991,137 is a division of application No. 09/638,314, filed on Aug. 14, 2000, now Pat. No. 6,921,776.
Application No. 09/638,314 is a continuation-in-part of application No. 09/238,345, filed on Jan. 27, 1999, now Pat. No. 6,187,766.
Application No. 09/238,345 is a division of application No. 09/111,927, filed on Jul. 8, 1998, now Pat. No. 6,011,024.
Application No. 09/111,927 is a continuation-in-part of application No. PCT/GB97/00444, filed on Feb. 17, 1997.
Application No. 10/991,137 is a continuation-in-part of application No. 09/125,255, filed on Aug. 14, 1998, now Pat. No. 6,239,169.

(30) Foreign Application Priority Data
Mar. 5, 1996 (GB) 9604709.7
Mar. 19, 1996 (GB) 9605725.2
Feb. 16, 1996 (GB) 9603325.3

(51) Int. Cl. A61K 31/353

(52) U.S. Cl. 514/456; 549/405

(57) ABSTRACT

The present invention relates to sulphamate compounds that are an inhibitor of both oestrone sulphatase activity and aromatase activity.

### REFERENCE TO RELATED APPLICATIONS

This application is a continuation-in-part of allowed U.S. application Ser. No. 10/327,500 filed Dec. 20, 2002, which is a divisional of U.S. application Ser. No. 09/638,315, filed Aug. 14, 2000, now U.S. Pat. No. 6,506,792, issued Jan. 14, 2003, as a continuation-in-part of U.S. application Ser. No. 09/238,345, filed Jan. 27, 1999, now U.S. Pat. No. 6,187,266, issued Feb. 13, 2001, which was a division of U.S. application Ser. No. 09/111,927, filed Jul. 8, 1998, now U.S. Pat. No. 6,011,024, issued Jan. 4, 2000, which in turn was a continuation-in-part of, inter alia, PCT patent application number PCT/GB97/00600, filed Mar. 4, 1997, designating the U.S., and claiming priority from United Kingdom patent applications 9604709.7 and 9605725.2, filed Mar. 5 and 19, 1996, respectively. PCT/GB97/00600 was published as WO 97/32872 on Sep. 12, 1997. U.S. application Ser. No. 09/142,194, filed Sep. 2, 1998 as the National Phase (35 USC 371) of PCT/GB97/00600 is now U.S. Pat. No. 6,083,978.

This application is also a continuation-in-part of U.S. application Ser. No. 10/991,137 filed Nov. 17, 2004, which is a division of allowed U.S. application Ser. No. 09/638,314, filed Aug. 14, 2000, now U.S. Pat. No. 6,921,776, issued Jul. 26, 2005, which is a continuation-in-part of U.S. application Ser. No. 09/238,345, filed Jan. 27, 1999, now U.S. Pat. No. 6,187,766, issued Feb. 13, 2001, which was a division of U.S. application Ser. No. 09/111,927, filed Jul. 8, 1998, now U.S. Pat. No. 6,011,024, issued Jan. 4, 2000, which in turn was a continuation-in-part of inter alia PCT patent application number PCT/GB97/00444, filed Feb. 17, 1997, designating the U.S., and claiming priority from United Kingdom patent application 9603325.3, filed Feb. 16, 1996. PCT/GB97/00444 was published as WO 97/30041 on Aug. 21, 1997. U.S. application Ser. No. 10/991,137 was also a continuation-in-part of allowed U.S. application Ser. No. 09/125,255, filed Aug. 14, 1998, now U.S. Pat. No. 6,239,169, issued May 29, 2001, as the National Phase (35 USC 371) of PCT/GB97/00444, Feb. 17, 1997, designating the U.S., published as WO 97/30041 on Aug. 21, 1997, and claiming priority from United Kingdom patent application 9603325.3, filed Feb. 16, 1996.

Each of these applications and patents and each document cited or referenced in each of these applications and patents, including during any prosecution (“application cited documents”), and each document cited or referenced in each of the application cited documents, are hereby incorporated herein by reference. In addition, each document cited in this text (“herein cited documents”) and each document cited or referenced in each of the herein cited documents, are hereby incorporated herein by reference.

The present invention relates to a compound.

In particular the present invention relates to a pharmaceutical composition comprising the compound.

Breast and endometrial cancers are major causes of death in Western women. In particular, tumours in endocrine-dependent tissues, such as the breast and endometrium, occur most frequently in postmenopausal women at a time when the ovaries have ceased their production of oestrogens.

Evidence suggests that oestrogens are the major mitogens involved in stimulating and promoting the growth of tumours in endocrine-dependent tissues, such as the breast and endometrium²¹. Although plasma oestrogen concentrations are similar in women with or without breast cancer, breast tumour oestrone and oestradiol levels are significantly higher than in normal breast tissue or blood. In addition, in postmenopausal women oestrogens continue to be produced by extraglandular production in adipose tissue but also in normal and malignant breast tissues²².

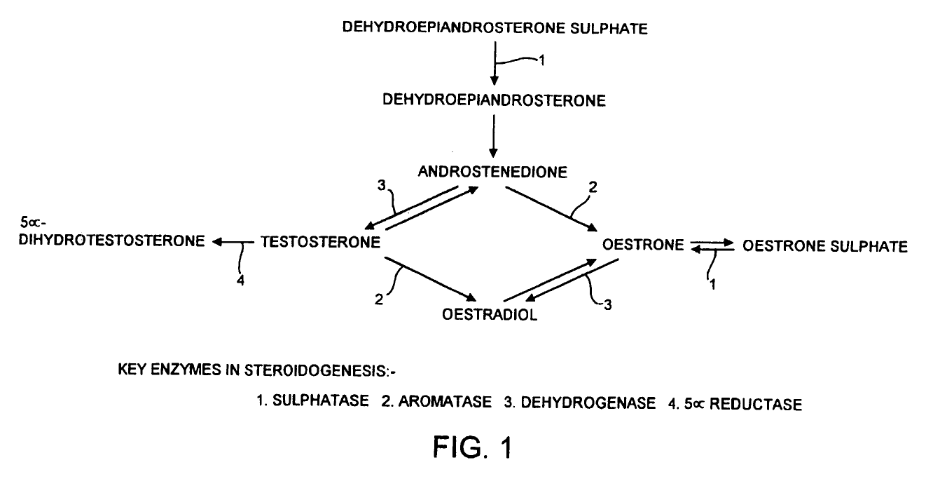

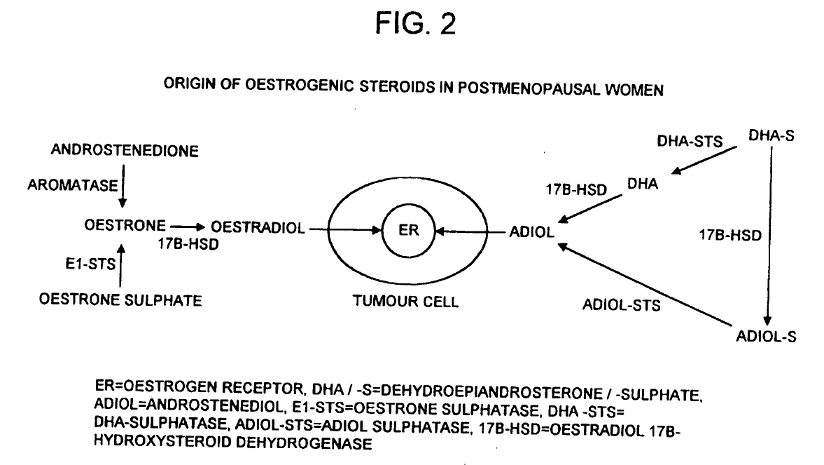

FIGS. 1 and 2 are schematic diagrams showing some of the enzymes involved in the in situ synthesis of oestrone from oestrone sulphate, oestradiol and androstenedione.

In FIG. 2, which schematically shows the origin of oestrogenic steroids in postmenopausal women, “ER” denotes Oestrogen Receptor, “DHA/-S” denotes Dehydroepiandrosterone/-Sulphate, “Adiol” denotes Androstenediol, “E1-STS” denotes Oestrone Sulphatase, “DHA-STS” denotes DHA-sulphatase, “Adiol-STS” denotes Adiol Sulphatase, and “17B-HSD” denotes Oestradiol 17B-hydroxysteroid dehydrogenase.

As can be seen, the main two enzymes that are involved in the peripheral synthesis of oestrogens are the aromatase enzyme and the enzyme oestrone sulphatase.

In short, the aromatase enzyme converts androstenedione, which is secreted in large amounts by the adrenal cortex, to oestrone. Recent reports have suggested that some flavones could inhibit aromatase activity^{35,36}.

Much of the oestrone so formed, however, is converted to oestrone sulphate (E1S) and there is now a considerable body of evidence showing that E1S in plasma and tissue acts as a reservoir for the formation of oestrone by the action of oestrone sulphatase²³.

In this regard, it is now believed that the oestrone sulphatase (E1-STS) pathway—i.e. the hydrolysis of oestrone sulphate to oestrone (E1S to E1) is the major source of oestrogen in breast tumour^{1,2}. This theory is supported by a modest reduction of plasma oestrogen concentration in postmenopausal women with breast cancer treated by aromatase inhibitors, such as aminoglutethimide and 4-hydroxyandrostenedione^{3,4} and also by the fact that plasma E1S concentration in these aromatase inhibitor-treated patients remains relatively high. The long half-life of E1S in blood (10-12 h) compared with the unconjugated oestrogens (20 min)⁵ and high levels of steroid sulphatase activity in liver and, normal and malignant breast tissues, also lend support to this theory⁶.

Thus, oestrogen formation in malignant breast and endometrial tissues via the sulphatase pathway makes a major contribution to the high concentration of oestrogens which are present in these tumours^{24,25}.

PCT/GB92/01587 teaches novel steroid sulphatase inhibitors and pharmaceutical compositions containing them for use in the treatment of oestrone dependent tumours, especially breast cancer. These steroid sulphatase inhibitors are sulphamate esters, such as N,N-dimethyl oestrone-3-sulphamate and, preferably, oestrone-3-sulphamate (otherwise known as “EMATE”).

EMATE is a potent E1-STS inhibitor as it displays more than 99% inhibition of E1-STS activity in intact MCF-7 cells at 0.1 ΦM. EMATE also inhibits the E1-STS enzyme in a time-dependent and concentration-dependent manner, thereby indicating that it acts as an active site-directed inactivator^{7,8}.

Although EMATE was originally designed for the inhibition of E1-STS, it also inhibits dehydroepiandrosterone sulphatase (DHA-STS), which is an enzyme that is believed to have a pivotal role in regulating the biosynthesis of the oestrogenic steroid androstenediol^{8,9}. This is of significance as there is now evidence to suggest that androstenediol may be of even greater importance as a promoter of breast tumour growths.

EMATE is also active in vivo as almost complete inhibition of rat liver E1-STS (99%) and DHA-STS (99%) activities resulted when it is administered either orally or subcutaneously¹¹. In addition, EMATE has been shown to have a memory enhancing effect in rats¹⁴. Studies in mice have suggested an association between DHA-STS activity and the regulation of part of the immune response. It is thought that this may also occur in humans^{15,16}. The bridging O-atom of the sulphamate moiety in EMATE is believed to be important for inhibitory activity. Thus, when the 3-O-atom is replaced by other heteroatoms—as in oestrone-3-N-sulphamate and oestrone-3-S-sulphamate—these analogues are weaker non-time-dependent inactivators¹².

Thus, EMATE is a potent steroid sulphatase inhibitor which blocks the hydrolysis of both E1S and DHA-S²⁹⁻³¹. This inhibitor, therefore, not only blocks the synthesis of oestrone from E1S but also the formation of androstenediol from DHA-S.

In addition to oestrone, the other major steroid with oestrogenic properties which is produced by postmenopausal women is androstenediol (see FIG. 2).

Androstenediol, although an androgen, can bind to the oestrogen receptor (ER) and can stimulate the growth of ER positive breast cancer cells and the growth of carcinogen-induced mammary tumours in the rat^{26,27}. Importantly, in postmenopausal women 90% of the androstenediol produced originates from the androgen dehydroepiandrosterone sulphate (DHA-S) which is secreted in large amounts by the adrenal cortex. DHA-S is converted to DHA by DHA sulphatase, which may be the same as, or different from, the enzyme, oestrone sulphatase, which is responsible for the hydrolysis of E1S²⁸.

During the last 10-15 years considerable research has also been carried out to develop potent aromatase inhibitors, some of which are currently undergoing clinical evaluation.

However, in three recent reports of postmenopausal women with breast cancer who received aromatase inhibitor therapy, plasma E1S concentrations remained between 400-1000 pg/ml³²⁻³⁴.

In summation therefore in situ synthesis of oestrogen is thought to make an important contribution to the high levels of oestrogens in tumours and therefore specific inhibitors of oestrogen biosynthesis are of potential value for the treatment of endocrine-dependent tumours.

Moreover, even though oestrogen formation in malignant breast and endometrial tissues via the sulphatase pathway makes a major contribution to the high concentration of oestrogens, there are still other enzymatic pathways that contribute to in vivo synthesis of oestrogen.

Thus, there is an urgent need to develop new therapies for the treatment of these cancers.

The present invention therefore seeks to overcome one or more of the problems associated with the prior art methods of treating breast and endometrial cancers.

According to a first aspect of the present invention there is provided a sulphamate compound suitable for use as an inhibitor of both oestrone sulphatase activity and aromatase activity.

In a highly preferred embodiment, the compound of the present invention is a non-steroidal compound.

According to a second aspect of the present invention there is provided a compound having the general formula II wherein F represents a phenolic ring structure (a first ring structure), J represents a third ring structure, I represents a phenolic ring structure (a second ring structure), G is an optional double bond, H is a link joining the second ring structure to the third ring structure, and Y represents a suitable second group; wherein any one of ring structures F, J and I has bound thereto a sulphamate group.

According to a third aspect of the present invention there is provided a compound according to the present invention for use as a pharmaceutical.

According to a fourth aspect of the present invention there is provided a compound according to the present invention for inhibiting oestrone sulphatase activity and aromatase activity.

According to a fifth aspect of the present invention there is provided a pharmaceutical composition comprising a compound according to the present invention; and a pharmaceutically acceptable carrier, excipient or diluent.

According to a sixth aspect of the present invention there is provided the use of a compound according to the present invention in the manufacture of a pharmaceutical for inhibiting oestrone sulphatase activity and aromatase activity.

According to a seventh aspect of the present invention there is provided a process for preparing a compound according to the present invention, the process comprising sulphating a flavone, isoflavone or a flavanone.

According to an eighth aspect of the present invention there is provided a process for preparing a compound according to the present invention, the process comprising sulphamaylating a flavone, isoflavone or a flavanone.

In one aspect, therefore, the present invention provides a compound, or a pharmaceutical composition comprising the same, that can affect, such as substantially inhibit, not only the oestrone sulphatase pathway—which pathway converts oestrone to and from oestradiol—but also the aromatase pathway—which pathway converts the androgen precursor androstenedione to oestrone.

This aspect of the present invention is advantageous because by the administration of one type of compound it is possible to block the synthesis of oestrone from both androstenedione and E1S.

In addition, the present invention is further advantageous in that it may also be possible to block the formation of androstenediol from DHA-S.

Hence, the present invention provides compounds that have considerable therapeutic advantages, particularly for treating breast and endometrial cancers.

The compounds of the present invention are different from those disclosed in the prior art because they can act as therapeutic agents that possess both aromatase and steroid sulphatase inhibitory properties.

In a preferred embodiment the compound of the present invention comprises a first ring structure and a sulphamoyl group, which first ring structure may be substituted and/or unsaturated.

Preferably the first ring structure is a phenolic ring structure, which phenolic ring may be substituted.

Preferably, the compound of the present invention further comprises a second ring structure, which second ring structure may be substituted and/or unsaturated.

Preferably the second ring structure is a phenolic ring structure, which phenolic ring may be substituted.

Preferably, the compound of the present invention further comprises a third ring structure which is intermediate the first ring structure and the second ring structure, which third ring structure may be substituted and/or unsaturated.

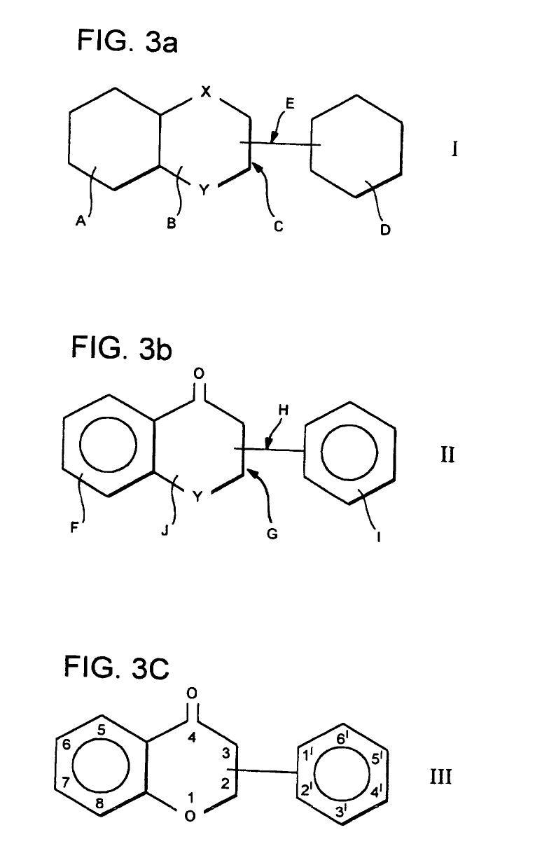

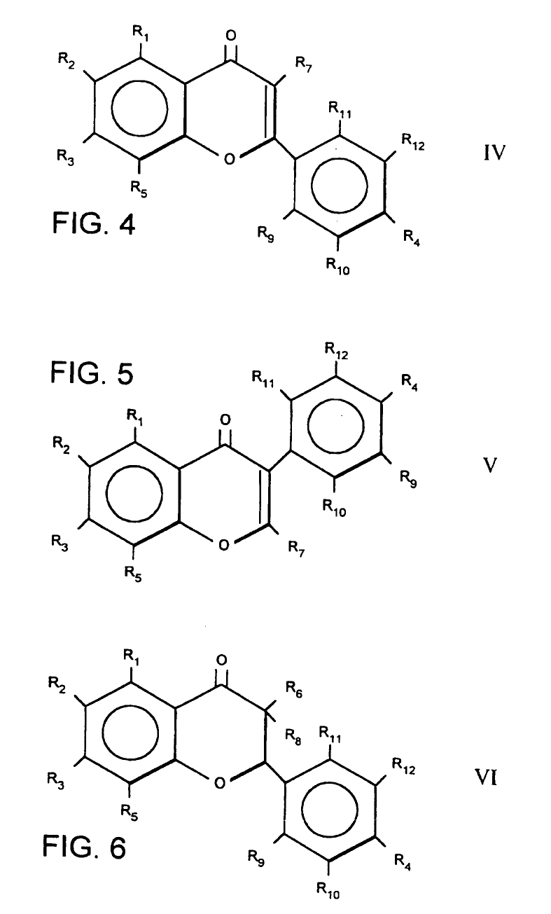

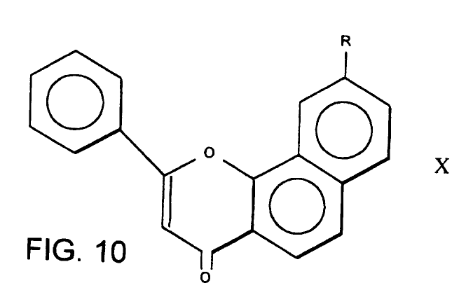

The present invention will now be described by reference to the Formulae presented in FIGS. 3-9.

In this regard, its is generally preferred that the compound of the present invention has the general formula I wherein A represents the first ring structure, B represents the third ring structure, D represents the second ring structure, C is an optional double bond, E is a link joining the second ring structure to the third ring structure, X represents a suitable first group, and Y represents a suitable second group; wherein any one of ring structures A, B and D is a phenolic ring; and wherein any one of ring structures A, B and D has bound thereto a sulphamate group.

Each of the ring structures can independently comprise from 3 to 20 atoms in the ring, preferably from 4 to 8 atoms in the ring. Preferably, ring A and ring D comprise 6 atoms in the ring.

Preferably, the first ring structure and the second ring structure are substituted.

Preferably, any one of ring structures A and D has bound thereto a sulphamate group.

Preferably, each of the first ring and the second ring is a homogeneous ring structure—i.e. the ring is made up of the same atoms.

Preferably, each of the first ring and the second ring comprises only carbon atoms in the ring.

Preferably, X is C═O.

Preferably, the compound of the present invention has the general formula II wherein F represents a phenolic ring structure (the first ring structure), J represents the third ring structure, I represents a phenolic ring structure (the second ring structure), G is an optional double bond, H is a link joining the second ring structure to the third ring structure, and Y represents a suitable second group; wherein any one of ring structures F, J and I has bound thereto a sulphamate group.

Preferably, the third ring structure is a heterogeneous ring structure—i.e. different atoms are in the ring.

Preferably, Y is O.

Preferably any one of the ring structures F and I has bound thereto a sulphamate group.

Preferably, link E or link H is a bond.

Preferably, the compound of the present invention is a sulphamate of any one of a flavone, an isoflavone or a flavanone.

Preferably, the compound of the present invention is any one of a compound of the general formula IV, a compound of the general formula V, or a compound of the general formula VI; wherein R₁-R₁₂ are independently selected from H, OH, a halogen, an amine, an amide, a sulphonamine, a sulphonamide, any other sulphur containing group, a saturated or unsaturated C₁₋₁₀ alkyl, an aryl group, a saturated or unsaturated C₁₋₁₀ ether, a saturated or unsaturated C₁₀ ester, a phosphorous containing group; and wherein at least one of R₁-R₁₂ is a sulphamate group.

Preferably, the sulphamate group has the general formula OSO₂NR₁₃R₁₄ wherein R₁₃ and R₁₄ are independently selected from H, OH, a halogen, a saturated or unsaturated C₁₋₁₀ alkyl, an aryl group, a saturated or unsaturated C₁₋₁₀ ether, a saturated or unsaturated C₁₋₁₀ ester.

Preferably, the compound of the present invention is any one of a compound of the general formula IV, a compound of the general formula V, or a compound of the general formula VI; wherein R₁-R₁₂ are independently selected from H, OH, OSO₂NR₁₃R₁₄, O—CH₃; wherein at least one of R₁-R₁₂ is OSO₂NR₁₃R₁₄, and wherein R₁₃ and R₁₄ are defined as above.

Preferably, at least one of R₁₃ and R₁₄ is H. Preferably, each of R₁₃ and R₁₄ is H.

Preferably, the compound of the present invention is a sulphamate of any one of the flavone of formula VII, the isoflavone of formula VIII, or the flavanone of formula IX.

Preferably, the compound of the present invention is the sulphamate of any one of formula VII, formula VIII or formula IX.

Preferably, the compound of the present invention is a sulphamate of any one of a flavone, an isoflavone or a flavanone; and wherein the sulphamoyl group is on the C4′ atom of the flavone, isoflavone or flavanone. The C4′ position has been shown in general Formula III according to the present invention.

Preferably, the compound of the present invention is a flavonoid or flavanoid sulphamate.

In summation the present invention provides compounds that avoid the need for polytherapy. In this regard, the compounds of the present invention can act as therapeutic agents that possess both aromatase and steroid sulphatase inhibitory properties.

Preferably, if the sulphamate group of the compound of the present invention were to be replaced with a sulphate group so as to form a sulphate compound then that sulphate compound would be hydrolysable by an enzyme having steroid sulphatase (E.C. 3.1.6.2) activity.

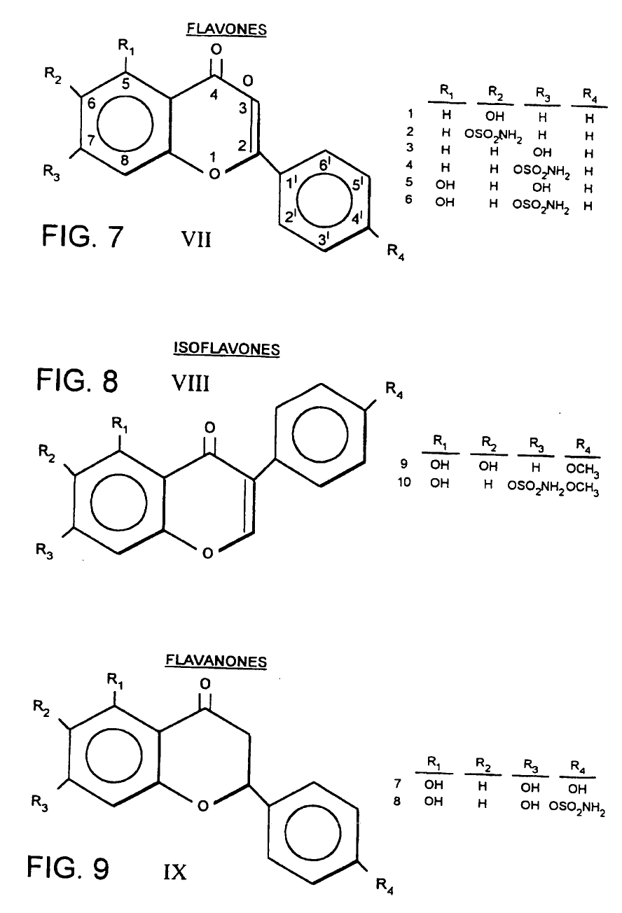

The compound of the present invention may have one or more sulphamate groups. For example, the compound may be a mono-sulphamate or a bis-sulphamate. For example, in FIGS. 7, 8 and 9 R₃ and R₄ may be each a sulphamate.

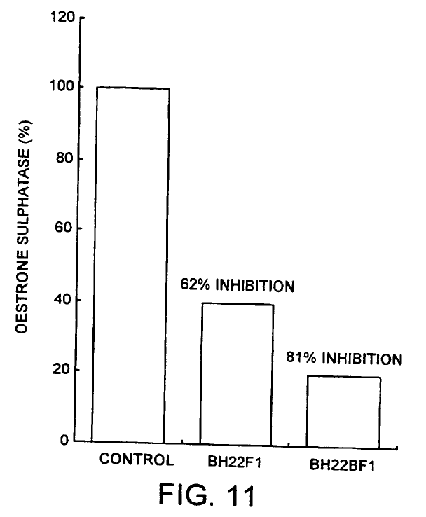

FIGS. 1 and 2 present schematic pathways; FIG. 3-10 present chemical formulae; and FIG. 11 presents a graph.

The present invention will now be described only by way of example.

Compounds Synthesised

The following sulphamate derivatives were synthesised from the following parent compounds:

| PARENT | SULPHAMATE |
|---|---|
| COMPOUND | COMPOUND |
| 1 | 2 |
| 3 | 4 |
| 5 | 6 |
| 7 | 8 |
| 9 | 10 |

wherein
1 = 6-hydroxy flavone
2 = flavone-6-sulphamate
3 = 7-hydroxy flavone
4 = flavone-7-sulphamate
5 = 5,7-dihydroxy flavone
6 = 5-hydroxy-flavone-7-sulphamate
7 = 5,7-dihydroxy-4′-hydroxy-flavone
8 = 5,7-dihydroxy flavanone-4′-flavanone sulphamate
9 = 5,7-dihydroxy-4′-methoxy-isoflavone
10 = 5-hydroxy-4′-methoxy-isoflavone-isoflavone-7-sulphamate

The formulae are presented in FIGS. 7-9.

Synthesis

The sulphamate derivatives were prepared essentially as described previously²⁹. In this regard, solutions of the appropriate flavone, isoflavone or flavanone were treated in anhydrous DMF with sodium hydride (1 equiv) at 0EC (under N₂). Sulphamoyl chloride (ca 1.5 equiv) was then added, and after the mixture was warmed to room temperature overnight and the reaction quenched, the crude product, after work up for each flavone, isoflavone or flavanone, was purified by flash chromatography and recrystallization. All compounds were fully characterised by spectroscopic and combustion analysis.

### Flavone 6-O-sulphamate (2)

6-Hydroxyflavone (1.0 g, 4.113 mmol) gave crude product (1.21 g) which was fractionated on silica (200 g) with ethyl acetate. Upon evaporation, the first fraction gave a creamy residue (760 mg, 58.2%) which was recrystallised in warm acetone/hexane (3:2) to give 2 as creamy rod-shaped crystals (557 mg). m.p. 190-191° C.; R_{f}s=0.71 (ethyl acetate), 0.51 (ethyl acetate/hexane, 2:1), νmax (KBr) 3260, 3040, 1620, 1600, 1580, 1370, 1180 cm⁻¹; δ_{H} (acetone-d₆) 6.917 (1H, s, C-3-H), 7.355 (2H, br s, exchanged with D₂O, —OSO₂NH₂), 7.64 (3H, m, C-3′-H, C-4′-H and C-5′-H), 7.75 (1H, dd, J_{C-8-H, C-7-H}=9 Hz and J_{C-5-H, C-7-H}=3 Hz, C-7-H), 7.87 ([H, d, J_{C-7-H, C-8-H}=9 Hz, C-8-H), 8.02 (1H, d, J_{C-7-H, C-5-H}=3 Hz, C-5-H) and 8.13 (2H, m, C-2′-H and C-6′-H). MS: m/z (E.I., rel. intensity) 317.0(11). 304.2(6), 238.0(96), 210.0(16), 187.1(14), 152.0(8), 136.0(100). Acc. MS (E.I.): m/z 317.0296, C₁₅H₁₁NO₅S requires 317.0358. Found C, 56.7; H, 3.44; N, 4.31, C₁₅H₁₁NO₅S requires C, 56.78; H, 3.49; N, 4.41%.

### Flavone 7-O-sulphamate (4)

7-Hydroxyflavone (700 mg, 2.938 mmol) gave crude product (770 mg) which was fractionated on silica (200 g) with ethyl acetate. Upon evaporation, the first fraction gave a light brown residue (132 mg) which was recrystallised in hot isopropyl alcohol to give 4 as white needle-shaped crystals (60 mg), m.p. 172-174° C. (dec.); R_{f}s=0.78 (ethyl acetate), 0.56 (ethyl acetate/hexane, 4.1); νmax (KBr) 3260, 3100, 1630, 1600, 1400, 1580, 1200, 1150 cm⁻¹; δ_{H} (DMSO-d₆/CDCl₃, ca. 1:20) 6.824 (1H, s, C-3-H), 7.396 (1H, dd, J_{C-5-H, C-6-H}=8.8 Hz and J_{C-8-H, C-6-H}=2.2 Hz, C-6-H), 7.47 (2H, br s, exchanged with D₂O, —OSO₂NH₂), 7.55 (3H, m, C-3′-H, C-4′-H and C-5′-H), 7.639 (1H, d, J_{C-6-H, C-8-H}=2.2 Hz, C-8-H), 7.92 (2H, m, C-2′-H and C-6′-H) and 8.220 (1H, d, J_{C-6-H, C-5-H}=8.8 Hz, C-5-H). Found: C, 56.5; H, 3.36; N, 4.19. C₁₅H₁₁NO₅S requires C, 56.78; H, 3.49; N, 4.41%.

### 5-Hydroxyflavone 7-O-Sulphamate (6)

5,7-Dihydroxyflavone (1,0 g, 3.933 mmol) gave crude product (1.13 g) which was fractionated on silica (200 g) with chloroform/acetone (8:1). Upon evaporation, the second fraction gave a yellow residue (324 mg, 24.7%) which was recrystallised in ethyl acetate/hexane (1:1) to give 6 as yellow crystals (213 mg), m.p. 195-200° C. (dec.); R_{f}s=0.21, 0.25 and 0.44 for chloroform/acetone 12:1, 8:1 and 4:1 respectively; νmax (KBr) 3360, 3250, 2925-2850, 1650, 1610, 1380 cm⁻¹, δ_{H} (acetone-d₆) 6.75, 6.98, 7.17 (3H, three s, C-3-H, C-6-H, C-8-H), 7.63 (2H, br s, exchanged with D₂O. —OSO₂NH₂), 7.65 (3H, m, C-3′-H, C-4′-H and C-5′-H), 8.15 (2H, d, J=7.7 Hz, C-2′-H and C-6′-H) and 13.0 (1H, br s, exchanged with D₂O, C-5-OH). MS: m/z (+ve ion FAB in m-NBA, rel. intensity) 440.1(10), 389.3(10), 334.1[100, (M+H)⁺], 288.1(17), 255.0(25, (M+H−79)⁺], 169.1 (30). MS: m/z (−ve ion FAB in m-NBA, rel. intensity) 499.0(30), 484.1[14, (M−2H+153)⁻], 475.1(20), 443.1(24), 332.1[100, (M−H)⁻], 308.1(28), 274.1(20), 253.1[50, (M−H−79)⁻], 195.1(24). Acc. MS (+ve ion FAB in m-NBA): m/z 334.0392, C₁₅H₁₂NO₆S requires 334.0385. Found: C, 54.0; H, 3.39; N, 4.21. C₁₅H₁₁NO₅S requires C, 54.03; H, 3.33; N, 4.20%.

### 5,7-Dihydroxyflavanone 4′-O-sulphamate (8)

4′,5,7-Trihydroxyflavanone (1.0 g, 3.675 mmol) gave crude product (965 mg) which was fractionated on silica (200 g) with ethyl acetate/hexane (4:1) to give a mixture of the starting flavanone and product. This mixture was further fractionated on silica (200 g) with chloroform/acetone (4:1) and upon evaporation, the second fraction gave a pale yellow oil (345 mg, 34%) which solidified on standing. Subsequent recrystallisation of this solid in ethyl acetate/hexane (1:1) gave 8 as white crystals (259 mg), m.p. 211-213° C.; R_{f}=0.21 (chloroform/acetone, 4:1); νmax (KBr) 3420, 3340, 3260, 3140, 1640, 1510, 1380, 1160 cm⁻¹; δ_{H} (acetone-d₆) 2.84 (1H, dd, J_{AB}=17.4 Hz and J_{ax, eq}=3.1 Hz, C-3-H_{B}), 3.19 (1H, dd, J_{BA}=16.9 Hz and J_{ax, eq}=12.8 Hz, C-3-H_{A}), 5.62 (1H, dd, J_{ax, eq}=3.1 Hz and J_{ax, eq}=12.8 Hz, C-2-H), 5.98 (1H, d, J=2.0 Hz, C-6-H or C-8-H), 6.01 (1H, d, J=2.0 Hz, C-6-H or C-8-H), 7.20 (2H, br s, exchanged with D₂O, —OSO₂NH₂), 7.40 (2H, d, J=8.7 Hz, C-2′-H and C-6′-H), 7.66 (2H, d, J=8.7 Hz, C-3′-H and C-5′-H, 9.65 (1H, br s, C-7-OH) and 12.15 (1H, s, C-5-OH). MS: m/z (+ve ion FAB in m-NBA, rel. intensity) 352.0[100, (M+H)⁻], 288.1(10), 272.1[14, (M−79)⁻], 255.2(9), 169.0(13). MS: m/z (−ve ion FAB in m-NBA, rel. intensity) 701.2(12), 606.2(10), 517.1(42), 504.1[20, (M+153)⁻], 473.2(10), 350.1[100, (M−H)⁻], 271.1[45, (M−H−79)⁻], 182.0(8). Acc. MS (+ve ion FAB in m-NBA): m/z 352.0496, C₁₅H₁₄NO₇S requires 352.0491.

Found: C, 51.1; H, 3.68; N, 3.98. C₁₅H₁₃NO₇S requires C, 51.28; H, 3.73; N, 3.99%.

### 5-Hydroxy-4′-methoxyisoflavone 7′-O-sulphamate (10)

5,7-Dihydroxy-4′-methoxyisoflavone (800 mg, 2.817 mmol) gave crude product (650 mg) which was fractionated on silica (200 g) with chloroform/acetone (8:1). Upon evaporation, the second fraction gave a yellow residue (266 mg, 26%) which was recrystallised in ethyl acetate/hexane (1:1) to give 10 as yellow crystals (211 mg), m.p. 184-188° C.; R_{f}s=0.22 and 0.59 for chloroform/acetone 8:1 and 4:1 respectively; νmax (KBr) 3300-3020, 1660, 1610, 1400 cm⁻¹; δ_{H} (acetone-d₆) 3.86 (3H, s, —OCH₃), 6.75 (1H, d, J=2.2 Hz, C-6-H or C-8-H), 7.04 (3H, m, C-6-H or C-8-H and C-3′-H and C-5′-H), 7.49 (2H, br s, exchanged with D₂O, —OSO₂NH₂), 7.58 (2H, d, J=7 Hz, C-2′-H and C-6′-H), 8.41 (1H, s, C-2-H), 13.05 (1H, br s, exchanged with D₂O, C-5-OH). MS: m/z (+ve ion FAB in m-NBA, rel. intensity) 393.3(12), 364.0[100, (M+H)⁻], 284.1[12, (M−79)⁻], 169.1(24), 134.0(22). MS: m/z (−ve ion FAB in m-NBA, rel. intensity) 529.1(25), 515.1[12, (M−H+153)⁻], 442.1(20), 362.1[100, (M−H)⁻], 308.1(34), 283.1[70, (M−H−79)⁻], 170.1(26). Acc. MS (+ve ion FAB in m-NBA): m/z 364.0494, C₁₆H₁₄NO₇S requires 364.0491. Found: C, 52.8; H, 3.65; N, 3.81. C₁₆H₁₃NO₇S requires C, 52.89; H, 3.61; N, 3.85%.

### 5-Hydroxy Isoflavone-4′,7-O,O-Disulphamate (11) and 5,7-Dihydroxy Isoflavone-4′-O-Sulphamate (12)

4′,5,7-Trihydroxy isoflavone (0.5 g, 1.85 mmol) upon sulphamoylation gave a crude product (0.65 g) which was fractionated on silica (200 g) with chloroform/acetone (4:1), and upon evaporation the third fraction gave a light yellow residue (0.329 g, 51%) which was recrystallized in ethylacetate/hexane (1:2) to give compound (11) as beige crystals (0.197 g); m.p=>198EC (dec); R_{f}s=0.14 and 0.24 for chloroform/acetone 4:1 and 2:1 respectively; v^{max} (KBr) 3460 (—NH₂), 1650 (C═O), 1400 (—SO₂N—) cm⁻¹; δ_{H} (acetone-d₆) 6.78 (1H, d, J=2.2 Hz, C-6-H or C-8-H, 7.03 (1H, d, J=2.2 Hz, C-8-H or C-6-H), 7.4 (4H, br s, exchanged with D₂O, C-4′-OSO₂NH₂ and C-7-OSO₂NH₂), 7.43 (2H, d, J=8.4 Hz, C-3′-H and C-5′-H or C-2′-H and C-6′-H and C-6′-H), 7.72 (2H, d, J=8.4 Hz, C-2′-H and C-6′-H or C-3′-H and C-5′-H), 8.51 (1H, s, C-2-H) and 12.93 (1H, s, C-5-OH). MS: m/z (+ve ion FAB in m-NBA, rel. intensity) 428.9 [100, (M+H)⁺], 350.0 [20, (M+H—SO₂NH₂)⁺], 272.1 [30, (M−H—SO₂NH₂)⁺]. MS: m/z (−ve ion FAB in m-NBA, rel. intensity) 426.9 [100, (M−H)⁻], 347.9 [95, (M−H—SO₂NH₂)⁻], 269.0 [30, (M−H—SO₂NH₂)⁻]. Acc. MS: m/z (FAB)⁺ 429.0083 C₁₅H₁₃N₂O₉S₂ requires 429.0063. Found C, 42.0; H, 2.91; N, 6.45; C₁₅H₁₂N₂O₉S₂ requires C, 42.06; H, 2.82; N, 6.54%.

The second fraction was collected and upon evaporation gave light yellow residue (0.112 g, 17%) which was recrystallized in ethylacetate/hexane (1:3) to give compound (12) as pale white crystals (0.068 g); m.p.=189-192EC R_{f}s=0.23 and 0.33 for chloroform/acetone 4:1 and 2:1 respectively; v^{max} (KBr) 3500-3300 (—NH₂), 3200 (H-bonded-OH), 1680 (C═O), 1610, 1400 (—SO₂N—)cm⁻¹; δ_{H} (acetone-d₆) 6.32 (1H, d, J=2.2 Hz, C-6-H or C-8-H), 6.46 (1H, d, J=2.2 Hz, C-8-H or C-6-H), 7.32 (2H, br s, exchanged with D₂O, —SO₂NH₂), 7.42 (2H, t, J=8.4 Hz, C-3′-H and C-5′-H or C-2′-H and C-6′-H, 7.69 (2H, d, J=8.4 Hz, C-2′-H and C-6′-H or C-3′-H and C-5′-H), 8.31 (1H, s, C-2-H), 9.53 (1H, s, C-7-OH) and 12.9 (1H, s, C-5-OH). MS: m/z (+ve ion FAB in m-NBA, rel. intensity) 350.0 [100, (M+H)⁺], 271.1 [15, (M+H—SO₂NH₂)⁺]. MS: m/z (−ve ion FAB in m-NBA, rel. intensity) 347.9 [100, (M−H)⁻], 269.0 [20, (M−H—SO₂NH₃)⁻]. Acc. MS: m/z (FAB)⁺ 350.0347 C₁₅H₁₂NO₇S requires 350.0335. Found C, 51.0; H, 3.16; N, 3.90; C₁₅H₁₁NO₇S requires C, 51.58; H, 3.17; N, 4.01%.

### Isoflavone-4′,7-O,O-Disulphamate (13)

4′,7-Dihydroxy isoflavone (0.45 g, 1.77 mmol) upon sulphamoylation gave a crude product (0.769 g) which was fractionated on silica (200 g) with chloroform/acetone (4:1), and upon evaporation the second fraction gave a pale white residue (0.553 g, 72%) which was recrystallized in acetone/hexane (1:2) to give the compound (13) as white crystals (0.327 g); m.p.>195EC (dec.); R_{f}a=0.21 and 0.40 for chloroform/acetone 4:1 and 2:1 respectively; v^{max} (KBr) 3400 (—NH₂), 1640 (C═O), 1360 (—SO₂N—) cm⁻¹. δ_{H} (DMSO-d₆), 7.37 (2H, d, J=8.8 Hz, C-3′-H and C-5′-H or C-2′-H and C-6′-H, 7.42 (1H, dd, J_{C-6-H, C-8-H}=2.2 Hz, J_{C-6-H, C-5-H}=8.8 Hz, C-6-H), 7.7 (2H, d, J=8.8 Hz, C-2′-H and C-6′-H or C-3′-H and C-5′-H), 8.09 (2H, br s, exchanged with D₂O, —OSO₂NH₂), 8.24 (1H, d, J=8.8 Hz, C-5-H, 8.36 (2H, br s, exchanged with D₂O, —OSO₂NH₂), 8.63 (1H, s, C-2-H). MS: m/z (+ve ion FAB in m-NBA, rel. intensity) 412.9 [100, (M+H)⁺], 334.0 [25, (M+H—SO₂NH₂)⁺], 255.1 [20, (M+H—SO₂NH₂)⁺]. MS: m/z (−ve ion FAB in m-NBA, rel. intensity) 410.9 [100, (M−H)⁻], 332.0 (70, (M−H—SO₂NH₂)⁻], 253.0 [30, (M−H—SO₂NH₂)⁻]. Acc. MS: m/z (FAB)⁺ 413.0119 C₁₅H₁₃N₂O₈S₂ requires 413.0113. Found C, 44.0; H, 2.94; N, 6.62; C₁₅H₁₂N₂O₈S₂ requires C, 43.69; H, 2.93; N, 6.79%.

Assay of Inhibition of Sulphatase and Aromatase Activities

Sulphatase inhibition was assessed using placental microsome (100,000 g) preparations or intact MCF-7 breast cancer cells as described previously^{29,30}. Placental microsomes were incubated with ³H E1S, adjusted to 20 μM with unlabelled substrate, in the absence or presence of inhibitor.

Placental microsomes were also used to assess the aromatase inhibitory properties of the flavanoid sulphamates using a tritiated water release assay³⁷. Further placental microsomes (200 μl) were incubated with [1β-³H]androstenedione, 60 nM and 1 mM NADPH in the absence or presence of inhibitor.

Inhibition of Sulphatase and Aromatase Activities

Inhibition of oestrone sulphatase and aromatase activities in placental microsomes by the flavanoid sulphamate derivatives is shown in the Table below.

| | | % | % |
|---|---|---|---|
| | CONCENTRATION | INHIBITION | INHIBITION |
| COMPOUND | μM | Sulphatase | Aromatase |
| Flavone-6- | 1 | 26.8 | 1 |
| sulphamate | 10 | 89.5 | 6.5 |
| Flavone-7- | 1 | — | 55 |
| sulphamate | 10 | — | 86 |
| | 50 | 56.3 | |
| | 100 | 75.3 | |
| 5-hydroxy | 1 | 8 | 5 |
| flavone-7- | 10 | 21 | 76 |
| sulphamate | | | |
| 5,7-dihydroxy | 0.1 | 30.4 | Not tested |
| flavanone 4′- | 1 | 79.1 | Not tested |
| sulphamate | 10 | 98.1 | Not tested |
| 5-hydroxy-4′- | 1 | 1 | 2 |
| methoxy- | 10 | 50.6 | 5 |
| isoflavone-7- | | | |
| sulphamate | | | |

From the results, it can be seen that potent inhibition of sulphatase and aromatase activities was detected. For sulphatase inhibition this ranged from 21% at 10 μM by 5-hydroxy flavone-7-sulphamate, to 98% by 5,7-dehydroxy flavanone-4′-sulphamate at 10 μM. Potent aromatase inhibition was also achieved ranging from 6.5% by flavone-6-sulphamate at 10 μM to 86% by flavone-7-sulphamate at 10 μM.

Further In Vitro Testing

The following Table presents in vitro data for three isoflavones that were tested.

IN VITRO ACTIVITY

| | | % Inhibition | |
|---|---|---|---|
| | Concentration | | Placental |
| Compound | (μM) | MCF-7 Cells | Microsomes |
| Isoflavone 5-hydroxy-4′,7- | 0.1 | 28 | nd |
| bissulphamate | 1.0 | 90 | nd |
| | 10.0 | 99 | 93 |
| Isoflavone 5,7-dihydroxy- | 0.1 | 23 | nd |
| 4′-sulphamate | 1.0 | 83 | nd |
| | 10.0 | 99 | 75 |
| Isoflavone-4′,7- | 0.1 | 89 | nd |
| bissulphamate | 1.0 | 99 | nd |
| | 10.0 | 99 | 99 |

nd = not done

In Vivo Testing

FIG. 11 presents in vivo inhibition of oestrone sulphatase activity in rat liver for two isoflavones according to the present invention. In this regard, BH22F1=5-hydroxy isoflavone-4′,7-bissulphamate; BH22BF1=5,7-dihydroxy isoflavone-4′-sulphamate.

Compounds were administered as a single 10 mg/Kg dose. Oestrone sulphatase activity was assayed in tissue samples obtained 24 h after drug administration.

### FURTHER EXAMPLES

The present invention will now be described only by way of example. However, it is to be understood that the examples also present preferred compounds of the present invention, as well as preferred routes for making same and useful intermediates in the preparation of same.

Synthetic Routes

Compounds in accordance with the present invention were synthesised in accordance with the synthetic routes and schemes.

Compounds of Formula III

### 4-([1,2,4]Triazol-4-ylamino)benzonitrile (LWO02023)

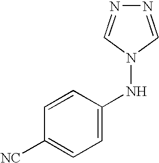

To a mixture of potassium tert-butoxide (6.7 g, 59.47 mmol) in anhydrous methyl sulfoxide (DMSO, 20 mL) was added at 10-15° C. portionwise a solution of 4-amino-4H-1,2,4-triazole (5.0 g, 59.47 mmol) in anhydrous DMSO (10 mL). After stirring the resulting thick light yellow suspension at room temperature under nitrogen for 60 min, this was cooled to ice/water temperature and a solution of 4-fluorobenzonitrile (3.60 g, 29.74 mmol) in anhydrous DMSO (10 mL) was added dropwise over a period of 5 min. The orange suspension that formed was stirred at room temperature under nitrogen for 1 h before it was poured into water (500 mL). The pH of the clear yellow mixture that formed was brought to neutral by using 5M HCL followed by saturated aqueous sodium bicarbonate solution if required. This mixture was allowed to stand at room temperature uncovered for 7 days at which yellow crystals were deposited. Upon filtration, washings exhaustively with water and air-drying overnight, 4-([1,2,4]triazol-4-ylamino)benzonitrile (2.08 g, 11.23 mmol, 37.8%) was collected; m.p. 200-204° C.; δ_{H} (400 MHz, DMSO-d₆) 6.55 (2H, AA′BB′), 7.69 (2H, AA′BB′), 8.85 (2H, s, C3′-H and C5′-H) and 10.23 (1H, br s, exchanged with D₂O, NH).

### 4-[(4-Benzyloxybenzyl)-[1,2,4]triazol-4-ylamino]benzonitrile (LWO02029)

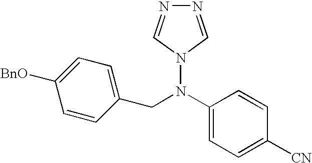

To a stirred solution of LWO02023 (700 mg, 3.780 mmol) in DMF (10 mL) at ice/water temperature was added sodium hydride (60% in mineral oil, 151 mg, 3.780 mmol). After stirring at room temperature under an atmosphere of nitrogen for 30 min, 4-benzyloxybenzyl chloride (968 mg, 4.158 mmol) was added in one portion and the resulting orange/brown mixture was heated at 80-90° C. for 3 h. The yellow suspension thus formed at room temperature was diluted with water (200 mL) and the white precipitate that formed was collected, washed exhaustively with water and air-dried to give 4-[(4-benzyloxybenzyl)-[1,2,4]triazol-4-ylamino]benzonitrile (1.35 g, 3.539 mmol, 94%) as white powder; m.p. 206-211° C.; R_{f} 0.37 (neat ethyl acetate), c.f. 0.83 (4-benzyloxybenzyl chloride); δ_{H} (400 MHz, DMSO-d₆) 4.98 (2H, s CH₂N), 5.06 (2H, s, CH₂O), 6.77 (2H, AA′BB′), 6.95 (2H, AA′BB′), 7.21 (2H, AA′BB′), 7.30-7.46 (5H, m, Bn), 7.76 (2H, AA′BB′) and 8.75 (2H, s, C3′-H and C5′-H); LRMS (FAB+): 763.3[7, (2M+H)⁺], 382.2[100, (M+H)⁺], 313.1[48, (M+H-triazole)⁺]; (FAB−): 687.3[28, (M+2NBA)⁻], 534.2[100, (M+NBA)⁻]; HRMS (FAB+) 382.16648 C₂₃H₂₀N₅O requires 382.16679.

### 4-[(4-Hydroxylbenzyl)-[1,2,4]triazol-4-ylamino]benzonitrile (LWO02030, STX265)

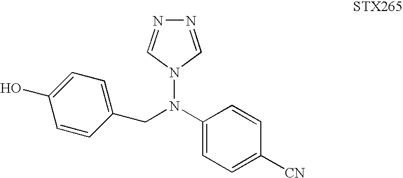

To a stirred solution of 4-[(4-benzyloxybenzyl)-[1,2,4]triazol-4-ylamino]benzonitrile (705 mg, 1.848 mmol) in distilled THF (120 mL) was added in succession absolute ethanol (30 mL) and Pd/C (10%, 40 mg). The black suspension was then stirred under an atmosphere of hydrogen (balloon) for 3 days. Upon removal by filtration and washings of the supported catalyst exhaustively with distilled THF, the filtrate was evaporated to give a slight wet light yellow residue (529 mg). The crude was recrystallised from DMF/ethyl acetate (1:10, 33 mL) to give 4-[(4-hydroxylbenzyl)-[1,2,4]triazol-4-ylamino]benzonitrile as yellow crystals (138 mg, 473.7 mmol, 25.6%); m.p. 228-230° C.; R_{f} 0.24 (neat ethyl acetate), c.f. 0.40 (S.M.); δ_{H} (400 MHz, DMSO-d₆) 4.91 (2H, s CH₂N), 6.67 (2H, AA′BB′), 6.77 (2H, AA′BB′), 7.06 (2H, AA′BB′), 7.76 (2H, AA′BB′), 8.71 (2H, s, C3′-H and C5′-H) and 9.49 (1H, s, exchanged with D₂O, OH); LRMS (FAB+): 583.3[9, (2M+H)⁺], 445.2[13, (M+H+NBA)⁺], 292.2[100, (M+H)⁺], 223.1[50, (M-triazole)⁺]; (FAB−): 444.2[36, (M+NBA)⁻], 184.1[100, (M-C₇H₇O)⁻]; HRMS (FAB+) 292.11871 C₁₆H₁₄N₅O requires 292.11984.

### Sulfamic acid 4-{[(4-cyanophenyl)-[1,2,4]triazol-4-ylamino]methyl}phenyl ester (LWO02031, STX258)

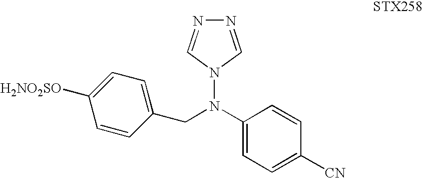

To a stirred solution of 4-[(4-hydroxylbenzyl)-[1,2,4]triazol-4-ylamino]benzonitrile (265 mg, 715.4 μmol) in anhydrous N,N-dimethylacetamide (DMA, 20 mL) was added at room temperature a solution of sulfamoyl chloride in toluene (ca. 0.68 M, 3.6 mL) and the resulting mixture stirred under an atmosphere of nitrogen overnight. Ethyl acetate (100 mL) was added to the reaction mixture and the organic layer that separated was washed with brine (100 mL, 4×50 mL), dried (MgSO4) and evaporated to give a light brown syrup/residue (ca. 400 mg). This crude was fractionated by flash chromatography (chloroform/methanol, 7:1 to 3.5:1, gradient) and the third fraction that isolated gave sulfamic acid 4-{[(4-cyanophenyl)-[1,2,4]triazol-4-ylamino]methyl}phenyl ester as a pale beige residue (150 mg, 405.0 μmol, 57%); m.p. 80-95° C.; R_{f} 0.57 (chloroform/methanol, 5:1), c.f. 0.67 (S.M.); δ_{H} (400 MHz, DMSO-d₆) 5.10 (2H, s CH₂N), 6.74 (2H, AA′BB′), 7.23 (2H, AA′BB′), 7.42 (2H, AA′BB′), 7.77 (2H, AA′BB′), 8.03 (2H, br s, exchanged with D₂O, H₂NSO₂) and 8.85 (2H, s, C3′-H and C5′-H); LRMS (FAB+): 371.1[100, (M+H)⁺], 302.1[30, (M-triazole)⁺]; (FAB−): 523.2[30, (M+NBA)⁻], 369.1[100, (M−H)⁻], 184.1[34, (M-Bn-OSO₂NH₂)⁻]; HRMS (FAB+) 371.09116 C₁₆H₁₅N₆O₃S requires 371.09264.

### 2-(4-Benzyloxyphenyl)ethanol (LWO02057)

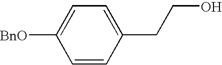

To a stirred solution of 4-hydroxyphenethyl alcohol (3.0 g, 22.16 mmol) in anhydrous DMF (50 mL) at ice/water temperature was added sodium hydride (60% in mineral oil, 886 mg, 22.16 mmol). After stirring at room temperature for 10 min, benzyl bromide (3.86 g, 22.16 mmol) was added and the reaction mixture was heated at 50° C. for 30 min. Upon cooling to room temperature, ethyl acetate (250 mL) was added and the organic layer that separated was washed with brine (500 mL, 4×100 mL), dried (MgSO₄) and evaporated to give a white residue (6.05 g). The crude was first dissolved in hot isopropanol (10 mL) and hexane (10 mL) was then added dropwise. Upon cooling, 2-(4-benzyloxyphenyl)ethanol was isolated as soft white crystals (2.45 g, 10.73 mmol). A second crop of the product (2.04 g, 8.936 mmol, total yield: 89%) was obtained from the residue of the mother liquor upon recrystallisation from hot hexane (ca. 150 mL); R_{f} 0.71 (neat ethyl acetate), 0.63 (S.M.); δ_{H} (400 MHz, DMSO-d₆) 2.64 (2H, t, J7.2 Hz, CH₂CH₂OH), 3.54 (2H, m, reduced to t after D₂O exchange, CH₂CH₂OH), 4.59 (1H, t, J5.2 Hz, exchanged with D₂O, OH), 5.06 (2H, S, CH₂O), 6.90 (2H, AA′BB′), 7.11 (2H, AA′BB′) and 7.28-7.46 (5H, m, Bn); LRMS (FAB+): 228.0[94, M⁺], 91.0[100, Bn⁺].

### 1-Bromo-2-(4-benzyloxyphenyl)ethane (LWO02060)

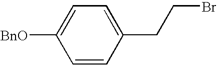

To a stirred solution of 2-(4-benzyloxyphenyl)ethanol (2.02 g, 8.848 mmol) in anhydrous distilled THF (30 mL) at ice/water temperature and under an atmosphere of nitrogen was added phosphorus tribromide (2.47 g, 8.848 mmol). After stirring at room temperature for 30 min, the reaction mixture was evaporated and the pale yellow liquid thus obtained was diluted with ethyl acetate (100 mL). The organic layer that separated was washed with brine (100 mL, 4×50 mL), dried (MgSO₄) and evaporated to give a light orange brown syrup (3.21 g) which turned light yellow upon standing overnight. This crude was fractionated by flash chromatography (chloroform/ethyl acetate, 1:2 to 1:1, gradient) and the first fraction that separated gave 1-bromo-2-(4-benzyloxyphenyl)ethane as a yellow solid (990 mg, 3.40 mmol, 38.4%); R_{f} 0.82 (neat ethyl acetate), 0.68 (S.M.); δ_{H} (400 MHz, DMSO-d₆) 3.04 (2H, t, J 7.2 Hz, CH₂CH₂Br), 3.67 (2H, t, J 7.4 Hz, CH₂CH₂Br), 5.08 (2H, S, CH₂O), 6.95 (2H, AA′BB′), 7.20 (2H, AA′BB′) and 7.30-7.48 (5H, m, Bn); LRMS (FAB+): 289.9(9), 73.0(100), (FAB−) 233.9(100).

### 4-{[2-(4-Benzyloxyphenyl)ethyl]-[1,2,4]triazol-4-yl-amino}benzonitrile (LWO02061)

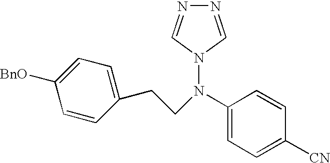

To a stirred solution of 4-([1,2,4]triazol-4-ylamino)benzonitrile (549 mg, 2.966 mmol) in DMF (10 mL) at ice/water temperature was added sodium hydride (60% in mineral oil, 137 mg, 3.426 mmol). After stirring at room temperature under an atmosphere of nitrogen for 30 min, 1-bromo-2-(4-benzyloxyphenyl)ethane (950 mg, 3.263 mmol) was added in one portion and the resulting dark brown mixture was heated at 70° C. for 3 h. The reddish brown mixture that formed was diluted with ice/water (200 mL) and the precipitate that formed was collected, washed exhaustively with water and air-dried to give a light orange yellow residue (1.21 g). This crude was fractionated by flash chromatography (dry loading, ethyl acetate as eluant) and the second fraction that separated gave 4-{[2-(4-benzyloxyphenyl)ethyl]-[1,2,4]triazol-4-yl-amino}benzonitrile as a yellow syrup which solidified upon standing overnight at room temperature to a light yellow wax (590 mg, 1.492 mmol, 50.3%); R_{f} 0.40 (neat ethyl acetate), c.f. 0.85 (1-bromo-2-(4-benzyloxyphenyl)ethane); δ_{H} (400 MHz, DMSO-d₆) 2.74 (2H, t, J˜7.6 Hz, CH₂CH₂N), 4.04 (2H, t, J˜7.4 Hz, CH₂CH₂N), 5.08 (2H, s, CH₂O), 6.59 (2H, AA′BB′), 6.94 (2H, AA′BB′), 7.20 (2H, AA′BB′), 7.28-7.46 (5H, m, Bn), 7.70 (2H, AA′BB′) and 8.83 (2H, s, C3′-H and C5′-H); LRMS (FAB+): 396.1[100, (M+H)⁺], 369.3[5, (M-CN)⁺], 91.0[82, Bn⁺]; (FAB−): 701.4[25, (M+2NBA)⁻], 548.3[100, (M+NBA)⁻], 441.2[35, (M+NBA-OBn)⁻]; HRMS (FAB+) 396.18192 C₂₄H₂₂N₅O requires 396.18244.

### 4-{[2-(4-Hydroxyphenyl)ethyl]-[1,2,4]triazol-4-yl-amino}benzonitrile (LWO02063, STX290)

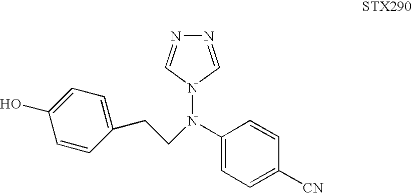

To a stirred solution of 4-{[2-(4-benzyloxyphenyl)ethyl]-[1,2,4]triazol-4-yl-amino}benzonitrile (536 mg, 1.355 mmol) in distilled THF (15 mL) was added in succession absolute ethanol (30 mL) and Pd/C (10%, 54 mg). The black suspension was then stirred under an atmosphere of hydrogen (balloon) for 3 days. Upon removal by filtration and washings of the supported catalyst exhaustively with distilled THF, the filtrate was evaporated to give a pale yellow syrup which solidified upon standing at room temperature to a light yellow wax (192 mg). This crude was fractionated by flash chromatography (ethyl acetate) and the second fraction that separated gave 4-{[2-(4-hydroxyphenyl)ethyl]-[1,2,4]triazol-4-yl-amino}benzonitrile as a pale yellow waxy residue (115 mg, 376.6 μmol, 28%); R_{f} 0.46 (neat ethyl acetate), c.f. 0.59 (S.M.); δ_{H} (400 MHz, DMSO-d₆) 2.70 (2H, t, J=7.4 Hz, CH₂CH₂N), 4.01 (2H, t, J˜7.6 Hz, CH₂N), 6.59 (2H, AA′BB′), 6.69 (2H, AA′BB′), 7.06 (2H, AA′BB′), 7.71 (2H, AA′BB′), 8.78 (2H, s, C3′-H and C5′-H) and 9.29 (1H, s, exchanged with D₂O, OH); LRMS (FAB+): 611.2[12, (2M+H)⁺], 459.1[8, (M+H+NBA)⁺], 306.0[100, (M+H)⁺]; (FAB−): 763.5[18, (2M+NBA)⁻], 609.4[45, (2M−H)⁻], 184.0[100, (HOPhCH₂CH₂)⁻]; HRMS (FAB+) 306.13477 C₁₇H₁₆N₅O requires 306.13549.

### Sulfamic acid 4-{2-[(4-cyanophenyl)-[1,2,4]triazol-4-ylamino]ethyl}phenyl ester (LWO02066, STX273)

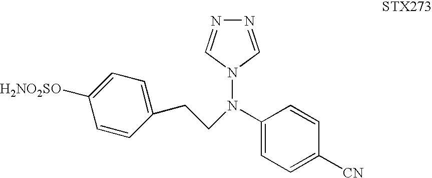

To a stirred solution of 4-{[2-(4-hydroxyphenyl)ethyl]-[1,2,4]triazol-4-yl-amino}benzonitrile (81 mg, 265.2 μmol) in anhydrous N,N-dimethylacetamide (DMA, 5 mL) was added at room temperature a solution of sulfamoyl chloride in toluene (ca. 0.68 M, 1.2 mL) and the resulting mixture was stirred under an atmosphere of nitrogen overnight. Ethyl acetate (50 mL) was then added to the reaction mixture and the organic layer that separated was washed with brine (100 mL, 4×50 mL), dried (MgSO4), filtered and evaporated to give a light brown syrup/residue (ca. 100 mg). This crude was dissolved in acetone (20 mL) and then concentrated to approximately 3 mL. Hexane (1.5 mL) was then added dropwise and upon standing gave sulfamic acid 4-{2-[(4-cyanophenyl)-[1,2,4]triazol-4-ylamino]ethyl}phenyl ester as white crystals (52 mg, 135.3 μmol, 51%); R_{f} 0.33 (ethyl acetate), c.f. 0.41 (S.M.); δ_{H} (400 MHz, DMSO-d₆) 2.84 (2H, t, J 7 Hz, CH₂CH₂N), 4.09 (2H, t, J˜7 Hz, CH₂N), 6.63 (2H, AA′BB′), 7.20 (2H, AA′BB′), 7.38 (2H, AA′BB′), 7.72 (2H, AA′BB′), 7.97 (2H, br s, exchanged with D₂O, OSO₂NH₂) and 8.87 (2H, s, C3′-H and C5′-H); LRMS (FAB+): 385.0[100, (M+H)⁺]; (FAB−): 537.2[40, (M+NBA)⁻], 383.1[100, (M−H)⁻]; HRMS (FAB+) 385.10752 C₁₇H₁₇N₆O₃S requires 385.10829.

### 1-Benzyloxy-4-(2-bromoethoxy)benzene (LWO02068)

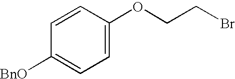

This compound was prepared from 4-(benzyloxy)phenol and 1,2-dibromoethane in the same manner as described by Zhou et. Al. (1999) J. Med. Chem. 42: 2993-3000.

### 4-{[2-(4-Benzyloxyphenoxy)ethyl]-[1,2,4]triazol-4-yl-amino}benzonitrile (LWO02075)

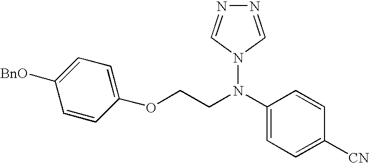

To a stirred solution of 4-([1,2,4]triazol-4-ylamino)benzonitrile (1.0 g, 5.40 mmol) in DMF (15 mL) at ice/water temperature was added sodium hydride (60% in mineral oil, 238 mg, 5.94 mmol). After stirring at room temperature under an atmosphere of nitrogen for 10 min, 1-benzyloxy-4-(2-bromoethoxy)benzene (1.82 g, 5.94 mmol) was added in one portion and the resulting dark brown mixture was heated at 50° C. for 18 h. The reaction mixture was then passed through a short column of silica and the filter cake was washed with ethyl acetate (10×20 mL). The combined filtrate was washed with brine (200 mL, 4×50 mL), dried (MgSO₄), filtered and evaporated to give a light yellow brown residue (2.28 g). This crude was recrystallised from hot ethyl acetate to give 4-{[2-(4-benzyloxyphenoxy)ethyl]-[1,2,4]triazol-4-yl-amino}benzonitrile (690 mg, 1.677 mmol) as fluffy creamy crystals. A second crop of the product (915 mg, 2.224 mmol, total yield, 72%) was obtained from the residue of the mother liquor upon recrystallisation from hot ethyl acetate and hexane; m.p. 160-162° C.; R_{f} 0.45 (neat ethyl acetate), c.f. 0.89 (1-benzyloxy-4-(2-bromoethoxy)benzene); δ_{H} (400 MHz, DMSO-d₆) 4.08 (2H, m, CH₂), 4.23 (2H, m, CH₂), 5.03 (2H, s, CH₂O), 6.67 (2H, AA′BB′), 6.82 (2H, AA′BB′), 6.93 (2H, AA′BB′), 7.28-7.46 (5H, m, Bn), 7.74 (2H, AA′BB′) and 8.91 (2H, s, C3′-H and C5′-H). Found C, 69.7; H, 5.16; N, 16.7; C₂₄H₂₁N₅O₂ requires C, 70.06; H, 5.14; N, 17.02%.

### 4-{[2-(4-Hydroxyphenoxy)ethyl]-[1,2,4]triazol-4-yl-amino}benzonitrile (LWO02076, STX291)

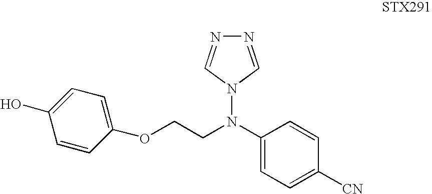

To a stirred solution of 4-{[2-(4-benzyloxyphenoxy)ethyl]-[1,2,4]triazol-4-yl-amino}benzonitrile (670 mg, 1.628 mmol) in distilled THF (70 mL) was added in succession absolute ethanol (30 mL) and Pd/C (10%, 67 mg). The black suspension was then stirred under an atmosphere of hydrogen (balloon) for 3 days. Upon removal by filtration and washings of the supported catalyst exhaustively with distilled THF, the filtrate was evaporated to give a light yellow residue (491 mg). This crude was dissolved in hot acetone (25 mL) and hexane (15 mL) was then added dropwise. Upon cooling, 4-{[2-(4-hydroxyphenoxy)ethyl]-[1,2,4]triazol-4-yl-amino}benzonitrile was separated as light green yellow crystals (310 mg, 964.1 μmol, 59%); m.p. 184-195° C.; R_{f} 0.31 (neat ethyl acetate), c.f. 0.45 (S.M.); δ_{H} (400 MHz, DMSO-d₆) 4.03 (2H, t, J 4.6-5.1 Hz, CH₂), 4.21 (2H, t, J 5.1 Hz, CH₂), 6.62-6.73 (6H, m, Ar), 7.73 (2H, AA′BB′), 8.89 (2H, s, C3′-H and C5′-H) and 8.95 (1H, s, exchanged with D₂O, OH); LRMS (FAB+): 643.2[12, (2M−H)⁺], 4675.1[100, (M+H+NBA)⁺], 322.1[100, (M+H)⁺], 253.1[20, (M-triazole)⁺]; (FAB−): 795.1[10, (2M+NBA)⁻], 641.2[30, (2M−H)⁻], 474.2[90, (M+NBA)⁻], 320.1 [100, (M−H)⁻]; HRMS (FAB+) 322.12984 C₁₇H₁₆N₅O₂ requires 322.13040.

### Sulfamic acid 4-{2-[(4-cyanophenyl)-[1,2,4]triazol-4-yl-amino]ethoxy}phenyl ester (LWO02077, STX292)

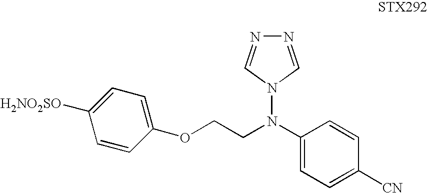

To a stirred solution of 4-{[2-(4-hydroxyphenoxy)ethyl]-[1,2,4]triazol-4-yl-amino}benzonitrile (212 mg, 659.7 μmol) in anhydrous N,N-dimethylacetamide (DMA, mL) was added at room temperature a solution of sulfamoyl chloride in toluene (ca. 0.68 M, 2 mL) and the resulting mixture was stirred under an atmosphere of nitrogen for 1.5 h. Ethyl acetate (100 mL) was then added to the reaction mixture and the organic layer that separated was washed with brine (100 mL, 4×50 mL), dried (MgSO4), filtered and evaporated to give a white fluffy residue (299 mg). This crude was fractionated by flash chromatography (ethyl acetate, then ethyl acetate/acetone 2:1 after the second fraction was eluted) and upon evaporation of the third fraction that isolated gave sulfamic acid 4-{2-[(4-cyanophenyl)-[1,2,4]triazol-4-yl-amino]ethoxy}phenyl ester as white fluffy residue (218 mg, 544.4 μmol, 83%); R_{f} 0.21 (ethyl acetate), c.f. 0.30 (S.M.); δ_{H} (400 MHz, DMSO-d₆) 4.14 (2H, m, CH₂), 4.28 (2H, m, CH₂), 6.68 (2H, AA′BB′), 6.94 (2H, AA′BB′), 7.18 (2H, AA′BB′), 7.75 (2H, AA′BB′), 7.89 (2H, br s, exchanged with D₂O, OSO₂NH₂) and 8.93 (2H, s, C3′-H and C5′-H); LRMS (FAB+): 401.0[100, (M+H)⁺]; (FAB−): 799.1[8, (2M−H)⁻], 553.2[35, (M+NBA)⁻], 399.1[100, (M−H)⁻]; HRMS (FAB+) 401.10471 C₁₇H₁₇N₆O₄S requires 401.10320.

### 1-Benzyloxy-4-(4-bromobutoxy)benzene (LWO02064)

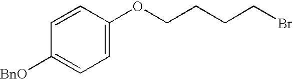

To a solution of 4-(benzyloxy)phenol (3.0 g, 15.13 mmol) and 1,4-dibromobutane (16.34 g, 75.65 mmol) in acetonitrile (25 mL) was added anhydrous potassium carbonate (5.23 g, 37.83 mmol). The suspension was then refluxed for 18 h. After cooling to room temperature, the suspension was filtered through a short column of silica and the filter cake washed exhaustively with ethyl acetate. The filtrate was evaporated to give a yellow liquid (17.63 g) which upon standing gave a mass of white crystals. These crystals were then triturated with hexane and collected by filtration. Upon drying in the air, 1-benzyloxy-4-(4-bromobutoxy)benzene was obtained as creamy crystals (2.87 g, 8.561 mmol, 57%); m.p. 73-75° C.; R_{f} 0.84 (chloroform), c.f. 0.16 (S.M.); δ_{H} (400 MHz, DMSO-d₆) 1.80 (2H, m, CH₂), 1.95 (2H, m, CH₂), 3.60 (2H, t, J 6.6 Hz, CH₂Br), 3.92 (2H, t, J 6.2 Hz, OCH₂CH₂), 5.03 (2H, s, CH₂O), 6.86 (2H, AA′BB′), 6.92 (2H, AA′BB′) and 7.28-7.46 (5H, m, Bn). Found C, 60.8; H, 5.71; C₁₇H₁₉BrO₂ requires C, 60.91; H, 5.71%.

### 4-{[4-(4-Benzyloxyphenoxy)butyl]-[1,2,4]triazol-4-ylamino}benzonitrile (LWO02065)

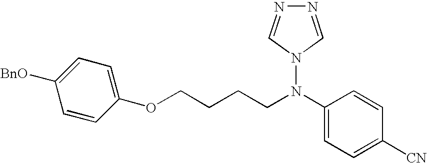

To a stirred solution of 4-([1,2,4]triazol-4-ylamino)benzonitrile (1.0 g, 5.40 mmol) in DMF (15 mL) at ice/water temperature was added sodium hydride (60% in mineral oil, 238 mg, 5.94 mmol). After stirring at room temperature under an atmosphere of nitrogen for 10 min, 1-benzyloxy-4-(4-bromobutoxy)benzene (1.99 g, 5.94 mmol) was added in one portion and the resulting dark brown mixture was heated at 50° C. for 18 h. The cooled reaction mixture was then passed through a short column of silica and the filter cake was washed with ethyl acetate (10×20 mL). The combined filtrate was washed with brine (200 mL, 4×50 mL), dried (MgSO₄), filtered and evaporated to give a yellow orange residue (2.50 g). This crude was dissolved in hot ethyl acetate (10 mL) and hexane (2 mL) was then added dropwise. Upon cooling to room temperature, 4-{[4-(4-benzyloxyphenoxy)butyl]-[1,2,4]triazol-4-ylamino}benzonitrile was obtained as creamy/pale yellow crystals (1.42 g, 3.231 mmol). A second crop of the product (363 mg, 825.9 μmol, total yield, 75%) was obtained from the residue of the mother liquor upon recrystallisation from hot ethyl acetate; m.p. 124.5-126.5° C.; R_{f} 0.45 (neat ethyl acetate), c.f. 0.88 (1-benzyloxy-4-(4-bromobutoxy)benzene); δ_{H} (400 MHz, DMSO-d₆) 1.58 (2H, quasi quintet, CH₂), 1.78 (2H, quasi quintet, CH₂), 3.90 (4H, quasi q, OCH₂CH₂CH₂CH₂N), 5.03 (2H, s, CH₂O), 6.65 (2H, AA′BB′), 6.84 (2H, AA′BB′), 6.92 (2H, AA′BB′), 7.28-7.45 (5H, m, Bn), 7.72 (2H, AA′BB′) and 8.98 (2H, s, C3′-H and C5′-H). Found C, 71.05; H, 5.74; N, 15.8; C₂₆H₂₅N₅O₂ requires C, 71.05; H, 5.73; N, 15.93%.

### 4-{[4-(4-Hydroxyphenoxy)butyl]-[1,2,4]triazol-4-ylamino}benzonitrile (LWO02067, STX287)

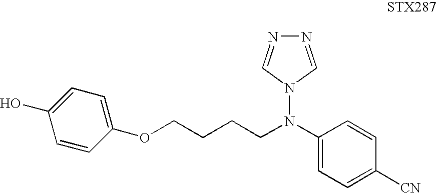

To a stirred solution of 4-{[4-(4-benzyloxyphenoxy)butyl]-[1,2,4]triazol-4-ylamino}benzonitrile (800 mg, 1.820 mmol) in distilled THF (10 mL) was added in succession absolute ethanol (30 mL) and Pd/C (10%, 80 mg). The black suspension was then stirred under an atmosphere of hydrogen (balloon) for 2 days. Upon removal by filtration and washings of the supported catalyst exhaustively with distilled THF, the filtrate was evaporated to give a light yellow frothy residue/syrup (481 mg). This crude was fractionated by flash chromatography (ethyl acetate to acetone, gradient) and the second fraction that separated upon evaporation gave a soft pale yellow residue (323 mg) which was further purified by recrystallisation from acetone/hexane to give 4-{[4-(4-hydroxyphenoxy)butyl]-[1,2,4]triazol-4-ylamino}benzonitrile as fine pale yellow crystals (280 mg, 801.4 μmol, 44%); m.p. 156-159° C.; R_{f} 0.42 (neat ethyl acetate), c.f. 0.51 (S.M.); δ_{H} (400 MHz, DMSO-d₆) 1.58 (2H, m), 1.76 (2H, m), 3.87 (4H, m, OCH₂CH₂CH₂CH₂N), 6.61-6.75 (6H, m, Ar), 7.73 (2H, AA′BB′), 8.91 (1H, s, exchanged with D₂O, OH) and 8.99 (2H, s, C3′-H and C5′-H). Found C, 65.4; H, 5.54; N, 19.6; C₁₉H₁₉N₅O₂ requires C, 65.32; H, 5.48; N, 20.04%.

### Sulfamic acid 4-{4-[(4-cyanophenyl)-[1,2,4]triazol-4-ylamino]butoxy}phenyl ester (LWO02069, STX288)

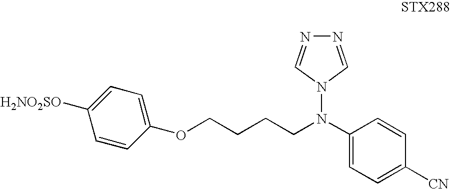

To a stirred solution of 4-{[4-(4-hydroxyphenoxy)butyl]-[1,2,4]triazol-4-ylamino}benzonitrile (210 mg, 601 μmol) in anhydrous N,N-dimethylacetamide (DMA, 5 mL) was added at room temperature a solution of sulfamoyl chloride in toluene (ca. 0.68 M, 1.8 mL) and the resulting mixture was stirred under an atmosphere of nitrogen for 1.5 h. Ethyl acetate (100 mL) was then added to the reaction mixture and the organic layer that separated was washed with brine (50 mL, 4×20 mL), dried (MgSO4), filtered and evaporated to give a creamy residue (291 mg) which was purified by recrystallisation from acetone/hexane to give sulfamic acid 4-{4-[(4-cyanophenyl)-[1,2,4]triazol-4-ylamino]butoxy}phenyl ester as pale yellow crystals (218 mg, 508.8 mmol, 85%); m.p. 164-172° C.; R_{f} 0.43 (ethyl acetate), c.f. 0.48 (S.M.); δ_{H} (400 MHz, DMSO-d₆) 1.60 (2H, m, CH₂), 1.82 (2H, m, CH₂), 3.90 (2H, t, J 7.4 Hz, CH₂), 3.98 (2H, t, J 6.2 Hz, CH₂), 6.66 (2H, AA′BB′), 6.97 (2H, AA′BB′), 7.18 (2H, AA′BB′), 7.73 (2H, AA′BB′), 8.91 (2H, br s, exchanged with D₂O, OSO₂NH₂) and 8.99 (2H, s, C3′-H and C5′-H); LRMS (FAB+): 429.0[100, (M+H)⁺]; (FAB−): 427.1[100, (M−H)⁻]; HRMS (FAB+) 429.13567 C₁₉H₂₁N₆O₄S requires 429.13450.

### 3-Bromo-4-hydroxybenzaldehyde (LWO02081)

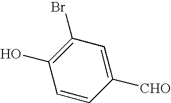

To a stirred solution of 4-hydroxybenzaldehyde *8.0 g, 64.20 mmol) in chloroform (400 mL) at 40° C. was added portionwise a solution of bromine (3.3 mL) in chloroform (10 mL). The resulting reddish brown mixture was stirred at 40° C. for 2 h, cooled and evaporated to give a purple residue which was dissolved in ethyl acetate (200 mL). The organic layer that separated was washed with brine (4×100 mL), dried (MgSO₄), filtered and evaporated to give a light pink/brown residue (12.95 g). The crude was purified by recrystallisation from hot toluene twice to give 3-bromo-4-hydroxybenzaldehyde as light orange/brown crystals (8.88 g, 44.17 mmol, 69%); m.p. 115-128° C.; R_{f} 0.42 (chloroform/ethyl acetate, 4:1), c.f. 0.36 (S.M.); δ_{H} (400 MHz, DMSO-d₆) 7.11 (1H, d, J 8.2 Hz, C5-H), 7.76 (1H, dd, J 1.95 and 8.2 Hz, C6-H), 8.04 (1H, d, J 2 Hz, C2-H), 9.78 (1H, s, CHO) and 11.53 (1H, s, exchanged with D₂O, OH). This product was used for the next reaction without further purification.

### 4-Benzyloxy-3-bromobenzaldehyde (LWO02082)

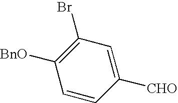

To a stirred solution of 3-bromo-4-hydroxybenzaldehyde (8.0 g, 39.80 mmol) in anhydrous DMF (50 mL) at ice/water temperature was added sodium hydride (60% in mineral oil, 1.67 g, 41.79 mmol). After stirring at room temperature for 10 min, benzyl bromide (7.64 g, 43.78 mmol) was added and the reaction mixture was heated at 80° C. for 2 h. Upon cooling to room temperature, ethyl acetate (300 mL) was added and the organic layer that separated was washed with brine (500 mL, 4×50 mL), dried (MgSO₄), filtered and evaporated to give a light beige residue (13.09 g). The crude was purified by recrystallisation from isopropanol/hexane to give 4-benzyloxy-3-bromobenzaldehyde as fine light yellow crystals (9.52 g, 32.70 mmol, 82%); m.p. 95-96.5° C. [Lit.¹ (ethanol), m.p. 95° C.]; R_{f} 0.75 (ethyl acetate/hexane, 1:1), 0.52 (S.M.); δ_{H} (400 MHz, DMSO-d₆) 5.35 (2H, s, CH₂O), 7.33-7.53 (6H, m, Bn and C5-H), 7.93 (1H, dd, J 2 and 8.4 Hz, C6-H), 8.13 (1H, d, J 1.9 Hz, C2-H) and 9.87 (1H, s, CHO).
¹ Buu-Hoi et. Al. (1953) J. Org. Chem. 18: 121-125.

### 4-Benzyloxy-3-bromophenol (LWO02085)

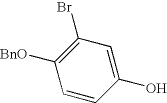

To a stirred solution of 4-benzyloxy-3-bromobenzaldehyde (4.5 g, 15.46 mmol) in chloroform (30 mL) at room temperature was added m-chloroperoxybenzoic acid (57-86%, 5.62 g) and the resulting suspension was stirred for 4 h. Ethyl acetate (200 mL) was then added and the organic layer that separated was washed with saturated aqueous sodium bicarbonate (1×100 mL, 3×50 mL) followed by brine (2×50 mL), dried (MgSO₄), filtered and evaporated to give a clear brown oil which upon standing at room temperature overnight gave some light yellow deposits (6.05 g). To this crude in methanol (45 mL) at room temperature was added 1 M NaOH (aq) (30 mL). After stirring for 2 h, the resulting brown mixture was acidified with 5 M HCl followed by dilution with ethyl acetate (200 mL). The organic layer that separated was washed with brine (100 mL, 4×50 mL), dried (MgSO₄), filtered and evaporated to give a brown oil (5.33 g). Upon fractionation by flash chromatography (ethyl acetate/hexane, 1:2 to 1:1 gradient), the fourth fraction that isolated gave 4-benzyloxy-3-bromophenol as a light golden yellow oil which solidified upon standing at room temperature to form a light brown wax (3.92 g, 14.04 mmol, 91%); R_{f} 0.48 (ethyl acetate/hexane, 1:2), 0.61 (4-benzyloxy-3-bromobenzaldehyde); δ_{H} (400 MHz, DMSO-d₆) 5.07 (2H, s, CH₂O), 6.72 (1H, dd, J 2.7 and 8.9 Hz, C6-H), 6.98 (1H, d, J 3.1 Hz, C2-H), 7.02 (1H, d, J 8.9 Hz, C5-H), 7.28-7.50 (5H, m, Bn) and 9.39 (1H, br s, exchanged with D₂O, OH); LRMS (FAB+): 278.0[35, (⁷⁹M)⁺], 91.0[100, Bn⁺]; (FAB−): 433.1[83, (⁸¹M+NBA)⁻], 276.9[84, (⁷⁹M−H)⁻], 187.9 [100, (⁸¹M−H-Bn)⁻]; HRMS (FAB+) 277.99390 C₁₃H₁₁ ⁷⁹BrO₂ requires 277.99424, (FAB+) 279.99213 C₁₃H₁₁ ⁸¹BrO₂ requires 279.99219. The product was not further purified before use.

### 2-(4-Benzyloxy-3-bromophenoxy)ethanol (LWO02086)

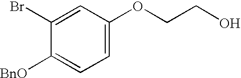

To a solution of 4-benzyloxy-3-bromophenol (2.0 g, 7.165 mmol) in anhydrous DMF (10 mL) at room temperature was added anhydrous potassium carbonate (1.04 g) followed by 2-bromoethanol (990 mg, 7.523 mmol). The resulting suspension was stirred under an atmosphere of nitrogen at 80° C. overnight. After cooling, ethyl acetate (100 mL) was added and the organic layer that separated was washed with brine (150 mL, 4×50 mL), dried (MgSO₄), filtered and evaporated to give a brown syrup (2.51 g). This crude was fractionated by flash chromatography (chloroform/ethyl acetate, 6:1 to 2:1 gradient) and the third fraction that separated upon evaporation gave 2-(4-benzyloxy-3-bromophenoxy)ethanol (1.58 g, 4.889 mmol, 68%); R_{f} 0.49 (chloroform/ethyl acetate, 4:1), c.f. 0.68 (S.M.); δ_{H} (400 MHz, DMSO-d₆) 3.67 (2H, ˜q, J˜5 Hz, CH₂OH), 3.94 (2H, t, J 5.1 Hz, CH₂CH₂OH), 4.86 (1H, t, J 5.5 Hz, exchanged with D₂O, OH), 5.13 (2H, s, CH₂O), 6.92 (1H, dd, J 3.1 and 8.9 Hz, C6-H), 7.12 (1H, d, J 9 Hz, C5-H), 7.19 (1H, d, J 3.1 Hz, C2-H) and 7.30-7.50 (5H, m, Bn); LRMS (FAB+): 322.0[45, (⁷⁹M)⁺], 91.0[100, Bn⁺]; (FAB−): 475.2[24, (⁷⁹M+NBA)⁻], 323.1 [100, (⁸¹M−H)⁻], 231.9(100); HRMS (FAB+) 324.01846 C₁₅H₁₅⁸¹BrO₃ requires 324.01841.

### 2-Bromo-1-(4-benzyloxy-3-bromophenoxy)ethane (LWO02087)

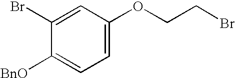

To a solution of 2-(4-benzyloxy-3-bromophenoxy)ethanol (1.44 g, 4.456 mmol) in anhydrous dichloromethane (15 mL) at ice/water temperature was added carbon tetrabromide (1.88 g, 5.570 mmol) followed by triphenylphosphine (1.77 g, 6.684 mmol) portionwise over a period of 5 min. After stirring under an atmosphere of nitrogen at ice/water temperature for 15 min, the reaction mixture was evaporated to give a pale orange syrup (5.22 g). This crude was fractionated by flash chromatography (chloroform/ethyl acetate, 4:1) and the first fraction that separated upon evaporation gave 2-bromo-1-(4-benzyloxy-3-bromophenoxy)ethane as a pale yellow liquid (1.81 g, 4.688 mmol); R_{f} 0.79 (chloroform/ethyl acetate, 4:1), c.f. 0.32 (S.M.); δ_{H} (400 MHz, DMSO-d₆) 3.76 (2H, t, J 5.5 Hz, CH₂Br), 4.28 (2H, ˜t, J 4-5 Hz, OCH₂CH₂), 5.14 (2H, s, CH₂O), 6.96 (1H, dd, J 3.1 and 8.9 Hz, C6-H), 7.13 (1H, d, J 8.9 Hz, C5-H), 7.24 (1H, d, J 2.7 Hz, C2-H) and 7.30-7.50 (5H, m, Bn); LRMS (FAB+): 385.9[36, M⁺], 91.0[100, Bn⁺]; (FAB−): 385.0[29, (M−H)⁻], 231.9(100); HRMS (FAB+) 385.93362 C₁₅H₁₄O₂⁷⁹Br⁸¹Br requires 385.93401.

### 4-{[2-(4-Benzyloxy-3-bromophenoxy)ethyl]-[1,2,4]triazol-4-ylamino}benzonitrile (LWO02088)

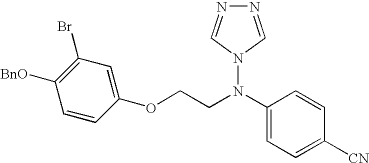

To a stirred solution of 4-([1,2,4]triazol-4-ylamino)benzonitrile (741 mg, 4.001 mmol) in anhydrous DMF (10 mL) at ice/water temperature was added sodium hydride (60% in mineral oil, 176 mg, 4.401 mmol). After stirring at room temperature under an atmosphere of nitrogen for 10 min, a solution of 2-bromo-1-(4-benzyloxy-3-bromophenoxy)ethane (1.70 g, 4.401 mmol) in DMF (5 mL) was added. The resulting mixture was heated at 60° C. for 2 h, then cooled and diluted with ethyl acetate (500 mL). The organic layer that separated was washed with brine (200 mL, 4×100 mL), dried (MgSO₄), filtered and evaporated to give a light brown residue (2.04 g). This crude was purified by recrystallisation from hot ethyl acetate and upon cooling gave 4-{[2-(4-benzyloxy-3-bromophenoxy)ethyl]-[1,2,4]triazol-4-ylamino}benzonitrile as fluffy pale beige powder (1.20 g, 2.447 mmol, 61%); m.p. 163-166° C.; R_{f} 0.30 (ethyl acetate), c.f. 0.75 [2-bromo-1-(4-benzyloxy-3-bromophenoxy)ethane]; δ_{H} (400 MHz, DMSO-d₆) 4.11 (2H, t, J 4-5 Hz, CH₂N), 4.23 (2H, t, J 4-5 Hz, CH₂O), 5.13 (2H, s, CH₂O), 6.66 (2H, AA′BB′), 6.86 (1H, dd, J 2.7 and 8.9 Hz, C6′-H), 7.12 (1H, d, J=8.9 Hz, C5′-H), 7.16 (1H, d, J=2.7 Hz, C2′-H), 7.30-7.48 (5H, m, Bn), 7.74 (2H, AA′BB′) and 8.94 (2H, s, C3″—H and C5″-H). Found C, 58.6; H, 4.17; N, 14.38; C₂₄H₂₀BrN₅O₂ requires C, 58.79; H, 4.11; N, 14.28%.

### 4-{[2-(3-Bromo-4-hydroxyphenoxy)ethyl]-[1,2,4]triazol-4-ylamino}benzonitrile (STX300) and 4-{[2-(4-Hydroxy-phenoxy)ethyl]-[1,2,4]triazol-4-ylamino}benzonitrile (STX291) (LWO02089)

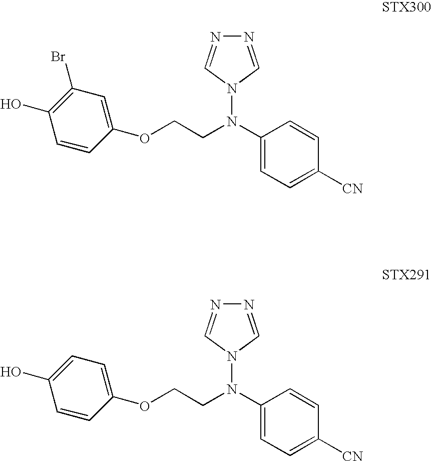

To a stirred solution of 4-{[2-(4-benzyloxy-3-bromophenoxy)ethyl]-[1,2,4]triazol-4-ylamino}benzonitrile (902 mg, 1.839 mmol) in distilled THF (45 mL) was added in succession absolute ethanol (45 mL) and Pd/C (10%, 90 mg). The black suspension was then stirred under an atmosphere of hydrogen (balloon) for 2 days. Upon removal by filtration and washings of the supported catalyst exhaustively with distilled THF, the filtrate was evaporated to give a beige residue (860 mg). This crude in DMF (10 mL) was fractionated by flash chromatography (ethyl acetate/acetone, 4:1 to acetone, gradient) and the third fraction that separated upon evaporation gave a wet creamy residue which was triturated with ether (50 mL). The precipitate that formed was filtered, washed exhaustively with water and air-dried overnight to give LWO02089 as off-white powder (453 mg); R_{f} 0.40 (ethyl acetate), c.f. 0.50 (S.M.); δ_{H} (400 MHz, DMSO-d₆) 4.03 (˜0.4H, t, J 4.6-5.1 Hz, CH₂N of STX291), 4.06 (1.6H, t, J 4.6-5.1 Hz, CH₂N of STX300), 4.21 (2H, t, J 4.6-5.1 Hz, OCH₂CH₂N of STX291 and STX300), 6.65 (˜2H, AA′BB′ of STX291 and STX300), 6.71 (˜0.4H, AA′BB′ of STX291), 6.74 (˜0.8H, dd, J 2.8-3.1 and 8.8 Hz, C6′-H of STX300), 6.85 (˜0.8H, d, J 8.9 Hz, C5′-H of STX300), 7.04 (0.8H, d, J 3.1 Hz, C2′-H of STX300), 7.74 (2H, AA′BB′ of STX291 and STX300), 8.91 (˜0.4H, s, C3″-H and C5″-H of STX291), 8.93 (˜1.6H, s, C3″-H and C5″-H of STX300), 8.98 (˜0.2H, br s, exchanged with D₂O, OH of STX291) and 9.74 (˜0.8H, br s, exchanged with D₂O, OH of STX300) This implies that the product contains approx. 20% of STX291; LRMS (FAB+): 553.0[18, (⁷⁹M+H+NBA)⁺], 475.1[12, (STX291+H+NBA)⁺], 400.0[100, (⁷⁹M+H)⁺], 322.1[85, (STX291+H)⁺], 252.1[50, (⁷⁹M-⁷⁹Br-triazole)⁺]; (FAB−): 552.0[60, (⁷⁹M+NBA)⁻], 475.3[32, (STX291+H+NBA)⁻], 398.1[100, (⁷⁹M−H)⁻]; HRMS (FAB+) 400.04045 C₁₇H₁₅⁷⁹BrN₅O₂ requires 400.04091, (FAB+) 322.12936 C₁₇H₁₆N₅O₂ requires 322.13040.

### Sulfamic acid 2-bromo-4-{2-[(4-cyanophenyl)-[1,2,4]triazol-4-ylamino]ethoxy}phenyl ester (STX301) and Sulfamic acid 4-{2-[(4-cyanophenyl)-[1,2,4]triazol-4-ylamino]ethoxy}phenyl ester (STX292) (LWO02090)

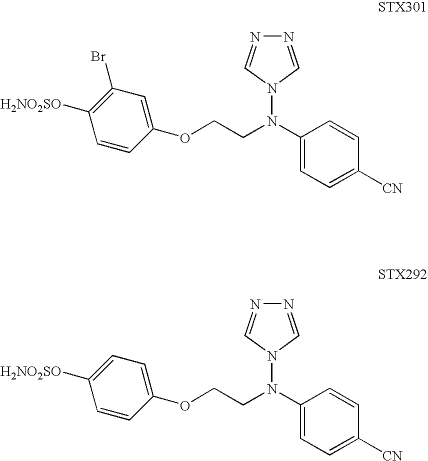

To a stirred solution of LWO02089 (289 mg, 699.5 μmol) in anhydrous N,N-dimethylacetamide (DMA, 10 mL) was added at room temperature a solution of sulfamoyl chloride in toluene (ca. 0.59 M, 4.7 mL) and the resulting mixture was stirred under an atmosphere of nitrogen overnight. Ethyl acetate (100 mL) was then added to the reaction mixture and the organic layer that separated was washed with brine (100 mL, 4×50 mL), dried (MgSO4), filtered and evaporated to give a white fluffy residue (347 mg). This crude in ethyl acetate was filtered through a short column of silica and the combined filtrate upon evaporation gave LWO02090 as a white fluffy residue (310 mg, 646.8 mmol, 92.5%); R_{f} 0.29 (ethyl acetate), c.f. 0.35 (S.M.); δ_{H} (400 MHz, DMSO-d₆) 4.14 (˜0.4H, t, J 4.6-5.1 Hz, CH₂N of STX292), 4.17 (˜1.6H, t, J 4.7 Hz, CH₂N of STX301), 4.27 (2H, t, J 3.9-4.6 Hz, OCH₂CH₂N of STX292 and STX301), 6.68 (2H, AA′BB′ of STX292 and STX301), 6.91-6.99 (˜1.2H, mixture of AA′BB′ and dd of STX292 and C6′-H of STX301), 7.18 (0.4H, AA′BB′ of STX292), 7.25 (0.8H, d, J=3.1 Hz, C2′-H of STX301), 7.37 (˜0.8H, d, J=8.9 Hz, C5′-H of STX301), 7.75 (2H, AA′BB′ of STX292 and STX301), 7.91 (0.4H, br s, exchanged with D₂O, SO₂NH₂ of STX292), 8.16 (˜1.6H, br s, exchanged with D₂O, SO₂NH₂ of STX301), 8.94 (˜0.4H, s, C3″-H and C5″-H of STX292) and 8.99 (˜1.6H, s, C3″-H and C5″-H of STX301) This implies that the product contains approx. 20% of STX292; LRMS (FAB+): 481.0[100, (⁸¹M+H)⁺], 401.1[73, (STX292+H)⁺], (FAB−): 631.1[35, (79M+NBA)⁻], 477.1[100, (⁷⁹M−H)⁻], 399.1[64, (STX292-H)⁻]; HRMS (FAB+) 479.01430 C₁₇H₁₅⁷⁹BrN₆O₄S requires 479.01371, (FAB+) 401.10349 C₁₇H₁₇N₆O₄S requires 401.10320.

### 6-Benzyloxy-naphthalene-2-carboxylic acid benzyl ester (JRL01001)

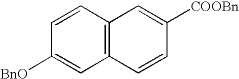

To a stirred suspension of NaH (60%, 1.37 g, 34.3 mmol) in DMF (40 mL) at 0° C. under nitrogen was added 6-hydroxy-2-naphthoic acid (3.0 g, 15.6 mmol). The resulting brown mixture was stirred for 30 min before benzyl bromide (5.99 g, 34.3 mmol) was added. After stirring overnight, the reaction mixture was poured into water and the aqueous layer was extracted with ethyl acetate (3×100 mL). The combined organic extracts was washed with brine (4×100 mL), dried (Na₂SO₄) and evaporated to give the crude product which upon fractionation with flash chromatography (hexane/ethyl acetate, 10:1) gave JRL01001 as a white solid (4.3 g, 75%); R_{f} (hexane/ethyl acetate, 10:1) 0.42; δ_{H} (400 MHz, CDCl₃) 5.19 (2H, s), 5.41 (2H, s), 7.20-7.60 (12H, m), 7.74 (1H, d, J 8.6 Hz), 7.85 (1-H, d, J 9.0 Hz), 8.06 (1H, dd, J 1.9 and 8.6 Hz) and 8.57 (1H, s); δ_{C} (100 MHz, CDCl₃) 67.1 (t), 70.5 (t), 107.2 (d), 120.2 (d), 125.5 (s), 126.3 (d), 127.2 (d), 127.5 (d), 128.2 (s), 128.4 (d), 128.5 (d), 128.6 (d), 128.9 (d), 131.2 (d), 136.5 (s), 136.7 (s), 137.4 (s), 158.6 (s), 166.8 (s).

### (6-Benzyloxy-naphthalen-2-yl)-methanol (JRL01003)

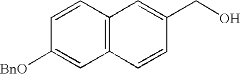

To a stirred suspension of LiAlH₄ (240 mg, 6.0 mmol) in THF (100 mL) at room temperature under nitrogen was added a solution of JRL01001 (1.99 g, 5.4 mmol) in THF. After 2 h of stirring at which time no starting material was detected by TLC, the reaction mixture was evaporated. The residue obtained was treated with ethyl acetate (100 mL) and the organic layer resulted was washed with dilute ammonium chloride solution (50 mL) and brine (3×50 mL), dried (Na₂SO₄) and evaporated. The crude product thus obtained was recrystallised from hot ethanol to give JRL01003 as white solid (1.16 g, 81%); m.p. 138.0-138.5° C.; R_{f} 0.26 (hexane/ethyl acetate, 2:1); δ_{H} (400 MHz, CDCl₃) 1.82 (1H, t, J 5.9 Hz), 4.79 (2H, d, J 5.9 Hz), 5.16 (2H, s) and 7.20-7.80 (11H, m); δ_{C} (100 MHz, CDCl₃) 65.8 (t), 70.3 (t), 107.3 (d), 119.5 (d), 125.7 (d), 126.0 (d), 127.4 (d), 127.7 (d), 128.2 (d), 128.8 (d), 129.1 (s), 129.6 (d), 134.2 (s), 136.2 (s), 137.0 (s), 157.0 (s). Found: C, 81.77; H, 6.11; C₁₈H₁₆O₂ requires C, 81.79; H, 6.10%.

### 2-Benzyloxy-6-bromomethyl-naphthalene (JRL01006)

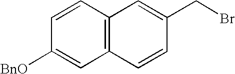

To a stirred solution of JRL01003 (1.29 g, 4.9 mmol) in dry CH₂Cl₂ (60 mL) at 0° C. under nitrogen was added PBr₃ (1.33 g, 4.9 mmol). A white suspension was formed initially but it turned into a pale yellow mixture subsequently. After stirring the reaction mixture for 2 h at 0° C. and at room temperature for 1 h, it was poured onto ice/water. The organic layer was separated and the aqueous layer extracted with dichloromethane (3×50 mL). The combined organic extracts was dried (Na₂SO₄) and evaporated to give JRL01006 as a white solid (1.51 g, 94%); R_{f} 0.28 (hexane/ethyl acetate, 10:1); δ_{H}(400 MHz, CDCl₃) 4.65 (2H, s), 5.17 (2H, s), 7.10-7.30 (2H, m), 7.30-7.50 (6H, m) and 7.68-7.78 (3H, m); LRMS (FAB+) 327.9 [25, (M+H)⁺], 247 (45), 91.0 [100, (Bn⁺)].

### 4-[(6-Benzyloxy-naphthalen-2-ylmethyl)-[1,2,4]triazol-4-yl-amino]-benzonitrile (JRL01010)

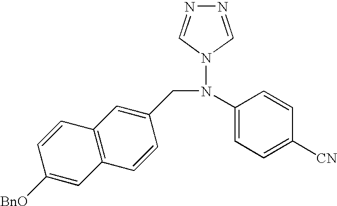

To a stirred solution of NaH (60%, 144 mg, 3.6 mmol) in DMF (10 mL) at 0° C. under nitrogen was added 4-([1,2,4]triazol-4-ylamino)benzonitrile (LWO02023, 667 mg, 3.6 mmol) in DMF (10 mL). A white suspension/mixture was resulted initially but it turned orange subsequently. After stirring at 40-50° C. for 1 h under nitrogen, the reaction mixture was cooled to room temperature and JRL01006 (1.21 g, 3.7 mmol) was added. The solution was stirred at room temperature overnight under nitrogen and then diluted with dichloromethane (100 mL). The organic layer was washed with brine (4×50 mL), dried (Na₂SO₄) and evaporated to give JRL0110 as a pale yellow solid (994 mg, 64%); R_{f} 0.42 (ethyl acetate); δ_{H} (400 MHz, CDCl₃) 5.00 (2H, s), 5.20 (2H, s), 6.72 (2H, AA′BB′), 7.20-7.80 (13H, m) and 8.10 (2H, s); LRMS (FAB+) 432.1 [70, (M+H)⁺], 363.1 [50, (M-triazole)⁺], 247.1 (35), 91.0 [100, (Bn)⁺].

### 4-[(6-Hydroxy-naphthalen-2-ylmethyl)-[1,2,4]triazol-4-yl-amino]-benzonitrile (JRL01012, STX 335)

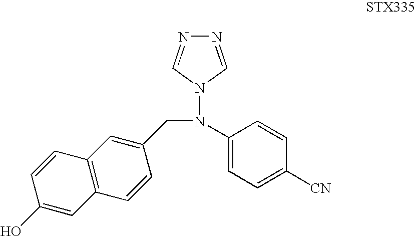

To a stirred solution of JRL01010 (906 mg, 2.1 mmol) in THF/MeOH (1:1, 180 mL) was added Pd—C (10%, 250 mg). After stirring the suspension under an atmosphere of hydrogen (balloon) overnight, it was filtered through Celite and the filtrate evaporated to give a grey crude product. Upon trituration in hot ethyl acetate, the pale grey solid that resulted was filtered and dried to give JRL01012 (STX335, 444 mg, 62%); m.p. 281-284° C.; R_{f} 0.26 (ethyl acetate); δ_{H} (400 MHz, DMSO-d₆) 5.18 (2H, s), 6.80 (2H, AA′BB′), 7.07 (2H, m), 7.34 (1H, d, J 8.2 Hz), 7.60-7.70 (3H, m), 7.76 (2H AA′BB′), 8.80 (2H, s) and 9.79 (1H, s); LRMS (FAB) 342.1 [100, (M+H)⁺], 274.1 [37, (M+H-triazole)⁺], 158.0 (25); HRMS (FAB+) 342.13594, C₂₀H₁₆N₅O requires 342.13549.

### Sulfamic acid 6-{[(4-cyano-phenyl)-[1,2,4]triazol-4-yl-amino]-methyl}-naphthalen-2-yl ester (JRL01014, STX 336)

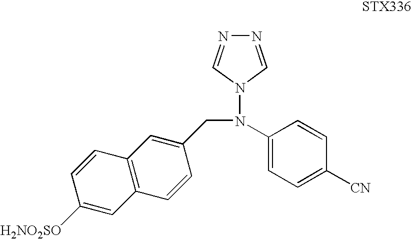

To a stirred solution of JRL01012 (181 mg, 530 μmol) in DMA (2 mL) under nitrogen was added sulfamoyl chloride (1.2 mmol). After stirring overnight at room temperature, the reaction mixture was diluted with ethyl acetate (30 mL). The organic layer was then washed with brine (4×30 mL), dried (Na₂SO₄) and evaporated to give JRL01014 (STX336, 189 mg, 85%) as a white solid. An amount of 70 mg of this solid was recrystallised from acetone/hexane to obtain white crystals (40 mg); m.p. 125-127° C.; R_{f} 0.23 (ethyl acetate); δ_{H} (400 MHz, DMSO-d₆) 5.23 (2H, s), 6.80 (2H, AA′BB′), 7.42 (1H, dd, J 2.3 and 9.0 Hz), 7.55 (1H, br dd, J 8.6 Hz), 7.74-7.82 (3H, m), 7.85 (1H, s), 7.94 (2H, m), 8.04 (2H, br s, H₂NSO₂O) and 8.84 (2H, s); LRMS (FAB+) 421.1 [100, (M+H)⁺], 352.0 [58, (M-triazole)⁺], 341.1 (10), 236.0 (25), 158.0 (10); HRMS (FAB+) 421.10766, C₂₀H₁₇N₆O₃S requires 421.10829.

### 3-Benzyoxybenzyl bromide (OBS01018)

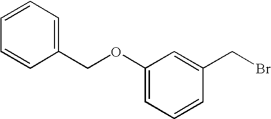

Using the procedure reported by K. Thakkar et al., J. Med. Chem., 1993, 36 (20), 2950.

Phosphorus tribromide (1.96 mL, 20.6 mmol) was added to a solution of 3-benzyloxybenzyl alcohol (4.29 g, 20 mmol) in anhydrous dichloromethane (90 mL) at 0° C. The mixture was stirred at 0° C. for 2 h and then at room temperature for 1 h. The reaction was poured onto ice/water (400 mL) and allowed to warm to room temperature.

The aqueous solution was extracted with Et₂O (5×100 mL) and the combined ethereal solution dried (MgSO₄). Concentration in vacuo gave a light yellow oil which crystallised on standing to give OBS01018 as colourless needles (4.93 g, 89%). TLC [SiO₂, EtOAc-n-hexane (1:1)] R_{f}=0.9; m.p. 55-56° C. [Lit. (Petroleum ether): 55° C.]; ¹H-NMR (400 MHz, CDCl₃) 4.44 (2H, s), 5.05 (2H, s); 6.78 (1H, d, J=2), 6.93 (1H, m), 7.22 (1H, t, J=8), 7.40 (5H, m).

### 4-[(3-Benzyloxybenzyl)(4-cyanophenyl)amino]-4H-[1,2,4]triazole (OBS01019, STX675)

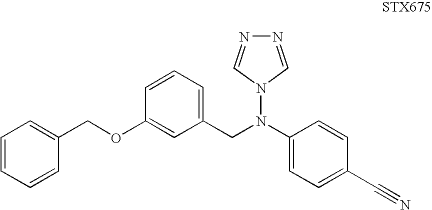

To a suspension of NaH (60% dispersion in oil, 0.22 g, 5.4 mmol) in anhydrous DMF (20 mL) at room temperature was added a solution of 4-[(4-cyanophenyl)amino]-4H-[1,2,4]triazole (see LWO02023) (1.0 g, 5.4 mmol) in anhydrous DMF (4 mL) and the mixture stirred under a positive flow of dry nitrogen for 1 h. The orange-yellow suspension was then treated with a solution of OBS01018 (1.57 g, 5.66 mmol) in anhydrous DMF (5 mL) and the mixture was stirred at room temperature overnight. The mixture was transferred to a separating funnel and diluted with EtOAc (100 mL). The organic layer was washed with water (4×100 mL), brine (100 mL), and dried (MgSO₄). Concentration in vacuo gave a residue which was recystallised from i-PrOH to give OBS01019 as a white solid (0.58 g, 28%). TLC [SiO₂, EtOAc-n-hexane (1:1)] R_{f}=0.15 (blue fluorescence at 254 nm); ¹H-NMR (400 MHz, CDCl₃) 4.85 (2H, s), 5.03 (2H, s); 6.62 (2H, AA′BB′), 6.77 (1H, d, J=7.8), 6.79 (1H, d, J=2.4), 6.96 (1H, dd, J=7.8, 2.4), 7.26 (1H, t, J=7.8), 7.34-7.37 (5H, m), 7.57 (2H, AA′BB′), 8.04 (2H, s); LC-MS: t_{R}=6.81 min, M+H=382 (Waters 2790 Alliance HPLC/ZQ MicroMass spectrometer with PDA detector using APCI, gradient elution: 5:95 MeCN/H₂O-95:5 MeCN/H₂O over 10 mins then 95:5 MeCN/H₂O-5:95 MeCN/H₂O using Waters “Symmetry” C18 (packing: 3.5 μm), 100 mm column); HPLC (Waters 717+ Autosampler with PDA detector, using Waters “Symmetry” C18 (packing: 3.5 μm), 4.6×150 mm column, 90:10 MeOH/H₂O) t_{R}=2.27 min (98% purity).

### 4-[(3-Hydroxybenzyl)(4-cyanophenyl)amino]-4H-[1,2,4]triazole (OBS01022, STX333)

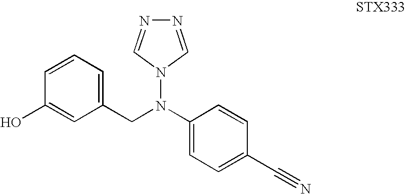

To a solution of OBS01019 (0.4 g, 1.05 mmol) in THF-MeOH (1:1) (20 mL) was added a slurry of Pd—C (10%, 0.24 g, 2.26 mmol) in THF (2 mL) and the suspension stirred under an atmosphere of hydrogen (balloon) for 21 h. The suspension was filtered through Celite and the combined filtrates concentrated in vacuo to give a brown residue which solidified on standing. Recrystallisation from EtOH gave OBS01022 (STX333) as a white solid (0.27 g, 88%). TLC [SiO₂, EtOAc-n-hexane (1:1)] R_{f}=0.6; ¹H-NMR (400 MHz, d₆-DMSO) 4.97 (2H, s), 6.68 (2H, d, AA′BB′), 6.72-6.75 (3H, m), 7.11 (1H, m), 7.76 (2H, AA′BB′), 8.77 (2H, s), 9.49 (1H, bs). MS (FAB+)=292 (M+H, 100%), 223 (M+H-triazole, 42); Acc. MS for C₁₆H₁₃N₅O (Required, 292.1192; Found, 292.1198); HPLC (Waters 717+ Autosampler with PDA detector, using Waters “Symmetry” C18 (packing: 3.5 μm), 4.6×150 mm column, 70:30 MeOH/H₂O) t_{R}=2.22 min (92% purity).

### 4-[(3-O-Sulfamoylbenzyl)(4-cyanophenyl)amino]-4H-[1,2,4]triazole (OBS01030, STX334)

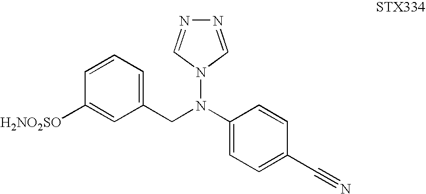

To an ice-cooled solution of OBS01022 (100 mg, 340 μmol) in DMA (2 mL) was added sulfamoyl chloride (0.59 M solution toluene, 1.2 mL, 0.69 mmol) in DMA (2 mL) and the mixture stirred under nitrogen overnight. The mixture was diluted with EtOAc (30 mL) and washed with brine (3×100 mL). The organic solution was dried (Na₂SO₄) and concentrated in vacuo to give a colourless residue which solidified on standing. Recrystallisation from acetone-petroleum ether (40-60) gave OBS01030 (STX334) as a colourless solid (0.06 g, 47%). TLC [SiO₂, EtOAc-n-hexane (1:1)] R_{f}=0.55; ¹H-NMR (400 MHz, d₆-DMSO) 5.11 (2H, s), 6.74 (2H, d, J=8.6), 7.20-7.27 (3H, m), 7.40 (1H, t, J=7.8), 7.77 (2H, d, J=8.6), 7.98 (2H, br s), 8.81 (2H, s). MS (FAB+)=371 (M+H, 100%), 302 (M+H-triazole, 28); Acc. MS for C₁₆H₁₅N₆O₃S (Required, 371.0946; Found, 371.0926); HPLC (Waters 717+ Autosampler with PDA detector, using Waters “Symmetry” C18 (packing: 3.5 μm), 4.6×150 mm column, 70:30 MeOH/H₂O) t_{R}=2.13 min (99% purity).

### 3,4-Bis(benzyloxy)benzaldehyde (OBS01058)

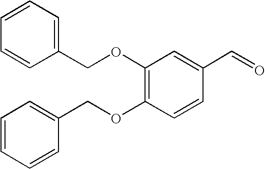

Using the procedure reported by A. F. Barrero et al., Tetrahedron, 1998, 54, 5635. To a suspension of 3,4-dihydroxybenzaldehyde (6.9 g, 50 mmol) and potassium carbonate (14.5 g) in acetone (150 mL) was added benzyl bromide (11.96 mL, 101 mmol) and the mixture heated at reflux for 15 h. The reaction was cooled and the solvent removed in vacuo to give a light brown residue. The residue was redissolved in Et₂O (200 mL), washed with water (3×200 mL) and dried (Na₂SO₄). Concetration in vacuo gave a pale yellow solid which was recrystallised from EtOH to give OBS01058 as a white powder (13.84 g, 87%). TLC [SiO₂, EtOAc-n-hexane (1:1)] R_{f}=0.7; m.p. 93-94° C. [Lit. (EtOH): 93-94° C.]; ¹H-NMR (400 MHz, d₆-DMSO) 5.22 (2H, s), 5.28 (2H, s), 7.29 (1H, d, J=8.4), 7.30-7.48 (10H, m), 7.51 (1H, d, J=2), 7.55 (1H, dd, J=2, 8.4), 9.81 (1H, s).

### 3,4-Bis(benzyloxy)benzyl alcohol (OBS01060)

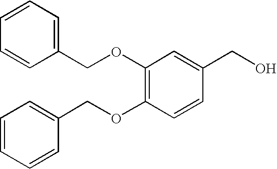 [0171] Reference: L. Lisowski et al., Bioorg. Med. Chem. Lett., 2001, 11(16), 2205

To a solution of OBS01058 (3.18 g, 10 mmol) in anhydrous THF (50 mL) was added sodium borohydride (378 mg, 10 mmol) and the mixture stirred at room temperature for 4 h. The reaction was quenched with cautious addition of water (CARE !!), filtered through Celite and concentrated in vacuo to give a yellow oil. The oil was recrystallised from MeOH to give OBS01060 as colourless needles (2.97 g, 93%). TLC [SiO₂, EtOAc-n-hexane (1:1)] R_{f}=0.45; m.p. 65-67° C. [Lit. (MeOH): 65-66° C.]; ¹H-NMR (400 MHz, CDCl₃) 4.58 (2H, d, J=5), 5.16 (2H, s), 5.17 (2H, s), 6.86 (1H, dd, J=2, 8.2), 6.91 (1H, d, J=8.2), 7.00 (1H, d, J=2), 7.29-7.39 and 7.43-7.47 (10H, m), OH signal too broad to be observed.

### 3,4-Bis(benzyloxy)benzyl bromide (OBS01061)

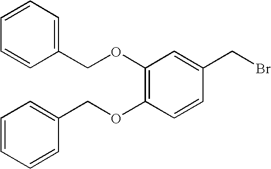 [0174] Reference: K. Thakkar et al., J. Med. Chem., 1993, 36(20), 2950.

Phosphorus tribromide (0.98 mL, 10.3 mmol) was added to a solution of OBS01060 (3.20 g, 10 mmol) in anhydrous DCM (45 mL) at 0° C. The mixture was stirred at 0° C. for 2 h and then at room temperature for 1 h. The reaction was poured onto ice water (200 mL) and allowed to warm to room temperature. The aqueous solution was extracted with Et₂O (5×50 mL) and the combined ethereal solution dried (MgSO₄). Concentration in vacuo gave a colourless oil which solidified on standing to give OBS01061 as a white solid (3.10 g, 78%). TLC [SiO₂, EtOAc-n-hexane (1:1)] R_{f}=0.89; m.p. 72-75° C. [Lit: 72-75° C.]; ¹H-NMR (400 MHz, CDCl₃) 4.53 (2H, s), 5.15 (2H, s), 5.17 (2H, s), 6.83 (1H, dd, J=2, 8.2), 6.90 (1H, d, J=8.2), 6.99 (1H, d, J=2), 7.52 (10H, m).

### 4-[(3,4-Bis(benzyloxy)benzyl)(4-cyanophenyl)amino]-4H-[1,2,4]triazole (OBS01066, STX676)

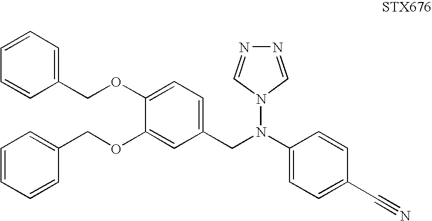

To a suspension of NaH (60% dispersion in oil, 0.22 g, 5.4 mmol) in anhydrous DMF (20 mL) at room temperature was added a solution of 4-[(4-cyanophenyl)amino]-4H-[1,2,4]triazole (1.0 g, 5.4 mmol) in anhydrous DMF (4 mL) and the mixture stirred under nitrogen for 1 h. The orange-yellow suspension was then treated with a solution of OBS01061 (2.07 g, 5.67 mmol) in anhydrous DMF (5 mL) and the mixture stirred at room temperature overnight. The mixture was transferred to a separating funnel and diluted with EtOAc (100 mL). The organic layer was washed with water (4×100 mL), brine (100 mL), and dried (MgSO₄). Concentration in vacuo gave a residue which was recystallised from EtOH to give OBS01066 (STX676) as an off-white solid (1.66 g, 63%). TLC [SiO₂, EtOAc-n-hexane (1:1)] R_{f}=0.1 (blue fluorescence at 254 nm); ¹H-NMR (400 MHz, d₆-DMSO) 4.92 (2H, s), 5.06 (2H, s), 5.07 (2H, s), 6.72-6.76 (3H, m); 6.94 (1H, d, J=8.2), 7.01 (1H, d, J=2.2), 7.28-7.42 (10H, m), 7.74 (2H, AA′BB′), 8.65 (2H, s). MS (FAB+) 488 (M+H, 45%), 419 (M+H-triazole, 21), 91 (Bn, 100%); Acc MS for C₃₀H₂₆N₅O₂ (Required, 488.2096; Found, 488.2087); LC-MS (Waters 2790 Alliance HPLC/ZQ MicroMass spectrometer with PDA detector using APCI), t_{R} (gradient elution: 5:95 MeCN/H₂O-95:5 MeCN/H₂O over 10 mins then 95:5 MeCN/H₂ O-5:95 MeCN/H₂O using Waters “Symmetry” C18 (packing: 3.5 μm), 100 mm column)=7.68 min (M+H=488); HPLC (Waters 717+ Autosampler with PDA detector, using Waters “Symmetry” C18 (packing: 3.5 μm), 4.6×150 mm column, 90:10 MeOH/H₂O) t_{R}=2.43 min (98% purity).

### 4-[(3,4-Bis(hydroxybenzyl)(4-cyanophenyl)amino]-4H-[1,2,4]triazole (OBS01067, STX355)

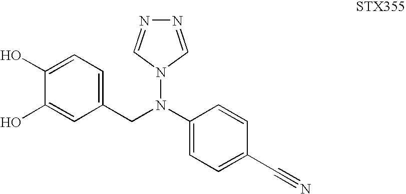

To a solution of OBS01066 (0.98 g, 2.01 mmol) in THF-MeOH (1:1) (20 mL) was added a slurry of Pd—C (10%, 0.10 g) in THF (2 mL) and the mixture stirred under an atmosphere of hydrogen (balloon) for 24 h. The suspension was filtered through Celite and the combined filtrates concentrated in vacuo to give a brown residue. Recrystallisation from MeOH gave OBS01067 (STX355) as a white solid (0.14 g, 21%). TLC [SiO₂, EtOAc-n-hexane (1:1)] R_{f}=0.6; ¹H-NMR (400 MHz, d₆-DMSO) 4.84 (2H, s), 6.50 (1H, d, J=8.2), 6.62 (2H, m), 6.75 (2H, AA′BB′), 7.75 (2H, AA′BB′), 8.68 (2H, s); LC-MS (Waters 2790 Alliance HPLC/ZQ MicroMass spectrometer with PDA detector using APCI), t_{R} (gradient elution: 5:95 MeCN/H₂O-95:5 MeCN/H₂O over 10 mins then 95:5 MeCN/H₂O-5:95 MeCN/H₂O using Waters “Symmetry” C18 (packing: 3.5 μm), 100 mm column)=7.68 min (M+H=488); HPLC (Waters 717+Autosampler with PDA detector, using Waters “Symmetry” C18 (packing: 3.5 μm), 4.6×150 mm column, 70:30 MeOH/H₂O) t_{R}=3.02 min (99% purity).

### 3-Methoxy-4-benzyloxybenzaldehyde (OBS01056)

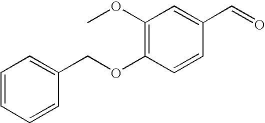

Using the procedure reported by A. I. Meyers et al., Heterocycles, 1989, 295.

To a solution of vanillin (7.7 g, 50.67 mmol) in EtOH (40 mL) was added potassium carbonate (7.9 g, 57.35 mmol) and benzyl bromide (6.0 mL, 50.67 mmol) and the mixture stirred at room temperature overnight. The reaction was filtered through Celite and the filtrates concentrated in vacuo. The residue was redissolved in DCM (250 mL), washed with aqueous NaOH (5% w/v, 2×100 mL) and the organic layer dried (Na₂SO₄). Concentration in vacuo and recrystallisation of the residue obtained from EtOH gave OBS01056 as a white powder (11.16 g, 91%). TLC [SiO₂, EtOAc-n-hexane (1:1)] R_{f}=0.8; m.p. 63-64° C. [Lit. (MeOH): 63-64° C.]; ¹H-NMR (400 MHz, CDCl₃) 3.95 (3H, s), 5.25 (2H, s), 6.99 (1H, m), 7.33-7.45 (7H, m), 9.84 (1H, s)

### 3-Methoxy-4-benzyloxybenzyl alcohol (OBS01063)

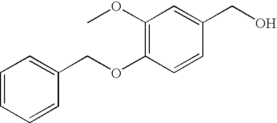

Using the procedure reported by A. van Oeveran et al., J. Org. Chem., 1994, 59 (20), 5999.

To a solution of OBS01056 (5.0 g, 20.64 mmol) in anhydrous DCM (25 mL) was added a suspension of sodium borohydride (0.97 g, 25.59 mmol) in MeOH (12 mL) and the mixture stirred at room temperature for 18 h. The reaction was poured into water (50 mL) (CARE !!) and extracted with DCM (3×50 mL) and dried (MgSO₄). Concentration in vacuo gave a white residue. Recrystallisation from Et₂O-petroleum ether gave OBS01063 as colorless needles (4.91 g, 97%). TLC [SiO₂, EtOAc-n-hexane (1:1)] R_{f}=0.49; m.p. 72-74° C. [Lit. (Et₂O-Pet. ether): 72-73° C.]; ¹H-NMR (400 MHz, d₆-DMSO) 3.74 (3H, s), 4.39 (2H, d, J=5.7), 5.05 (2H, s), 5.08 (1H, t, J=5.7, OH), 6.84 (1H, dd, J=2, 8.2), 6.91 (1H, d, J=8.2), 7.01 (1H, d, J=2), 7.31-7.46 (5H, m).

### 3-Methoxy-4-benzyloxybenzyl bromide (OBS01070)

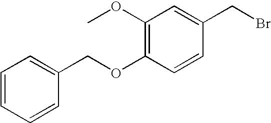

Adapting the procedure reported by A. van Oeveran et al., J. Org. Chem., 1994, 59 (20), 5999.

Phosphorus tribromide (0.98 mL, 10.3 mmol) was added to a solution of OBS01063 (2.44 g, 10 mmol) in anhydrous DCM (20 mL) at 0° C. The mixture was stirred at 0° C. for 2 h and then at room temperature for 1 h. The reaction was poured onto ice water (400 mL) and allowed to warm to room temperature. The aqueous solution was extracted with DCM (5×100 mL) and the combined organic extracts dried (MgSO₄). Concentration in vacuo gave an off-white residue which was recrystallised from n-hexane to give OBS01070 as colourless needles (2.52 g, 82%). TLC [SiO₂, EtOAc-n-hexane (1:1)] R_{f}=0.92; m.p. 73-74° C. [Lit. (Pet. ether): 73° C.]; ¹H-NMR (400 MHz, CDCl₃) 3.91 (3H, s), 4.49 (2H, s), 5.16 (2H, s), 6.81 (1H, d, J=8.2), 6.88 (1H, dd, J=8.2, 2), 6.94 (1H, d, J=2), 7.28-7.44 (5H, m).

### 4-[(3-Benzyloxy-4-methoxybenzyl)(4-cyanophenyl)amino]-4H-[1,2,4]triazole (OBS01071, STX677)

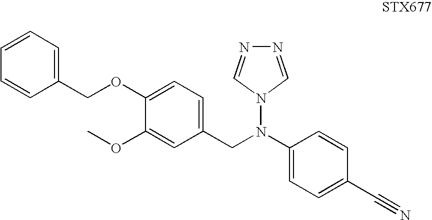

To a suspension of NaH (60% dispersion in oil, 0.22 g, 5.4 mmol) in anhydrous DMF (20 mL) at room temperature was added a solution of 4-[(4-cyanophenyl)amino]-4H-[1,2,4]triazole (1.0 g, 5.68 mmol) in anhydrous DMF (4 mL) and the mixture stirred under nitrogen for 1 h. The orange-yellow suspension was then treated with a solution of OBS01071 (1.66 g, 5.4 mmol) in anhydrous DMF (5 mL) and the mixture stirred at room temperature overnight. The mixture was transferred to a separating funnel and diluted with EtOAc (200 mL). The organic layer was washed with water (4×200 mL), brine (200 mL), and dried (MgSO₄). Concentration in vacuo gave a residue which was recystallised from i-PrOH to give OBS01071 (STX677) as an off-white powder (769 mg, 35%). TLC [SiO₂, EtOAc-n-hexane (1:1)] R_{f}=0.15 (blue fluorescence at 254 nm); 1H-NMR (400 MHz, d₆-DMSO) 3.80 (3H, s), 4.79 (2H, s), 5.12 (2H, s), 6.64 (1H, d, J=2), 6.66 (1H, dd, J=8.2, 2), 6.70 (2H, AA′BB′), 6.81 (1H, d, J=8.2), 7.29-7.42 (5H, m), 7.59 (2H, AA′BB′), 8.06 (2H, s); MS (FAB+)=412 (M+H, 100%), 343 (40), 275 (20), 227 (20); Acc MS for C₂₄H₂₂N₅O₂ (Required, 412.1779; Found, 412.1773); LC-MS (Waters 2790 Alliance HPLC/ZQ MicroMass spectrometer with PDA detector using APCI), t_{R} (gradient elution: 5:95 MeCN/H₂O-95:5 MeCN/H₂O over 10 mins then 95:5 MeCN/H₂O-5:95 MeCN/H₂O using Waters “Symmetry” C18 (packing: 3.5 μm), 100 mm column)=6.61 min (M+H=412); HPLC (Waters 717+ Autosampler with PDA detector, using Waters “Symmetry” C18 (packing: 3.5 μm), 4.6×150 mm column, 90:10 MeOH/H₂O) t_{R}=2.19 min (95% purity).

### 4-[(3-Hydroxy-4-methoxybenzyl)(4-cyanophenyl)amino]-4H-[1,2,4]triazole (OBS01076, STX362)

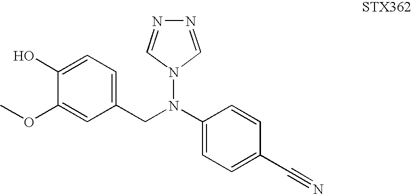

To a solution of OBS01071 (411 mg, 999 μmol) in THF-MeOH (1:1) (20 mL) was added a slurry of Pd—C (10%. 42 mg) in THF (2 mL) and the mixture stirred under an atmosphere of hydrogen (balloon) for 48 h. The mixture was filtered through Celite and the combined filtrates concentrated in vacuo to give a brown residue which solidified on standing. Recrystallisation from i-PrOH gave OBS01076 (STX362) as a colourless powder (232 mg, 72%). TLC [SiO₂, EtOAc-n-hexane (1:1)] R_{f}=0.52; ¹H-NMR (400 MHz, d₆-DMSO) 3.71 (3H, s), 4.90 (2H, s), 6.61 (1H, dd, J=7.8, 2), 6.65 (1H, d, J=7.8), 6.79 (2H, AA′BB′), 6.80 (1H, d, J=2), 7.76 (2H, AA′BB′), 8.72 (2H, s), 9.06 (1H, br s, OH); MS (FAB+)=322 (M+H, 100%), 253 (64); Acc MS for C₁₇H₁₆N₅O₂ (Required, 322.1303; Found, 322.1304); HPLC (Waters 717+ Autosampler with PDA detector, using Waters “Symmetry” C18 (packing: 3.5 μm), 4.6×150 mm column, 80:20 MeOH/H₂O) t_{R}=2.59 min (95% purity).

### 4-[(4-Methoxy-3-O-sulfamoylbenzyl)(4-cyanophenyl)amino]-4H-[1,2,4]triazole (OBS01135, STX660)

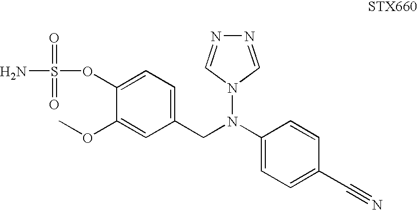

To an ice-cooled solution of OBS01076 (100 mg, 310 μmol) in DMA (10 mL) was added sulfamoyl chloride (0.69 M solution toluene, 2.71 mL—the toluene was removed in vacuo (not allowing the temperature of the water bath to exceed 30° C.) prior to addition, 8.1 mmol) and the mixture stirred under nitrogen overnight. The mixture was diluted with EtOAc (100 mL) and washed with water (3×100 mL) and brine (100 mL). The organic solution was dried (MgSO₄) and concentrated in vacuo to give OBS01135 (STX660) as a white residue which was precipitated from EtOAc solution by addition of n-hexane (40 mg, 32%). TLC [SiO₂, EtOAc-n-hexane (1:1)] R_{f}=0.52; ¹H-NMR (400 MHz, d₆-DMSO) 3.77 (3H, s), 5.06 (2H, s), 6.76 (2H, AA′BB′), 6.89 and 6.91 (1H, dd, J=8.2, 2.3), 7.08 (1H, d, J=2.3), 7.23 (1H, d, J=8.2), 7.78 (2H, AA′BB′), 7.95 (2H, s), 8.87 (2H, s); LC-MS (Waters 2790 Alliance HPLC/ZQ MicroMass spectrometer with PDA detector using APCI), t_{R} (gradient elution: 5:95 MeCN/H₂O-95:5 MeCN/H₂O over 10 min then 95:5 MeCN/H₂O-5:95 MeCN/H₂O using Waters “Symmetry” C18 (packing: 3.5 μm), 100 mm column)=4.29 min (M+H=401); HPLC (Waters 717+Autosampler with PDA detector, using Waters “Symmetry” C18 (packing: 3.5 μm), 4.6×150 mm column, 80:20 MeOH/H₂O) t_{R}=3.95 min (88% purity).

### 4-Methoxy-3-benzyloxybenzaldehyde (OBS01054)

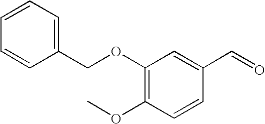

Using the procedure reported by A. I. Meyers et al., Heterocycles, 1989, 295.

To a suspension of isovanillin (7.7 g, 50.67 mmol) in water (50 mL) was added potassium hydroxide (3.4 g, 60 mmol) and the mixture stirred for 0.25 h. The now homogenous solution was then treated with benzyl bromide (6.0 mL, 50.67 mmol) and the mixture heated at reflux for 5 h. The reaction was diluted with DCM (200 mL), washed with water (2×100 mL) and brine (100 mL) and dried (Na₂SO₄). Concentration in vacuo and recrystallisation of the yellow residue obtained from EtOH gave OBS01054 as a colourless powder (11.28 g, 92%). TLC [SiO₂, EtOAc-n-hexane (1:1)] R_{f}=0.8; m.p. 62-63° C. [Lit. (EtOH): 62° C.]; ¹H-NMR (400 MHz, d₆-DMSO) 3.88 (3H, s), 5.17 (2H, s), 7.21 (1H, d, J=8.2), 7.33-7.49 (6H, m), 7.58 (1H, d, J=8.2), 9.83 (1H, s).

### 4-Methoxy-3-benzyloxybenzyl alcohol (OBS01062)

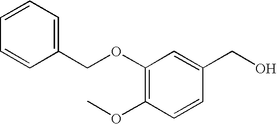

Using the procedure reported by A. I. Meyers et al., Heterocycles, 1989, 295.

To a solution of OBS01054 (5.0 g, 20.64 mmol) in EtOH (60 mL) was added sodium borohydride (1.23 g, 32.40 mmol) and the mixture stirred at room temperature for 18 h. The reaction was heated at relux for 1 h, cooled and decanted into aqueous ammonium chloride (50% w/v, 250 mL) (CARE !!). The aqueous solution was extracted with Et₂O (3×100 mL), washed with brine (100 mL) and dried (Na₂SO₄). Concentration in vacuo gave a white residue. Recrystallisation from EtOAc-petroleum ether gave OBS01062 as colorless needles (4.91 g, 97%). TLC [SiO₂, EtOAc-n-hexane (1:1)] R_{f}=0.48; m.p. 73-74° C. [Lit. (EtOAc-Pet. ether): 73° C.]; ¹H-NMR (400 MHz, d₆-DMSO) 3.74 (3H, s), 4.39 (2H, d, J=5.7), 5.05 (2H, s), 5.08 (1H, t, J=5.7, OH), 6.84 (1H, dd, J=2, 8.2), 6.91 (1H, d, J=8.2), 7.01 (1H, d, J=2), 7.33-7.49 (5H, m).

### 4-Methoxy-3-benzyloxybenzyl bromide (OBS01068)

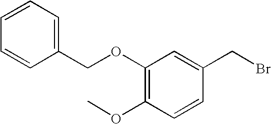

Using the procedure reported by A. I. Meyers et al., Heterocycles, 1989, 295.

Phosphorus tribromide (0.98 mL, 10.3 mmol) was added to a solution of OBS01062 (2.44 g, 10 mmol) in anhydrous THF (20 mL) at 0° C. The mixture was stirred at 0° C. for 2 h and then at room temperature for 1 h. Concentration in vacuo gave an off-white residue which was recrystallised from DCM-n-hexane to give OBS01068 as a white powder (2.95 g, 96%). TLC [SiO₂, EtOAc-n-hexane (1:1)] R_{f}=0.9; m.p. 86-88° C. [Lit. (DCM-hexane): 86-87° C.]; ¹H-NMR (400 MHz, d₆-DMSO) 3.74 (3H, s), 4.39 (2H, s), 5.05 (2H, s), 6.83 (1H, dd, J=2, 8.2), 6.90 (1H, d, J=8.2), 7.01 (1H, d, J=2), 7.30-7.47 (5H, m).

### 4-[(3-Benzyloxy-4-methoxybenzyl)(4-cyanophenyl)amino]-4H-[1,2,4]triazole (OBS01069)

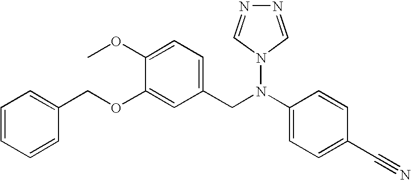

To a suspension of NaH (60% dispersion in oil, 220 mg, 5.4 mmol) in anhydrous DMF (20 mL) at room temperature was added a solution of 4-[(4-cyanophenyl)amino]-4H-[1,2,4]triazole (1.0 g, 5.68 mmol) in anhydrous DMF (4 mL) and the mixture stirred under nitrogen for 1 h. The orange-yellow suspension was then treated with a solution of OBS01068 (1.66 g, 5.4 mmol) in anhydrous DMF (5 mL) and the mixture stirred at room temperature overnight. The mixture was transferred to a separating funnel and diluted with EtOAc (200 mL). The organic layer was washed with water (4×200 mL), brine (200 mL), and dried (MgSO₄). Concentration in vacuo gave a residue which was recystallised from EtOH to give OBS01069 as an off-white powder (970 mg, 44%). TLC [SiO₂, EtOAc-n-hexane (1:1)] R_{f}=0.12 (blue fluorescence at 254 mm); ¹H-NMR (400 MHz, CDCl₃) 3.89 (3H, s), 4.72 (2H, s), 5.12 (2H, s), 6.59 (2H, AA′BB′), 6.62 (1H, d, J=2), 6.64 (1H, dd, J=7.8, 2), 6.79 (1H, d, J=7.8), 7.28-7.34 (5H, m), 7.57 (2H, AA′BB′), 7.74 (2H, s); MS (FAB+) 412 (M+H, 100%), 343 (51), 227 (72); Acc MS for C₂₄H₂₂N₅O₂ (Required, 412.1776; Found, 412.1774).

### 4-[(3-Hydroxy-4-methoxybenzyl)(4-cyanophenyl)amino]-4H-[1,2,4]triazole (OBS01080, STX363)

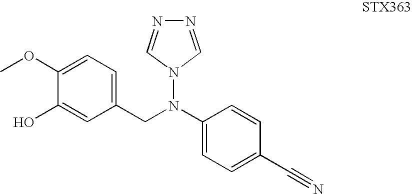

To a solution of OBS01069 (411 mg, 999 μmol) in THF-MeOH (1:1) (20 mL) was added a slurry of Pd—C (10%, 42 mg) in THF (2 mL) and the suspension stirred under an atmosphere of hydrogen (balloon) for 48 h. The suspension was filtered through Celite and the combined filtrates concentrated in vacuo to give a brown residue. Recrystallisation from i-PrOH gave OBS01080 (STX363) as a white powder (164 mg, 51%). TLC [SiO₂, EtOAc-n-hexane (1:1)] R_{f}=0.52; ¹H-NMR (400 MHz, d₆-DMSO) 3.71 (3H, s); 4.88 (2H, s), 6.62 (1H, dd, J=8.2, 1.95), 6.68 (1H, d, J=1.95), 6.74 (2H, AA′BB′), 6.80 (1H, d, J=8.2), 7.74 (2H, AA′BB′), 8.70 (2H, s), 9.00 (1H, s, OH); MS (FAB+) 322 (M+H, 100%), 253 (61); Acc MS for C₁₇H₁₆N₅O₂ (Required, 322.1304; Found, 322.1304); HPLC (Waters 717+ Autosampler with PDA detector, using Waters “Symmetry” C18 (packing: 3.5 μm), 4.6×150 mm column, 90:10 MeOH/H₂O) t_{R}=2.03 min (99% purity).

### 4-[(4-Methoxy-3-O-sulfamoylbenzyl)(4-cyanophenyl)amino]-4H-[1,2,4]triazole (OBS01137, STX661)

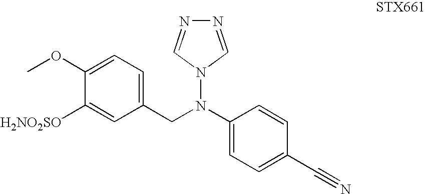

To an ice-cooled solution of OBS01080 (100 mg, 310 μmol) in DMA (10 mL) was added sulfamoyl chloride (0.69 M solution toluene, 2.71 mL—the toluene was removed in vacuo (not allowing the temperature of the water bath to exceed 30° C.) prior to addition, 8.1 mmol) and the mixture stirred under nitrogen overnight. The mixture was diluted with EtOAc (100 mL) and washed with water (3×100 mL) and brine (100 mL). The organic layer was dried (MgSO₄) and concentrated in vacuo to give OBS01137 (STX661) as a colourless residue which was precipitated from EtOAc solution by addition of n-hexane (40 mg, 32%). TLC [SiO₂, EtOAc-n-hexane (1:1)] R_{f}=0.53; ¹H-NMR (400 MHz, d₆-DMSO) 3.77 (3H, s); 5.00 (2H, s), 6.79 (2H, AA′BB′), 7.06 (1H, d, J=8.6), 7.12 and 7.14 (1H, dd, J=8.2, 2.3), 7.27 (1H, d, J=2), 7.77 (2H, AA′BB′), 7.94 (2H, s), 8.75 (2H, s); LC-MS (Waters 2790 Alliance HPLC/ZQ MicroMass spectrometer with PDA detector using APCI), t_{R} (gradient elution: 5:95 MeCN/H₂O-95:5 MeCN/H₂O over 10 mins then 95:5 MeCN/H₂O-5:95 MeCN/H₂O using Waters “Symmetry” C18 (packing: 3.5 μm), 100 mm column)=4.70 min (M+H=401); HPLC (Waters 717+ Autosampler with PDA detector, using Waters “Symmetry” C18 (packing: 3.5 μm), 4.6×150 mm column, 80:20 MeOH/H₂O) t_{R}=2.04 min (88% purity).

### 3-Bromo-4-hydroxybenzaldehyde (OBS01057)

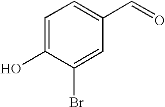

Adapting the procedure reported by S. Kelly et al., Helv. Chim. Acta., 1989, 72, 594.

To a ice-cooled solution of 4-hydroxybenzaldehyde (30.0 g, 245.67 mmol) in glacial AcOH (120 mL) was added bromine (12.6 mL, 257.61 mmol). The mixture was stirred at room temperature overnight, diluted with water (600 mL) and extracted with DCM (3×120 mL). The combined organic fractions were washed with water (600 mL), dilute sodium hydrogen carbonate solution (2×600 mL) and brine (600 mL). The organic layer was dried (Na₂SO₄) and concentrated in vacuo to give a brown residue. Recrystallisation from toluene gave OBS01057 as a pink-brown crystalline solid (26.3 g, 53%). TLC [SiO₂, EtOAc-n-hexane (1:1)] R_{f}=0.6; m.p. 124-126° C. [Lit. (CHCl₃): 125-126° C.]; ¹H-NMR (400 MHz, CDCl₃) 6.31 (1H, s, OH), 7.15 (1H, d, J=8.8), 7.77 (1H, dd, J=8.8, 2), 8.04 (1H, d, J=2), 9.83 (1H, s, CHO).

### 3-Bromo-4-benzoyloxybenzaldehyde (OBS01072)

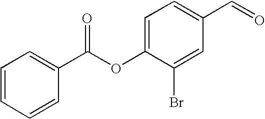

To a solution of OBS01057 (8.0 g, 40.0 mmol) in EtOAc (100 mL) was added NEt₃ (5.58 mL, 40.0 mmol) and the mixture stirred at room temperature for 0.5 h. Benzoyl chloride (4.64 mL, 40.0 mmol) was then added and the reaction stirred at room temperature for 5 h. The precipitated NEt₃.HCl was filtered off and the organic solution dried (Na₂SO₄). Concentration in vacuo gave a grey residue. Recrystallisation from EtOAc-petroleum ether gave OBS01072 as a yellow solid (10.9 g, 89%). TLC [SiO₂, EtOAc-n-hexane (1:1)] R_{f}=0.82; ¹H-NMR (400 MHz, CDCl₃) 7.50 (1H, d, J=8.2), 7.54, 7.56 and 7.58 (2H, tt, J=7.4, 1.4), 7.68, 7.70 and 7.71 (1H, tt, J=7.4, 1.4), 7.91 and 7.93 (1H, dd, J=8.2, 1.8), 8.20 (1H, d, J=1.8), 8.24 and 8.27 (2H, dt, J=8.2, 1.4), 9.98 (1H, s, CHO).

### 3-Bromo-4-benzoyloxybenzyl alcohol (OBS01074)

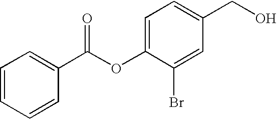

To a solution of OBS01072 (6.10 g, 19.99 mmol) in anhydrous THF (25 mL) was added sodium borohydride (1.13 g, 29.99 mmol) and the mixture stirred at room temperature for 4 h. The reaction was quenched with cautious addition of water (CARE !!), filtered through Celite and dried (MgSO₄). Concentration in vacuo gave a yellowish oil which crystallised on standing. Recrystallisation from Et₂O gave 3-bromo-4-benzoyloxybenzyl alcohol as colorless needles (5.89 g, 96%). TLC [SiO₂, EtOAc-n-hexane (1:1)] R_{f}=0.5; ¹H-NMR (400 MHz, CDCl₃) □=4.71 (2H, s), 7.26 (1H, d, J=8.2), 7.36 (1H, dd, J=8.2, 1.95), 7.51, 7.53 and 7.55 (2H, tt, J=8.2, 1.6), 7.64-7.68 (2H, m), 8.24 (1H, t, J=1.95), 8.26 (1H, t, J=1.95).

### 3-Bromo-4-benzoyloxybenzyl bromide (OBS01089)

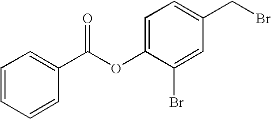

Phosphorus tribromide (0.98 mL, 10.3 mmol) was added to a solution of OBS01074 (3.07 g, 10.0 mmol) in anhydrous dichloromethane (45 mL) at 0° C. The mixture was stirred at 0° C. for 2 h and then at room temperature for 1 h. The reaction was poured onto ice water (400 mL) and allowed to warm to room temperature. The aqueous solution was extracted with diethyl ether (5×100 mL) and the combined ethereal solution dried (MgSO₄). Concentration in vacuo gave a yellow oil. Purification by gravity column chromatography [SiO₂, EtOAc-petroleum ether (1:7)] give OBS01089 as colourless needles (3.22 g, 87%). TLC [SiO₂, EtOAc-n-hexane (1:1)] R_{f}=0.87; ¹H-NMR (400 MHz, CDCl₃) 4.47 (2H, s), 7.27 (1H, d, J=8.6), 7.41 (1H, dd, J=8.6, 2.3), 7.52, 7.54 and 7.56 (2H, tt, J=8.2, 1.56), 7.66, 7.67 and 7.69 (1H, tt, J=8.2, 1.56), 7.70 (1H, d, J=2.3), 8.24 (1H, t, J=1.2), 8.26 (1H, t, J=1.2).

### 4-[(4-Benzoyloxy-3-bromobenzyl)(4-cyanophenyl)amino]-4H-[1,2,4]triazole (OBS01131)

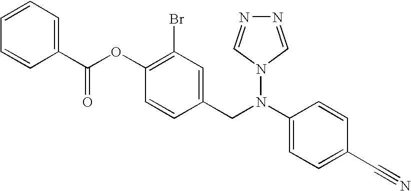

To a suspension of NaH (60% dispersion in oil, 220 mg, 5.4 mmol) in anhydrous DMF (20 mL) at room temperature was added a solution of 4-[(4-cyanophenyl)amino]-4H-[1,2,4]triazole (1.0 g, 5.68 mmol) in anhydrous DMF (4 mL) and the mixture stirred under nitrogen for 1 h. The orange-yellow suspension was then treated with a solution of OBS01089 (2.00 g, 5.4 mmol) in anhydrous DMF (5 mL) and the mixture stirred at room temperature overnight. The mixture was transferred to a separating funnel and diluted with EtOAc (200 mL). The organic layer was washed with water (4×200 mL), brine (200 mL), and dried (MgSO₄). Concentration in vacuo gave a residue which was recrystallised from i-PrOH to give OBS01131 as a colourless solid (1.99 g, 78%). TLC [SiO₂, EtOAc (100%)] R_{f}=0.5 (blue fluorescence at 254 nm); ¹H-NMR (400 MHz, d₆-DMSO) 5.14 (2H, s), 6.75 (1H, t, J=2.7), 6.78 (1H, t, J=2.7), 7.44 (2H, d, J=8.2), 7.61-7.66 (2H, m), 7.76-7.80 (4H, m), 8.14 (1H, t, J=2.7), 8.16 (1H, t, J=2.7), 8.92 (2H, s); MS (FAB+) 474 (M, 100%), 405 (38); Acc MS for C₂₃H₁₆N₅O₂Br (Required, 474.0569; Found, 474.0566).

### 4-[(3-Bromo-4-hydroxybenzyl)(4-cyanophenyl)amino]-4H-[1,2,4]triazole (OBS01132, STX405)

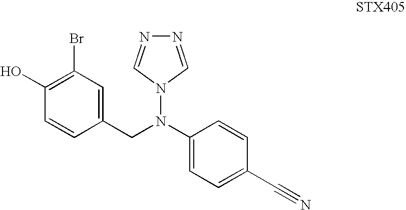

To a suspension of OBS01131 (2.0 g, 4.22 mmol) in MeOH (10 mL) was added potassium hydroxide (1.42 g, 25.3 mmol) and the mixture stirred at room temperature for 2 h. The solvents were removed in vacuo and he pH of the alkaline slurry was adjusted to ˜pH 6-7 by treatment with saturated sodium hydrogen carbonate solution. The colourless precipitate was filtered off, washed with the minimum of cold water and boiled in i-PrOH to give OBS01091 (STX405) as a colourless powder (1.20 g, 77%). TLC [SiO₂, EtOAc-n-hexane (1:1)] R_{f}=0.43; ¹H-NMR (400 MHz, d₆-DMSO) 4.93 (2H, s), 6.77 (2H, AA′BB′), 6.85 (1H, d, J=8.2), 7.06 (1H, dd, J=8.2, 1.95), 7.41 (1H, d, J=1.95), 7.76 (2H, AA′BB′), 8.76 (2H, s), 10.35 (1H, br s, OH); MS (FAB+) 370 (M, 100%), 301 (55), 260 (67), 242 (64); Acc MS for C₁₆H₁₂N₅OBr (Required, 370.0314; Found, 370.0304); HPLC (Waters 717+ Autosampler with PDA detector, using Waters “Symmetry” C18 (packing: 3.5 μm), 4.6×150 mm column, 94:6 MeOH/H₂O) t_{R}=2.73 min (96% purity).

### 4-[(3-Bromo-4-O-sulfamoylbenzyl)(4-cyanophenyl)amino]-4H-[1,2,4]triazole (OBS01141, STX681)

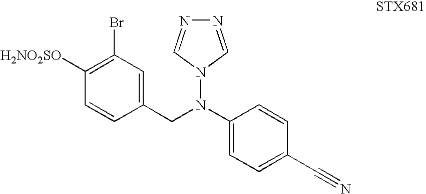

To an ice-cooled solution of OBS01132 (500 mg, 1.35 mmol) in DMA (10 mL) was added sulfamoyl chloride (0.69 M solution toluene, 11.7 mL—the toluene was removed in vacuo (not allowing the temperature of the water bath to exceed 30° C.) prior to addition, 8.1 mmol) and the mixture stirred under nitrogen overnight. The mixture was diluted with EtOAc (100 mL) and washed with water (3×100 mL) and brine (100 mL). The organic layer was dried (MgSO₄) and concentrated in vacuo to give a colourless residue which was precipitated from EtOAc solution by addition of n-hexane (463 mg). The white solid (200 mg) was then purified by gradient elution gravity column chromatography [SiO₂, EtOAc-n-hexane (1:4)-EtOAc (100%)] to give OBS01141 (STX681) as a white solid (107 mg, 44% after chromatography). TLC [SiO₂, EtOAc-n-hexane (1:1)] R_{f}=0.42; ¹H-NMR (400 MHz, d₆-DMSO) 5.17 (2H, s), 6.80 (2H, AA′BB′), 7.50 (2H, s), 7.78 (1H, s), 7.84 (2H, AA′BB′), 8.38 (2H, s), 8.96 (2H, s); MS (FAB+) 412 (M+H, 100%), 343 (40), 275 (20), 227 (20); Acc MS for C₂₄H₂₂N₅O₂ (Required, 412.1779; Found, 412.1773); LC-MS (Waters 2790 Alliance HPLC/ZQ MicroMass spectrometer with PDA detectors using APCI), t_{R} (gradient elution: 5:95 MeCN/H₂O-95:5 MeCN/H₂O over 10 mins then 95:5 MeCN/H₂O-5:95 MeCN/H₂O using Waters “Symmetry” C18 (packing: 3.5 μm), 100 mm column)=5.52 min (M+H=448); HPLC (Waters 717+ Autosampler with PDA detector, using Waters “Symmetry” C18 (packing: 3.5 μm), 4.6×150 mm column, 90:10 MeOH/H₂O) t_{R}=1.89 min (99% purity). HPLC cannot differentiate OBS01091 (STX 405) and OBS01141 (STX 681). LC-MS/¹H-NMR indicates that sample of OBS01141 (STX 681) [M+H]=450 contains ca. 6.66% of starting material OBS01091 (STX 405) [M+H]=371.

### 4-[(2-Bromo-ethyl)-[1,2,4]triazol-4-yl-amino]-benzonitrile (CAB03031)

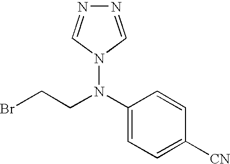

Sodium hydride (60%, 240 mg, 6.0 mmol) was added to a solution of 4-([1,2,4]triazol-4-ylamino)-benzonitrile (926 mg, 5.0 mmol) in DMSO (25 mL) at r.t. The mixture was stirred for 1 hour at this temperature and 1,2-dibromethane (5 mL) was added. The reaction mixture was stirred overnight and ethyl acetate (100 mL) was added. The mixture was transferred into a separation funnel and extracted with water (twice 100 mL) and brine (20 mL). The organic layer was dried over sodium sulphate and concentrated under reduced pressure (water bath temperature <30° C.). The resulting orange oil was mixed with diethyl ether (100 mL) and filtered through a layer of silica (ca. 5 cm). The silica was washed with more diethyl ether (ca. 100 mL) to remove the excess of 1,2-dibromoethane; the crude product was washed from the silica with acetone (120 mL). The acetone solution was concentrated under reduced pressure and the residue was purified by column-chromatography (eluent: ethyl acetate) to give the title compound as a white solid. Yield: 628 mg (43%). ¹H-NMR (400 MHz, d6-DMSO) δ=3.61 (t, J=6.2 Hz, 2H), 4.30 (t, J=6.4 Hz, 2H), 6.64 (d, J=9.0 Hz), 7.74 (d, 9.0 Hz, 2H), 8.97 (s, 2H). LRMS (FAB+):292.0 (100, [M+H]⁺).

### 4-{[2-(4-Hydroxy-phenylsulfanyl)-ethyl]-[1,2,4]triazol-4-yl-amino}-benzonitrile (CAB02137, STX456)

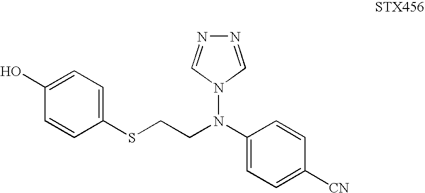

A mixture of 4-[(2-bromo-ethyl)-[1,2,4]triazol-4-yl-amino]-benzonitrile (CAB03031, 146 mg, 0.50 mmol), 4-hydroxythiophenol (126 mg, 1.0 mmol) and potassium carbonate (138 mg, 1.0 mmol) in DMF (10 mL) was stirred overnight at room temperature. The mixture was transferred into a separation funnel and ethyl acetate (50 mL) and water (50 mL) were added. The organic layer was separated, washed with brine (30 mL), dried over sodium sulphate and concentrated under reduced pressure. The residue was crystallised from methanol. Yield: 116 mg (69%) colourless crystals. ¹H-NMR (400 MHz, d6-DMSO) δ=2.99 (t, J=7.0 Hz, 2H), 3.93 (t, J=7.0 Hz, 2H), 6.48 (d, =9.0 Hz, 2H), 6.73 (d, J=8.6 Hz, 2H), 7.21 (d, J=8.6 Hz, 2H), 7.68 (d, J=9.0 Hz, 2H), 8.89 (s, 2H), 9.66 (s, 1H, —OH). ¹³C-NMR (100 MHz, d₆-DMSO) δ=30.62, 53.07, 102.92, 113.36, 116.86, 119.59, 122.71, 134.07, 134.49, 144.07, 151.17, 157.83. LRMS (FAB+): 338.2 (100, [M+H]⁺). Found: C, 60.6; H, 4.57; N, 20.6%; C₁₇H₁₅N₅OS (337.4) requires C, 60.52; H, 4.48; N, 20.76%.

### 4-{[2-(3-Hydroxy-phenylsulfanyl)-ethyl]-[1,2,4]triazol-4-yl-amino}-benzonitrile (CAB02149, STX512)

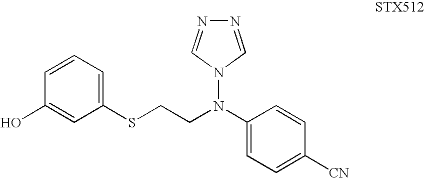

A mixture of 4-[(2-bromo-ethyl)-[1,2,4]triazol-4-yl-amino]-benzonitrile (CAB02148=CAB03031, 146 mg, 0.50 mmol), 3-(tert-butyl-dimethylsiloxy)-thiophenol (240 mg, 1.0 mmol) and potassium carbonate (276 mg, 2.0 mmol) in DMF (10 mL) was stirred for 48 hours at room temperature. The mixture was transferred into a separation funnel and ethyl acetate (50 mL) and water (50 mL) were added. The organic layer was separated, washed with brine (30 mL), dried over sodium sulphate and concentrated under reduced pressure. The residue was crystallised from methanol. Yield: 93 mg (55%) colourless crystals. ¹H-NMR (400 MHz, d₆-DMSO) δ=3.14 (t, J=7.0 Hz, 2H), 4.02 (t, J=7.0 Hz, 2H), 6.53 (d, J=9.0 Hz, 2H), 6.62 (ddd, J=7.8, 2.0, 0.8 Hz, 1H), 6.70-6.72 (m, 2H), 7.10 (dd, J=7.8, 7.8, 1H), 7.70 (d, J=9.0 Hz, 2H), 8.94 (s, 2H), 9.60 (s, 1H, —OH). ¹³C-NMR (100 MHz, d₆-DMSO) δ=29.53, 52.46, 102.38, 112.86, 113.57, 115.36, 119.00, 130.11, 133.94, 135.82, 143.50, 150.71, 157.90, 169.59. LRMS (FAB+): 338.2 (100, [M+H]⁺). Found: C, 60.6; H, 4.53; N, 20.8%; C₁₇H₁₅N₅OS (337.4) requires C, 60.52; H, 4.48; N, 20.76%.

### 4-[(3-Bromo-propyl)-[1,2,4]triazol-4-yl-amino]-benzonitrile (CAB02180, STX595)

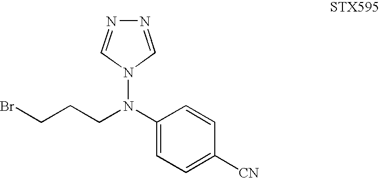

Sodium hydride (60%, 240 mg, 6.0 mmol) was added to a solution of 4-([1,2,4]triazol-4-ylamino)-benzonitrile (926 mg, 5.0 mmol) in DMSO (25 mL) at r.t. The mixture was stirred for 1 hour at this temperature and 1,3-dibromopropane (5 mL) was added. The reaction mixture was stirred overnight and ethyl acetate (100 mL) was added. The mixture was transferred into a separation funnel and extracted with water (twice 100 mL) and brine (20 mL). The organic layer was dried over sodium sulphate and concentrated under reduced pressure (water bath temperature <30° C.). The resulting orange oil was mixed with diethyl ether (100 mL) and filtered through a layer of silica (ca. 5 cm). The silica was washed with more diethyl ether (ca. 100 mL) to remove the excess of 1,3-dibromopropane, the crude product was washed from the silica with acetone (120 mL). The acetone solution was concentrated under reduced pressure and the residue was purified by column-chromatography (eluent: ethyl acetate) to give the title compound as a white solid. Yield: 792 mg (52%). ¹H-NMR (400 MHz, d₆-DMSO) δ=2.01 (tt, J=7.0, 6.6 Hz, 2H), 3.64 (t, J=6.6 Hz, 2H), 3.95 (t, J=7.0 Hz, 2H), 6.62 (d, J=9.0 Hz, 2H), 7.74 (d, J=9.0 Hz, 2H), 9.02 (s, 2H). ¹³C-NMR (100 MHz, d₆-DMSO) δ=30.38, 32.13, 52.72, 103.17, 113.83, 119.74, 134.63, 144.14, 151.65. LRMS (FAB+): 306.0 (100, [M+H]⁺).

### 4-{[3-(4-Hydroxy-phenylsulfanyl)-propyl]-[1,2,4]triazol-4-yl-amino}-benzonitrile (CAB0182, STX596)

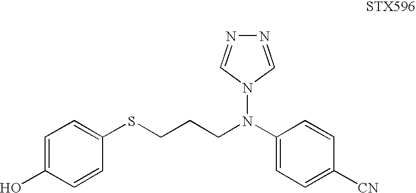

A mixture of 4-[(2-bromo-propyl)-[1,2,4]triazol-4-yl-amino]-benzonitrile (CAB02180, 146 mg, 0.50 mmol), 4-hydroxy-thiophenol (240 mg, 1.0 mmol) and potassium carbonate (276 mg, 2.0 mmol) in DMF (10 mL) was stirred for 48 hours at room temperature. The mixture was transferred into a separation funnel and ethyl acetate (50 mL) and water (50 mL) were added. The organic layer was separated, washed with brine (30 mL), dried over sodium sulphate and concentrated under reduced pressure. The residue was purified by column chromatography (eluent: ethyl acetate, Rf: 0.31) to give an yellow oil, which was crystallised from methanol. Yield: 211 mg (60%). ¹H-NMR (400 MHz, d₆-DMSO) δ=1.64 (tt, J=7.0, 7.0 Hz, 2H), 2.89 (t, J=7.0 Hz, 2H), 3.92 (t, J=7.0 Hz, 2H), 6.57 (d, J=9.0 Hz, 2H), 6.72 (d, J=9.0 Hz, 2H), 7.20 (d, J=9.0 Hz, 2H), 7.71 (d, J=9.0 Hz, 2H), 8.96 (s, 2H), 9.59 (s, 1H, —OH). LRMS (FAB+): 352.1 (100, [M+H]⁺).

### Sulfamic acid 4-{3-[(4-cyano-phenyl)-[1,2,4]triazol-4-yl-amino]-propylsulfanyl}-phenyl ester (CAB02184, STX597)

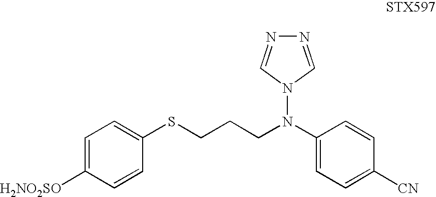

Sulphamoyl chloride solution in toluene (3 mL, 0.7 M, 2.1 mmol) was concentrated under reduced pressure (30° C. water bath temperature) to ca. 0.5 mL volume. The residue was cooled to 0° C. (ice bath) and N,N-dimethylacetamide (5 mL) was added. 4-{[3-(4-hydroxy-phenylsulfanyl)-propyl]-[1,2,4]triazol-4-yl-amino}-benzonitrile (CAB02182, 140 mg, 0.40 mmol) was added to the colourless solution and the mixture was stirred for 18 hours at room temperature. Ethyl acetate (50 mL) and water (50 mL) were added to the solution, the organic layer was separated, washed with water (2×30 mL) and brine (20 mL), dried over sodium sulphate and concentrated under reduced pressure. The residue was dissolved in a small amount of acetone and precipitated by addition of hexane. The precipitate was filtered off and dried under high vacuum. Yield: 139 mg (81%) light yellow powder. ¹H-NMR (400 MHz, d₆-DMSO) δ=1.74 (tt, J=7.0, 7.0 Hz, 2H), 3.97 (t, J=7.0 Hz, 2H), 6.62 (d, J=9.0 Hz, 2H), 7.22 (d, J=8.6 Hz, 2H), 7.43 (d, J=8.6 Hz, 2H), 7.74 (d, J=9.0 Hz, 2H), 8.03 (s, 2H, —NH₂), 9.00 (s, 2H). LRMS (FAB+): 431.1 (100, [M+H]⁺).

### 4-[(4-Bromo-butyl)-[1,2,4]triazol-4-yl-amino]-benzonitrile (CAB03001,STX602)

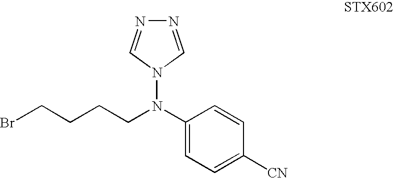

Sodium hydride (60%, 240 mg, 6.0 mmol) was added to a solution of 4-([1,2,4]triazol-4-ylamino)-benzonitrile (926 mg, 5.0 mmol) in DMSO (25 mL) at r.t. The mixture was stirred for 1 hour at this temperature and 1,4-dibromobutane (5 mL, ca 42 mmol) was added. The reaction mixture was stirred overnight and ethyl acetate (100 mL) was added. The mixture was transferred into a separation funnel and extracted with water (twice 100 mL) and brine (20 mL). The organic layer was dried over sodium sulphate and concentrated under reduced pressure (water bath temperature <30° C.). The resulting orange oil was mixed with diethyl ether (100 mL) and filtered through a layer of silica (ca. 5 cm). The silica was washed with more diethyl ether (ca. 100 mL) to remove the excess of 1,4-dibromobutane; the crude product was washed from the silica with acetone (120 mL). The acetone solution was concentrated under reduced pressure and the residue was purified by column-chromatography (eluent: ethyl acetate) to give the title compound as a white solid. Yield: 984 mg (61%). ¹H-NMR (400 MHz, d6-DMSO) δ=1.53-1.60 (m, 2H), 1.87-1.94 (m, 2H), 3.56 (t, J=6.6 Hz, 2H), 3.87 (t, J=7.4 Hz, 2H), 6.64 (d, J=9.0 Hz, 2H), 7.73 (d, J=9.0 Hz, 2H), 8.99 (s, 2H). LRMS (FAB+): 320.0 (100, [M+H]⁺)

### 4-{[4-(4-Hydroxy-phenylsulfanyl)-butyl]-[1,2,4]triazol-4-yl-amino}-benzonitrile (CAB03007)

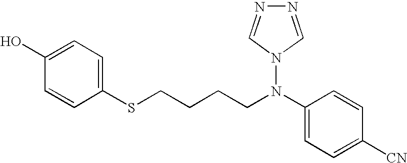

Potassium carbonate (500 mg) was added to a solution of 4-[(2-bromo-butyl)-[1,2,4]triazol-4-yl-amino]-benzonitrile (CAB03001, 384 mg, 1.2 mmol) and 4-hydroxy-thiophenol (227 mg, 1.8 mmol) in DMF (20 mL). The mixture was stirred for 18 hours at r.t., diluted with ethyl acetate (100 mL) and extracted with water (two times 30 mL) and brine (30 mL). The organic layer was dried over sodium sulphate and concentrated under reduced pressure. The residue was purified by column chromatography (eluent: ethyl acetate, R_{f}: 0.30). The resulting oil was dissolved in a small amount of ethyl acetate and the product was precipitated by addition of hexane. Yield: 289 mg (66%). ¹H-NMR (400 MHz, d6-DMSO) δ=1.48-1.64 (m, 4H) 2.81 (t, J=6.6 Hz, 2H), 3.81 (t, J=6.8 Hz, 2H), 6.62 (d, J=9.0 Hz, 2H), 6.72 (d, J=8.6 Hz, 2H), 7.19 (d, J=9.0 Hz, 2H), 7.72 (d, J=9.0, 2H), 8.94 (s, 2H), 9.56 (s, 1H, —OH). LRMS (FAB+): 366.1 (100, [M+H]⁺).

### 4-[(5-Bromo-pentyl)-[1,2,4]triazol-4-yl-amino]-benzonitrile (CAB03005)

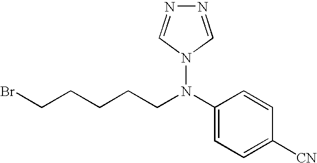

Sodium hydride (60%, 240 mg, 6.0 mmol) was added to a solution of 4-([1,2,4]triazol-4-ylamino)-benzonitrile (926 mg, 5.0 mmol) in DMSO (25 mL) at r.t. The mixture was stirred for 1 hour at this temperature and 1,5-dibromopentane (5 mL) was added. The reaction mixture was stirred overnight and ethyl acetate (100 mL) was added. The mixture was transferred into a separation funnel and extracted with water (twice 100 mL) and brine (20 mL). The organic layer was dried over sodium sulphate and concentrated under reduced pressure (water bath temperature <30° C.). The resulting orange oil was mixed with diethyl ether (100 mL) and filtered through a layer of silica (ca. 5 cm). The silica was washed with more diethyl ether (ca. 100 mL) to remove the excess of 1,5-dibromopentane. The crude product was washed from the silica with acetone (120 mL). The acetone solution was concentrated under reduced pressure and the residue was purified by column-chromatography (eluent: ethyl acetate) to give the title compound as a white solid. Yield: 1.01 g (60%). ¹H-NMR (400 MHz, d6-DMSO) δ=1.40-1.52 (m, 2H), 1.75-1.85 (m, 2H), 3.52 (t, J=6.6 Hz, 2H), 3.78-3.89 (m, 2H), 6.64 (d, J=9.0 Hz, 2H), 7.72 (d, J=9.0 Hz, 2H), 8.97 (s, 2H). LRMS (FAB+): 334.1 (100, [M+H]⁺).

### 4-{[5-(4-Hydroxy-phenylsulfanyl)-pentyl]-[1,2,4]triazol-4-yl-amino}-benzonitrile (CAB03014, STX698)

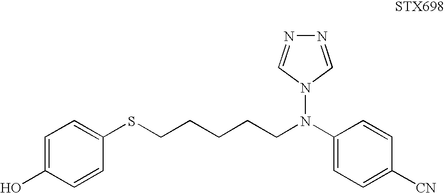

A mixture of 4-[(2-Bromo-pentyl)-[1,2,4]triazol-4-yl-amino]-benzonitrile (CAB03005, 500 mg, 1.5 mmol), 4-hydroxy-thiophenol (378 mg, 3.0 mmol) and potassium carbonate (414 mg, 3.0 mmol) in DMF (10 mL) was stirred for 12 hours at room temperature. The mixture was transferred into a separation funnel and ethyl acetate (50 mL) and water (50 mL) were added. The organic layer was separated, washed with brine (30 mL), dried over sodium sulphate and concentrated under reduced pressure. The residue was purified by column chromatography (eluent: ethyl acetate, R_{f}: 0.43). The resulting yellow oil was dissolved in ethyl acetate and precipitated by addition of hexane to give an light yellow powder. Yield: 338 mg (59%). ¹H-NMR (400 MHz, d6-DMSO) δ=1.40-1.54 (m, 6H), 2.77 (t, J=6.8 Hz, 2H), 3.79 (t, J=6.6 Hz, 2H), 6.61 (d, J=9.0 Hz, 2H), 6.72 (d, J=9.0 Hz, 2H), 7.20 (d, J=9.0 Hz, 2H), 7.72 (d, J=9.0 Hz, 2H), 8.96 (s, 2H), 9.55 (s, 1H. —OH). ¹³C-NMR (100 MHz, d6-DMSO) δ=25.66, 26.67, 29.35, 35.56, 53.86, 103.06, 113.89, 116.80, 119.84, 124.54, 133.39, 134.47, 144.09, 151.89, 157.26. LRMS (FAB+): 380.0 (100, [M+H]⁺). HRMS (FAB+) 380.15355 C₂₀H₂₂N₅OS requires 380.154507.

### Sulfamic acid 4-{5-[(4-cyano-phenyl)-[1,2,4]triazol-4-yl-amino]-pentylsulfanyl}-phenyl ester (CAB03025, STX699)

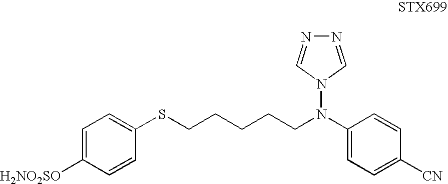

Sulphamoyl chloride solution in toluene (3 mL, 0.7 M, 2.1 mmol) was concentrated under reduced pressure (30° C. water bath temperature) to ca. 0.5 mL volume. The residue was cooled to 0° C. (ice bath) and N,N-dimethylacetamide (5 mL) was added. 4-{[3-(4-hydroxy-phenylsulfanyl)-pentyl]-[1,2,4]triazol-4-yl-amino}-benzonitrile (CAB03014, 95 mg, 0.25 mmol) was added to the colourless solution and the mixture was stirred for 18 hours at room temperature. Ethyl acetate (50 mL) and water (50 mL) were added to the solution, the organic layer was separated, washed with water (two times 30 mL) and brine (20 mL), dried over sodium sulphate and concentrated under reduced pressure. The residue was dissolved in a small amount of acetone and precipitated by addition of hexane. The precipitate was filtered off and dried under high vacuum. Yield: 103 mg (90%) light yellow powder. ¹H-NMR (270 MHz, d6-DMSO) δ=1.35-1.63 (m, 6H), 2.95 (t, J=7.0 Hz, 2H), 3.75-3.85 (m, 2H), 6.62 (d, J=9.0 Hz, 2H), 7.20 (d, J=9.0 Hz, 2H), 7.37 (d, J=9.0 Hz, 2H), 7.71 (d, J=9.0 Hz, 2H), 7.99 (s, 2H, —NH₂), 8.96 (s, 2H). LRMS (FAB+): 459.1 (100, [M+H]⁺).

### 4-[(10-Bromo-decyl)-[1,2,4]triazol-4-yl-amino]-benzonitrile (CAB03008)

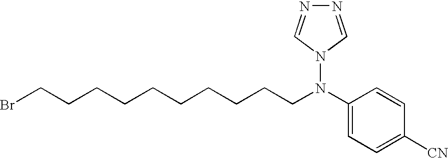

Sodium hydride (60%, 240 mg, 6.0 mmol) was added to a solution of 4-([1,2,4]triazol-4-ylamino)-benzonitrile (926 mg, 5.0 mmol) in DMSO (25 mL) at r.t. The mixture was stirred for 1 hour at this temperature and 1,10-dibromodecane (5 mL) was added. The reaction mixture was stirred overnight and ethyl acetate (100 mL) was added. The mixture was transferred into a separation funnel and extracted with water (twice 100 mL) and brine (20 mL). The organic layer was dried over sodium sulphate and concentrated under reduced pressure (water bath temperature <30° C.). The resulting orange oil was mixed with diethyl ether (100 mL) and filtered through a layer of silica (ca. 5 cm). The silica was washed with more diethyl ether (ca. 100 mL) to remove the excess of 1,10-dibromodecane. The crude product was washed from the silica with acetone (120 mL). The acetone solution was concentrated under reduced pressure and the residue was purified by column-chromatography (eluent: ethyl acetate) to give the title compound as a pale yellow solid. Yield: 1.32 g (65%). ¹H-NMR (400 MHz, d₆-DMSO) δ=1.16-1.40 (m, 14H), 1.76 (tt, J=7.0, 7.0 Hz, 2H), 3.50 (t, J=6.6 Hz, 2H), 3.79 (t, J=7.0 Hz, 2H), 6.61 (d, J=9.0 Hz, 2H), 7.70 (d, J=9.0 Hz, 2H), 8.95 (s, 2H).

### 4-{[10-(4-Hydroxy-phenylsulfanyl)-decyl]-[1,2,4]triazol-4-yl-amino}-benzonitrile (CAB03011, STX625)

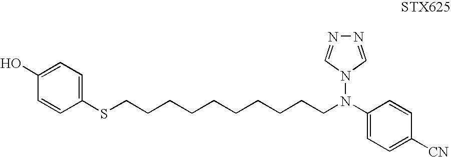

A mixture of 4-[(2-Bromo-decyl)-[1,2,4]triazol-4-yl-amino]-benzonitrile (CAB03008, 607 mg, 1.5 mmol), 4-hydroxy-thiophenol (378 mg, 3.0 mmol) and potassium carbonate (414 mg, 3.0 mmol) in DMF (10 mL) was stirred for 12 hours at room temperature. The mixture was transferred into a separation funnel and ethyl acetate (50 mL) and water (50 mL) were added. The organic layer was separated, washed with brine (30 mL), dried over sodium sulphate and concentrated under reduced pressure. The residue was purified by column chromatography (eluent: ethyl acetate, R_{f}: 0.71). The resulting yellow solid was dissolved in acetone and precipitated by addition of hexane to give a light yellow powder. Yield: 401 mg (59%). ¹H-NMR (400 MHz, d6-DMSO) δ=1.16-1.52 (m, 16H), 2.76 (t, J=7.2 Hz, 2H), 3.80 (t, J=7.2 Hz, 2H), 6.62 (d, J=9.0 Hz, 2H), 6.72 (d, J=8.6 Hz, 2H), 7.19 (d, J=8.6 Hz, 2H), 7.72 (d, J=9.0 Hz, 2H), 8.97 (s, 2H), 9.53 (s, 1H, —OH). LRMS (FAB+): 450.2 (100, [M+H]⁺). HRMS (FAB+) 450.23208 C₂₅H₃₂N₅OS requires 450.232758.

### Sulfamic acid 4-{10-[(4-cyano-phenyl)-[1,2,4]triazol-4-yl-amino]-decylsulfanyl}-phenyl ester (CAB03012, STX655)

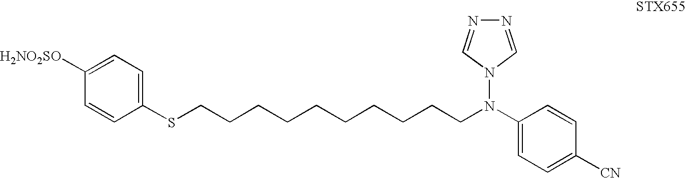

Sulphamoyl chloride solution in toluene (3 mL, 0.7 M, 2.1 mmol) was concentrated under reduced pressure (30° C. water bath temperature) to ca. 0.5 mL volume. The residue was cooled to 0° C. (ice bath) and N,N-dimethylacetamide (5 mL) was added. 4-{[3-(4-hydroxy-phenylsulfanyl)-decyl]-[1,2,4]triazol-4-yl-amino}-benzonitrile (CAB03011, 100 mg, 0.22 mmol) was added to the colourless solution and the mixture was stirred for 18 hours at room temperature. Ethyl acetate (50 mL) and water (50 mL) were added to the solution, the organic layer was separated, washed with water (two times 30 mL) and brine (20 mL), dried over sodium sulphate and concentrated under reduced pressure. The residue was dissolved in a small amount of acetone and precipitated by addition of diethyl ether and hexane. The precipitate was filtered off and dried under high vacuum. Yield: 104 mg (88%) light yellow powder. ¹H-NMR (400 MHz, d6-DMSO) δ=1.20-1.41 (m, 14H), 1.56 (tt, J=7.0 Hz, 2H), 2.96 (t, J=7.0 Hz, 2H), 3.80 (t, J=7.0 Hz, 2H), 6.62 (d, J=9.0 Hz, 2H), 7.21 (d, J=9.0 Hz, 2H), 7.37 (d, J=9.0 Hz, 2H), 7.71 (d, J=9.0 Hz, 2H), 7.99 (s, 2H, —NH₂), 8.95 (s, 2H). LRMS (FAB+): 529.2 (100, [M+H]⁺). HRMS (FAB+) 529.20425 C₂₅H₃₃N₆O₃S₂ requires 529.205558.

### 4-Benzyloxy-3,5-dichloro-benzoic acid methyl ester (CAB02115)

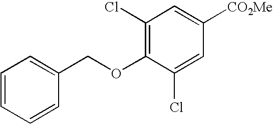

A mixture of 3,5-dichloro-4-hydroxy benzoic acid methyl ester (5.525 g, 25 mmol), benzyl bromide (5.13 g, 30 mmol) and potassium carbonate (6.91 g, 50 mmol) in N,N-dimethylformamide (100 mL) was stirred overnight at room temperature. The reaction mixture was poured into crushed ice (ca. 300 g) and the product was extracted with ethyl acetate (2×100 mL). The combined organic layers were dried over sodium sulphate and concentrated under reduced pressure. The solid residue was recrystallised from ethyl acetate and hexane. Yield: 7.47 g (96%) fine white needles. ¹H-NMR (400 MHz, CDCl₃) δ=3.93 (s, 3H, —OCH₃), 5.12 (s, 2H, —CH₂Ph), 7.35-7.44 (m, 3H), 7.54-7.58 (m, 2H), 8.01 (s, 2H). ¹³C-NMR (100 MHz, CDCl₃) δ=52.66, 75.12, 127.18, 128.41, 128.48, 129.79, 130.13, 135.57, 154.57, 164.39.

### (4-Benzyloxy-3,5-dichloro-phenyl)-methanol (CAB02117)

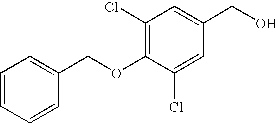

A solution of 4-benzyloxy-3,5-dichloro-benzoic acid methyl ester (CAB02115, 7.20 g, 23.14 mmol) in THF (20 mL) was added slowly with a syringe to a suspension of lithium aluminium hydride (1.50 g, 39.5 mmol) in THF (40 mL). The reaction mixture was stirred for 30 minutes at room temperature and then carefully quenched by addition of 2N sodium hydroxide solution in water. After 20 minutes stirring the colour of the mixture turned from grey to white. The white precipitate was filtered off, the filtrate was dried over sodium sulphate and concentrated under reduced pressure. The residue was recrystallised from dichloromethane/hexane. Yield: 5.83 g (89%) white powder. ¹H-NMR (400 MHz, CDCl₃) δ=1.85 (t, J=5.9 Hz, 1H, —OH), 4.63 (d, J=5.9 Hz, 2H), 5.04 (s, 2H), 7.33 (s, 2H), 7.35-7.44 (m, 3H), 7.55-7.59 (m, 2H). ¹³C-NMR (100 MHz, CDCl₃) δ=64.05, 75.03, 127.36, 128.70, 128.75, 129.91, 136.39, 138.64, 150.32.

### 2-Benzyloxy-5-bromomethyl-1,3-dichloro-benzene (CAB02118)

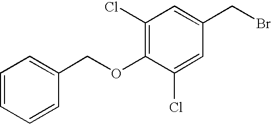

Phosphorous tribromide (2 mL) was added to a solution of (4-benzyloxy-3,5-dichloro-phenyl)-methanol (CAB02117, 5.50 g, 19.4 mmol) in dichloromethane (80 mL) at 0° C. The mixture was stirred at this temperature for 2 h, diluted with diethyl ether (150 mL), transferred into a separation funnel and washed with water (2×50 mL) and brine (50 mL). The organic layer was dried over magnesium sulphate and concentrated under reduced pressure. The residue was recrystallised from dichloromethane/hexane. ¹H-NMR (400 MHz, CDCl₃) δ=4.39 (s, 2H, —CH₂Br), 5.05 (s, 2H, —CH₂Ph), 7.37 (s, 2H), 7.38-7.44 (m, 3H), 7.54-7.58 (m, 2H). ¹³C-NMR (100 MHz, CDCl₃) δ=31.48, 75.39, 128.73, 129.68, 129.70, 129.72, 130.06, 135.46, 136.27, 151.23.

### 4-[(4-Benzyloxy-3,5-dichloro-benzyl)-[1,2,4]triazol-4-yl-amino]-benzonitrile (CAB02119, STX434)

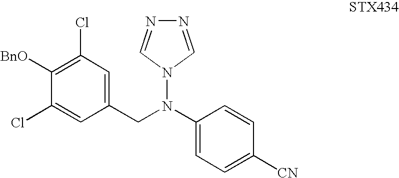

Sodium hydride (60%, 200 mg, 5.0 mmol) was added to a solution of 4-([1,2,4]triazol-4-ylamino)-benzonitrile (926 mg, 5.0 mmol) in DMF (20 mL) at r.t. The mixture was stirred for 1 h at this temperature and 2-benzyloxy-5-bromomethyl-1,3-dichloro-benzene (CAB02118, 1.73 g, 5.0 mmol) was added. The reaction mixture was stirred overnight and ethyl acetate (75 mL) was added. The mixture was transferred into a separation funnel and washed with water (2×100 mL) and brine (20 mL). The organic layer was dried over sodium sulphate and concentrated under reduced pressure. The residue was suspended in 2-propanol (20 mL) and heated to reflux for 5 minutes. The white solid was filtered off after cooling to room temperature and dried under high vacuum. Yield: 1.76 g (78%).

¹H-NMR (400 MHz, d6-DMSO) δ=5.00 (s, 2H), 5.07 (s, 2H), 6.74 (d, J=9.0 Hz, 2H), 7.36-7.44 (m, 3H), 7.47-7.50 (m, 2H), 7.50 (s, 2H), 7.77 (d, J=9.0 Hz, 2H), 8.92 (s, 2H). ¹³C-NMR (100 MHz, CDCl₃) δ=55.94, 74.59, 102.96, 113.61, 118.80, 128.21, 128.27, 128.63, 128.81, 133.29, 133.75, 135.69, 143.13, 149.62, 150.88. LRMS (FAB+): 450.1 (100, [M+H]⁺).

### 4-[(3,5-Dichloro-4-hydroxy-benzyl)-[1,2,4]triazol-4-yl-amino]-benzonitrile (CAB02120, STX435)

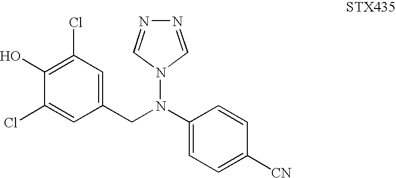

Palladium on charcoal (100 mg, 10% Pd) was added to a solution of 4-[(4-benzyloxy-3,5-dichloro-benzyl)-[1,2,4]triazol-4-yl-amino]-benzonitrile (CAB02119, 1.13 g, 2.50 mmol) in MeOH/THF/EtOAc (30 mL/30 mL/40 mL). The mixture was stirred under hydrogen atmosphere (balloon) for 18 h at room temperature. The reaction mixture was filtered through celite and the clear colourless filtrate was concentrated under reduced pressure. The residue was suspended in 2-propanol (20 mL) and heated to reflux for 5 minutes. The white solid was filtered off after cooling to room temperature and dried under high vacuum. Yield: 306 mg (34%). ¹H-NMR (400 MHz, d6-DMSO) δ=4.97 (s, 2H, —CH₂Ar), 6.76 (d, J=9.0 Hz, 2H), 7.31 (s, 2H), 7.77 (d, J=9.0 Hz, 2H), 8.85 (s, 2H), 10.29 (s, 1H, —OH). ¹³C-NMR (100 MHz, CDCl₃) δ=56.57, 103.76, 114.55, 119.69, 122.89, 128.14, 129.46, 134.58, 144.03, 149.46, 151.85. LRMS (FAB+): 360.0 (100, [M+H]⁺).

### 4-Benzyloxy-3-chloro-benzoic acid methyl ester (CAB02121)

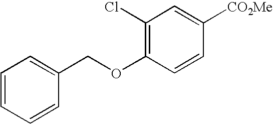

A mixture of 3-chloro-4-hydroxy benzoic acid methyl ester (4.665 g, 25 mmol), benzyl bromide (5.13 g, 30 mmol) and potassium carbonate (6.91 g, 50 mmol) in N,N-dimethylformamide (50 mL) was stirred overnight at room temperature. The reaction mixture was poured onto crushed ice (ca. 300 g) and the product was extracted with ethyl acetate (2×100 mL). The combined organic layers were dried over sodium sulphate and concentrated under reduced pressure. The residue was dissolved in dichloromethane and precipitated by addition of hexane. Yield: 6.47 g (94%) white powder. ¹H-NMR (400 MHz, CDCl₃) δ=3.90 (s, 3H, —OCH₃), 5.23 (s, 2H, —CH₂Ar), 6.99 (d, J=9.0 Hz, 1H), 7.31-7.48 (m, 5H), 7.89 (dd, J=9.0, 2.3 Hz, 1H), 8.08 (d, J=2.3 Hz, 1H).

### (4-Benzyloxy-3-chloro-phenyl)-methanol (CAB02127)

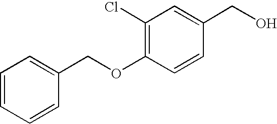

A solution of 4-benzyloxy-3-chloro-benzoic acid methyl ester (CAB02121, 4.15 g, 15.0 mmol) in THF (30 mL) was added slowly with a syringe to a suspension of lithium aluminium hydride (1.0 g, 26.3 mmol) in THF (30 mL). The reaction mixture was stirred for 30 minutes at room temperature and then carefully quenched by addition of 2N sodium hydroxide solution in water. After 20 minutes stirring the colour of the mixture turned from grey to white. The white precipitate was filtered off, the filtrate was dried over sodium sulphate and concentrated under reduced pressure to give an colourless oil, which was used without further purification. Yield: 3.61 g (97%). ¹H-NMR (400 MHz, CDCl₃) δ=2.12 (s, 1H, —OH), 4.56 (s, 2H), 5.16 (s, 2H), 6.93 (d, J=8.6 Hz, 1H), 7.14 (dd, J=8.6, 2.3 Hz, 1H), 7.31-7.48 (m, 6H).

### 1-Benzyloxy-4-bromomethyl-2-chloro-benzene (CAB02128)

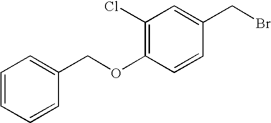

Phosphorous tribromide (2 mL) was added to a solution of (4-benzyloxy-3-chloro-phenyl)-methanol (CAB02127, 3.40 g, 13.7 mmol) in dichloromethane (50 mL) at 0° C. The mixture was stirred at this temperature for 1 h, diluted with diethyl ether (100 mL), transferred into a separation funnel and washed with water (50 mL) and brine (50 mL). The organic layer was dried over magnesium sulphate and concentrated under reduced pressure to give a white, analytical pure solid. Yield: 4.27 g (100%).

¹H-NMR (400 MHz, CDCl₃) δ=4.44 (s, 2H, —CH₂Br), 5.17 (s, 2H), 6.92 (d, J=8.6 Hz, 1H), 7.21 (dd, J=8.6, 2.3 Hz, 1H), 7.31-7.48 (m, 6H).

### 4-[(4-Benzyloxy-3-chloro-benzyl)-[1,2,4]triazol-4-yl-amino]-benzonitrile (CAB02129, STX446)

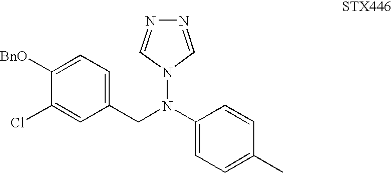

Sodium hydride (60%, 200 mg, 5.0 mmol) was added to a solution of 4-([1,2,4]triazol-4-ylamino)-benzonitrile (926 mg, 5.0 mmol) in DMF (20 mL) at r.t. The mixture was stirred for 1 hour at this temperature and 1-benzyloxy-4-bromomethyl-2-chloro-benzene (CAB02128, 1.56 g, 5.0 mmol) was added. The reaction mixture was stirred overnight and ethyl acetate (75 mL) was added. The mixture was transferred into a separation funnel and washed with water (two times 100 mL) and brine (20 mL). The organic layer was dried over sodium sulphate and concentrated under reduced pressure. The residue was suspended in 2-propanol (20 mL) and heated to reflux for 5 minutes. The white solid was filtered off after cooling to room temperature and dried under high vacuum. Yield: 1.38 g (66%). ¹H-NMR (400 MHz, d6-DMSO) δ=4.99 (s, 2H), 5.17 (s, 2H), 6.76 (d, J=9.0 Hz, 2H), 7.16 (d, J—8.6 Hz, 1H), 7.20 (dd, J=8.6, 2.2 Hz, 1H), 7.32-7.47 (m, 6H), 7.76 (d, J=9.0 Hz, 2H), 8.81 (s, 2H). LRMS (FAB+): 416.1 (100, [M+H]⁺).

### 4-[(3-Chloro-4-hydroxy-benzyl)-[1,2, 4]triazol-4-yl-amino]-benzonitrile (CAB02130, STX447)

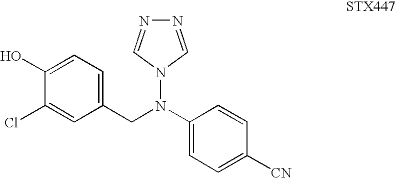

Palladium on charcoal (50 mg, 10% Pd) was added to a solution of 4-[(4-benzyloxy-3-chloro-benzyl)-[1,2,4]triazol-4-yl-amino]-benzonitrile (CAB02129, 1.04 g, 2.50 mmol) in MeOH/THF/EtOAc (25 mL/25 mL/25 mL). The mixture was stirred under hydrogen atmosphere (balloon) for 18 hours at room temperature. The reaction mixture was filtered through celite and the clear colourless filtrate was concentrated under reduced pressure. The residue was suspended in 2-propanol (20 mL) and heated to reflux for 5 minutes. The white solid was filtered off after cooling to room temperature and dried under high vacuum. Yield: 484 mg (59%). ¹H-NMR (400 MHz, d6-DMSO) δ=4.93 (s, 2H), 6.76 (d, J=9.0 Hz, 2H), 6.86 (d, J=8.6 Hz, 1H), 7.02 (dd, J=8.2, 2.0 Hz, 1H), 7.27 (d, J=2.0 Hz, 1H), 7.76 (d, J=9.0 Hz, 2H), 8.77 (s, 2H), 10.29 (s, 1H, —OH). ¹³C-NMR (100 MHz, d₆-DMSO) δ=56.87, 103.55, 114.48, 117.28, 119.73, 120.25, 126.72, 129.19, 130.91, 134.56, 144.02, 152.03, 153.50. LRMS (FAB+): 325.0 (100, [M+H]⁺).

### Sulfamic acid 2-chloro-4-{[(4-cyano-phenyl)-[1,2,4]triazol-4-yl-amino]-methyl}-phenyl ester (CAB03015, STX694)

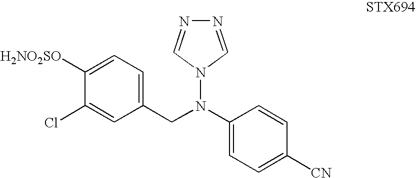

Sulphamoyl chloride solution in toluene (3 mL, 0.7 M, 2.1 mmol) was concentrated under reduced pressure (30° C. water bath temperature) to ca. 0.5 mL volume. The residue was cooled to 0° C. (ice bath) and N,N-dimethylacetamide (5 mL) was added. 4-[(3-Chloro-4-hydroxy-benzyl)-[1,2,4]triazol-4-yl-amino]-benzonitrile (CAB02130, 163 mg, 0.50 mmol) was added to the colourless solution and the mixture was stirred for 18 h at room temperature. Ethyl acetate (50 mL) and water (30 mL) were added to the solution, the organic layer was separated, washed with water (2×30 mL) and brine (20 mL), dried over sodium sulphate and concentrated under reduced pressure. The residue was dissolved in a small amount of acetone and precipitated by addition of hexane. The precipitate was filtered off and dried under high vacuum. Yield: 59 mg (29%) white powder. ¹H-NMR (400 MHz, d6-DMSO) δ=5.11 (s, 2H), 6.72 (d, J=9.0 Hz, 2H), 7.38 (dd, J=8.2, 2.0 Hz, 1H), 7.44 (d, J=8.2 Hz, 1H), 7.59 (d, J=2.0 Hz, 1H), 7.77 (d, J=9.0 Hz, 2H), 8.31 (s, 2H, —NH₂), 8.91 (s, 2H). LRMS (FAB+): 405.0 (100, [M+H]⁺). HRMS (FAB+) 405.05349 C₁₆H₁₄N₆O₃SCl requires 405.053663

### Benzoic acid 2-chloro-5-methyl-phenyl ester (CAB02124)

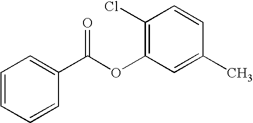

Benzoyl chloride (3.51 g, 25 mmol) was added dropwise to a solution of 2-chloro-5-methylphenol (3.92 g, 27.5 mmol) and triethylamine (5 mL) in dichloromethane (100 mL), The mixture was stirred for 18 hours at room temperature and concentrated under reduced pressure. Diethylether (200 mL) and water (100 mL) were added. The organic layer was separated and extracted with 2N NaOH (2×100 mL) and brine (50 mL), dried over sodium sulphate and concentrated under reduced pressure. The resulting colourless oil solidified in the freezer. Yield: 5.82 g (94%). ¹H-NMR (400 MHz, CDCl₃) δ=2.38 (s, 3H, —CH₃), 7.05 (d, J=8.2 Hz, 1H), 7.11 (s, 1H), 7.36 (d, J=8.2 Hz, 1H), 7.51-7.56 (m, 2H), 7.64-7.7.70 (m, 1H), 8.22-8.28 (m, 2H). ¹³C-NMR (100 MHz, CDCl₃) δ=21.41, 123.97, 124.58, 128.07, 128.85, 129.15, 130.07, 130.55, 134.01, 138.38, 146.98, 164.51.

### Benzoic acid 5-bromomethyl-2-chloro-phenyl ester (CAB02138)

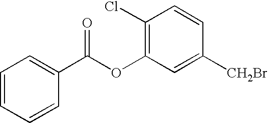

A mixture of benzoic acid 2-chloro-5-methyl-phenyl ester (CAB02124, 2.47 g, 10.0 mmol), N-bromo-succinimide (1.96 g, 11.0 mmol) and dibenzoyl peroxide (10 mg) in carbon tetrachloride (25 mL) was heated to reflux for 1 hour (TLC-control). After cooling to room temperature water (50 mL) and diethyl ether (100 mL) were added. The organic layer was separated, washed with brine (20 mL), dried over sodium sulphate and concentrated under reduced pressure. The residue was purified by column chromatography (EtOAc:hexane, 1:40, Rf: 0.21). Yield: 2.012 g (62%) colourless oil, which solidified after a few hours (contains ca. 10% benzoic acid 5-dibromomethyl-2-chloro-phenyl ester).

¹H-NMR (400 MHz, CDCl₃) δ=4.48 (s, 2H, —CH₂Br), 7.27 (dd, J=8.2, 2.0 Hz, 1H), 7.36 (d, J=2.0 Hz, 1H), 7.46 (d, J=8.2 Hz, 1H), 7.50-7.58 (m, 2H), 7.64-7.71 (m, 1H), 8.22-8.28 (m, 2H). LRMS (FAB+): 325.0 (81, [M+H]⁺), 327.0 (100, [M+H]⁺).

### Benzoic acid 2-chloro-5-{[(4-cyano-phenyl)-[1,2,4]triazol-4-yl-amino]methyl}-phenyl ester (CAB02139)

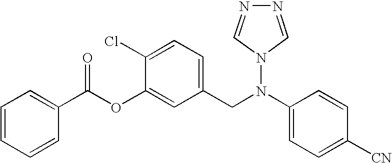

Sodium hydride (60%, 200 mg, 5.0 mmol) was added to a solution of 4-([1,2,4]triazol-4-ylamino)-benzonitrile (926 mg, 5.0 mmol) in DMF (20 mL) at r.t. The mixture was stirred for 1 hour at this temperature and benzoic acid 5-bromomethyl-2-chloro-phenyl ester (CAB02138, 1.63 g, 5.0 mmol) was added. The reaction mixture was stirred overnight and ethyl acetate (75 mL) was added. The mixture was transferred into a separation funnel and washed with water (2×50 mL) and brine (20 mL). The organic layer was dried over sodium sulphate and concentrated under reduced pressure. The residue was purified by column chromatography (EtOAc, Rf:0.31) to give a white solid. Yield: 1.773 g (82%). ¹H-NMR (400 MHz, CDCl₃) δ=4.93 (s, 2H), 6.66 (d, 9.0 Hz, 2H), 7.06 (dd, J=8.2, 2.0 Hz, 1H), 7.27 (d, J=2.0 Hz, 1H), 7.45 (d, J=8.2 Hz, 1H), 7.47-7.58 (m, 4H), 7.60-7.69 (m, 1H), 8.16-8.21 (m, 2H), 8.24 (s, 2H). LRMS (FAB+): 430.1 (100, [M+H]⁺).

### 4-[(4-Chloro-3-hydroxy-benzyl)-[1,2, 4]triazol-4-yl-amino]-benzonitrile (CAB02141, STX483)

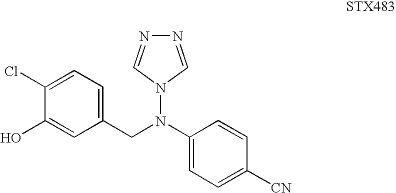

A solution of benzoic acid 2-chloro-5-{[(4-cyano-phenyl)-[1,2,4]triazol-4-yl-amino]methyl}-phenyl ester (CAB02139, 1.13 g, 2.63 mmol) and sodium methoxide (500 mg) in methanol (20 mL) and water (5 mL) was heated to reflux for 30 minutes. After cooling to room temperature most of the solvent was removed under reduced pressure and concentrated sodium bicarbonate solution (20 mL) and ethyl acetate (50 mL) were added. The organic layer was separated, dried over sodium sulphate and the solvent was removed under reduced pressure. The resulting white powder was refluxed in ethyl acetate (10 mL, product did not dissolve completely). After cooling to room temperature the product was filtered off and dried under high vacuum. Yield: 412 mg (48%) white powder. ¹H-NMR (400 MHz, d6-DMSO) δ=4.97 (s, 2H), 6.71-6.75 (m, 3H), 6.84 (d, J=2.0 Hz, 1H), 7.25 (d, J=8.2 Hz, 1H), 7.75 (d, J=9.0 Hz, 2H), 8.76 (s, 2H), 10.21 (s, 1H, —OH). LRMS (FAB+): 326.1 (100, [M+H]⁺).

### Sulfamic acid 2-chloro-5-{[(4-cyano-phenyl)-[1,2,4]triazol-4-yl-amino]-methyl}-phenyl ester (CAB02176, STX559)

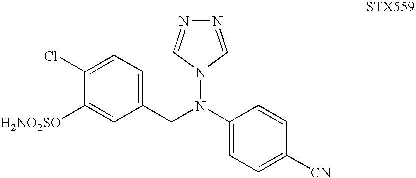

Sulphamoyl chloride solution in toluene (3 mL, 0.7 M, 2.1 mmol) was concentrated under reduced pressure (30° C. water bath temperature) to ca. 0.5 mL volume. The residue was cooled to 0° C. (ice bath) and N,N-dimethylacetamide (5 mL) was added. 4-[(4-Chloro-3-hydroxy-benzyl)-[1,2,4]triazol-4-yl-amino]-benzonitrile (CAB02141, 200 mg, 0.614 mmol) was added to the colourless solution and the mixture was stirred for 18 hours at room temperature. Ethyl acetate (50 mL) and water (30 mL) were added to the solution, the organic layer was separated, washed with water (2×30 mL) and brine (20 mL), dried over sodium sulphate and concentrated under reduced pressure. The residue was dissolved in a small amount of acetone and precipitated by addition of diethyl ether and hexane. The precipitate was filtered off and dried under high vacuum. Yield: 136 mg (55%) white powder. ¹H-NMR (400 MHz, d6-DMSO) δ=5.11 (s, 2H), 6.75 (d, J=9.0 Hz, 2H), 7.25 (dd, J=8.2, 2.0 Hz, 1H), 7.46 (d, J=2.0 Hz, 1H), 7.54 (d, J=8.2 Hz, 1H), 7.77 (d, J=9.0 Hz, 2H), 8.32 (s, 2H, —NH₂), 8.82 (s, 2H). LRMS (FAB+): 87.0 (100), 404.9 (40, [M+H]⁺). HRMS (FAB+): 405.05338 C₁₆H₁₄N₆O₃SCl requires 405.053663.

### 4-Benzyloxy-3-chloro-5-methoxy-benzoic acid benzyl ester (CAB02162)

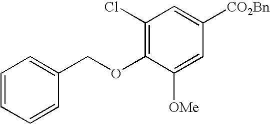

A mixture of 5-chloro-vanillic acid (4.05 g, 20 mmol), benzyl bromide (8.55 g, 50 mmol) and potassium carbonate (6.90 g, 50 mmol) in DMF (60 mL) was stirred at room temperature for 18 hours. The mixture was transferred into a separation funnel and ethyl acetate (100 mL) and water (100 mL) were added, the organic layer was separated, washed with water (2×50 mL) and brine (50 mL), dried over sodium sulphate and concentrated under reduced pressure. The residue was crystallised from dichloromethane/hexane. Yield: 7.34 g (96%) colourless needles. ¹H-NMR (400 MHz, CDCl₃) δ=3.92 (s, 3H, —OCH₃), 5.13 (s, 2H), 5.36 (s, 2H), 7.31-7.51 (m, 10H), 7.53 (d, J=2.0 Hz, 1H), 7.73 (d, J=2.0 Hz, 1H). LRMS (FAB+): 91.0 (100), 382.1 (15, [M+H]⁺).

### (4-Benzyloxy-3-chloro-5-methoxy-phenyl)-methanol (CAB02170)

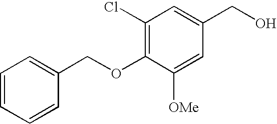

A solution of 4-benzyloxy-3-chloro-5-methoxy-benzoic acid benzyl ester (CAB02162, 3.83 g, 10.0 mmol) in THF (20 mL) was added dropwise with a syringe to a suspension of lithium aluminium hydride (500 mg, 13.15 mmol) in THF (30 mL). The mixture was stirred at room temperature for 30 minutes and 2N NaOH (5 mL) was added. The mixture was stirred for another hour, the aluminium salts were filtered off, the filtrate was dried over sodium sulphate and concentrated under reduced pressure. The resulting oil was heated to 120° C. under high vacuum in a kugelrohr-destillation-apparatus to remove benzyl alcohol for 10 h. The product was obtained as a light yellow oil and was used without any further purification. Yield: 2.70 g (97%). ¹H-NMR (400 MHz, CDCl₃) δ=2.09 (2, 1H, —OH), 3.86 (s, 3H, —OCH₃), 4.56 (s, 2H), 5.02 (s, 2H), 6.83 (d, J=2.0 Hz, 1H), 6.95 (d, J=2.0 Hz, 1H), 7.31-7.41 (m, 3H), 7.52-7.55 (m, 2H).

### 2-Benzyloxy-1-chloro-5-chloromethyl-3-methoxy-benzene (CAB02174)

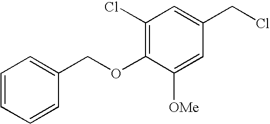

Thionyl chloride (2 mL) was added to solution of (4-benzyloxy-3-chloro-5-methoxy-phenyl)-methanol (CAB02170, 2.703 g, 9.7 mmol) in dichloromethane (10 mL). The solution was stirred for 1 h at room temperature, then diethyl ether (50 mL) and water (20 mL) were added. The organic layer was separated, washed with conc. sodium bicarbonate solution (10 mL), dried over sodium sulphate and concentrated under reduced pressure. The residue was dissolved in dichloromethane (5 mL) and precipitated by addition of hexane (ca. 50 mL). Yield: 2.730 g (95%) white powder. ¹H-NMR (400 MHz, CDCl₃) δ=3.89 (s, 3H, —OCH₃), 4.52 (s, 2H), 5.07 (s, 2H), 6.87 (d, J=2.0 Hz, 1H), 7.03 (d, J=2.0 Hz, 1H), 7.33-7.43 (m, 3H), 7.53-7.58 (m, 2H). LRMS (FAB+): 91.0 (100), 296.0 (17, [M+H]⁺).

### 4-[(4-Benzyloxy-3-chloro-5-methoxy-benzyl)-[1,2,4]triazol-4-yl-amino]-benzonitrile (CAB02177, STX599)

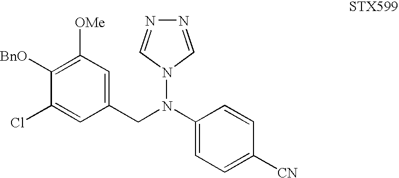

Sodium hydride (60%, 200 mg, 5.0 mmol) was added to a solution of 4-([1,2,4]triazol-4-ylamino)-benzonitrile (926 mg, 5.0 mmol) in DMF (20 mL) at r.t. The mixture was stirred for 1 h at this temperature and 2-benzyloxy-1-chloro-5-chloromethyl-3-methoxy-benzene (CAB02174, 1.49 g, 5.0 mmol) was added. The reaction mixture was stirred overnight and ethyl acetate (100 mL) and water (50 mL) were added. The organic layer was separated and washed with water (2×50 mL) and brine (30 mL). The organic layer was dried over sodium sulphate and concentrated under reduced pressure. The residue was suspended in 2-propanol (20 mL) and heated to reflux for 5 minutes. The white solid was filtered off after cooling to room temperature and dried under high vacuum. Yield: 1.76 g (79%).

¹H-NMR (400 MHz, d6-DMSO) δ=3.78 (s, 3H, —OCH₃), 4.80 (s, 2H), 5.05 (s, 2H), 6.52 (d, J=2.0 Hz, 1H), 6.67 (d, J=9.0 Hz, 2H), 6.89 (d, J=2.0 Hz, 1H), 7.32-7.40 (m, 3H), 7.46-7.50 (m, 2H), 7.61 (d, J=9.0, 2H), 8.75 (s, 2H). LRMS (FAB+): 91.0 (100), 446.0 (65, [M+H]⁺). HRMS (FAB+): 446.13840 C₂₄H₂₁N₅O₂Cl requires 446.138378.

### 4-[(3-chloro-4-hydroxy-5-methoxy-benzyl)-[1,2,4]triazol-4-yl-amino]-benzonitrile (CAB02179, STX600)

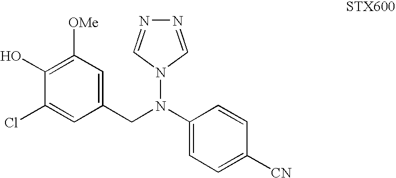

Palladium on charcoal (100 mg, 5% Pd) was added to a solution of 4-[(4-benzyloxy-3-chloro-5-methoxy-benzyl)-[1,2,4]triazol-4-yl-amino]-benzonitrile (CAB02177, 1.34 g, 3.0 mmol) in ethanol (60 mL) and THF (40 mL). The mixture was stirred under hydrogen atmosphere (balloon) for 24 h (TLC monitored), then filtered through celite and concentrated under reduced pressure. The residue was dried under high vacuum. Yield: 1.06 g (99%), white powder. ¹H-NMR (400 MHz, d6-DMSO) δ=3.77 (s, 3H, —OCH₃), 4.92 (s, 2H), 6.79 (d, J=9.0 Hz, 2H), 6.81 (d, J=1.8 Hz, 1H), 6.85 (d, J=1.8 Hz, 1H), 7.77 (d, J=9.0 Hz, 2H), 8.79 (s, 2H), 9.49 (s, 1H, —OH). LRMS (FAB+): 356.0 (100, [M+H]⁺). HRMS (FAB+): 356.09234 C₁₇H₁₅N₅O₂Cl requires 356.091428.

### Sulfamic acid 2-chloro-4-{[(4-cyano-phenyl)-[1,2,4]triazol-4-yl-amino]-methyl}-6-methoxy-phenyl ester (CAB02181, STX601)

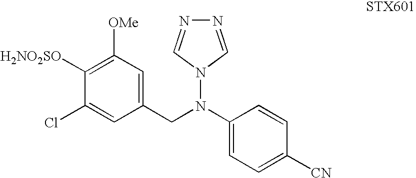

Sulphamoyl chloride solution in toluene (3 mL, 0.7 M, 2.1 mmol) was concentrated under reduced pressure (30° C. water bath temperature) to ca. 0.5 mL volume. The residue was cooled to 0° C. (ice bath) and N,N-dimethylacetamide (5 mL) was added. 4-[(3-Chloro-4-hydroxy-5-methoxy-benzyl)-[1,2,4]triazol-4-yl-amino]-benzonitrile (CAB02179, 212 mg, 0.596 mmol) was added to the colourless solution and the mixture was stirred for 18 hours at room temperature. Ethyl acetate (50 mL) and water (30 mL) were added to the solution, the organic layer was separated, washed with water (2×30 mL) and brine (20 mL), dried over sodium sulphate and concentrated under reduced pressure. The residue was dissolved in a small amount of acetone and precipitated by addition of hexane. The precipitate was filtered off and dried under high vacuum. Yield: 219 mg (84%) white powder. ¹H-NMR (400 MHz, d6-DMSO) δ=3.78 (s, 3H, —OCH₃), 6.74 (d, J=9.0 Hz, 2H), 7.04 (d, J=2.0 Hz, 1H), 7.07 (d, J=2.0 Hz, 1H), 7.77 (d, J=9.0 Hz, 2H), 7.98 (s, 2H, —NH₂), 8.91 (s, 2H). LRMS (FAB+): 435.0 (100, [M+H]⁺). HRMS (FAB+): 435.06476 C₁₇H₁₆N₆O₄SCl requires 435.064228.

### 4-Benzyloxy-3-fluoro-benzaldehyde (CAB03016)

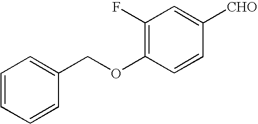

A mixture of 3-fluoro-4-hydroxy benzaldyde (4.90 g, 35.0 mmol), benzyl bromide (6.84 g, 40.0 mmol, 4.80 mL) and potassium carbonate (9.66 g, 70.0 mmol) in DMF (50 mL) was strirred for 18 h at room temperature. The reaction mixture was transferred into a separation funnel and ethyl acetate (100 mL) and water (50 mL) were added. The organic layer was separated, washed with water (2×50 mL) and brine (30 mL), dried over sodium sulphate and concentrated under reduced pressure. The white solid residue was recrystallised from dichloromethane/hexane. Yield: 7.65 g (95%). ¹H-NMR (400 MHz, CDCl₃) δ=5.24 (s, 2H), 7.12 (dd, J=8.2, 8.2 Hz, 1H), 7.34-7.48 (m, 5H), 7.59-7.66 (m, 2H), 9.85 (d, J=2.0 Hz, 1H, —CHO). LRMS (FAB+): 91 (100), 231.1 (100, [M+H]⁺).

### (4-Benzyloxy-3-fluoro-phenyl)-methanol (CAB03017)

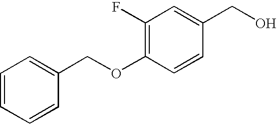

Sodium borohydride (500 mg, 13.2 mmol) was added to a solution of 4-benzyloxy-3-fluoro-benzaldehyde (CAB03016, 7.32 g, 31.8 mmol) in ethanol (40 mL) and THF (40 mL) at 0° C. The clear and colourless solution was allowed to warm to room temperature and stirred for 12 h at this temperature. Ethyl acetate (150 mL) and water (50 mL) were added to the solution, the organic layer was separated, washed with water (2×50 mL) and brine (50 mL), dried over sodium sulphate and concentrated under reduced pressure. The white solid residue was dissolved in dichloromethane and precipitated by addition of hexane. The white powder was filtered off and dried under high vacuum. Yield: 7.16 g (97%). ¹H-NMR (400 MHz, CDCl₃) δ=1.71 (s, 1H, —OH), 4.61 (s, 2H, —CH₂OH), 5.15 (s, 2H), 6.97 (dd, J=8.6, 8.6 Hz, 1H), 7.02 (dd, J=8.6, 1.9 Hz, 1H), 7.13 (dd, J=11.7, 1.9 Hz, 1H), 7.30-7.46 (m, 5H). LRMS (FAB+): 91 (100), 215.1 (40), 232.1 (100, [M]⁺).

### 1-Benzyloxy-4-chloromethyl-2-fluoro-benzene (CAB03018)

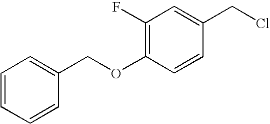

Thionyl chloride (5 mL) was added to solution of (4-benzyloxy-3-fluoro-phenyl)-methanol (CAB03017, 6.80 g, 29.28 mmol) in dichloromethane (50 mL). The solution was stirred for 1 h at room temperature and concentrated under reduced pressure. Then diethyl ether (100 mL) and water (20 mL) were added. The organic layer was separated, washed with conc. sodium bicarbonate solution (10 mL), dried over sodium sulphate and concentrated under reduced pressure. The residue was dissolved in dichloromethane (5 mL) and precipitated by addition of hexane (ca. 50 mL). The precipitate was filtered off and dried under high vacuum. Yield: 7.01 g (95%) white powder. ¹H-NMR (400 MHz, CDCl₃) δ=4.52 (s, 2H, —CH₂Cl), 5.15 (s, 2H), 6.96 (dd, J=8.2, 8.2 Hz, 1H), 7.04-7.06 (m, 1H), 7.15 (dd, J=11.7, 2.4 Hz, 1H), 7.31-7.45 (m. 5H). LRMS (FAB+): 91 (100), 215.0 (10), 250.0 (16, [M]⁺).

### 4-[(4-Benzyloxy-3-fluoro-benzyl)-[1,2,4]triazol-4-yl-amino]-benzonitrile (CAB03019, STX695)

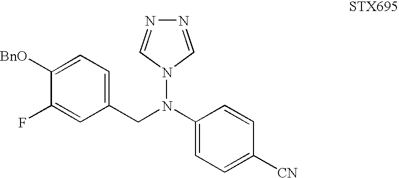

Sodium hydride (60%, 400 mg, 10.0 mmol) was added to a solution of 4-([1,2,4]triazol-4-ylamino)-benzonitrile (1.852 g, 10.0 mmol) in DMF (50 mL) at room temperature. The mixture was stirred for 1 h at this temperature and 1-benzyloxy-4-chloromethyl-2-fluoro-benzene (CAB03018, 2.51 g, 10.0 mmol) was added. The reaction mixture was stirred overnight and ethyl acetate (200 mL) and water (50 mL) were added. The mixture was transferred into a separation funnel and washed with water (2×50 mL) and brine (30 mL). The organic layer was dried over sodium sulphate and concentrated under reduced pressure. The residue was suspended in 2-propanol (40 mL) and heated to reflux for 5 minutes. The white solid was filtered off after cooling to room temperature and dried under high vacuum. Yield: 3.12 g (78%). ¹H-NMR (400 MHz, d6-DMSO) δ=4.97 (s, 2H), 5.12 (s, 2H), 6.73 (d, J=9.0 Hz, 2H), 7.01 (dd, J=8.2, 1.2 Hz, 1H), 7.16 (dd, J=8.6, 8.6 Hz 1H), 7.21 (dd, J=8.6, 2.4 Hz, 1H), 7.30-7.44 (m, 5H), 7.75 (d, J=9.0 Hz, 2H), 8.80 (s, 2H). LRMS (FAB+): 400.1 (100, [M+H]⁺).

HRMS (FAB+): 400.15800 C₂₃H₁₉N₅OF requires 400.157364.

### 4-[(3-Fluoro-4-hydroxy-benzyl)-[1,2,4]triazol-4-yl-amino]-benzonitrile (CAB03020, STX696)

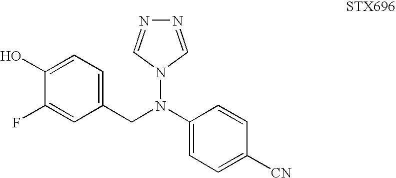

4-[(4-Benzyloxy-3-fluoro-benzyl)-[1,2,4]triazol-4-yl-amino]-benzonitrile (CAB03019, 2.83 g, 7.09 mmol) was dissolved in a mixture of ethanol (50 mL), THF (50 mL) and ethyl acetate (50 mL) by heating and palladium on charcoal (150 mg, 5% Pd) was added. The mixture was stirred under hydrogen-atmosphere (balloon) for 18 h, filtered through a 3 cm layer of celite and concentrated under reduced pressure. The residue was suspended in 2-propanol (30 mL), the mixture was heated to reflux for 5 minutes. After cooling room temperature the white precipitate was filtered off and dried under high vacuum. Yield: 2.13 g (97%). ¹H-NMR (400 MHz, d₆-DMSO) δ=4.93 (s, 2H), 6.75 (d, J=9.0 Hz, 2H), 6.82-6.89 (m, 2H), 7.07-7.12 (m, 1H), 7.76 (d, J=9.0 Hz, 2H), 8.77 (s, 2H), 9.95 (s, 1H, —OH).

### Sulfamic acid 4-{[(4-cyano-phenyl)-[1,2,4]triazol-4-yl-amino]-methyl}-2-fluoro-phenyl ester (CAB03021, STX700)

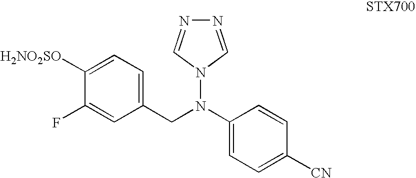

Sulphamoyl chloride solution in toluene (3 mL, 0.7 M, 2.1 mmol) was concentrated under reduced pressure (30° C. water bath temperature) to ca. 0.5 mL volume. The residue was cooled to 0° C. (ice bath) and N,N-dimethylacetamide (5 mL) was added. 4-[(3-Fluoro-4-hydroxy-benzyl)-[1,2,4]triazol-4-yl-amino]-benzonitrile (CAB03020, 220 mg, 0.71 mmol) was added to the colourless solution and the mixture was stirred for 4 h at room temperature. Ethyl acetate (50 mL) and water (30 mL) were added to the solution, the organic layer was separated, washed with water (2×25 mL) and brine (25 mL), dried over sodium sulphate and concentrated under reduced pressure. The residue was dissolved in acetone (5 mL) and precipitated by addition of diethyl ether. The precipitate was filtered off and dried under high vacuum. Yield: 228 mg (83%) white powder. ¹H-NMR (400 MHz, d6-DMSO) δ=5.11 (s, 2H), 6.71 (d, J=9.0 Hz, 2H), 7.23 (dd, 8.2, 1.2 Hz, 1H), 7.39 (dd, J=8.2, 8.2 Hz, 1H), 7.43 (dd, J=11.1, 2.1 Hz, 1H), 7.77 (d, J=9.0 Hz, 2H), 8.28 (s, 2H, —NH₂), 8.92 (s, 2H). LRMS (FAB+): 389.1 (100, [M+H]⁺). HRMS (FAB+): 389.08298 C₁₆H₁₄N₆O₃SF requires 389.083214.

### Benzoic acid 2-fluoro-5-methyl-phenyl ester (CAB02145)

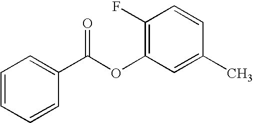

Benzoyl chloride (4.22 g, 30 mmol) was added dropwise with a syringe to a solution of 2-fluoro-5-methylphenol (3.784 g, 30 mmol) and triethylamine (5 mL) in dichloromethane (50 mL), The mixture was stirred for 18 h at room temperature and concentrated under reduced pressure. Diethyl ether (200 mL) and water (100 mL) were added, the organic layer was separated and extracted with 2N NaOH (2×30 mL) and brine (20 mL), dried over sodium sulphate and concentrated under reduced pressure to give a white solid. Yield: 6.601 g (96%). ¹H-NMR (400 MHz, CDCl₃) δ=2.36 (s, 3H, —CH₃), 7.01-7.12 (m, 3H), 7.49-7.55 (m, 2H), 7.63-7.68 (m, 1H), 8.19-8.23 (m, 2H). LRMS (FAB+): 231.1 (100, [M+H]⁺).

### Benzoic acid 5-bromomethyl-2-fluoro-phenyl ester (CAB02146)

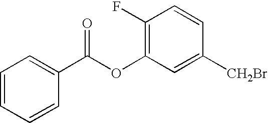

A mixture of benzoic acid 2-fluoro-5-methyl-phenyl ester (CAB02145, 2.47 g, 10.0 mmol), N-bromo-succinimide (1.96 g, 11.0 mmol) and dibenzoylperoxide (10 mg) in carbon tetrachloride (25 mL) was heated to reflux for 2 h (TLC monitored). After cooling to room temperature, water (50 mL) and diethyl ether (100 mL) were added. The organic layer was separated, washed with brine (20 mL), dried over sodium sulphate and concentrated under reduced pressure. The residue was purified by column chromatography (EtOAc:hexane, 1:25, Rf: 0.28). Yield: 1.80 g (58%), white solid. ¹H-NMR (400 MHz, CDCl₃) δ=4.48 (s, 2H, —CH₂Br), 7.18 (dd, J=9.8, 8.6 Hz, 1H), 7.28 (ddd, J=8.6, 4.3, 2.3 Hz, 1H), 7.34 (dd, J=7.0, 2.3 Hz, 1H), 7.50-7.55 (m, 2H), 7.64-7.69 (m, 1H), 8.18-8.23 (m, 2H). LRMS (FAB+): 229.1 (95), 309.0 (100, [M+H]⁺). Found: C, 54.1; H, 3.22%; C₁₄H₁₀BrFO₂ (309.13) requires C, 54.39; H, 3.26%.

### Benzoic acid 5-{[(4-cyano-phenyl)-[1,2,4]triazol-4-yl-amino]methyl}-2-fluoro-phenyl ester (CAB02147)

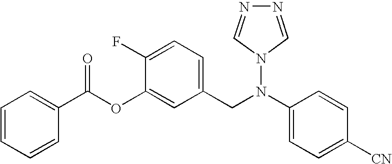

Sodium hydride (60%, 200 mg, 5.0 mmol) was added to a solution of 4-([1,2,4]triazol-4-ylamino)-benzonitrile (926 mg, 5.0 mmol) in DMF (20 mL) at r.t. The mixture was stirred for 1 h at this temperature and benzoic acid 5-bromomethyl-2-fluoro-phenyl ester (CAB02146, 1.55 g, 5.0 mmol) was added. The reaction mixture was stirred overnight and ethyl acetate (75 mL) and water (50 mL) were added. The mixture was transferred into a separation funnel and washed with water (2×50 mL) and brine (20 mL). The organic layer was dried over sodium sulphate and concentrated under reduced pressure.

The residue was purified by column chromatography (EtOAc, Rf: 0.32) to give a white solid. Yield: 1.16 g (56%). ¹H-NMR (400 MHz, CDCl₃) δ=4.91 (s, 2H), 6.67 (d, J=9.0 Hz, 2H), 7.07 (ddd, J=8.2, 4.3, 2.0 Hz, 1H), 7.18 (dd, J=9.4, 8.6 Hz, 1H), 7.24 (dd, J=7.0, 2.3 Hz, 1H), 7.49-7.56 (m, 2H), 7.58 (d, J=9.0 Hz, 2H), 7.62-7.70 (m, 1H), 8.15-8.20 (m, 2H), 8.21 (s, 2H). LRMS (FAB+): 414.2 (100, [M+H]⁺).

### 4-[(4-Fluoro-3-hydroxy-benzyl)-[1, 2, 4]triazol-4-yl-amino]-benzonitrile (CAB02154, STX488)

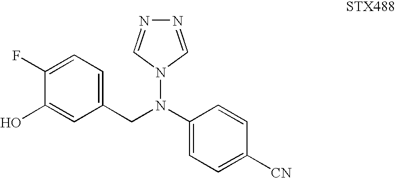

A solution of sodium hydroxide (250 mg, 6.25 mmol) in water (5 mL) was added to a solution of benzoic acid 5-{[(4-cyano-phenyl)-[1,2,4]triazol-4-yl-amino]methyl}-2-fluoro-phenyl ester (CAB02147, 958 mg, 2.32 mmol) in methanol (10 mL). The solution was heated to reflux for 5 minutes and concentrated under reduced pressure. Water (10 mL) was added and the milky suspension was neutralised (pH 7-8) with 2N hydrochloric acid. The white precipitate was filtered off, washed with a small amount of water (5 mL) and dried under high vacuum. Yield: 476 mg (66%). ¹H-NMR (400 MHz, d6-DMSO) δ=4.95 (s, 2H), 6.70 (ddd, J=11.4, 8.4, 2.4 Hz, 1H), 6.75 (d, J=9.0 Hz, 2H), 6.84 (dd, J=8.4, 2.4 Hz, 1H), 7.06 (dd, J=11.3, 8.4 Hz, 1H), 7.76 (d, J=9.0 Hz, 2H), 8.75 (s, 2H), 9.90 (s, 1H, —OH). LRMS (FAB+): 310.1 (100, [M+H]⁺).

### 4-Benzyloxy-3-trifluoromethyl-benzoic acid (CAB03046)

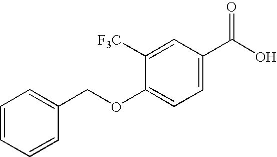

Sodium hydride (60%, 1.80 g, 45 mmol) was added to a solution of 4-fluoro-3-trifluoromethyl benzoic acid (4.162 g, 20 mmol) and benzyl alcohol (3.25 g, 30 mmol) in DMSO (50 mL). The mixture was stirred overnight at room temperature, poured into water (50 mL) and acidified with concentrated hydrochloric acid. The white precipitate was filtered off, dissolved in ethyl acetate (ca. 50 mL), dried over sodium sulphate and concentrated under reduced pressure. The residue was recrystallised from ethyl acetate/hexane. Yield: 4.252 g (72%). ¹H-NMR (400 MHz, CDCl₃) δ=5.37 (s, 2H), 7.32-7.48 (m, 6H), 8.12 (d, J=2.0 Hz, 1H), 8.18 (dd, J=8.6, 2.0 Hz, 1H), 13.16 (br s, 1H, —COOH). LRMS (FAB+): 91.1 (100), 297.1(18, [M+H]⁺).

### (4-Benzyloxy-3-trifluoromethyl-phenyl)-methanol (CAB03047)

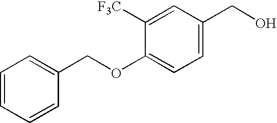

4-Benzyloxy-3-trifluoromethyl-benzoic acid (CAB03046, 3.555 g, 12 mmol) in THF (20 mL) was added dropwise to a suspension of lithium aluminium hydride (1.0 g, 26.3 mmol) in THF (20 mL). The mixture was stirred for another 30 minutes at room temperature and then quenched by addition of 2N NaOH (5 mL). The white preciptate was filtered off and washed with dichloromethane (1.00 mL), the filtrate was dried over sodium sulphate and concentrated under reduced pressure. The resulting oil was crystallised from diethyl ether/hexane. Yield: 3.31 g (98%). ¹H-NMR (400 MHz, CDCl₃) δ=1.72 (t, J=5.9 Hz, 1H, —OH), 4.66 (d, J=5.9 Hz, 2H, —CH₂OH), 5.21 (s, 2H, —CH₂Ph), 7.02 (d, J=8.2 Hz, 1H), 7.30-7.48 (m, 6H), 7.61 (d, J=2.3 Hz, 1H). LRMS (FAB+): 91.1 (100), 265.2 (45), 282.2 (40, [M+H]⁺).

### 1-Benzyloxy-4-chloromethyl-2-trifluoromethyl-benzene (CAB03050)

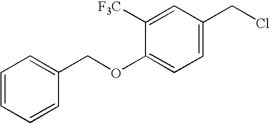

Thionyl chloride (2.0 mL) was added to solution of (4-benzyloxy-3-trifluoromethyl-phenyl)-methanol (CAB03047, 3.10 g, 11.0 mmol) in dichloromethane (20 mL). The solution was stirred for 1 h at room temperature and concentrated under reduced pressure. Then diethyl ether (100 mL) and water (20 mL) were added. The organic layer was separated, washed with conc. sodium bicarbonate solution (10 mL), dried over sodium sulphate and concentrated under reduced pressure. The resulting oil solidified after a few minutes and was dried under high vacuum. Yield: 3.25 g (98%). ¹H-NMR (400 MHz, CDCl₃) δ=4.57 (s, 2H, —CH₂Cl), 5.22 (s, 2H, —OCH₂Ph), 7.02 (d, J=8.5 Hz, 1H), 7.31-7.52 (m, 6H), 7.63 (d, J=2.0 Hz, 1H). LRMS (FAB+): 91.1 (100), 265.2 (8), 300.1 (10, [M]⁺).

### 4-[(4-Benzyloxy-3-trifluoromethyl-benzyl)-[1,2,4]triazol-4-yl-amino]-benzonitrile (CAB03054, STX719)

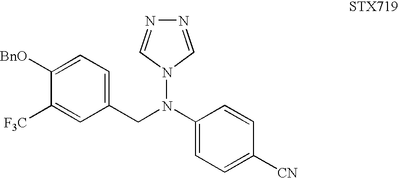

Sodium hydride (60%, 200 mg, 5.0 mmol) was added to a solution of 4-([1,2,4]triazol-4-ylamino)-benzonitrile (926 mg, 5.0 mmol) in DMF (50 mL) at room temperature. The mixture was stirred for 1 h at this temperature and 1-benzyloxy-4-chloromethyl-2-trifluoromethyl-benzene (CAB03050, 1.50 g, 5.0 mmol) was added. The reaction mixture was stirred overnight and ethyl acetate (100 mL) and water (30 mL) were added. The mixture was transferred into a separation funnel and washed with water (2×30 mL) and brine (20 mL). The organic layer was dried over sodium sulphate and concentrated under reduced pressure. The residue was suspended in 2-propanol (40 mL) and heated to reflux for 5 minutes. The white solid was filtered off after cooling to room temperature and dried under high vacuum. Yield: 1.87 g (83%). ¹H-NMR (400 MHz, d6-DMSO) δ=5.05 (s, 2H), 5.23 (s, 2H), 6.78 (d, J=9.0 Hz, 2H), 7.26 (d, J=8.6 Hz, 1H), 7.30-7.44 (m, 5H), 7.51 (dd, J=8.6, 2.0 Hz, 1H), 7.56 (d, J=2.0 Hz, 1H), 7.77 (d, J=9.0 Hz, 2H), 8.79 (s, 2H). LRMS (FAB+): 450.2 (100, [M+H]⁺). HRMS (FAB+): 450.15404 C₂₄H₁₉N₅OF₃ requires 450.154170

### 4-[(4-Hydroxy-3-trifluoromethyl-benzyl)-[1,2,4]triazol-4-yl-amino]-benzonitrile (CAB03059, STX781)

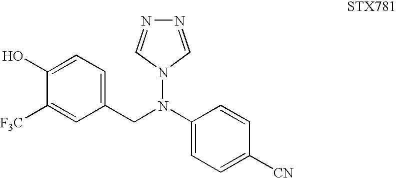

Palladium on charcoal (100 mg, 10% Pd) was added to a solution of 4-[(4-benzyloxy-3-trifluoromethyl-benzyl)-[1,2,4]triazol-4-yl-amino]-benzonitrile (CAB03054, 1.75 g, 3.89 mmol) in EtOH/THF/MeCN (50 mL/50 mL/30 mL). The mixture was stirred under hydrogen atmosphere (balloon) for 18 h at room temperature. The reaction mixture was filtered through celite and the clear colourless filtrate was concentrated under reduced pressure. The residue was suspended in 2-propanol (20 mL) and heated to reflux for 5 minutes. The white solid was filtered off after cooling to room temperature and dried under high vacuum. Yield: 1.31 g (94%). ¹H-NMR (400 MHz, d6-DMSO) δ=4.98 (s, 2H), 6.78 (d, J=9.0 Hz, 2H), 6.91 (d, J=8.6 Hz, 1H), 7.31 (dd, J=8.6, 2.0 Hz, 1H), 7.39 (d, J=2.0 Hz, 1H), 7.76 (d, J=9.0 Hz, 2H), 8.74 (s, 2H), 10.66 (s, 1H, —OH). LRMS (FAB+): 360.2 (100, [M+H]⁺).

### 3-Benzyloxy-phenol (JRL01015)

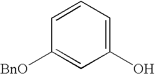

To a stirred solution of resorcinol (7.05 g, 63.4 mmol) in DMF (100 mL) at 0° C. under nitrogen was added NaH (60%, 2.54 g, 63.4 mmol). After stirring for 30 min, benzyl bromide (7.72 mL, 63.4 mmol) was added and the resulting mixture was stirred for 4 h at room temperature. The reaction mixture was diluted with ethyl acetate (300 mL) and the organic layer separated washed with brine (300 mL, 4×100 mL), dried (Na₂SO₄), filtered and evaporated to give the crude product which was fractionated by flash chromatography (hexane/EtOAc 3:1) to give JRL01015 as a pale yellow solid (4.06 g, 32%); R_{f} 0.50 (Hexane/EtOAc 3:1); ¹H (400 MHz CDCl₃) 4.97 (1H, s OH), 5.01 (2H, s, CH₂), 6.42 (1H, dd, J 2.3 and 8.0 Hz), 6.47 (1H, t, J 2.3 Hz), 6.56 (1H, dd, J 2.3 and 8.0 Hz), 7.12 (1H, t, J 8.0 Hz) and 7.28-7.46 (5H, m).

### 5-Benzyloxy-2-nitro-phenol (JRL01017A)

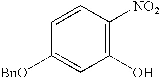

To a stirred solution of JRL01015 (4.0 g, 20.0 mmol) in AcOH (40 mL) at 5-10° C. was added HNO₃ (69%, 2.74 g, 30.0 mmol) in AcOH (1.80 g, 30.0 mmol). After stirring for 4 h at room temperature, the reaction mixture was diluted EtOAc (150 mL) and the organic layer separated washed with brine (200 mL, 4×100 mL), dried (Na₂SO₄), filtered and evaporated to give the crude product which was fractionated by a flash chromatography (hexane/EtOAc 3:1) to give JRL01017 as a yellow solid (1.88 g, 38.5%); R_{f} 0.65 (hexane/EtOAc 3:1); ¹H (400 MHz, CDCl₃) 5.13 (2H, s, CH₂), 6.59 (1H, dd, J 2.4 and 9.0 Hz), 6.62 (1H, d, J 2.4 Hz), 7.34-7.45 (5H, m), 8.04 (1H, d, J 9.0 Hz) and 11.00 (1H, s, OH).

### 2-Amino-5-benzyloxy-phenol (JRL01022)

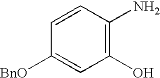

To a stirred solution of JRL01017 (500 mg, 2.04 mmol) in EtOH/H₂O (1:1, 50 mL) was added sodium hydrosulfite (Na₂S₂O₄, ˜85%, 1.67 g, 8.16 mmol) and the yellow suspension resulted was heated at 75° C. After 1 h at this temperature, the reaction mixture had become colorless and it was then cooled to room temperature. The reaction mixture was diluted with EtOAc (100 mL) and the organic layer separated washed with brine (4×50 mL), dried (Na₂SO₄), filtered and evaporated to give the crude product which was purified by a flash chromatography (hexane/EtOAc 3:1) to give JRL01022 as a dark pink solid (418 mg, 95%); R_{f} 0.50 (hexane/EtOAc, 3:1); ¹H (400 MHz, DMSO-d₆) ˜4.0 (2H, v br s), 4.90 (2H, s), 6.24 (1H, dd, J 2.7 and 8.4 Hz), 6.37 (1H, d, J 2.7 Hz), 6.49 (1H, d, J 8.4 Hz), 7.28-7.46 (5H, m) and 11.0 (1H, v br s).

### 6-Benzyloxy-2-bromomethyl-benzooxazole (JRL01026)

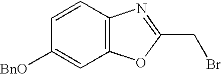

To a stirred mixture of JRL01022 (773 mg, 3.60 mmol) in trimethylsilylpolyphosphate (PPSE)/1,2-dichlorobenzene (1:5, 60 mL) under nitrogen was added bromoacetic acid (400 mg, 2.88 mmol) and the resulting purple mixture was heated at 150° C. for 1 h. After cooling to room temperature, the reaction mixture was diluted with EtOAc (100 mL) and the organic layer separated washed with brine (4×50 mL), dried (Na₂SO₄), filtered and evaporated to an oil which was fractionated by flash chromatography (hexane/EtOAc 5:1) to give JRL01026 as a white solid (369 mg, 40%); R_{f} 0.28 (hexane/EtOAc 5:1); ¹H (400 MHz, CDCl₃) 4.55 (2H, s), 5.10 (2H, s), 7.03 (1H, dd, J 2.2 and 8.6 Hz), 7.10 (1H, d, J 2.2 Hz), 7.30-7.46 (5H, m) and 7.59 (1H, d, J 8.6 Hz).

### 4-[(6-Benzyloxy-benzooxazol-2-ylmethyl)-[1, 2, 4]triazol-4-yl-amino]-benzonitrile (JRL01029)

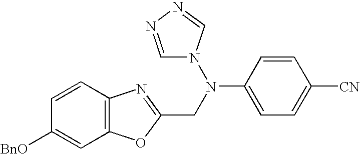

To a stirred solution of NaH (60%, 12 mg, 300 mmol) in DMF (10 mL) at 0° C. under nitrogen was added 4-[(1,2,4)triazol-4-amino]benzonitrile (55 mg, 300 mmol) in DMF (10 mL). After stirring at 40-50° C. under nitrogen for 1 h, the orange reaction mixture was cooled to room temperature and JRL01026 (100 mg, 310 μmol) was added. The resulting mixture was stirred overnight at room temperature under nitrogen. After diluting the reaction mixture with CH₂Cl₂ (50 mL), the organic layer was washed with brine (100 mL, 3×50 mL), dried (Na₂SO₄), filtered and evaporated to give a crude product which was fractionated by flash chromatography (EtOAc) to give JRL01029 as a white solid (80 mg, 63%); R_{f} 0.24 (EtOAc); ¹H (400 MHz, CDCl₃) 5.11 (2H, s), 5.19 (2H, s), 6.59 (2H AA′BB′; 7.05 (1H, dd, J 2.2 and 8.8 Hz), 7.11 (1H, d, J 2.2 Hz), 7.30-7.60 (8H, m) and 8.65 (2H, s).

### 4-[(6-Hydroxy-benzooxazol-2-ylmethyl)-[1,2,4]triazol-4-yl-amino]-benzonitrile (JRL01035, STX357)

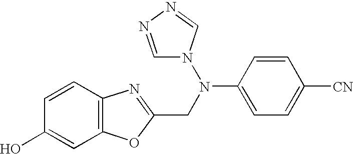

To a stirred solution of JRL01029 (235 mg, 5.56 mmol) in THF/MeOH 1:1 (60 mL) was added Pd—C 10% (65 mg) and the resulting suspension was stirred under an atmosphere of H₂ (balloon) overnight. After filtration on celite, the filtrate collected was evaporated to give a grey solid which was purified by trituration in hot EtOAc to produce JRL01035 as a white solid (128 mg, 69%); R_{f} 0.38 (Acetone/EtOAc, 1:2); ¹H (400 MHz, DMSO-d, 400 MHz) 5.47 (2H, s), 6.70 (2H, d, J 9 Hz), 6.80 (1H, dd, J 2.1 and 8.6 Hz), 7.02 (1H, d, J 2.1 Hz), 7.48 (1H, d, J 8.6 Hz), 7.74 (2H, d, J 9 Hz), 8.95 (2H, s) and 9.86 (1H, br s).

### 4-Benzyloxy-phenol (JRL01016)

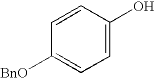

To a stirred solution of hydroquinone (7.00 g, 63.4 mmol) in DMF (100 mL) at 0° C. under nitrogen was added NaH (60%, 2.54 g, 63.4 mmol). After stirring for 30 min, benzyl bromide (7.72 mL, 63.4 mmol) was added and the resulting mixture was stirred for 4 h at room temperature. The reaction mixture was diluted with ethyl acetate (300 mL) and the organic layer separated was washed with brine (300 mL, 4×100 mL), dried (Na₂SO₄), filtered and evaporated to give the crude product which was fractionated by flash chromatography (hexane/EtOAc 3:1) to give JRL01016 as a white solid (3.11 g, 25%); R_{f} 0.35 (Hexane/EtOAc 3:1); ¹H (400 MHz CDCl₃) 4.61 (1H, s OH), 5.01 (2H, s, CH₂), 6.74 (2H, AA′BB′), 6.85 (2H, AA′BB′), and 7.28-7.44 (5H, m).

### 4-Benzyloxy-2-nitro-phenol (JRL01023)

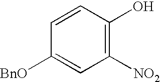

To a stirred solution of JRL01016 (500 mg, 2.50 mmol) in ethylene glycol dimethyl ether (10 mL) at −50° C. under nitrogen was added in one portion tetraborofluorate nitronium in sulpholane (0.5 M, 5.1 mL, 2.55 mmol). After 1 h of stirring at −50° C., the reaction mixture at room temperature was filtered through a short silica column. The eluate collected was evaporated to give the crude product which was fractionated by flash chromatography (hexane/EtOAc 10:1). The first fractionation gave some pure JRL01023 as a yellow solid. The mixture of fractions retrieved from the first column was fractionated again to give more JRL01023 (total amount isolated: 186 mg, combined yield: 30%); R_{f} 0.33 (hexane/EtOAc, 10:1); ¹H (400 MHz, CDCl₃) 5.05 (2H, s), 7.09 (1H, d, J 9.0 Hz), 7.28 (1H, dd, J 3.0 and 9.0 Hz), 7.32-7.44 (5H, m), 7.59 (1H, d, J 3.0 Hz) and 10.34 (1H, s, OH).

### 2-Amino-4-benzyloxy-phenol (JRL01028)

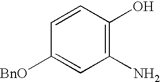

To a stirred solution of JRL01023 (1.33 g, 5.44 mmol) in EtOH/H₂O (1:1, 150 mL) was added sodium hydrosulfite (Na₂S₂O₄, ˜85%, 4.45 g, 21.74 mmol) and the yellow suspension resulted was heated at 75° C. After 1 h at this temperature, the reaction mixture had become colorless and it was then cooled to room temperature. The reaction mixture was diluted with EtOAc (150 mL) and the organic layer separated washed with brine (4×100 mL), dried (Na₂SO₄), filtered and evaporated to give the crude product which was purified by a flash chromatography (hexane/EtOAc 2:1) to give JRL01028 as a brown solid (700 mg, 62%); R_{f} 0.20 (hexane/EtOAc, 2:1); ¹H (400 MHz, DMSO-d₆) 4.54 (2H, s), 4.90 (2H, s), 6.02 (1H, dd, J 2.8 and 8.6 Hz), 6.28 (1H, d, J 2.8 Hz), 6.52 (1H, d, J 8.6 Hz), 7.25-7.45 (5H, m) and 8.50 (1H, s).

### 5-Benzyloxy-2-bromomethyl-benzooxazole (JRL01030)

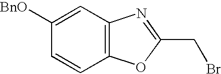

To a stirred mixture of JRL01028 (663 mg, 3.08 mmol) in trimethylsilylpolyphosphate (PPSE)/1,2-dichlorobenzene (1:5, 60 mL) under nitrogen was added bromoacetic acid (333 mg, 2.40 mmol) and the resulting purple mixture was heated at 150° C. for 1 h. After cooling to room temperature, the reaction mixture was diluted with EtOAc (100 mL) and the organic layer separated washed with brine (4×50 mL), dried (Na₂SO₄), filtered and evaporated to an oil which was fractionated by flash chromatography (hexane/EtOAc 7:1) to give JRL01026 as a red solid (228 mg, 30%); R_{f} 0.21 (hexane/EtOAc 7:1); ¹H (400 MHz, CDCl₃) 4.56 (2H, s), 5.10 (2H, s), 7.06 (1H, dd, J 2.4 and 8.8 Hz), 7.25 (1H, d, J 2.4 Hz) and 7.30-7.46 (6H, m).

### 2-Methyl-benzothiazol-6-ol (JRL01040)

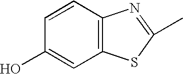

BBr₃ (1M in CH₂Cl₂, 7.7 mL, 7.7 mmol) was slowly added to a stirred solution of 6-methoxybenzothiazol (950 mg, 5.14 mmol) in dichloromethane (30 mL) at 0° C. under nitrogen and the reaction mixture became a dark brown suspension. After stirring at room temperature overnight, the reaction was quenched with ice/brine and diluted with EtOAc (100 mL). The organic layer that separated was washed with brine (4×50 mL), dried (Na₂SO₄), filtered and evaporated to give light brown residue which was fractionated by flash chromatography (hexane/EtOAc 3:1) to give JRL01040 as a white powder (600 mg, 71%); R_{f} 0.16 (hexane/EtOAc 3:1); ¹H (400 MHz, acetone-d) 2.72 (3H, s), 6.99 (1H, dd, J 2.4 and 8.6 Hz), 7.35 (1H, d, J 2.4 Hz), 7.70 (1H, d, J 8.6 Hz) and 8.70 (1H, br s).

### 6-Benzyloxy-2-methyl-benzothiazole (JRL01053)

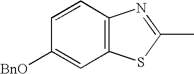

To a stirred solution of JRL01040 (1.47 g, 8.93 mmol) in DMF (30 mL) at 0° C. under nitrogen was added NaH (60%, 393 mg, 9.83 mmol). After stirring for 30 min, benzyl bromide (1.2 mL, 9.83 mmol) was added and the resulting mixture was stirred for 4 h at room temperature. The reaction mixture was diluted with ethyl acetate (100 mL) and the organic layer separated washed with brine (100 mL, 4×50 mL), dried (Na₂SO₄), filtered and evaporated to give the crude product which was fractionated by flash chromatography (hexane/EtOAc 6:1) to give JRL01053 as a pale yellow solid (2.20 g, 96%); R_{f} 0.15 (Hexane/EtOAc 6:1); ¹H (400 MHz CDCl₃) 2.75 (3H, s), 5.06 (2H, s), 7.10 (1H, dd, J 2.2 and 8.8 Hz) 7.28-7.45 (6H, m) and 7.82 (1H, d, J 9 Hz).

### 6-Benzyloxy-2-bromomethyl-benzothiazole (JRL01071)

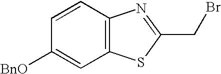

To a stirred solution of JRL01053 (2.1 g, 8.22 mmol) in CCl₄ (50 mL) was added NBS (1.55 g, 8.64 mmol) and dibenzoyl peroxide (32 mg). The pale yellow suspension was refluxed for 2 h, cooled to room temperatue and filtered. The filtrate was diluted with EtOAc (100 mL), washed with NaOH (5%, 1×100 mL) and then brine (3×50 mL) dried (Na₂SO₄), filtered and evaporated to give the crude product which was fractionated by flash chromatography (Hexane/EtOAc 7:1) to give JRL01071 as a white solid (1.05 g, 39%); m.p. 93-96° C.; R_{f} 0.23 (hexane/EtOAc 7:1); ¹H (400 MHz, CDCl₃) 4.76 (2H, s), 5.10 (2H, s), 7.15 (1H, dd, J 2.7 and 9.0 Hz), 7.30-7.46 (6H, m) and 7.89 (1H, d, J 9.0 Hz); LRMS (FAB+) 333.9 [55, (M+H)⁺], 255.0 [17, (M+H−79Br)⁺], 91.0 {100, Bn⁺]; HRMS (FAB+) 333.98873, C₁₅H₁₃BrNOS requires 333.99012.

### 4-[(6-Benzyloxy-benzothiazol-2-ylmethyl)-[1,2,4]triazol-4-yl-amino]-benzonitrile (JRL01074)

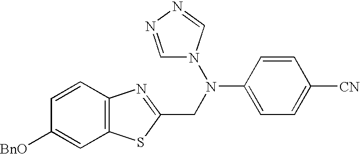

To a stirred mixture of NaH (60%, 57 mg, 1.42 mol) in DMF (5 mL) at 0° C. under nitrogen was added 4-[(1,2,4)triazol-4-amino]benzonitrile (264 mg, 1.42 mol) in DMF (5 mL). After stirring at 40-50° C. under nitrogen for 1 h, the orange reaction mixture was cooled to room temperature and JRL01071 (500 mg, 1.50 mol) was added. The resulting mixture was stirred overnight at room temperature under nitrogen. After diluting the reaction mixture with CH₂Cl₂ (50 mL), the organic layer was washed with brine (100 mL, 3×50 mL), dried (Na₂SO₄), filtered and evaporated to give a crude product which was fractionated by flash chromatography (EtOAc) to give JRL01071 as a pale yellow solid (320 mg, 51%); R_{f} 0.26 (EtOAc); ¹H (400 MHz, CDCl₃) 5.12 (2H, s), 5.30 (2H, s), 6.60 (2H AA′BB′), 7.18 (1H, dd, J 2.6 and 9.0 Hz), 7.30-7.50 (6H, m), 7.54 (2H AA′BB′), 7.85 (1H, d, J 9.0 Hz) and 8.57 (2H, s).

### 5-Benzyloxy-2-methyl-benzothiazole (JRL01052)

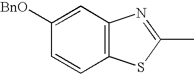

To a stirred solution of 2-Methylbenzothiazol-5-ol (4.0 g, 23.48 mmol) in DMF (40 mL) at 0° C. under nitrogen was added NaH (60%, 1.03 g, 25.75 mmol). After stirring for 30 min, benzyl bromide (3.2 mL, 25.83 mmol) was added and the resulting mixture was stirred for 4 h at room temperature. The reaction mixture was diluted with ethyl acetate (300 mL) and the organic layer separated washed with brine (300 mL, 4×100 mL), dried (Na₂SO₄), filtered and evaporated to give the crude product which was fractionated by flash chromatography (hexane/EtOAc 6:1) to give JRL01052 as a pale yellow solid (5.63 g, 94%); R_{f} 0.15 (Hexane/EtOAc 6:1); ¹H (400 MHz CDCl₃) 2.80 (3H, s), 5.14 (2H, s), 7.00-7.70 (8H, m).

### 5-Benzyloxy-2-bromomethyl-benzothiazole (JRL01064)

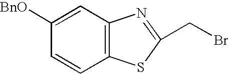

To a stirred solution of JRL01052 (3.6 g, 14.10 mmol) in CCl₄ (100 mL) was added NBS (2.66 g, 14.80 mmol) and dibenzoyl peroxide (55 mg). The pale yellow suspension was refluxed for 2 h, cooled to room temperatue and filtered. The filtrate was diluted with EtOAc (200 mL), washed with NaOH (5%, 1×200 mL) and then brine (3×100 mL) dried (Na₂SO₄), filtered and evaporated to give the crude product which was fractionated by flash chromatography (hexane/EtOAc 7:1) to give JRL01064 as a white solid with (1.81 g, 38%); m.p. 87-89° C.; R_{f} 0.25 (hexane/EtOAc 7:1); ¹H (400 MHz, CDCl₃) 4.77 (2H, s), 5.13 (2H, s), 7.14 (1H, dd, J 2.5 and 8.6 Hz), 7.30-7.48 (5H, m), 7.55 (1H, d, J 2.5 Hz) and 7.71 (1H, d, J 8.6 Hz); LRMS (FAB+) 333.9 [50, (M+H)⁺], 255.0 [17, (M+H-⁷⁹Br)⁺], 91.0 {100, Bn⁺]; HRMS (FAB+) 333.98946, C₁₅H₁₃BrNOS requires 333.99012.

### 4-[(5-Benzyloxy-benzothiazol-2-ylmethyl)-[1,2,4]triazol-4-yl-amino]-benzonitrile (JRL01078)

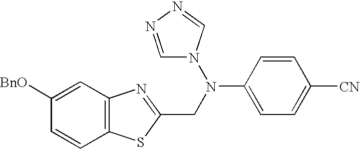

A suspension of JRL01064 (500 mg, 1.5 mmol), 4-[(1,2,4)triazol-4-amino]benzonitrile (277 mg, 1.5 mmol) and anhydrous potassium carbonate (207 mg, 1.5 mmol) in acetonitrile (10 mL) was stirred at room temperature under nitrogen overnight. The resulting reaction mixture was diluted with EtOAc (50 mL) and the organic layer was washed with brine (4×50 mL), dried (Na₂SO₄), filtered and evaporated to give the crude product which was fractionated by flash chromatography (EtOAc/acetone. 4:1) to give JRL01078 as a yellow solid (98 mg, 15%); R_{f} 0.24 (EtOAc); ¹H (400 MHz, CDCl₃) 5.12 (2H, s), 5.33 (2H, s), 6.58 (2H AA′BB′; 7.13 (1H, dd, J 2.6 and 9.0 Hz), 7.30-7.48 (5H, m), 7.50-7.56 (3H, m), 7.71 (1H, d, J 9.0 Hz) and 8.61 (2H, s).

### 4-(3-Hydroxy-propylsulfanyl)-phenol (CAB02029)

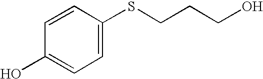

4-Hydroxythiophenol (6.31 g, 50 mmol) was dissolved in ethanol (100 mL) and potassium tert.-butoxide (6.72 g, 60 mmol) was added. The mixture was stirred stirred until a clear, yellow solution was obtained. Then 3-chloro-1-propanol (4.20 mL, 50 mmol) was added with a syringe. The reaction mixture was stirred overnight at room temperature, the potassium chloride precipitate was filtered off and the filtrate was concentrated under reduced pressure. The residue was dissolved in ethyl acetate (150 mL) and the organic layer was extracted with water (2×100 mL) and brine (100 mL), dried over sodium sulphate and concentrated under reduced pressure. The resulting oil was dissolved in dichloromethane (50 mL) and hexane (100 mL) and left standing open overnight. The solid product was filtered off and dried under high vacuum. Yield: 5.34 g (58%) pale yellow solid.

¹H-NMR (400 MHz, CD₃OD) δ=1.71-1.77 (m, 2H), 2.82-2.86 (m, 2H), 3.60-3.64 (m, 2H), 6.73 (d, J=8.6 Hz, 2H), 7.24 (d, J=8.6 Hz, 2H).

### 3-(4-Benzyloxy-phenylsulfanyl)-propan-1-ol (CAB02032)

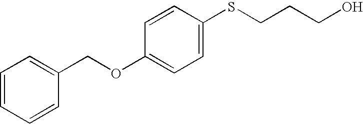

4-(3-Hydroxy-propylsulfanyl)-phenol (CAB02029, 3.686 g, 20 mmol) was dissolved in ethanol (50 mL) and potassium tert-Butoxide (2.80 g, 25 mmol) and benzyl bromide (3.0 mL, ca. 25 mmol) were added. The mixture was stirred overnight at room temperature, the precipitated potassium bromide was filtered off and the filtrate was concentrated under reduced pressure. The resulting yellow solid was dissolved in ethyl acetate (100 mL), the solution was washed with water (100 mL) and brine (100 mL), dried over sodium sulphate and concentrated under reduced pressure. The residue was dissolved in dichloromethane (ca. 10 mL) and the product was precipitated by addition of hexane (ca. 200 mL). The crystalline product was collected and dried under high vacuum. Yield: 4.481 g (82%) colourless, small plates. ¹H-NMR (400 MHz, CDCl₃) δ=1.45 (br s, 1H, —OH), 1.82-1.87 (m, 2H), 2.84 (t, J=7.4 Hz, 2H), 3.74-3.78 (m, 2H), 5.05 (s, 2H, —OCH₂Ph), 6.90-6.93 (m, 2H), 7.31-7.44 (m, 7H).

### 1-Bromo-3-(4-benzyloxy-phenylsulfanyl)-propane (CAB02037)

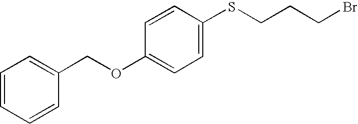

Triphenylphosphine (7.90 g, 30.0 mmol) was added to a solution of 3-(4-benzyloxy-phenylsulfanyl)-propan-1-ol (CAB02032, 4.12 g, 15.0 mmol) and carbon tetrabromide (4.98 g, 18.0 mmol) in dichloromethane (120 mL) at 0° C. (ice/water bath). The reaction mixture was allowed to warm up to room temperature and was stirred for another hour.

The solution was transferred into a separation funnel and conc. NaHCO₃-solution (50 mL) was added. The organic layer was separated, dried over sodium sulphate ad concentrated under reduced pressure. The residue was dissolved in ethyl acetate (25 mL) and hexane (100 mL) was added with stirring. The precipitated triphenylphosphine oxide was filtered off and washed with more EtOAc/hexane-mixture (1:4, 100 mL). The organic solutions were concentrated and the residue was purified by column chromatography (ethyl acetate/hexane, 1:10, Rf: 0.48). Yield: 4.96 g (98%) colourless oil, which becomes a solid after a couple of days. ¹H-NMR (400 MHz, CDCl₃) δ=2.07 (tt, J=7.0, 7.0 Hz, 2H), 2.95 (t, J=7.0 Hz, 2H), 3.50 (t, J=7.0 Hz, 2H), 5.04 (s, 2H, —OCH₂Ph), 6.92 (d, J=9.0 Hz, 2H), 7.27-7.44 (m, 7H). ¹³C-NMR (100.5 MHz, CDCl₃) δ=32.30, 32.50, 34.41, 70.44, 115.84, 126.15, 127.72, 128.31, 128.86, 133.65, 136.89, 158.43.

### 4-{[3-(4-Benzyloxy-phenylsulfanyl)-propyl]-[1,2,4]triazol-4-yl-amino}-benzonitrile (CAB02038)

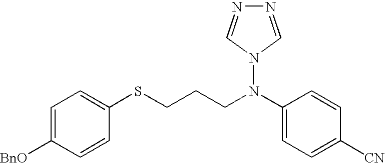

Sodium hydride (60%, 200 mg, 5.0 mmol) was added to a solution of 4-([1,2,4]triazol-4-ylamino)-benzonitrile (926 mg, 5.0 mmol) in DMF (10 mL) at 0° C. The mixture was stirred for 30 min at 50° C., cooled to room temperature and 1-bromo-3-(4-benzyloxy-phenylsulfanyl)-propane (CAB02037, 1.686 g, 5.0 mmol) was added. The reaction mixture was stirred for 15 h and ethyl acetate (100 mL) was added. The mixture was transferred into a separation funnel and extracted with water (2×50 mL) and brine (20 mL). The organic layer was dried over sodium sulphate and concentrated under reduced pressure. The residue was purified by column-chromatography (eluent: ethyl acetate, Rf: 0.41). Yield: 1.724 g (78%) colourless oil. The oil was crystallised from a small amount of methanol. Yield: 1.517 g (68%). ¹H-NMR (400 MHz, CDCl₃) δ=1.84 (tt, J=6.4, 6.4 Hz, 2H), 2.90 (t, J=6.4 Hz, 2H), 3.90 (t, J=6.4 Hz, 2H), 5.06 (s, 2H, —OCH₂Ph), 6.52 (d, J=9.0 Hz, 2H), 6.93 (d, J=9.0 Hz, 2H), 7.31 (d, J=9.0 Hz, 2H), 7.33-7.44 (m, 5H), 7.52 (d, J=9.0 Hz, 2H), 8.26 (s, 2H). LRMS (FAB+): 442.2 (100, [M+H]⁺)

### 4-{[3-(4-Benzyloxy-benzenesulfonyl)-propyl]-[1,2,4]triazol-4-yl-amino}-benzonitrile (CAB02168)

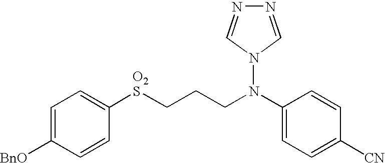

m-Chloroperbenzoic acid (259 mg, 1.50 mmol) was added to a solution of 4-([3-(4-Benzyloxy-phenylsulfanyl)-propyl-[1,2, 4]triazol-4-yl-amino}-benzonitrile (CAB02038, 221 mg, 0.50 mmol) in dichloromethane (10 mL) at room temperature. The mixture was stirred for 1 h, then ethyl acetate (50 mL) and concentrated NaHCO₃-solution (20 mL) were added. The mixture was transferred into a separation funnel, the organic layer was separated, washed with brine (20 mL), dried over sodium sulphate and concentrated under reduced pressure. The residue was purified by column chromatography (ethyl acetate, R_{f}: 0.22). Yield: 186 mg (78%) pale yellow foam. ¹H-NMR (400 MHz, CDCl₃) δ=2.08 (tt, J=7.0, 7.0 Hz, 2H), 3.15 (t, J=7.0 Hz, 2H), 4.04 (t, J=7.0 Hz, 2H), 5.16 (s, 2H, —OCH₂Ph), 6.61 (d, J=9.0 Hz, 2H), 7.11 (d, J=9.0 Hz, 2H), 7.34-7.44 (m, 5H), 7.58 (d, J=9.0 Hz, 2H), 7.81 (d, J=9.0 Hz, 2H), 8.30 (s, 2H). LRMS (FAB+): 474.1 (100, [M+H]⁺).

HRMS (FAB+) 474.16010 C₂₅H₂₄N₅O₃S requires 474.159987

### 4-{[3-(4-Hydroxy-benzenesulfonyl)-propyl]-[1,2,4]triazol-4-yl-amino}-benzonitrile (CAB02169, STX541)

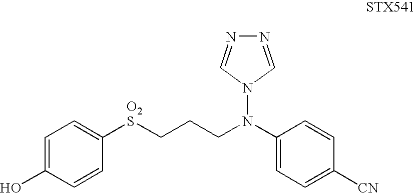

Palladium on charcoal (50 mg, 10% Pd) was added to a solution of 4-{[3-(4-Benzyloxy-benzenesulfonyl)-propyl]-[1,2,4]triazol-4-yl-amino}-benzonitrile (CAB02168, 118 mg, 0.25 mmol) in THF (10 mL) and ethanol (10 mL). The mixture was stirred under hydrogen atmosphere (balloon) for 18 h at room temperature. The reaction mixture was filtered through celite and the clear colourless filtrate was concentrated under reduced pressure. The residue was crystallised from acetone/water. Yield: 68 mg (71%) colourless crystals. ¹H-NMR (400 MHz, d6-DMSO) δ=1.68-1.76 (m, 2H), 3.38 (t, J=7.6 Hz, 2H), 3.92 (t, J=7.2 Hz, 2H), 6.57 (d, J=9.0 Hz, 2H), 6.95 (d, J=9.0 Hz, 2H), 7.68 (d, J=9.0 Hz, 2H), 7.73 (d, J=9.0 Hz, 2H), 8.97 (s, 2H), 10.64 (s, 1H, —OH). LRMS (FAB+): 384.0 (100, [M+H]⁺). HRMS (FAB+): 384.11248 C₁₈H₁₈N₅O₃S requires 384.11304.

### Bis-(4,4′-benzyloxy)phenyl-chloromethane (CAB02062)

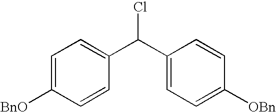

Thionylchloride (3.0 mL) was added to bis-(4-benzyloxy-phenyl)-methanol (1.982 g, 5.0 mmol). The resulting pink solution was stirred at room temperature until the production of sulphur dioxide and hydrogen chloride ceased (ca. 1.5 h). The excess of thionyl chloride was removed under reduced pressure, the crude product was used without any further purification. Yield: 2.075 g (100%). ¹H-NMR (400 MHz, CDCl₃) δ=5.06 (s, 4H, 2×—OCH₂Ph), 6.11 (s, 1H, Ar₂CHCl), 6.94 (d, J=8.6 Hz, 4H), 7.30-7.46 (m, 14H).

### 4-{[Bis-(4-benzyloxy-phenyl)-methyl]-[1,2,4]triazol-4-yl-amino}-benzonitrile (CAB02068)

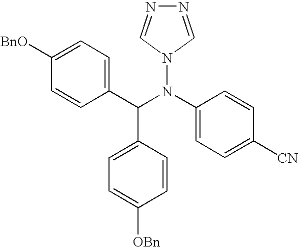

Sodium hydride (60%, 200 mg, 5.0 mmol) was added to a solution of 4-([1,2,4]triazol-4-ylamino)-benzonitrile (926 mg, 5.0 mmol) in DMF (10 mL) at 0° C. The mixture was stirred for 1 h at room temperature and Bis-(4,4′-benzyloxy)phenyl-chloromethane (CAB02062, 2.075 g, 5.0 mmol) was added. The reaction mixture was stirred for 15 h and ethyl acetate (100 mL) was added. The mixture was transferred into a separation funnel and extracted with water (2×50 mL) and brine (20 mL). The organic layer was dried over sodium sulphate and concentrated under reduced pressure. The residue was purified by column-chromatography (ethyl acetate, R_{f}: 0.41). Yield: 1.319 g (47%) white solid. ¹H-NMR (400 MHz, CDCl₃) δ=5.02 (s, 4H, 2×—OCH₂Ph), 6.28 (s, 1H), 6.54 (d, J=9.0 Hz, 2H), 6.90 (d, J=8.6 Hz, 4H), 7.06 (d, J=8.6 Hz, 4H), 7.30-7.44 (m, 10H), 7.51 (d, J=9.0 Hz, 2H), 7.88 (s, 2H). ¹³C-NMR (100.5 MHz, CDCl₃) δ=69.89, 70.44, 104.68, 113.48, 115.63, 118.93, 127.76, 128.39, 128.86, 129.31, 129.76, 134.19, 136.56, 143.92, 150.39, 159.19. LRMS (FAB+): 564.2 (100, [M+H]⁺).

### 4-{[Bis-(4-hydroxy-phenyl)-methyl]-[1,2,4]triazol-4-yl-amino}-benzonitrile (CAB02070, STX340)

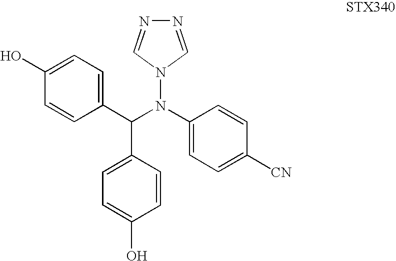

4-{[Bis-(4-benzyloxy-phenyl)-methyl]-[1, 2, 4]triazol-4-yl-amino}-benzonitrile (CAB02068, 564 mg, 1.0 mmol) was dissolved in ethanol (50 mL) and palladium on charcoal (50 mg, 10% Pd) was added. The mixture was stirred under hydrogen atmosphere (balloon) for 48 h (TLC monitored) until all starting material was consumed.

The Pd/C was filtered off (celite) and the solution was concentrated under reduced pressure. A yellow solid was obtained, which was dissolved in ethyl acetate (10 mL) upon heating. After cooling to room temperature the white precipitate was filtered off and dried under high vacuum. Yield: 312 mg (81%). ¹H-NMR (400 MHz, d6-DMSO) δ=5.74 (s, 2H, 2×—OH), 6.28 (s, 1H), 6.53 (d, J=9.0 Hz, 2H), 6.61 (d, J=8.6 Hz, 4H), 7.10 (d, J=8.6 Hz, 4H), 7.65 (d, J=9.0 Hz, 2H), 8.83 (s, 2H). LRMS (FAB+): 199.1 (100), 384.1 (50, [M+H]⁺). LRMS (FAB−): 184.1 (100), 382.1 (41, [M−H]⁻).

### 4-{[Bis-(4-sulphamoyloxy-phenyl)-methyl]-[1,2,4]triazol-4-yl-amino}-benzonitrile (CAB02075)

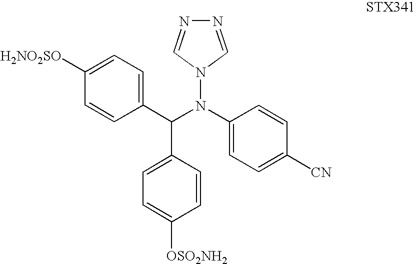

Sulphamoyl chloride solution in toluene (5 mL, 0.7 M, 3.5 mmol) was concentrated under reduced pressure (30° C. water bath temperature) to ca. 1 mL volume. The residue was cooled to 0° C. (ice bath) and N,N-dimethylacetamide (5 mL) was added. 4-([Bis-(4-hydroxy-phenyl)-methyl]-[1,2,4]triazol-4-yl-amino}-benzonitrile (CAB02070, 250 mg, 0.65 mmol) was added to the colourless solution and the mixture was stirred for 18 h at room temperature. Ethyl acetate (50 mL) and water (30 mL) were added to the solution, the organic layer was separated, washed with water (2×30 mL) and brine (20 mL), dried over sodium sulphate and concentrated under reduced pressure to give a white solid. The solid was dissolved in ethyl acetate and precipitated by addition of hexane. The white powder was filtered off and dried under high vacuum. Yield: 299 mg (85%).

¹H-NMR (400 MHz, d6-DMSO) δ=6.67 (s, 1H), 6.67 (d, J=8.6 Hz, 2H), 7.17 (d, J=8.6 Hz, 4H), 7.47 (d, J=8.6 Hz, 4H), 7.70 (d, J=8.6 Hz, 2H), 8.02 (s, 4H, 2×—NH₂), 8.77 (s, 2H). LRMS (FAB+): 542.1 (50, [M+H]⁺). LRMS (FAB−): 184.1 (100), 540.0 (85, [M−H]⁻).

### Dimethylsulfamic acid 4-formyl-phenyl ester (JRL01114) (We followed the synthetic method described in the 1957 German patent no. 1 016 256)

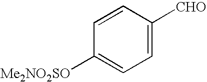

A stirred solution of 4-hydroxybenzaldehyde (1.0 g, 8.02 mmol) in N,N-dimethylcyclohexylamine (7 mL) was heated to 90-95° C. and at this temperature ClSO₂NMe₂ (0.87 mL, 8.02 mL) was added dropwise. The reaction mixture was then heated at 90-95° C. for 3 h. After cooling to room temperatue, EtOAc (100 mL) was added and the organic layer was washed with 1M hydrochloric acid (2×100 mL) and then brine (3×50 mL), dried (Na₂SO₄), filtered and evaporated to give a yellow oil which solidified to a yellow wax upon storage in the refrigerator (1.82 g, 99%); ¹H (270 MHz, CDCl₃) 3.08 (6H, s), 7.46 (2H AA′BB′), 7.94 (2H AA′BB′) and 10.0 (1H, s).

### Dimethyl-sulfamic acid 4-hydroxymethyl-phenyl ester (JRL01115)

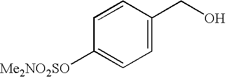

To a stirred solution of JRL01114 (1.81 g, 7.90 mmol) in THF (50 mL) was added at 0° C. NaBH₄ (305 mg, 7.90 mmol). After 2 h of stirring, the initial yellow mixture became a white suspension and the reaction mixture was then poured into ice/water (˜100 mL). The aqueous layer was extracted with chloroform (4×50 mL). The combined organic extracts was dried (Na₂SO₄), filtered and evaporated to give a yellow oil (1.0 g) which was purified by flash chromatography (ethyl acetate) to give JRL01115 as a clear pale yellow oil (820 mg, 45%); ¹H (400 MHz, CDCl₃) 1.76 (˜1H, br s, exchanged with D₂O), 2.98 (6H, s), 4.70 (2H, s), 7.27 (2H AA′BB′) and 7.39 (2H AA′BB′); LRMS (FAB+) 385.1 [7, (M+H+NBA)⁺], 231.0 (50, M⁺), 214.0 [100, (M+H—H₂O)⁺], 202.0 (10); HRMS (FAB+) 231.05557, C₉H₁₃NO₄S requires 231.05653.

### Dimethyl-sulfamic acid 4-chloromethyl-phenyl ester (LWO02144)

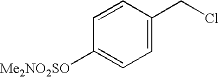

To a solution of JRL01115 (725 mg, 3.135 mmol) in dichloromethane (10 mL) at ice/water temperature was added thionyl chloride (0.35 mL, 4.703 mmol). After stirring at room temperature for 1 h, the volatiles were removed from the reaction mixture and the oil resulted co-evaporated 3 times with chloroform (3×30 mL) to give to LWO02144 as a yellow oil (767 mg, 98%); R_{f} 0.52 (EtOAc/hexane, 1:1), c.f. 0.19 (JRL01115); ¹H (400 MHz, CDCl₃) 2.99 (6H, s, NMe₂), 4.58 (2H, s), 7.28 (2H AA′BB′) and 7.42 (2H, AA′BB′); LRMS (FAB+) 403.0 [18, (M+H+NBA)⁺], 391.2 (21), 249.9 [100, (M+H)⁺], 214.0 [45, (M+H—Cl)⁺], 113.0 (17); HRMS (FAB+) 249.02239, C₉H₁₂ClNO₃S requires 249.02264. LWO02144 was very pure and hence was used without further purification.

### Dimethyl-sulfamic acid 4-{[(4-cyano-phenyl)-[1,2,4]triazol-4-yl-amino]-methyl}-phenyl ester (LWO02145, STX636)

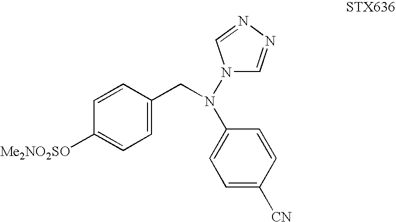

To a stirred solution of 4-[(1,2,4)triazol-4-amino]benzonitrile (538 mg, 2.903 mol) in anhydrous DMF (10 mL) at ice/water temperature was added MaH (60%, 128 mg, 3.193 mmol). The pale orange brown mixture that formed was stirred under nitrogen at 50° C. for 10 min. After cooling to room temperature, LWO02144 (725 mg, 2.903 mmol) in DMF (total 5 mL) was added to the reaction mixture. The resulting orange/brown suspension was stirred and heated at 50-60° C. for 4 h. The cooled reaction mixture was diluted with ethyl acetate (100 mL) and the organic layer that separated washed with brine (100 mL, 4×50 mL), dried (MgSO₄), filtered and evaporated to give an orange/brown syrup (1.15 g). This crude product was fractionated on silica by flash chromatography (neat ethyl acetate initially followed by neat acetone after the first fraction has been collected) and the second fraction that collected upon evaporation gave LWO02145 as a clear bright yellow syrup which solidified upon standing at room temperature overnight to give a light yellow wax (803 mg, 69%); m.p. crystals broke up and scattered intensively at around 150° C. and beyond, crystals melted at 195-202° C.; R_{f} 0.36 (EtOAc), c.f. 0.13 (LWO02144); ¹H (400 MHz, CDCl₃) 2.98 (6H, s, NMe₂), 4.90 (2H, s), 6.68 (2H AA′BB′), 7.27 (4H, m), 7.60 (2H, AA′BB′) and 8.13 (2H, s, triazole-H); LRMS (FAB+) 399.2 [100, (M+H)⁺], 330.1 [43, (M-triazole)⁺]; HRMS (FAB+) 399.12458, C₁₈H₁₉N₆O₃S requires 399.12394. Found: C, 53.9; H, 4.62; N, 22.7%; C₁₈H₁₈N₆O₃S requires C, 54.26; H, 4.55; N, 21.09%

### 3-Bromo-4-(N,N-dimethylsulfamoyl)benzaldehyde (OBS02001)

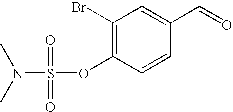

To a solution of OBS01057 (6.0 g, 30 mmol) in N,N-dimethylcyclohexylamine (30 mL) at 80-90° C., was added N,N-dimethylsulfamoyl chloride (3.79 mL, 35.27 mmol). The mixture was stirred at this temperature for 4 h, transferred to a separating funnel and diluted with EtOAc (100 mL). The organic layer was washed with water (2×200 mL), 6M HCl (aq.) (200 mL), brine (2×200 mL), dried (Na₂SO₄) and filtered. Concentration in vacuo of the filtrates gave an orange oil which solidified on standing. The product was stirred in n-hexane, filtered and air-dried to give OBS02001 as a pale yellow solid (8.21 g, 89%). TLC [SiO₂, EtOAc-n-hexane (1:1)] R_{f}=0.9; ¹H-NMR (270 MHz, CDCl₃)=3.09 (6H, s), 7.68 (1H, d, J=8.4), 7.84 (1H, dd, J=1.8, 8.4), 8.12 (1H, d, J=1.8), 9.93 (1H, s).

### 3-Bromo-4-(N,N-dimethylsulfamoyl)benzyl alcohol (OBS02002)

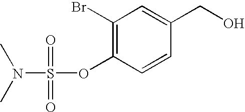

To a solution of OBS02001 (6.0 g, 19.47 mmol) in anhydrous THF (50 mL) was added sodium borohydride (0.81 g, 21.42 mmol). The mixture was stirred at room temperature for 4 h, quenched with water (CARE!!) and filtered through a Celite pad. The filtrate was concentrated in vacuo and re-dissolved in DCM (200 mL). The organic layer was washed with brine (2×200 mL), dried (Na₂SO₄) and filtered. Concentration in vacuo of the filtrates gave OBS02002 as a pale yellow oil (5.54 g, 92%). TLC [SiO₂, EtOAc-n-hexane (1:1)] R_{f}=0.47; ¹H-NMR (270 MHz, CDCl₃)=2.97 (6H, s), 3.14 (1H, bs, OH), 4.51 (2H, s), 7.19 (1H, dd, J=1.5, 8.1), 7.37 (1H, d, J=8.4), 7.52 (1H, d, J=1.8).

### 3-Bromo-4-(N,N-dimethylsulfamoyl)benzyl chloride (OBS02003)

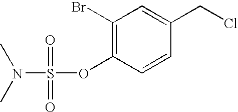

To a solution of OBS02002 (5.0 g, 16.12 mmol) in anhydrous DCM (50 mL) was added thionyl chloride (1.76 mL, 24.18 mmol). The mixture was stirred at room temperature for 2 h and the volatiles removed in vacuo. The residue was re-dissolved and co-evaporated three times with DCM (3×20 mL) to give a yellow oil which solidified on standing. The solid was stirred in n-hexane, filtered and air-dried to give OBS02003 as an off-white solid (5.01 g, 95%). TLC [SiO₂, EtOAc-n-hexane (1:1)] R_{f}=0.79; ¹H-NMR (270 MHz, CDCl₃)=3.05 (6H, s), 4.51 (2H, s), 7.32 (1H, dd, J=2.2, 8.4), 7.48 (1H, d, J=8.4), 7.62 (1H, d, J=2.2).

### Dimethylsulfamic acid 2-bromo-4-{[(4-cyano-phenyl)-[1,2,4]triazol-4-ylamino]methyl}phenyl ester (OBS02005, STX732)

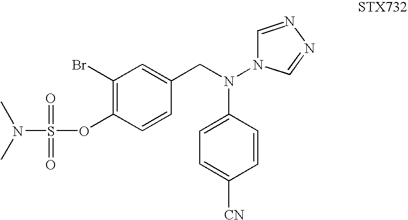

To a suspension of NaH (60% dispersion in oil, 0.44 g, 11.36 mmol) in anhydrous DMF (30 mL) at room temperature was added a solution of 4-[(4-cyanophenyl)amino]-4H-[1,2,4]triazole (2.0 g, 10.8 mmol) in anhydrous DMF (10 mL) and the mixture stirred under nitrogen for 1 h. The orange-yellow suspension was then treated with a solution of OBS02003 (3.73 g, 11.36 mmol) in anhydrous DMF (5 mL) and the mixture stirred at 80-90° C. overnight. The mixture was transferred to a separating funnel and diluted with EtOAc (200 mL). The organic layer was washed with water (4×200 mL), brine (200 mL), dried (MgSO₄) and filtered. Concentration in vacuo of the combined filtrates gave a residue which was recystallised from i-PrOH to give OBS02005 as a colourless solid (3.34 g, 65%). TLC [SiO₂, EtOAc (100%)] R_{f}=0.32 (blue fluorescence at 254 nm); Anal. Calcd. for C₁₉H₁₇N₆SO₃Br: C, 45.3; H, 17.6; N, 3.6%; Found: C, 45.4; H, 17.5; N, 3.6%; ¹H-NMR (400 MHz, d⁶-DMSO)=2.98 (6H, s), 5.11 (2H, s), 6.75 (2H, AA′BB′), 7.43 (2H, s), 7.74 (1H, s), 7.78 (2H, AA′BB′), 8.88 (2H, s); ¹³C-NMR (400 MHz, d⁶-DMSO)=39.4 (2×CH₃), 56.9 (CH₂), 103.8, 114.5 (2×CH), 116.1, 119.7, 123.9 (CH), 129.9 (CH), 134.3 (CH), 134.6 (2×CH), 136.0, 144.0 (2×CH), 146.9, 151.7; MS (FAB+)=477 (100%), 410 (30), 274 (22), 113 (32); Acc. MS for C₁₈H₁₇N₆SO₃Br (Required, 477.03445; Found, 477.03282); LC-MS (Waters 2790 Alliance HPLC/ZQ MicroMass spectrometer with PDA detector using APCI), t_{R} (gradient elution: 5:95 MeCN/H₂O-95:5 MeCN/H₂O over 10 min then 95:5 MeCN/H₂O-5:95 MeCN/H₂O using Waters “Symmetry” C18 (packing: 3.5 μm), 100 mm column)=6.24 min (M+H=478.19); HPLC (Waters 717+ Autosampler with PDA detector, using Waters “Symmetry” C18 (packing: 3.5 μm), 4.6×150 mm column, 90:10 MeOH/H₂O) t_{R}=1.98 min (99.8% purity).

### 3,5-Dibromo-4-(N,N-dimethylsulfamoyl)benzaldehyde (OBS02013)

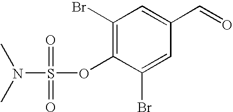

To a solution of 3,5-dibromo-4-hydroxybenzaldehyde (5.0 g, 17.86 mmol) in N,N-dimethylcyclohexylamine (30 mL) at 80-90° C., was added N,N-dimethylsulfamoyl chloride (30 mL). The mixture was stirred at this temperature for 4 h, transferred to a separating funnel and diluted with EtOAc (100 mL). The organic layer was washed with water (2×200 mL), 6M HCl (aq.) (200 mL), brine (2×200 mL), dried (Na₂SO₄) and filtered. Concentration in vacuo of the combined filtrates gave a dark amber oil which solidified on standing. The product was stirred in n-hexane, filtered and air-dried to give OBS02013 as a pale cream solid (6.09 g, 88%). TLC [SiO₂, EtOAc-n-hexane (1:1)] R_{f}=0.73; ¹H-NMR (270 MHz, CDCl₃)=3.14 (6H, s), 8.06 (2H, s), 9.88 (1H, s).

### 3,5-Dibromo-4-(N,N-dimethylsulfamoyl)benzyl alcohol (OBS02015)

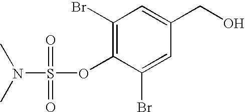

To a solution of OBS02013 (5.5 g, 14.21 mmol) in anhydrous THF (50 mL) was added sodium borohydride (0.59 g, 15.63 mmol). The mixture was stirred at room temperature for 4 h, quenched with water (CARE!!) and filtered through a Celite pad. The filtrate was concentrated in vacuo and re-dissolved in DCM (200 mL). The organic layer was washed with brine (2×200 mL), dried (Na₂SO₄) and filtered. Concentration in vacuo of the combined filtrates gave OBS02015 as a pale yellow oil (5.21 g, 94%). TLC [SiO₂, EtOAc-n-hexane (1:1)] R_{f}=0.55; ¹H-NMR (270 MHz, CDCl₃)=1.97 (1H, bs, OH), 3.11 (6H, s), 4.63 (2H, s), 7.55 (2H, t, J=1.5).

### 3,5-Dibromo-4-(N,N-dimethylsulfamoyl)benzyl chloride (OBS02018)

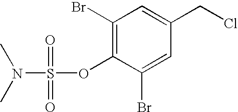

To a solution of OBS02015 (3.93 g, 10.10 mmol) in anhydrous DCM (50 mL) was added thionyl chloride (1.11 mL, 15.15 mmol). The mixture was stirred at room temperature for 2 h and the volatiles removed in vacuo. The residue was re-dissolved and co-evaporated three times with DCM (3×20 mL) to give OBS02018 as a brown oil which solidified on standing (3.93 g, 96%). TLC [SiO₂, EtOAc-n-hexane (1:1)] R_{f}=0.83; ¹H-NMR (270 MHz, CDCl₃)=3.11 (6H, s), 4.46 (2H, s), 7.59 (2H, s).

### Dimethylsulfamic acid 2,6-dibromo-4-{[(4-cyano-phenyl)-[1,2,4]triazol-4-ylamino]methyl}phenyl ester (OBS02019, STX740)

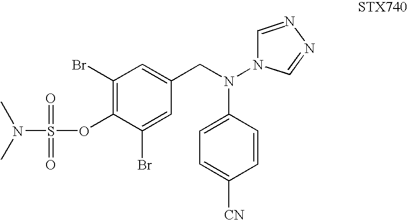

To a suspension of NaH (60% dispersion in oil, 0.22 g, 5.67 mmol) in anhydrous DMF (20 mL) at room temperature was added a solution of 4-[(4-cyanophenyl)amino]-4H-[1,2,4]triazole (1.0 g, 5.4 mmol) in anhydrous DMF (5 mL) and the mixture stirred under nitrogen for 1 h. The orange-yellow suspension was then treated with a solution of OBS02018 (2.31 g, 5.67 mmol) in anhydrous DMF (5 mL) and the mixture stirred at 80-90° C. overnight. The mixture was transferred to a separating funnel and diluted with EtOAc (200 mL). The organic layer was washed with water (4×200 mL), brine (200 mL), and dried (MgSO₄). Concentration in vacuo gave a residue which was recystallised from i-PrOH to give OBS02019 as a white solid (2.06 g, 69%). TLC [SiO₂, EtOAc (100%)] R_{f}=0.53 (blue fluorescence at 254 nm); Anal. Calcd. for C₁₈H₁₆N₆SO₃Br₂: C, 53.3; H, 4.7; N, 19.6%; Found: C, 53.1; H, 4.7; N, 19.3%; ¹H-NMR (270 MHz, d⁶-DMSO)=3.02 (6H, s), 5.09 (2H, s), 6.81 (2H, AA′BB′), 7.73 (2H, s), 7.76 (2H, AA′BB′), 8.93 (2H, s); ¹³C-NMR (400 MHz, d⁶-DMSO)=39.3 (2×CH₃), 56.7 (CH₂), 103.9, 114.5 (2×CH), 118.7, 119.7, 133.6 (2×CH), 134.6 (2×CH), 137.6, 144.0, 145.1 (2×CH), 151.7; MS (FAB)=557 (M+H, 100%), 488 (20), 113 (28); Acc. MS for C₁₈H₁₆N₆SO₃Br₂ (Required, 556.9442; Found, 556.9429); LC-MS (Waters 2790 Alliance HPLC/ZQ MicroMass spectrometer with PDA detector using APCI), t_{R} (gradient elution: 5:95 MeCN/H₂O-95:5 MeCN/H₂O over 10 mins then 95:5 MeCN/H₂O-5:95 MeCN/H₂O using Waters “Symmetry” C18 (packing: 3.5 μm), 100 mm column)=6.46 min (M+2H=558.17); HPLC (Waters 717+ Autosampler with PDA detector, using Waters “Symmetry” C18 (packing: 3.5 μm), 4.6×150 mm column, 90:10 MeOH/H₂O) t_{R}=2.01 min (98.7% purity).

### 3-Bromo-4-(N,N-dimethylsulfamoyl)-5-methoxybenzaldehyde (OBS02022)

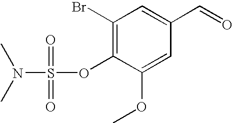

To a solution of 5-bromovanillin (3.0 g, 12.98 mmol) in N,N-dimethylcyclohexylamine (30 mL) at 80-90° C., was added N,N-dimethylsulfamoyl chloride (1.64 mL, 15.26 mmol). The mixture was stirred at this temperature for 4 h, transferred to a separating funnel and diluted with EtOAc (100 mL). The organic layer was washed with water (2×200 mL), 6M HCl (aq.) (200 mL), brine (2×200 mL), dried (Na₂SO₄) and filtered. Concentration in vacuo of the combined filtrates gave a golden-brown oil which solidified on standing. The product was stirred in n-hexane, filtered and air-dried to give OBS02022 as a cream solid (3.22 g, 73%). TLC [SiO₂, EtOAc-n-hexane (1:1)] R_{f}=0.88; ¹H-NMR (270 MHz, CDCl₃)=3.07 (6H, s), 3.96 (3H, s), 7.43 (1H, d, J=1.8), 7.68 (1H, d, J=1.8), 9.87 (1H, s).

### 3-Bromo-4-(N,N-dimethylsulfamoyl)-5-methoxybenzyl alcohol (OBS02023)

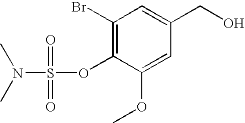

To a solution of OBS02022 (3.0 g, 8.87 mmol) in anhydrous THF (50 mL) was added sodium borohydride (0.37 g, 9.76 mmol). The mixture was stirred at room temperature for 4 h, quenched with water (CARE!!) and filtered through a Celite pad. The filtrate was concentrated in vacuo and re-dissolved in DCM (200 mL). The organic layer was washed with brine (2×200 mL), dried (Na₂SO₄) and filtered. Concentration in vacuo of the combined filtrates gave OBS02023 as a pale yellow oil (1.71 g, 57%). TLC [SiO₂, EtOAc-n-hexane (1:1)] R_{f}=0.68; ¹H-NMR (270 MHz, CDCl₃)=1.94 (1H, bs, OH), 3.07 (6H, s), 3.91 (3H, s), 4.65 (2H, s), 6.96 (1H, d, J=2), 7.17 (1H, d, J=2).

### 3-Bromo-4-(N,N-dimethylsulfamoyl)-5-methoxybenzyl chloride (OBS02026)

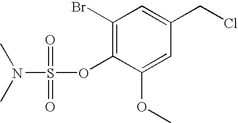

To a solution of OBS02023 (1.56 g, 4.59 mmol) in anhydrous DCM (50 mL) was added thionyl chloride (0.5 mL, 6.88 mmol). The mixture was stirred at room temperature for 2 h and the volatiles removed in vacuo. The residue was re-dissolved and co-evaporated three times with DCM (3×20 mL) to give OBS02026 as a brown oil (0.79 g, 48%). TLC [SiO₂, EtOAc-n-hexane (1:1)] R_{f}=0.89; ¹H-NMR (270 MHz, CDCl₃)=3.00 (6H, s), 3.85 (3H, s), 4.46 (2H, s), 6.92 (1H, d, J=1.8), 7.16 (1H, d, J=1.8).

### Dimethylsulfamic acid 2-bromo-4-{[(4-cyano-phenyl)-[1,2,4]triazol-4-ylamino]methyl}-6-methoxyphenyl ester (OBS02028, STX747)

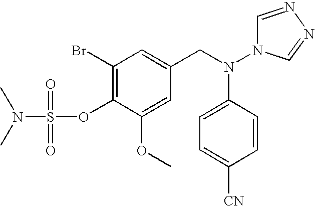

To a suspension of NaH (60% dispersion in oil, 0.07 g, 1.73 mmol) in anhydrous DMF (20 mL) at room temperature was added a solution of 4-[(4-cyanophenyl)amino]-4H-[1,2,4]triazole (0.31 g, 1.65 mmol) in anhydrous DMF (5 mL) and the mixture stirred under nitrogen for 1 h. The orange-yellow suspension was then treated with a solution of OBS02026 (0.62 g, 1.73 mmol) in anhydrous DMF (5 mL) and the mixture stirred at 80-90° C. overnight. The mixture was transferred to a separating funnel and diluted with EtOAc (200 mL). The organic layer was washed with water (4×200 mL), brine (200 mL), dried (MgSO₄) and filtered. Concentration in vacuo of the combined filtrates gave a residue which was recystallised from i-PrOH to give OBS02028 as an off-white solid (0.50 g, 59%). TLC [SiO₂, EtOAc (100%)] R_{f}=0.63 (blue fluorescence at 254 nm); Anal. Calcd. for C₁₉H₁₉N₆SO₄Br: C, 45.0; H, 3.8; N, 16.6%; Found: C, 44.9; H, 3.8; N, 15.8%; ¹H-NMR (400 MHz, d⁶-DMSO)=2.93 (6H, s), 3.86 (3H, s), 5.07 (2H, s), 6.77 (2H, AA′BB′), 7.11 (1H, d, J=1.6), 7.24 (1H, d, J=1.6), 7.79 (2H, AA′BB′), 8.9 (2H, s); ¹³C-NMR (400 MHz, d⁶-DMSO)=39.0 (2×CH₃), 57.31 (CH₂), 57.31 (CH₃), 103.9, 113.3 (CH), 114.5 (CH), 118.3, 119.7, 125.1 (CH), 134.6 (CH), 136.3, 137.3, 144.0 (CH), 152.0, 153.4; MS (FAB+)=509 (M+2H, 100%), 440 (31), 215 (19), 113 (19); Acc. MS for C₁₉H₁₉N₆SO₄Br (Required, 507.04323; Found, 507.04501); LC-MS (Waters 2790 Alliance HPLC/ZQ MicroMass spectrometer with PDA detector using APCI), t_{R} (gradient elution: 5:95 MeCN/H₂O-95:5 MeCN/H₂O over 10 mins then 95:5 MeCN/H₂O-5:95 MeCN/H₂O using Waters “Symmetry” C18 (packing: 3.5 μm), 100 mm column)=6.16 min (M+H=508.29); HPLC (Waters 717+ Autosampler with PDA detector, using Waters “Symmetry” C18 (packing: 3.5 μm), 4.6×150 mm column, 90:10 MeOH/H₂O) t_{R}=1.96 min (97.5% purity).

### 3-Chloro-4-(N,N-dimethylsulfamoyl)benzaldehyde (OBS02043)

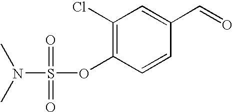

To a solution of 3-chloro-4-hydroxybenzaldehyde (6.0 g, 38.32 mmol) in N,N-dimethylcyclohexylamine (30 mL) at 80-90° C., was added N,N-dimethylsulfamoyl chloride (4.84 mL, 45.05 mmol). The mixture was stirred at this temperature for 4 h, transferred to a separating funnel and diluted with EtOAc (100 mL). The organic layer was washed with water (2×200 mL), 6M HCl (aq.) (200 mL), brine (2×200 mL), dried (Na₂SO₄) and filtered. Concentration in vacuo of the combined filtrates gave a brown oil which solidified on standing. The product was stirred in n-hexane, filtered and air-dried to give OBS02043 as a beige solid (5.36 g, 53%). TLC [SiO₂, EtOAc-n-hexane (1:1)] R_{f}=0.92; ¹H-NMR (270 MHz, CDCl₃)=3.06 (6H, s), 7.67 (1H, d, J=8.4), 7.79 (1H, dd, J==2.2, 8.4), 7.95 (1H, d, J=1.8), 9.93 (1H, s).

### 3-Chloro-4-(N,N-dimethylsulfamoyl)benzyl alcohol (OBS02046)

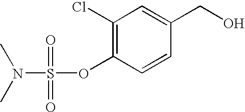

To a solution of OBS02043 (5.0 g, 18.96 mmol) in anhydrous THF (50 mL) was added sodium borohydride (0.79 g, 20.86 mmol). The mixture was stirred at room temperature for 4 h, quenched with water (CARE!!) and filtered through a Celite pad. The filtrate was concentrated in vacuo and re-dissolved in DCM (200 mL). The organic layer was washed with brine (2×200 mL), dried (Na₂SO₄) and filtered. Concentration in vacuo of the combined filtrates gave OBS02046 as a golden-brown oil (3.81 g, 76%). TLC [SiO₂, EtOAc-n-hexane (1:1)] R_{f}=0.64; ¹H-NMR (270 MHz, CDCl₃)=2.04 (1H, bs, OH), 3.05 (6H, s), 4.66 (2H, s), 7.25 (1H, dd, J=2.2, 8.4), 7.46 (1H, d, J=2.2), 7.47 (1H, d, J=8.1).

### 3-Chloro-4-(N,N-dimethylsulfamoyl)benzyl chloride (OBS02052)

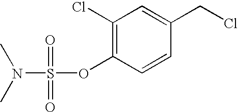

To a solution of OBS02046 (3.02 g, 11.37 mmol) in anhydrous DCM (50 mL) was added thionyl chloride (1.24 mL, 17.05 mmol). The mixture was stirred at room temperature for 2 h and the volatiles removed in vacuo. The residue was re-dissolved and co-evaporated three times with DCM (3×20 mL) to give OBS02052 as an amber oil (2.68 g, 83%). TLC [SiO₂, EtOAc-n-hexane (1:1)] R_{f}=0.91; ¹H-NMR (270 MHz, CDCl₃)=3.04 (6H, s), 4.52 (2H, s), 7.28 (1H, dd, J=2.2, 8.4), 7.47 (1H, d, J=8.5), 7.47 (1H, d, J=2.2).

### Dimethylsulfamic acid 2-chloro-4-{[(4-cyano-phenyl)-[1,2,4]triazol-4-ylamino]methyl}phenyl ester (OBS02054, STX787)

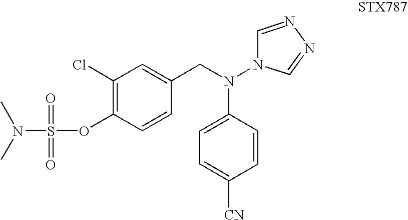

To a suspension of NaH (60% dispersion in oil, 0.34 g, 8.95 mmol) in anhydrous DMF (20 mL) at room temperature was added a solution of 4-[(4-cyanophenyl)amino]-4H-[1,2,4]triazole (1.66 g, 8.95 mmol) in anhydrous DMF (5 mL) and the mixture stirred under nitrogen for 1 h. The orange-yellow suspension was then treated with a solution of OBS02052 (2.67 g, 9.4 mmol) in anhydrous DMF (5 mL) and the mixture stirred at 80-90° C. overnight. The mixture was transferred to a separating funnel and diluted with EtOAc (200 mL). The organic layer was washed with water (4×200 mL), brine (200 mL), dried (MgSO₄) and filtered. Concentration in vacuo of the combined filtrates gave a residue which was recystallised from i-PrOH to give OBS02054 as a pale cream solid (2.03 g, 52%). TLC [SiO₂, EtOAc (100%)] R_{f}=0.53 (blue fluorescence at 254 nm); Anal. Calcd. for C₁₈H₁₇N₆SO₃Cl: C, 49.9; H, 4.0; N, 19.4%; Found: C, 49.7; H, 4.0; N, 19.2%; ¹H-NMR (270 MHz, CDCl₃)=3.01 (6H, s), 4.87 (2H, s), 6.62 (2H, AA′BB′), 7.15 (1H, dd, J=2.2, 8.4), 7.32 (1H, d, J=2.2), 7.46 (1H, d, J=8.4), 7.55 (2H, AA′BB′), 8.18 (2H, s); ¹³C-NMR (400 MHz, d⁶-DMSO)=38.3 (2×CH₃), 56.0 (CH₂), 102.9, 113.5 (2×CH), 118.7, 123.4 (CH), 125.8, 128.3 (CH), 130.3 (CH), 133.7 (2×CH), 134.9, 143.0 (2×CH), 144.7, 150.9; MS (FAB)=433 (M+H, 100%), 364 (37); Acc. MS for C₁₈H₁₇N₆SO₃Cl (Required M+H, 433.08476; Found M+H, 433.08496); LC-MS (Waters 2790 Alliance HPLC/ZQ MicroMass spectrometer with PDA detector using APCI), t_{R} (gradient elution: 5:95 MeCN/H₂O-95:5 MeCN/H₂O over 10 min then 95:5 MeCN/H₂O-5:95 MeCN/H₂O using Waters “Symmetry” C18 (packing: 3.5 μm), 100 mm column)=6.18 min (M+H=434.18); HPLC (Waters 717+Autosampler with PDA detector, using Waters “Symmetry” C18 (packing: 3.5 μm), 4.6×150 mm column, 90:10 MeOH/H₂O) t_{R}=1.98 min (99.9% purity).

### 4-(N,N-Dimethylsulfamoyl)-3-methoxybenzaldehyde (OBS02011)

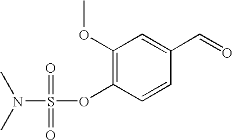

To a solution of vanillin (4.56 g, 30 mmol) in N,N-dimethylcyclohexylamine (30 mL) at 80-90° C., was added N,N-dimethylsulfamoyl chloride (3.79 mL, 35.27 mmol). The mixture was stirred at this temperature for 4 h, transferred to a separating funnel and diluted with EtOAc (100 mL). The organic layer was washed with water (2×200 mL), 6M HCl (aq.) (200 mL), brine (2×200 mL), dried (Na₂SO₄) and filtered. Concentration in vacuo of the combined filtrates gave a golden-yellow oil which solidified on standing. The product was stirred in n-hexane, filtered and air-dried to give OBS02011 as yellow plates (6.92 g, 89%). TLC [SiO₂, EtOAc-n-hexane (1:1)] R_{f}=0.65; ¹H-NMR (270 MHz, CDCl₃)=2.98 (6H, s), 3.93 (3H, s), 7.44 (1H, d, J=1.6), 7.48 (1H, dd, J=1.6, 8.1), 7.53 (1H, d, J=8.1), 9.92 (1H, s).

### 4-(N,N-Dimethylsulfamoyl)-3-methoxybenzyl alcohol (OBS02014)

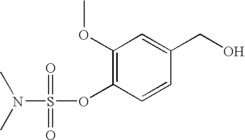

To a solution of OBS02011 (5.5 g, 21.21 mmol) in anhydrous THF (50 mL) was added sodium borohydride (0.88 g, 23.33 mmol). The mixture was stirred at room temperature for 4 h, quenched with water (CARE!!) and filtered through a Celite pad. The filtrate was concentrated in vacuo and re-dissolved in DCM (200 mL). The organic layer was washed with brine (2×200 mL), dried (Na₂SO₄) and filtered. Concentration in vacuo of the combined filtrates gave OBS02014 as a pale yellow oil (5.12 g, 92%). TLC [SiO₂, EtOAc-n-hexane (1:1)] R_{f}=0.18; ¹H-NMR (270 MHz, CDCl₃)=2.44 (1H, bs, OH), 2.92 (6H, s), 3.84 (3H, s), 4.60 (2H, s), 6.85 (1H, dd, J=2.2, 8.4), 6.97 (1H, d, J=1.8), 7.25 (1H, d, J=8.1).

### 4-(N,N-Dimethylsulfamoyl)-3-methoxybenzyl chloride (OBS02016)

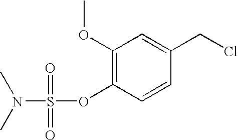

To a solution of OBS02014 (2.76 g, 10.55 mmol) in anhydrous DCM (50 mL) was added thionyl chloride (1.15 mL, 15.82 mmol). The mixture was stirred at room temperature for 2 h and the volatiles removed in vacuo. The residue was re-dissolved and co-evaporated three times with DCM (3×20 mL) to give OBS02016 as a brown oil (2.91 g, 99%). TLC [SiO₂, EtOAc-n-hexane (1:1)] R_{f}=0.84; ¹H-NMR (270 MHz, CDCl₃)=2.96 (6H, s), 3.90 (3H, s), 4.55 (2H, s), 6.95 (1H, dd, J=1.8, 8.0), 7.01 (1H, d, J=1.8), 7.32 (1H, d, J=8.0).

### Dimethylsulfamic acid 4-{[(4-cyano-phenyl)-[1,2,4]triazol-4-ylamino]methyl}-2-methoxyphenyl ester (OBS02017, STX739)

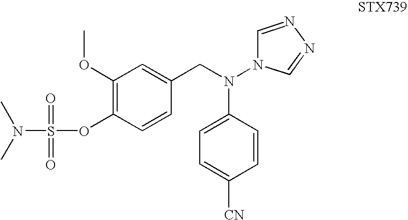

To a suspension of NaH (60% dispersion in oil, 0.22 g, 5.67 mmol) in anhydrous DMF (20 mL) at room temperature was added a solution of 4-[(4-cyanophenyl)amino]-4H-[1,2,4]triazole (1.0 g, 5.4 mmol) in anhydrous DMF (5 mL) and the mixture stirred under nitrogen for 1 h. The orange-yellow suspension was then treated with a solution of OBS02016 (1.59 g, 5.67 mmol) in anhydrous DMF (5 mL) and the mixture stirred at 80-90° C. overnight. The mixture was transferred to a separating funnel and diluted with EtOAc (200 mL). The organic layer was washed with water (4×200 mL), brine (200 mL), dried (MgSO₄) and filtered. Concentration in vacuo of the combined filtrates gave a residue which was recystallised from i-PrOH to give OBS02017 as a cream solid (1.07 g, 46%). TLC [SiO₂, EtOAc (100%)] R_{f}=0.26 (blue fluorescence at 254 nm); Anal. Calcd. for C₁₉H₂₀N₆SO₄: C, 38.9; H, 2.9; N, 15.1%; Found: C, 38.9; H, 3.0; N, 14.8%; ¹H-NMR (400 MHz, d⁶-DMSO)=2.83 (6H, s), 3.84 (3H, s), 5.07 (2H, s), 6.80 (2H, AA′BB′), 6.90 (1H, dd, J=2, 8), 7.12 (1H, d, J=2), 7.23 (1H, d, J=8), 7.79 (2H, AA′BB′), 8.83 (2H, s); ¹³C-NMR (400 MHz, d⁶-DMSO)=39.1 (2×CH₃), 56.8 (CH₃), 57.6 (CH₂), 103.7, 114.1 (2×CH), 114.5 (CH), 119.7, 121.5 (CH), 124.1 (CH), 134.6 (2×CH), 135.2, 138.8, 144.0 (2×CH), 151.8, 152.0; MS (FAB+)=429 (M+H, 100%), 360 (36), 321 (6), 244 (15), 113 (5); Acc. MS for C₁₉H₂₀N₆SO₄ (Required, 429.1341; Found, 429.1345); LC-MS (Waters 2790 Alliance HPLC/ZQ MicroMass spectrometer with PDA detector using APCI), t_{R} (gradient elution: 5:95 MeCN/H₂O-95:5 MeCN/H₂O over 10 min then 95:5 MeCN/H₂O-5:95 MeCN/H₂O using Waters “Symmetry” C18 (packing: 3.5 μm), 100 mm column)=5.83 min (M+2H=430.27); HPLC (Waters 717+ Autosampler with PDA detector, using Waters “Symmetry” C18 (packing: 3.5 μm), 4.6×150 mm column, 90:10 MeOH/H₂O) t_{R}=1.94 min (99.5% purity).

### 3-(N,N-Dimethylsulfamoyl)-4-methoxybenzaldehyde (OBS02049)

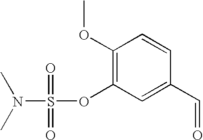

To a solution of isovanillin (4.56 g, 30 mmol) in N,N-dimethylcyclohexylamine (30 mL) at 80-90° C., was added N,N-dimethylsulfamoyl chloride (3.79 g, 35.27 mmol). The mixture was stirred at this temperature for 4 h, transferred to a separating funnel and diluted with EtOAc (100 mL). The organic layer was washed with water (2×200 mL), 6M HCl (aq.) (200 mL), brine (2×200 mL), dried (Na₂SO₄) and filtered. Concentration in vacuo of the combined filtrates gave a brown oil which solidified on standing. The product was stirred in n-hexane, filtered and air-dried to give OBS02049 as a pale yellow solid (6.91 g, 89%). TLC [SiO₂, EtOAc-n-hexane (1:1)] R_{f}=0.57; ¹H-NMR (270 MHz, CDCl₃)=2.98 (6H, s), 3.95 (3H, s), 7.07 (1H, d, J=8.4), 7.77 (1H, dd, J=1.8, 8.4), 7.83 (1H, d, J=2.2), 9.84 (1H, s).

### 3-(N,N-Dimethylsulfamoyl)-4-methoxybenzyl alcohol (OBS02053)

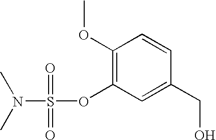

To a solution of OBS02049 (5.5 g, 21.21 mmol) in anhydrous THF (50 mL) was added sodium borohydride (0.88 g, 23.33 mmol). The mixture was stirred at room temperature for 4 h, quenched with water (CARE!!) and filtered through a Celite pad. The filtrate was concentrated in vacuo and re-dissolved in DCM (200 mL). The organic layer was washed with brine (2×200 mL), dried (Na₂SO₄) and filtered. Concentration in vacuo of the combined filtrates gave OBS02053 as a golden-yellow oil (3.26 g, 59%). TLC [SiO₂, EtOAc-n-hexane (1:1)] R_{f}=0.36; ¹H-NMR (270 MHz, CDCl₃)=1.73 (1H, bs, OH), 2.97 (6H, s), 3.88 (3H, s), 4.62 (2H, s), 6.95 (1H, d, J=8.4), 7.23 (1H, dd, J=2.2, 8.4), 7.36 (1H, d, J=2.2).

### 3-(N,N-Dimethylsulfamoyl)-4-methoxybenzyl chloride (OBS02058)

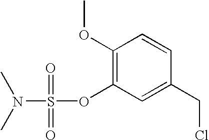

To a solution of OBS02053 (2.33 g, 8.89 mmol) in anhydrous DCM (50 mL) was added thionyl chloride (0.97 mL, 13.34 mmol). The mixture was stirred at room temperature for 2 h and the volatiles removed in vacuo. The residue was re-dissolved and co-evaporated three times with DCM (3×20 mL) to give OBS02058 as a light-sensitive brown oil (2.07 g, 83%); ¹H-NMR (270 MHz, CDCl₃)=2.98 (6H, s), 3.89 (3H, s), 4.54 (2H, s), 6.94 (1H, d, J=8.4), 7.25 (1H, dd, J=2.2, 8.4), 7.40 (1H, d, J=2.2).

### Dimethylsulfamic acid 5-{[(4-cyano-phenyl)-[1,2,4]triazol-4-ylamino]methyl}-2-methoxyphenyl ester (OBS02060, STX796)

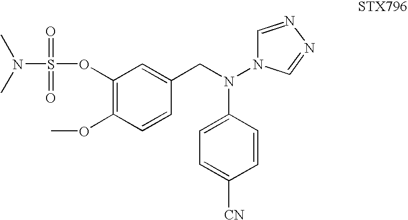

To a suspension of NaH (60% dispersion in oil, 0.28 g, 6.91 mmol) in anhydrous DMF (20 mL) at room temperature was added a solution of 4-[(4-cyanophenyl)amino]-4H-[1,2,4]triazole (1.28 g, 6.91 mmol) in anhydrous DMF (5 mL) and the mixture stirred under nitrogen for 1 h. The orange-yellow suspension was then treated with a solution of OBS02058 (2.03 g, 7.26 mmol) in anhydrous DMF (5 mL) and the mixture stirred at 80-90° C. overnight. The mixture was transferred to a separating funnel and diluted with EtOAc (200 mL). The organic layer was washed with water (4×200 mL), brine (200 mL), dried (MgSO₄) and filtered. Concentration in vacuo of the combined filtrates gave a residue which was recystallised from EtOAc-n-hexane to give OBS02060 as a pale cream solid (2.24 g, 76%); ¹H-NMR (270 MHz, CHCl₃)=2.88 (6H, s), 3.82 (3H, s), 4.79 (2H, s), 6.66 (2H, AA′BB′), 6.83 (1H, d, J=8.4), 6.94 (1H, dd, J=2.2, 8.4), 7.30 (1H, d, J=1.8), 7.53 (2H, AA′BB′), 8.12 (2H, s); ¹³C-NMR (270 MHz, CHCl₃)=38.7 (2×CH₃), 56.1 (CH₃), 57.2 (CH₂), 105.1, 112.9 (CH), 113.6 (2×CH), 118.5, 123.9 (CH), 125.7 (CH), 127.6 (CH), 133.9 (2×CH), 139.4, 142.6 (2×CH), 150.4, 151.8; MS (FAB+)=429 (M+H, 100%), 360 (50), 321 (6), 244 (43); Acc. MS for C₁₉H₂₀N₆SO₄ (Required, 429.1343; Found, 429.1345); LC-MS (Waters 2790 Alliance HPLC/ZQ MicroMass spectrometer with PDA detector using APCI), t_{R} (gradient elution: 5:95 MeCN/H₂O-95:5 MeCN/H₂O over 10 min then 95:5 MeCN/H₂O-5:95 MeCN/H₂O using Waters “Symmetry” C18 (packing: 3.5 μm), 100 mm column)=5.80 min (M+2H=430.27); HPLC (Waters 717+ Autosampler with PDA detector, using Waters “Symmetry” C18 (packing: 3.5 μm), 4.6×150 mm column, 90:10 MeOH/H₂O) t_{R}=1.96 min (99.8% purity).

Synthesis of STX258, 265, 273, 287, 288, 290, 291, 292

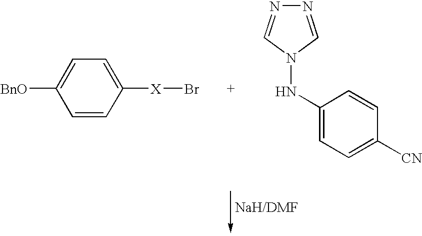

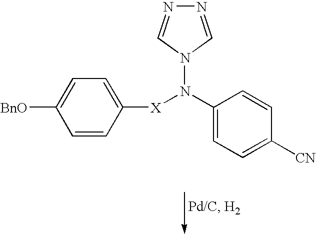

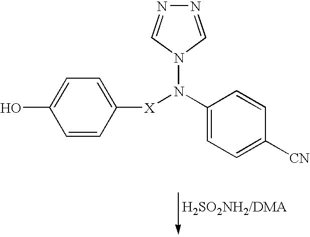

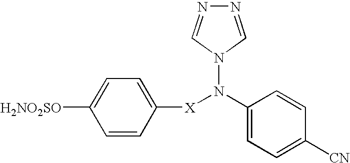

| X | |
|---|---|
| CH₂ | STX265 |
| CH₂CH₂ | STX290 |
| O(CH₂)₄ | STX287 |
| O(CH₂)₂ | STX291 |
| CH₂ | STX258 |
| CH₂CH₂ | STX273 |
| O(CH₂)₄ | STX288 |
| O(CH₂)₂ | STX292 |

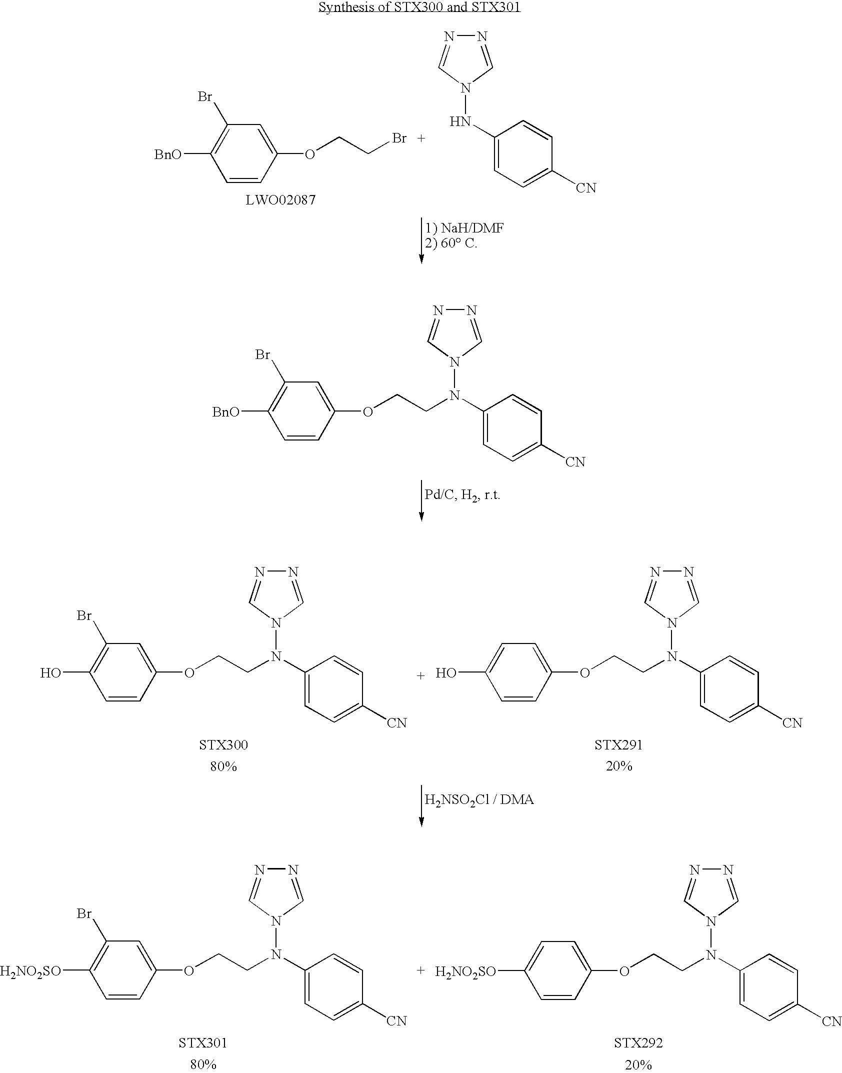 Compounds of Formula IV

### Bis-(4-Benzyloxyphenyl)methanone (LWO02007A)

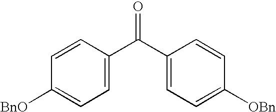

To a solution of 4,4′-dihydroxybenzophenone (5.0 g, 23.34 mmol) in anhydrous DMF (150 mL) at 0° C. was added sodium hydride (60% in mineral oil, 2.1 g, 51.35 mmol), in two portions. After stirring for 20 min at which no more evolution of hydrogen was observed, benzyl bromide (8.96 g, 51.35 mmol) was added. The resulting yellow suspension was then stirred under an atmosphere of nitrogen at 100° C. for 1 h. Upon cooling to room temperature, water (500 mL) was added to the suspension and the precipitate that formed was filtered and washed exhaustively with water. After air-drying overnight at room temperature, the white solid (10.1 g) that collected was recrystallised from hot toluene to give LWO02007A as white flaky plate crystals (8.92 g, 22.61 mmol, 96.9%); m.p. 188-190° C.

### Bis-(4-Benzyloxyphenyl)methanol (LWO02018)

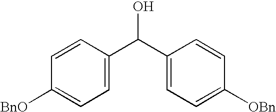

To a solution of LWO02007A (3.50 g, 8.873 mmol) in anhydrous THF (250 mL) at 0° C. was added a suspension of lithium aluminium hydride (95%, 425 mg, 10.65 mmol) in anhydrous THF (20 mL). After stirring for 30 min at room temperature, the grey suspension/mixture was concentrated and ethyl acetate (200 mL) was added the wet residue that obtained. The organic layer was washed with 1M HCl (200 mL), then brine (4×100 mL), dried (MgSO₄), filtered and evaporated to give LWO02018 as a white/pale yellow residue (3.56 g); m.p. 113-120° C.; δ_{H} (400 MHz, DMSO-d₆) 5.06 (4H, s, OCH₂), 5.58 (1H, d, J=4.3 Hz, CH), 5.67 (1H, d, J=4.3 Hz, exchanged with D₂O, OH), 6.93 (4H, AA′BB′), 7.23 (4H, AA′BB′) and 7.38 (10H, m, Bn).

### 1-[Bis-(4-Benzyloxyphenyl)methyl]-1H-[1,2,4]triazole (LWO02019)

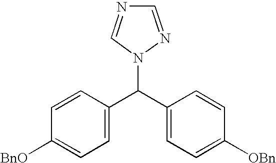

A mixture of LWO02018 (3.26 g, 8.222 mmol) and 1H-1,2,4-triazole (695 mg, 9.866 mmol) in toluene (350 mL) in the presence of p-toluenesulphonic acid (650 mg) was heated under Dean Stark conditions overnight. After cooling to room temperature and evaporation of solvent, the light yellow residue that obtained was dissolved in ethyl acetate (300 mL). The organic layer was washed with 1M NaOH (2×100 mL), then brine (3×50 mL), dried (MgSO₄), filtered and evaporated to give a light yellow/brown residue (3.36 g). This crude was dissolved in hot ethyl acetate (30 mL) and hexane (15 mL) was added portionwise. Upon cooling to room temperature, LWO02019A was obtained as yellow crystals (2.50 g, 5.586 mmol, 68%); m.p. 134-137° C.; δ_{H} (400 MHz, DMSO-d₆) 5.09 (4H, s, 2×OCH₂), 6.94 (1H, s, CH), 7.01 (4H, AA′BB′), 7.14 (4H, AA′BB′), 7.29-7.47 (10H, m, Bn), 8.03 (˜1H, s, C3′-H) and 8.53 (1H, s, C5′-H); LRMS (FAB+) 447.3[17, M⁺], 379.3[100, (M-triazole)⁺], 288,2[8, (M-triazole-Bn)⁺], 91.1[75, Bn⁺]; LRMS (FAB−): no peak was observed; HRMS (FAB+) 447.19588 C₂₉H₂₅N₃O₂ requires 447.19468. Found: C, 77.6; H, 5.63; N, 9.26; C₂₉H₂₅N₃O₂ requires C, 77.83; H, 5.63; N, 9.37.

### 1-[Bis-(4-Hydroxyphenyl)methyl]-1H-[1,2,4]triazole (LWO02020, STX267)

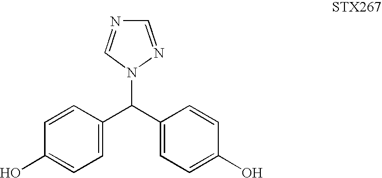

To a solution of LWO02019 (1.50 g, 3.356 mmol) in distilled THF (50 mL) was added methanol (30 mL) and Pd/C (10%, 75 mg). The black suspension was stirred at room temperature under an atmosphere of hydrogen (balloon) over weekend. After removal by filtration and exhaustive washings of the supported catalyst with distilled THF, the filtrate was evaporated to give a frothy light yellow residue (839 mg, 3.319 mmol, 93.5%). This crude was dissolved in hot THF (15 mL) and hexane (10 mL) was added portionwise. Upon cooling, LWO02020A was obtained as white crystals (483 mg); m.p. 230° C.; δ_{H} (400 MHz, DMSO-d₆) 6.73 (4H, AA′BB′), 6.79 (1H, s, CH), 6.99 (4H, AA′BB′), 8.01 (˜1H, s, C3′-H), 8.45 (1H, s, C5′-H) and 9.52 (˜2H, s, exchanged with D₂O, 2×OH); δ_{C} (100.4 MHz, DMSO-d₆) 65.2 (d, CH), 115.3 (d, Ar), 129.3 (d, Ar), 129.9 (s, Ar), 144.0 (d, C5′), 151.8 (d, C3′), 157.1 (s, Ar—OH). Found: C, 67.2; H, 5.08; N, 15.4; C₁₅H₁₃N₃O₂ requires C, 67.4; H, 4.90; N, 15.72.

### 1-[Bis-(4-sulfamoyloxyphenyl)methyl]-1H-[1,2,4]triazole (LWO02021, STX268)

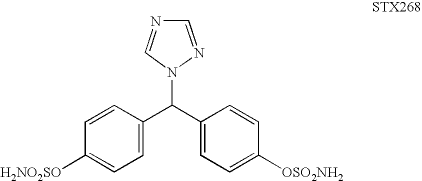

To a solution of LWO02020 (257 mg, 1.336 mmol) in N,N-dimethylacetamide (20 mL) at room temperature under an atmosphere of nitrogen was added sulfamoyl chloride in toluene (ca. 0.68 M, 7.8 mL). After stirring the reaction mixture overnight, it was diluted with ethyl acetate (100 mL). The organic layer that separated was washed with brine (100 mL, 4×50 mL), dried (MgSO₄), filtered and evaporated to give a light brown syrup/residue (612 mg). This crude was purified by flash chromatography (ethyl acetate) and the second fraction that collected gave LWO02021A as white residue (310 mg, 728.7 μmol, 54.5%); m.p. 70-85° C.; δ_{H} (400 MHz, DMSO-d₆) 7.19 (1H, s, CH), 7.30 (4H, AA′BB′), 7.36 (4H, AA′BB′), 8.03 (˜4H, br s, exchanged with D₂O, H₂NSO₂), 8.10 (˜1H, s, C3′-H) and 8.63 (1H, s, C5′-H); LRMS (FAB+): 851.2[6, (2M+H)⁺], 579.2[10, (M+H+NBA)⁺], 426.2[60, (M+H)⁺], 357.2[100, (M-triazole)⁺]; (FAB−): 849.1[16, (2M−H)⁻], 578.1[35, (M+NBA)⁻], 424.1[100, (M−H)⁻], 345.2[25, (M−H₂NSO₂)⁻]; HRMS (FAB+) 426.05452 C₁₅H₁₆N₅O₆S₂ requires 426.05420.

### 1-[Bis-(3-bromo-4-hydroxyphenyl)methyl]-1H-[1,2,4]triazole (JRL01105, STX356)

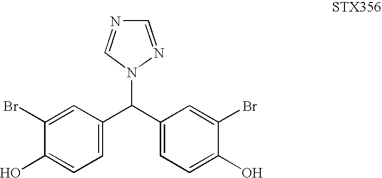

To a stirred solution of 1-[Bis-(4-hydroxyphenyl)methyl]-1H-[1,2,4]triazole (STX267, 500 mg, 1.87 mmol) in CH₂Cl₂/MeOH 1:1 (40 ml) at −78° C. under nitrogen, a solution of benzyltrimethylammonium tribromide (1.49 g, 3.74 mmol) in CH₂Cl₂/MeOH 1:1 (10 ml) was added dropwise over 45 min. The orange mixture was kept at 0° C. for 7 h and then at room temperature overnight, at which time the solution had become colorless. The reaction mixture was evaporated and the residue that obtained was dissolved in a mixture water (100 mL) and EtOAc (100 mL). The aqueous layer was separated and was further extracted with EtOAc (2×50 mL). The organic extracts were combined and washed with brine, dried (Na₂SO₄) and evaporated. The crude product was fractionated by flash chromatography (hexane/ethyl acetate, 1:3) and the second fraction that collected gave JRL01105 (STX356) (485 mg) as white solid at about 97% purity; δ_{H} (400 MHz, DMSO-d₆) 6.90 (1H, s, CH), 6.93 (2H, d, J=8.6 Hz), 7.03 (2H, dd, J 2.1 and 8.6 Hz), 7.30 (2H, d, J 2.1 Hz), 8.06 (1H, s), 8.56 (1H, s) and 10.45 (2H, br s, exchanged with D₂O, 2×OH). The main impurity was the mono-brominated derivative of STX356. A small quantity of this fraction was further purified by semi-preparative HPLC (Waters PrepLC RP18, 25×10 mm, flow rate: 10 mL/min, mobile phase: MeOH/H₂O, 60:40). The fraction with a retention time of 5.5 min was collected and upon evaporation gave a white solid; m.p. 198-205° C. (dec.). Found: C, 42.2; H, 2.65; N, 9.79; C₁₅H₁₁Br₂N₃O₂ requires C, 42.38; H, 2.61; N, 9.89.

### 1-[Bis-(3-bromo-4-sulfamoyloxyphenyl)methyl]-1H-[1,2,4]triazole (JRL01109, STX566)

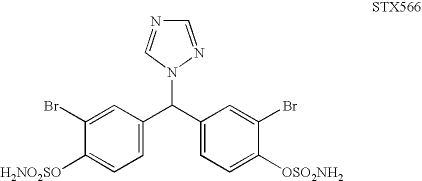

To a solution of JRL01105 (175 mg, 412 μmol) in N,N-dimethylacetamide (15 mL) at room temperature under an atmosphere of nitrogen was added sulfamoyl chloride (4.4 eq.). After stirring the reaction mixture overnight, it was diluted with ethyl acetate (30 mL) and the resulting mixture was washed with brine (50 mL, 4×20 mL), dried (MgSO₄), filtered and evaporated to give JRL01109 as a pale yellow residue (210 mg); m.p. 98-102° C.; δ_{H} (270 MHz, DMSO-d₆) 7.21 (1H, s, CH), 7.38 (2H, dd, J 2.1 and 8.4 Hz), 7.55 (2H, d, J=8.4 Hz), 7.66 (2H, d, J=2.1 Hz), 8.15 (1H, s), 8.32 (4H, br s) and 8.69 (1H, s); LRMS (FAB+): 584.0[10, (M+H)⁺], 515.0(10), 391.0[100, (M−2H₂NSO₂₀)⁺]; HRMS (FAB+): 583.87219 C₁₅H₁₄Br₂N₅O₆S₂ requires 583.87318.

### 1-[Bis-(3,5-dibromo-4-sulfamoyloxyphenyl)methyl]-1H-[1,2,4]triazole (LWO02128A, STX414)

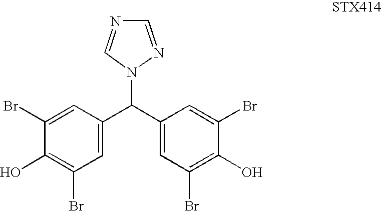

1-[Bis-(4-hydroxyphenyl)methyl]-1H-[1,2,4]triazole (450 mg, 1.684 mmol) was dissolved in hot acetic acid (100 mL). Upon cooling to ice/water temperature, potassium acetate (3.3 g, 33.67 mmol) was added to the yellow mixture followed by a solution of bromine in acetic acid dropwise (1.1 g/10 mL acetic acid, 7.5 mL, 5.061 mmol) over a period of 30 min. After stirring for another 30 min, water (20 mL) was added to the pale yellow gel/solid that resulted and the whole mixture was evaporated to give a wet pale yellow/beige residue. This crude was diluted with ethyl acetate (150 mL) and the blue green organic layer was washed with brine (4×100 mL), dried (MgSO₄), filtered and evaporated to give a slightly wet yellow residue (1.2 g). Upon standing in the round-bottomed flask unstoppered at room temperature overnight, a yellow/brown residue (850 mg) was obtained which upon trituration with acetone (10 mL) gave yellow deposits. After filtration and washing with more acetone, the pale yellow powder collected was air-dried to give LWO02128A (205 mg, 21%); m.p. 223-235° C. (dec.); ¹H (400 MHz, CDCl₃) 6.97 (1H, s, CH), 7.40 (4H, s, Ar), 8.11 (1H, s, triazole-H), 8.64 (1H, s, triazole-H) and 10.23 (2H, br s, exchanged with D₂O, 2×OH); LRMS (FAB+) 584.0 [48, (M+H)⁺], 513.0 [68, (M-triazole)⁺], 427.4 (95), 260.1 (88), 193.2 [100, ((M−4Br-triazole)⁺]; LRMS (FAB−) 579.8 (100, M⁻), 455.1 (40), 276.1 (70), 195.1 (60); HRMS (FAB−) 579.73119, C₁₅H₈⁷⁹Br₃⁸¹BrN₃O₂ requires 579.73295. 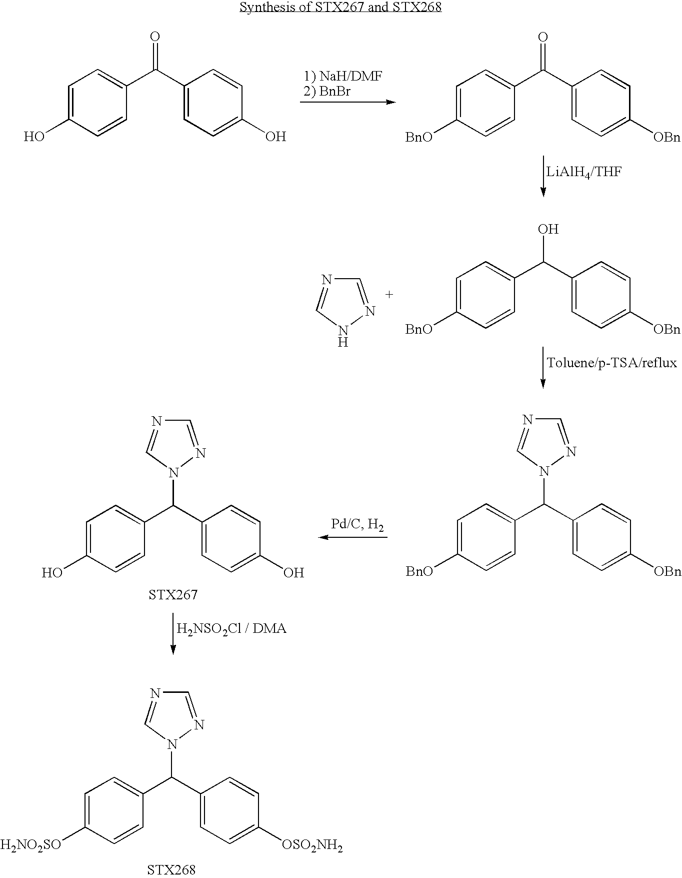
Compounds of Formula V

### 4-Benzyloxybenzyl chloride (LWO02011)

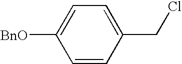

To a solution of 4-benzyloxybenzyl alcohol (5.0 g, 22.64 mmol) in dichloromethane (100 mL) at ice/water temperature was added dropwise thionyl chloride (2.5 mL, 33.96 mmol). The resulting clear pink/red solution was stirred at 0° C. for another 40 min before being evaporated to give a light green/yellow residue. After co-evaporation of the crude with dichloromethane three times, a creamy residue (5.77 g) was obtained which was dissolved in hot toluene (3 mL) and treated portionwise with hexane (60 mL). Upon standing at room temperature, LWO02011A was obtained as white crystals (2.89 g, 12.42 mmol). A second crop (LWO02011B, 1.41 g, 6.06 mmol, total yield: 81.6%) of the product was obtained from the residue of the mother liquor of the first crop after it has been recrystallised from hot hexane (20 mL); m.p. 74-80° C.; δ_{H} (400 MHz, DMSO-d₆) 4.72 (2H, s, CH₂Cl), 5.11 (2H, s, OCH₂), 7.01 (2H, m, Ar) and 7.39 (7H, m, Ar).

### 1-(4-Benzyloxybenzyl)-1H-[1,2,4]triazole (A) and 4-(4-Benzyloxybenzyl)-4H-[1,2,4]triazole (B) (LWO02013)

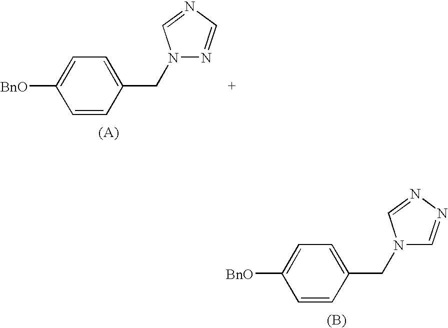

A suspension of (1H)-1,2,4-triazole (890 mg, 12.89 mmol), 4-benzyloxybenzyl chloride (2.0 g, 8.594 mmol), anhydrous potassium carbonate (1.19 g) in N,N-dimethylformamide (20 mL) was stirred at 90-95° C. for 4 h under an atmosphere of nitrogen. After cooling to room temperature, the reaction mixture was diluted with ethyl acetate (100 mL) and washed with 1M sodium hydroxide (150 mL) and then brine (4×50 mL). The organic layer was then dried (MgSO₄), filtered and evaporated to give a white residue (2.16 g). This crude was dissolved in hot ethyl acetate (15 mL) and hexane was added portionwise to the resulting solution. Upon cooling, LWO02013 was obtained as white crystals (1.34 g, 5.051 mmol). ¹H NMR has suggested LWO02013 contains a 1:1 mixture of the above isomers (A and B).

### 4-[1,2,4]Triazol-1-ylmethylphenol (LWO02015A, STX269)

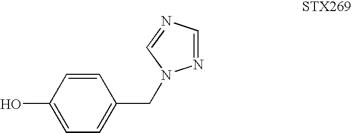

To a solution of LWO02013 (1.25 g, 4.711 mmol) in distilled THF (50 mL) was added absolute ethanol (10 mL) and Pd/C (10%, 70 mg). The resulting black suspension was stirred at room temperature for 72 h under an atmosphere of hydrogen (balloon). After removal by filtration and washings of the supported catalyst exhaustively with distilled THF, the filtrate was evaporated to give a white residue (580 mg) which was recrystallised from acetone/hexane to give LWO02015A as off white crystals (298 mg, 1.701 mmol). A second crop (LWO02015B, 77 mg, 439.5 μmol, total yield: 45.4%) of the product was obtained from the residue of the mother liquor when it was recrystallised in the same manner; m.p. 145-148° C. [Lit.¹ 143-146° C. (chloroform/Pet ether)]; δ_{H} (400 MHz, DMSO-d₆) 5.26 (2H, s, CH₂), 6.73 (2H, m, Ar), 7.13 (2H, m, Ar), 7.94 (1H, s, C3′-H), 8.58 (1H, s, C5′-H) and 9.50 (1H, s, exchanged with D₂O, OH); LRMS (FAB+): 351.2[10, (2M+H)⁺], 176.2[100, (M+H)⁺], 107.1[47, (M-Triazole)⁺], (FAB−): 481.3[30, (M+2NBA)⁻], 349.2[27, (2M−H)⁻], 328.2[100, (M+NBA)⁻], 221.2(23), 174.2 [100, (M−H)⁻];

HRMS (FAB+) 176.08183 C₉H₁₀N₃O requires 176.08239. Found: C, 61.6; H, 5.11; N, 23.6; C₉H₉N₃O requires C, 61.70; H, 5.18; N, 23.99.
¹ Abdreubu et. Al. (1989) Farmaco 44(9) 831-842.

### Sulfamic acid 4-[1,2,4]triazol-1-ylmethylphenyl ester (LWO02017A, STX270)

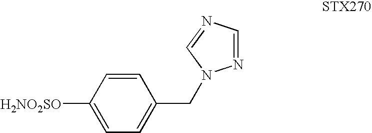

To a solution of LWO02015A (150 mg, 856.2 μmol) in distilled THF (10 mL) at 0° C. was added sodium hydride (60% in mineral oil, 36 mg, 899 mmol). After stirring for 10 min at which no more evolution of hydrogen was observed, sulfamoyl chloride in toluene (ca. 0.68M, 2.5 mL) was added under an atmosphere of nitrogen and the resulting thin white suspension was stirred at room temperature for 3 h. The reaction mixture was then diluted with ethyl acetate (70 mL) and washed with brine (100 mL, 3×50 mL). The organic layer that separated was dried (MgSO₄), filtered and evaporated to give a white residue (144 mg) which was recrystallised from acetone/hexane to give LWO02017A as white crystals (71 mg, 279.2 μmol, 32.6%); νₘₐₓ (KBr) 3352, 3127, 2880, 2646, 1509, 1369, 1183, 1158 cm⁻¹; δ_{H} (400 MHz, DMSO-d₆) 5.44 (2H, s, CH₂), 7.27 (2H, AA′BB′), 7.38 (2H, AA′BB′), 8.0 (˜2.7H, m, reduced to one proton, singlet, after D₂O exchange, C3′-H and H₂NSO₂) and 8.68 (1H, s, C5′-H); Found: C, 42.6; H, 4.01; N, 21.7; C₉H₁₀N₄O₃S requires C, 42.51; H, 3.96; N, 22.03. 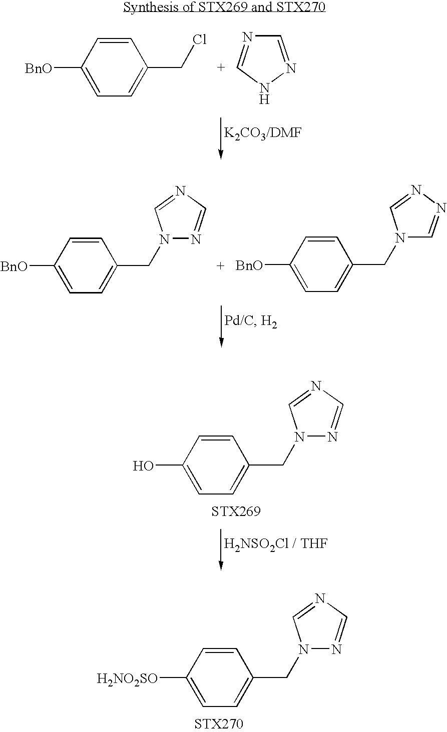
Biological Data

Compounds were tested for aromatase and steroid sulphatase inhibition in accordance with the following Protocols. Each compound in accordance with the present invention is found to inhibit steroid sulphatase and aromatase.

Assay For Determining Sts Activity Using Cancer Cells

Inhibition of Steroid Sulphatase Activity in JEG3 Cells

Steroid sulphatase activity is measured in vitro using intact JEG3 choriocarcinoma cells.

This cell line may be used to study the control of human breast cancer cell growth. It possesses significant steroid sulphatase activity (Boivin et al., J. Med. Chem., 2000, 43: 4465-4478) and is available in from the American Type Culture Collection (ATCC).

Cells are maintained in Minimal Essential Medium (MEM) (Flow Laboratories, Irvine, Scotland) containing 20 mM HEPES, 5% foetal bovine serum, 2 mM glutamine, non-essential amino acids and 0.075% sodium bicarbonate. Up to 30 replicate 25 cm2 tissue culture flasks are seeded with approximately 1×10⁵ cells/flask using the above medium. Cells are grown to 80% confluency and the medium is changed every third day.

Intact monolayers of JEG3 cells in triplicate 25 cm² tissue culture flasks are washed with Earle's Balanced Salt Solution (EBSS from ICN Flow, High Wycombe, U.K.) and incubated for 3-4 hours at 37° C. with 5 pmol (7×10⁵ dpm) [6,7-3H]oestrone-3-sulphate (specific activity 60 Ci/mmol from New England Nuclear, Boston, Mass., U.S.A.) in serum-free MEM (2.5 ml) together with oestrone-3-sulphamate (11 concentrations: 0; 1fM; 0.01 pM; 0.1 μM; 1 μM; 0.01 nM; 0.1 nM; 1 nM; 0.01 mM; 0.1 mM; 1 mM). After incubation each flask is cooled and the medium (1 ml) is pipetted into separate tubes containing [14C]oestrone (7×103 dpm) (specific activity 97 Ci/mmol from Amersham International Radiochemical Centre, Amersham, U.K.). The mixture is shaken thoroughly for 30 seconds with toluene (5 ml). Experiments have shown that >90% [14C] oestrone and <0.1% [3H]oestrone-3-sulphate is removed from the aqueous phase by this treatment. A portion (2 ml) of the organic phase is removed, evaporated and the 3H and 14C content of the residue determined by scintillation spectrometry. The mass of oestrone-3-sulphate hydrolysed was calculated from the 3H counts obtained (corrected for the volumes of the medium and organic phase used, and for recovery of [14C] oestrone added) and the specific activity of the substrate. Each batch of experiments includes incubations of microsomes prepared from a sulphatase-positive human placenta (positive control) and flasks without cells (to assess apparent non-enzymatic hydrolysis of the substrate). The number of cell nuclei per flask is determined using a Coulter Counter after treating the cell monolayers with Zaponin. One flask in each batch is used to assess cell membrane status and viability using the Trypan Blue exclusion method (Phillips, H. J. (1973) In: Tissue culture and applications, [eds: Kruse, D. F. & Patterson, M. K.]; pp. 406-408; Academic Press, New York).

Results for steroid sulphatase activity are expressed as the mean±1 S. D. of the total product (oestrone+oestradiol) formed during the incubation period (3-4 hours) calculated for 106 cells and, for values showing statistical significance, as a percentage reduction (inhibition) over incubations containing no oestrone-3-sulphamate. Unpaired Student's t-test was used to test the statistical significance of results.

Assay for Determining Sts Activity Using Placental Microsomes

Inhibition of Steroid Sulphatase Activity in Placental Microsomes

Sulphatase-positive human placenta from normal term pregnancies are thoroughly minced with scissors and washed once with cold phosphate buffer (pH 7.4, 50 mM) then re-suspended in cold phosphate buffer (5 ml/g tissue). Homogenisation is accomplished with an Ultra-Turrax homogeniser, using three 10 second bursts separated by 2 minute cooling periods in ice. Nuclei and cell debris are removed by centrifuging (4° C.) at 2000 g for 30 minutes and portions (2 ml) of the supernatant are stored at 20° C. The protein concentration of the supernatants is determined by the method of Bradford (Anal. Biochem., 72, 248-254 (1976)).

Incubations (1 ml) are carried out using a protein concentration of 100 mg/ml, substrate concentration of 20 mM [6,7-3H]oestrone-3-sulphate (specific activity 60 Ci/mmol from New England Nuclear, Boston, Mass., U.S.A.) and an incubation time of 20 minutes at 37° C. If necessary eight concentrations of compounds are employed: 0 (i.e. control); 0.05 mM; 0.1 mM; 0.2 mM; 0.4 mM; 0.6 mM; 0.8 mM; 1.0 mM. After incubation each sample is cooled and the medium (1 ml) was pipetted into separate tubes containing [14C]oestrone (7×103 dpm) (specific activity 97 Ci/mmol from Amersham International Radiochemical Centre, Amersham, U.K.). The mixture is shaken thoroughly for 30 seconds with toluene (5 ml). Experiments have shown that >90% [14C]oestrone and <0.1% [3H]oestrone-3-sulphate is removed from the aqueous phase by this treatment. A portion (2 ml) of the organic phase was removed, evaporated and the 3H and 14C content of the residue determined by scintillation spectrometry. The mass of oestrone-3-sulphate hydrolysed is calculated from the 3H counts obtained (corrected for the volumes of the medium and organic phase used, and for recovery of [14C]oestrone added) and the specific activity of the substrate.

Animal Assay Model for Determining Sts Activity

Inhibition of Oestrone Sulphatase Activity In Vivo

The compounds of the present invention may be studied using an animal model, in particular in ovariectomised rats. In this model compounds which are oestrogenic stimulate uterine growth.

The compound (0.1 mg/Kg/day for five days) is administered orally to rats with another group of animals receiving vehicle only (propylene glycol). At the end of the study samples of liver tissue were obtained and oestrone sulphatase activity assayed using 3H oestrone sulphate as the substrate as previously described (see PCT/GB95/02638).

Assay for Determining Aromatase Activity Using Jeg3 Cells

Aromatase activity is measured in JEG3 choriocarcinoma cells, obtained from the ATCC. This cell line possesses significant aromatase activity and is widely used to study the control of human aromatase activity (Bhatnager et al., J. Steroid Biochem. Molec. Biol. 2001, 76:199-202). Cells are maintained in Minimal Essential Medium (MEM, Flow Laboratories, Irvine, Scotland) containing 20 mM HEPES, 10% foetal bovine serum, 2 mM glutamine, non-essential amino acids and 0.075% sodium bicarbonate. Intact monolayers of JEG3 cells (2.5×10⁶ cells) in triplicate 25 cm² tissue culture flasks are washed with Earle's Balanced salt solution (EBSS, from ICN Flow, High Wycombe, UK) and incubated with [1β-³H] androstenedione (2-5 nM, 26 Ci/mmol, New England Nuclear, Boston, Mass., USA) for 30 min with inhibitors over the range of 10 pm-10 μM. During the aromatase reaction, ³H₂O is liberated which can he quantified using a liquid scintillation spectrometer (Beckman-Coulter, High Wycombe, Bucks. UK). This ³H₂O-release method has been widely used to measure aromatase activity (Newton et al., J. Steroid Biochem. 1986,24:1033-1039). The number of cell nuclei per flask is determined using a Coulter Counter after treating the cell monolayers with Z aponin.

Results for aromatase activity are expressed as the mean±1 S.D. of the product formed during the incubation period (30 min) calculated for 10⁶ cells and, for values showing a statistical significance, as a percentage reduction (inhibition) over incubations containing no aromatase inhibitor. Unpaired Student's t test was used to test the statistical significance of results. IC₅₀ values were calculated as the concentration of inhibitor required to obtain a 50% inhibition of aromatase activity.

Animal Assays for Determining Aromatase Activity

(i) Inhibition of PMSG-Induced Oestrogen Synthesis

The ability of compounds to inhibit aromatase activity in vivo was tested using a pregnant mare serum gonadotrophin (PMSG)-induced oestrogen synthesis assay. For this, female rats (250 g) were injected with PMSG (200 IU, s.c.). After 72 h rats were administered vehicle (propylene glycol) or various doses of test compounds orally. At 2 h after dosing blood samples were obtained by cardiac puncture (under anaesthesia). Plasma oestradiol levels were measured in control groups and groups receiving drugs. The efficacy of aromatase inhibition was determined by measurement of plasma oestradiol concentrations by radioimmunoassay. This method has been widely used to determine the effectiveness of aromatase inhibitors in vivo (Wouters et al., J. Steroid Biochem., 1989, 32:781-788).

(ii) Inhibition of Androstenedione Stimulated Uterine Growth in Ovariectomised Rats

Female rats (250 g) were ovariectomised and used to determine the effectiveness of aromatase inhibition on androstenedione stimulated uterine growth. Administration of androstenedione (30 mg/kg/d) for a 2-week period results in a significant increase in uterine growth in ovariectomised animals. This increase in uterine growth is stimulated by oestrogen which is derived from the administered androstenedione as a result of the action of the aromatase enzyme. By co-administration of compounds with androstenedione the extent of aromatase inhibition can be determined by measurements of uterine weights in treated and untreated animals.

The following in vitro data were recorded.

| | | AROMATASE | SULPHATASE |
|---|---|---|---|
| Compound | Structure | IC₅₀ (nM) | IC₅₀ (nM) |
| H340F1 | H340F1 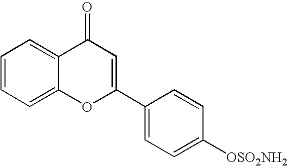 | (59% inhibition at 10 μM) IC₅₀ > 10 μM | 10 μM |
| H342F1 | H342F1 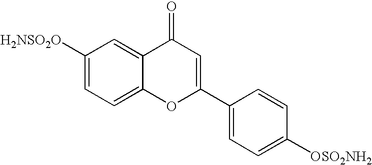 | (No inhibition seen) | 90 |
| STX258 | 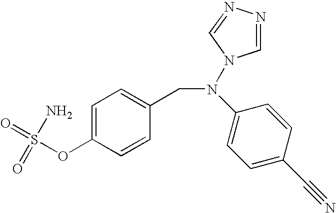 | 100 | 227 |
| STX265 | 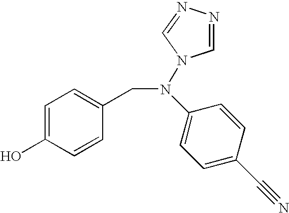 | 23 | n.d. |
| STX 268 | 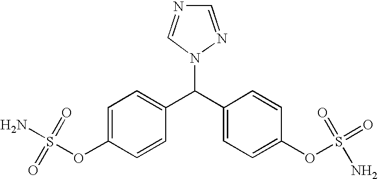 | 3044 | >10000 (31% at 10 μM) |
| STX269 | 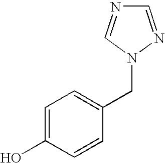 | 41% inhibition at 10 μM | n.d. |
| STX270 | 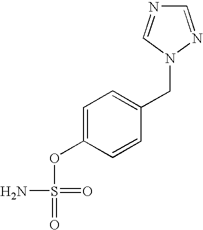 | 62% inhibition at 10 μM | 14% inhibition at 10 μM in placental microsomes |
| STX273 | 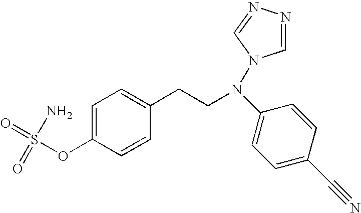 | 69% inhibition at 0.1 μM | n.d. |
| STX 287 | 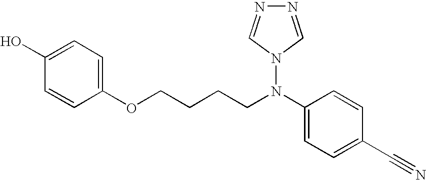 | 4.4 | n.d. |
| STX 288 | 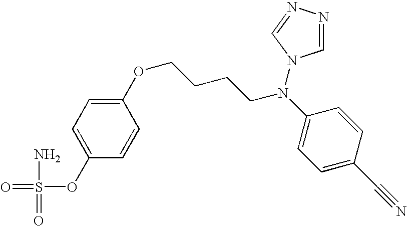 | 31 | >10 μM |
| STX 290 | 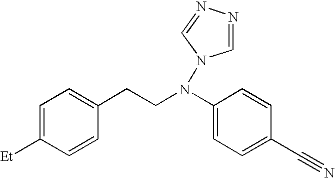 | 1.3 | n.d. |
| STX 291 | 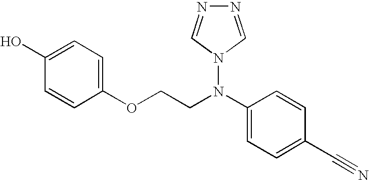 | 26 | n.d. |
| STX 292 | 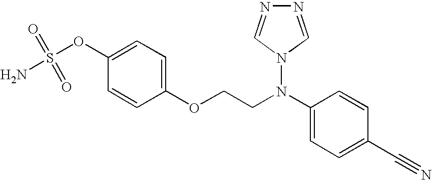 | 767 | >10 μM |
| STX 300 | 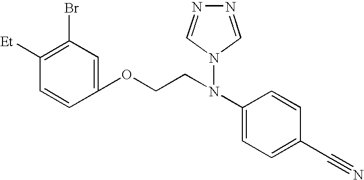 | 1.6 | n.d. |
| STX 301 | 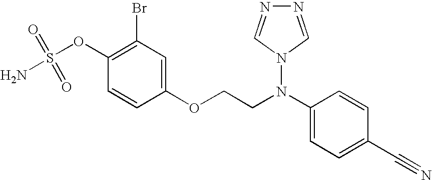 | 119 | n.d. |
| STX 310 | 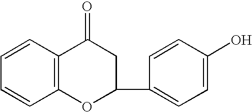 | (42% inhibition at 10 μM) IC₅₀ >10 μM | n.d. |
| STX 566 | 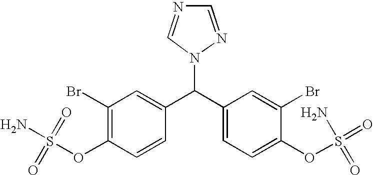 | 4.9 | 476 |
| STX 597 | 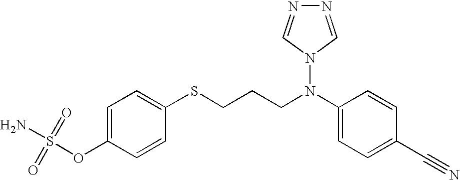 | 0.51 | >10000 |
| STX 636 | 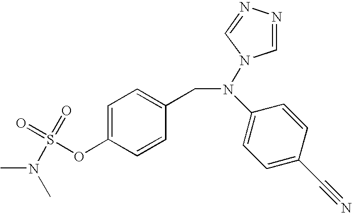 | 9 | >10000 (6.6% at 10 μM) |
| STX 681 | 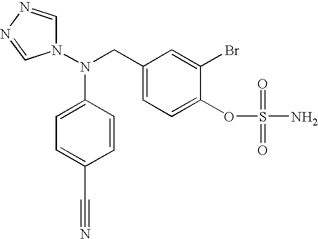 | 0.82 | 39 |
| STX 694 | 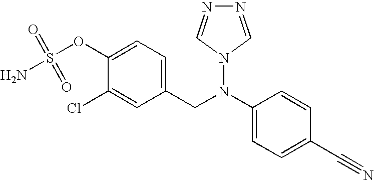 | 2.3 | 20 |
| STX 699 | 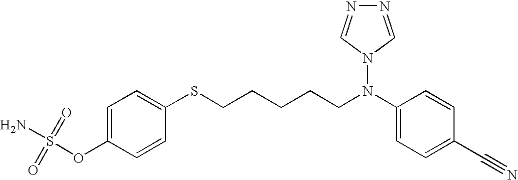 | 0.73 | 1000 |
| STX 700 | 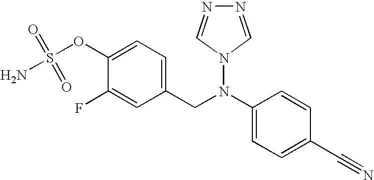 | 12 | 40 |
| STX 732 | 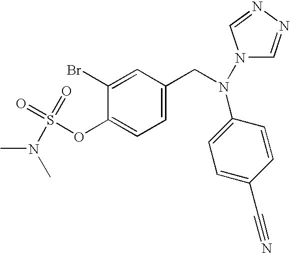 | 19 | >10000 (12% at 10 μM) |

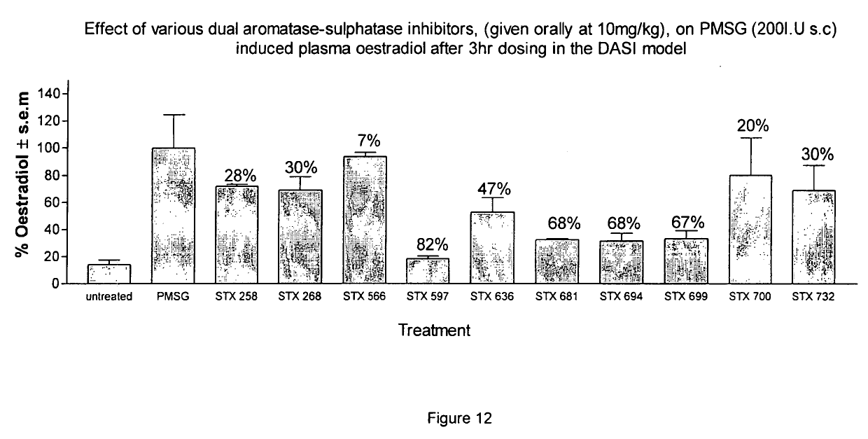

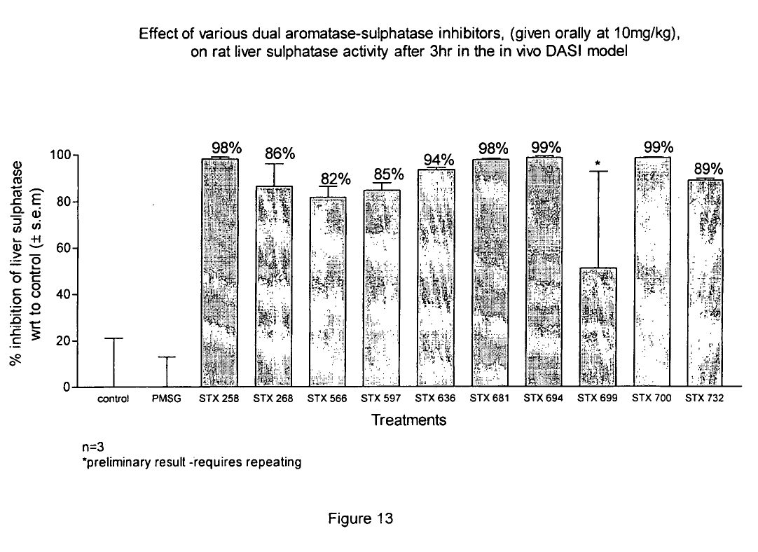

In vivo data were recorded using the above described aromatase and STS animals assays. The relevant compounds were administered and for each animal both aromatase and STS activities were determined. The data are shown in FIGS. 12 and 13.

The following compounds were also synthesised and tested.

Synthetic Routes

Compounds in accordance with the present invention were synthesised in accordance with the synthetic routes and schemes.

### EXPERIMENTAL

### 3,3′-Dihydroxybenzophenone (MW01012/PMW01016)

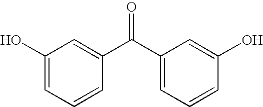

This was prepared as described by Wittig et al. [Chem. Berichte, 1947, 363].

### Bis-(3-hydroxyphenyl)methanol (MW01015/PMW01019)

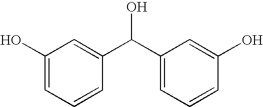

A solution of sodium borohydride (NaBH₄) (0.19 g, 5.14 mmol) in H₂O (5 mL) was added to an orange solution of 3,3′-dihydroxybenzophenone (1.00 g, 4.67 mmol) in EtOH (15 mL) and the resulting mixture was stirred for 1 h. After this time, the reaction mixture was added to ice-water (40 mL) containing conc. HCl (2.5 mL) and the solution was extracted with EtOAc (2×60 mL). The combined organic layers were washed with NaOH (3M, 2×50 mL) and the combined basic washes were acidified with conc. HCl. The organic components that appeared were extracted with EtOAc (2×100 mL). The combined EtOAc extracts were dried (MgSO₄) and the solvent was removed in vacuo to give the crude product as a brown oil which solidified on standing. Purification was achieved by recrystallisation from H₂O to give the title compound as a brown crystalline solid (0.86 g, 85%, mp 141-142° C.); δ_{H} (270 MHz, d⁶-DMSO) 9.27 (2H, br s, ArOH), 7.05 (2H, t, J=8.2, ArH), 6.84-6.72 (4H, m, ArH), 6.63-6.54 (2H, m, ArH), 5.72 (1H, br s, CHOH), 5.50 (1H, s, CHOH); δ_{C} (68 MHz, d⁶-DMSO) 157.1 (2×C), 147.2 (2×C), 128.9 (2×CH), 117.0 (2×CH), 113.6 (2×CH), 113.1 (2×CH), 74.1 (CH); LCMS (ES⁻) 215.0 ([M−H]⁻, 30%), 196.9 (10), 168.8 (15), 120.7 (100); HRMS (FAB+) Found 216.0785, C₁₃H₁₂O₃ requires 216.0786.

### 1-[Bis-(3-hydroxyphenyl)methyl]-1H-[1,2,4]triazole (PW01018/PMW01021, STX951)

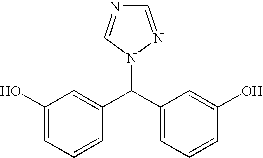

Bis-(3-hydroxyphenyl)methanol (0.75 g, 3.47 mmol), 1,2,4-triazole (0.48 g, 6.94 mmol), p-TSA (125 mg) and toluene (300 mL) were dissolved/suspended in toluene and heated at reflux with a Dean-Stark separator for 24 h. The reaction mixture was allowed to cool, and the solvent was removed in vacuo. The resulting residue was dissolved in EtOAc (75 mL) and the organic layer was washed with H₂O (3×75 mL), dried (MgSO₄) and the solvent was removed in vacuo to give a brown solid. The crude product was purified by flash column chromatography (EtOAc:Hexane 3:1) to give the title compound (0.82 g, 88%) as a pale yellow oil which formed a foam after a prolonged period under high vacuum; δ_{H} (270 MHz, d⁶-DMSO) 9.50 (2H, br s, ArOH), 8.55 (1H, s, NCHN), 8.06 (1H, s, NCHN), 7.16 (2H, t, J=7.9, ArH), 6.89 (1H, s, CH), 6.75-6.68 (2H, m, ArH), 6.67-6.60 (4H, m, ArH); δ_{C} (100 MHz, d⁶-DMSO) 158.0 (2×C), 152.3 (CH), 144.9 (CH), 141.0 (2×C), 130.2 (2×CH), 119.3 (2×CH), 115.6 (2×CH), 115.6 (2×CH), 66.2 (CH); LRMS (FAB+) 268.3 ([M+H]⁺, 100%), 199.2 (85); HRMS (FAB+) Found 268.1091, C₁₅H₁₄N₃O₂ requires 268.1086; HPLC (CH₃CN/H₂O, 90:10) t_{R}=1.79 min (purity: 99+%).

### 1-[Bis-(3-sulphamoyloxyphenyl)methyl]-1H-[1,2,4]triazole (PMW01023, STX1001)

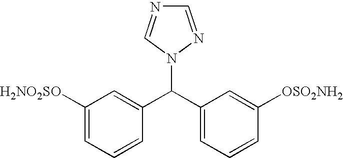

A solution of sulphamoyl chloride (H₂NSO₂Cl) in toluene (0.7 M, 8.8 mL) was concentrated in vacuo at 30° C. to furnish a yellow oil which solidified upon cooling in an ice bath. DMA (7 mL) and STX951 (0.30 g, 1.12 mmol) were subsequently added and the mixture was allowed to warm to room temperature and stirred overnight. The resulting yellow solution was added to brine (30 mL) and this was extracted with EtOAc (3×50 mL). The organic layers were combined, washed with H₂O (3×50 mL), dried (MgSO₄) and the solvent was removed in vacuo. The crude product was purified by flash column chromatography (CHCl₃:Acetone 1:1) to give the title compound STX1001 (0.42 g, 88%) as a pale yellow oil which formed a foam after a prolonged period under high vacuum; δ_{H} (270 MHz, d⁶-DMSO) 8.64 (1H, s, NCHN), 8.11 (1H, s, NCHN), 8.04 (4H, br s, 2×NH₂), 7.49 (2H, t, J=7.9, ArH), 7.36-7.16 (7H, m, ArH and CH); δ_{C} (100 MHz, d⁶-DMSO) 152.7 (2×C), 150.7 (CH), 145.2 (2×C), 140.9 (CH), 130.6 (2×CH), 126.8 (2×CH), 122.4 (4×CH), 64.8 (CH); LRMS (FAB+) 426.0 ([M+H]⁺, 100%), 357.0 (50), 85.1 (50); HRMS (FAB+) Found 426.0540, C₁₅H₁₆N₅O₆S₂ requires 426.0542; HPLC (CH₃CN/H₂O, 90:10) t_{R}=1.50 min (purity: 99+%).

### 5-Bromo-2-methoxyphenol

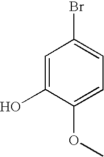

This was prepared as described by Van der May et al [J. Med. Chem 2001, 44, 2523].

### 2-Benzyloxy-4-bromo-1-methoxybenzene (PMW01028)

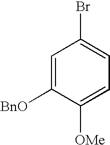

NaH, 60% dispersion in mineral oil (0.61 g, 15.3 mmol) was added in three equal portions to DMF (25 mL) pre-cooled to 0° C. 5-Bromo-2-methoxyphenol (2.60 g, 12.8 mmol) was then added in three portions, and the resulting brown solution was stirred for 20 min. Benzyl bromide (2.63 g, 15.3 mmol) was added and the reaction mixture was allowed to warm to room temperature and was stirred overnight resulting in the formation of a yellow solution. The reaction mixture was poured onto H₂O (40 mL) leading to the formation of a white precipitate which was extracted into EtOAc (4×50 mL). The combined organics were washed with H₂O (4×75 mL), brine (75 mL), dried (MgSO₄) and the solvent was removed in vacuo. The crude product was obtained as a white solid which was purified by recrystallisation from Et₂O to give the title compound (2.90 g, 77%, mp 109-110° C. [lit. 105-106° C. (Et₂O)]^{Aust. J. Chem., 1981, 34, 587}) as a white crystalline solid; δ_{H} (270 MHz, CDCl₃) 7.47-7.26 (5H, m, ArH), 7.07-7.00 (2H, m, ArH), 6.74 (1H, d, J=8.7, ArH), 5.10 (2H, s, CH₂), 3.85 (3H, s, CH₃), LRMS (FAB+) 294.0 ([M+H]⁺, 35%), 292.0 (35), 91.1 (100).

### Bis-(3-benzyloxy-4-methoxyphenyl)methanol (PMW01033/PMW01035)

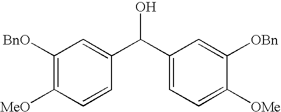

n-BuLi (1.6 M, 5.2 mL) was added to a solution of 2-benzyloxy-4-bromo-1-methoxybenzene (2.10 g, 7.2 mmol) in THF (28 mL) at −78° C. and the resulting yellow solution was stirred at this temperature for 1 h. A solution of 3-benzyloxy-4-methoxybenzaldehyde (1.74 g, 7.2 mmol) in THF (16 mL) was added dropwise at −78° C. and the reaction mixture was stirred at this temperature for 1.5 h and then at room temperature for 2 h. Water (100 mL) was added to the yellow solution and the resulting mixture was extracted with EtOAc (3×100 mL). The combined organics were dried (MgSO₄), and solvent was removed in vacuo to give a thick yellow oil which solidified after standing overnight. The crude product was purified by precipitation from EtOAc/hexane to give the title compound (1.70 g, 52%, mp 103-105° C.) as a white solid; δ_{H} (270 MHz, CDCl₃) 7.42-7.21 (10H, m, ArH), 6.89-6.79 (6H, m, ArH), 5.63 (1H, d, J=3.5, CHOH), 5.06 (4H, s, 2×CH₂), 3.86 (6H, s, 2×CH₃), 2.02 (1H, d, J=3.5, CHOH); δ_{C} (100 MHz, d⁶-DMSO) 149.2 (2×C), 148.2 (2×C), 137.2 (2×C), 136.6 (2×C), 128.7 (4×CH), 128.0 (2×CH), 127.6(4×CH), 119.5 (2×CH), 112.6(2×CH), 111.7 (2×CH), 75.8 (CH), 71.2 (2×CH₂), 56.4 (2×CH₃); LRMS (FAB+) 456.1 (M+, 45%), 439.1 (85), 349.2 (10), 243.1 (20), 91.1 (100); HRMS (FAB+) Found 456.1944, C₂₉H₂₈O₅ requires 456.1937.

### 1-[Bis-(3-benzyloxy-4-methoxyphenyl)methyl]-1H-[1,2,4]triazole (PMW01036/PMW01041)

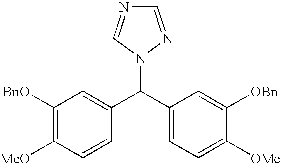

Bis-(3-benzyloxy-4-methoxyphenyl)methanol (1.60 g, 3.50 mmol), 1,2,4-triazole (0.48 g, 6.95 mmol) and p-TSA (160 mg) dissolved/suspended in toluene (230 mL) were heated at reflux with a Dean-Stark separator for 24 h. The reaction mixture was allowed to cool, and the solvent was removed in vacuo. The resulting residue was dissolved in EtOAc (100 mL) and the organic layer was washed with H₂O (3×100 mL), dried (MgSO₄) and the solvent was removed in vacuo. The crude product was purified using Flashmaster II (EtOAc/Hexane) to give the title compound (1.51 g, 84%, mp 96-99° C.) as a yellow oil which solidified after standing for a prolonged period; δ_{H} (400 MHz, CDCl₃) 7.95 (1H, s, NCHN), 7.67 (1H, s, NCHN), 7.34-7.24 (10H, m, ArH), 6.81 (2H, d, J=8.4, ArH), 6.58-6.48 (5H, m, ArH), 5.00 (4H, s, 2×CH₂), 3.84 (6H, s, 2×CH₃); δ_{C} (100 MHz, CDCl₃) 152.3 (CH), 149.9 (2×C), 148.2 (2×C), 143.4 (CH), 136.7 (2×C), 130.5 (2×C), 128.7 (4×CH), 128.1 (2×CH), 127.5 (4×CH), 121.1 (2×CH), 114.1 (2×CH), 118.8 (2×CH), 71.2 (2×CH₂), 67.4 (CH), 56.4 (2×CH₃); LRMS (FAB+) 507.2 (M⁺, 20%), 439.2 (100), 349.2 (15), 257.2 (15), 91.1 (35); HRMS (FAB+) Found 507.2159, C₃₁H₂₉N₃O₄ requires 507.2158; Found: C, 73.00%; H, 5.73%; N, 8.31%, C₃₁H₂₉N₃O₄ requires: C, 73.35%; H, 5.76%; N, 8.28%.

### 1-[Bis-(3-hydroxy-4-methoxyphenyl)methyl]-1H-[1,2,4]triazole (PMW01048 STX1003)

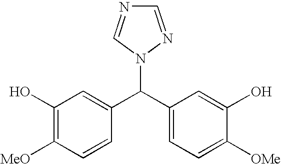

10% Pd/C (80 mg) was added to a solution of 1-[bis-(3-benzyloxy-4-methoxyphenyl)methyl]-1H-[1,2,4]triazole (1.65 g, 3.25 mmol) in THF/MeOH (1:1, 200 mL). The solution was stirred under an atmosphere of H₂ (provided by addition from a balloon) overnight. The excess H₂ was removed and the reaction mixture was filtered through Celite® washing with THF and MeOH, then the solvent was removed in vacuo. The crude product was purified using Flashmaster II (EtOAc/Hexane) to give the title compound STX1003 (0.86 g, 81%, mp 217.5-219° C.) as a white solid; δ_{H} (270 MHz, d⁶-DMSO) 9.08 (2H, s, 2×OH), 8.47 (1H, s, NCHN), 8.03 (1H, s, NCHN), 6.88 (2H, d, J=8.4, ArH), 6.76 (1H, s, CH), 6.64 (2H, s, ArH), 6.57 (2H, d, J=8.4, ArH), 3.74 (6H, s, 2×CH₃); δ_{C} (68 MHz, d⁶-DMSO) 151.8 (CH), 147.3 (2×C), 146.3 (2×C), 144.0 (CH), 130.1 (2×C), 118.8 (2×CH), 115.3 (2×CH), 111.9 (2×CH), 64.9 (CH), 55.6 (2×CH₃); LCMS (ES⁻) 326.2 ([M−H]⁻, 100%), 255.9 (70), 240.4 (60), 211.4 (70); HRMS (FAB⁺) Found 327.1226, C₁₇H₁₇N₃O₄ requires 327.1219; HPLC (CH₃CN/H₂O, 90:10) t_{R}=1.57 min (purity: 99+%); Found: C, 62.10%, H, 5.26%; N, 12.80%, C₁₇H₁₇N₃O₄ requires: C, 62.38%; H, 5.23%; N, 12.84%.

### 1-[Bis-(3-sulphamoyloxy-4-methoxyphenyl)methyl]-1H-[1,2,4]triazole (PMW01050 STX1007)

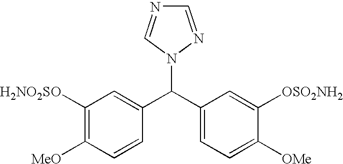

A solution of sulphamoyl chloride (H₂NSO₂Cl) in toluene (0.7 M, 5.8 mL) was concentrated in vacuo at 30° C. to furnish a yellow oil which solidified upon cooling in an ice bath. DMA (6 mL) and STX1003 (0.30 g, 0.92 mmol) were subsequently added and the mixture was allowed to warm to room temperature and stirred overnight. The resulting mixture was poured onto brine (50 mL) and this was extracted with EtOAc (3×50 mL). The organic layers were combined, washed with H₂O (2×50 mL) and brine (2×50 mL), dried (MgSO₄) and the solvent was removed in vacuo. The crude product was purified using Flashmaster II (EtOAc/Hexane) to give the title compound STX1007 (0.40 g, 90%) as a white foam; δ_{H} (270 MHz, d⁶-DMSO), 8.56 (1H, s, NCHN), 8.07 (1H, s, NCHN), 7.93 (4H, br s, 2×NH₂), 7.26-7.07 (6H, m, ArH), 7.00 (1H, s, CH), 3.80 (6H, s, 2×CH₃); δ_{C} (68 MHz, d⁶-DMSO) 151.8 (CH), 151.6 (2×C), 144.3 (CH), 138.4 (2×C), 131.0 (2×C), 127.1 (2×CH), 123.3 (2×CH), 113.4 (2×CH), 63.9 (CH), 56.0 (2×CH₃); LRMS (FAB+) 486.1 ([M+H]⁺, 20%), 417.0 (100), 338.1 (14), 257.1 (6); HRMS (FAB+) Found 485.0659, C₁₇H₁₉N₅O₈S₂ requires 485.0675; HPLC (CH₃CN/H₂O, 90:10) t_{R}=1.48 min (purity: 99+%).

### 4-[(3-Benzyloxybenzyl)(4-cyanophenyl)amino]-4H-[1,2,4]triazole

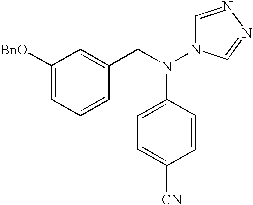

To a stirred suspension of NaH (60% dispersion in oil, 0.22 g, 5.4 mmol) in anhydrous DMF (5 mL) was added a solution of 4-[(4-cyanophenyl)amino]-4H-[1,2,4]triazole (1.0 g, 5.68 mmol) in anhydrous DMF (3 mL) and the mixture stirred at r.t. for 0.5 h. A solution of 3-benzyloxybenzyl bromide^{[Thakkar, 1993]} (1.57 g, 5.66 mmol) in anhydrous DMF (2 mL) was then added and the mixture heated at 80-90° C. overnight. The mixture was cooled, diluted with EtOAc (50 mL), washed with water (4×100 mL), brine (100 mL) and dried (Na₂SO₄). Concentration in vacuo an orange residue which was purified by chromatography [SiO₂, EtOAc/n-hexane (1:1)] and to give 4-[(3-benzyloxybenzyl)(4-cyanophenyl)amino]-4H-[1,2,4]triazole as a colourless solid (0.58 g, 28%) after crystallisation; δ_{H} (400 MHz, CDCl₃) 4.85 (s, 2H), 5.03 (s, 2H); 6.62 (AA′BB′, 2H), 6.77 (d, J=7.8, 1H), 6.79 (d, J=2.4, 1H), 6.96 (dd, J=7.8, 2.4, 1H), 7.26 (t, J=7.8, 1H), 7.34-7.37 (m, 5H), 7.57 (AA′BB′, 2H), 8.04 (s, 2H); LC-MS (APCI+) t_{R}=6.81 min, m/z=382 (M+H); HPLC t_{R}=2.27 min (98%).
Thakkar et al., J. Med. Chem. 1993, 36, 2950-2955.

### 4-[(3-Hydroxybenzyl)(4-cyanophenyl)amino]-4H-[1,2,4]triazole (STX333)

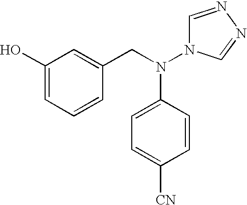

To a solution of 4-[(3-Benzyloxybenzyl)(4-cyanophenyl)amino]-4H-[1,2,4]triazole (0.4 g, 1.05 mmol) in anhydrous THF/MeOH (20 mL) was added Pd—C (10% by wt., 0.04 g).

The black suspension was then stirred under an atmosphere of hydrogen (balloon) for 16 h. The catalyst was removed by filteration through Celite® and exhaustively washed with THF. The filtrate was concentrated in vacuo to give a beige residue. Recrystallisation from EtOH gave STX333 as a colourless solid (0.27 g, 88%) after recrystallisation; δ_{H} (400 MHz, DMSO-d₆) 4.97 (s, 2H), 6.68 (AA′BB′, 2H), 6.72-6.75 (m, 3H), 7.11 (m, 1H), 7.76 (AA′BB′, 2H), 8.77 (s, 2H), 9.49 (br s, 1H—exchanges with D₂O); LRMS (FAB+) m/z (rel. intensity) 292 (100%, [M+H]), 223 (42, [M+H-triazole]); HPLC t_{R}=2.22 min, (96%); HRMS (FAB+) Found 292.1198; C₁₆H₁₃N₅O requires 292.1192;

### 4-[(3-O-Sulfamoylbenzyl)(4-cyanophenyl)amino]-4H-[1,2,4]triazole (STX334)

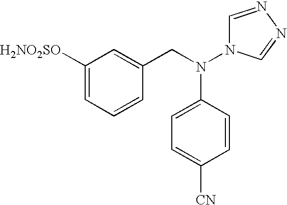

To an ice-cooled solution of STX333 (0.1 g, 0.34 mmol) in anhydrous DMA (2 mL) was added sulfamoyl chloride (0.59 M solution in toluene, 1.2 mL—the toluene was removed in vacuo [not allowing the temperature of the water bath to exceed 30° C.] prior to addition, 0.69 mmol) and the mixture stirred (under a positive flow of dry nitrogen) overnight. The mixture was diluted with EtOAc (25 mL), washed with water (3×50 mL) and brine (50 mL) and concentrated in vacuo (not allowing the temperature of the water bath to exceed 30° C.). The residue was gave STX334 as a colourless solid (0.06 g, 47%) after crystallisation from CH₂C₁₂/acetone; δ_{H} (400 MHz, DMSO-d₆) 5.11 (s, 2H), 6.74 (AA′BB′, 2H), 7.20-7.27 (m, 3H), 7.40 (1H, t, J=7.8), 7.77 (AA′BB′, 2H), 7.98 (br s, 2H—exchanges with D₂O), 8.81 (s, 2H); LRMS (FAB+) m/z (rel. intensity) 371 (100%, [M+H]), 302 (28, [M+H-triazole]); HPLC t_{R}=2.13 min (99%); HRMS (FAB+) Found 371.0926; C₁₆H₁₅N₆O₃S requires 371.0946.

### 3-Benzoyloxy-4-fluorotoluene

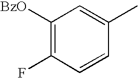

To a solution of 4-fluoro-3-hydroxytoluene (3.784 g, 30 mmol)) in CH₂Cl₂ (50 mL) was added NEt₃ (5 mL) and the mixture stirred at room temperature for 0.5 h. Benzoyl chloride (4.22 mL, 30 mmol) was then added and the reaction stirred at room temperature for 18 h. The reaction was concentrated in vacuo and the residue redissolved in Et₂O (200 mL), washed with water (100 mL), 2N NaOH (2×30 mL) and brine (20 mL). The ethereal layer was dried (Na₂SO₄) and concentrated in vacuo to give 3-benzoyloxy-4-fluorotoluene as a white solid (6.601 g, 96%), which was used without futher purification. δ_{H} (400 MHz, CDCl₃) 2.36 (s, 3H), 7.01-7.12 (m, 3H), 7.49-7.55 (m, 2H), 7.63-7.68 (m, 1H), 8.19-8.23 (m, 2H); LRMS (FAB+) m/z (rel. intensity) 231.1 (100, [M+H]⁺)

### 3-Benzoyloxy-4-fluorobenzyl bromide

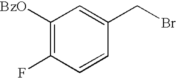

Nb. Commercial N-bromosuccinimide (NBS) was recrystallised from water and thoroughly dried in vacuo (24 h) prior to use (mp. 180-182° C., lit. 180-183° C.). To a solution of 3-benzoyloxy-4-fluorotoluene (2.47 g, 10 mmol) in anhydrous carbon tetrachloride (25 mL) was added finely powdered NBS (1.96 g, 11 mmol) and benzoylperoxide (0.01 g). The mixture heated at reflux for 2 h, cooled and diluted with Et₂O (100 mL) and water (50 mL). The ethereal layer was washed with brine (20 mL) and dried (Na₂SO₄). Concetration in vacuo and subsequent purification by flash column chromatography [SiO₂, EtOAc/n-hexane (1:25)] gave 3-benzoyloxy-4-fluorobenzyl bromide as a white solid (1.80 g, 58%); δ_{H} (400 MHz, CDCl₃) 4.48 (s, 2H), 7.18 (dd, J=9.8, 8.6, 1H), 7.28 (ddd, J=8.6, 4.3, 2.3, 1H), 7.34 (dd, J=7.0, 2.3, 1H), 7.50-7.55 (m, 2H), 7.64-7.69 (m, 1H), 8.18-8.23 (m, 2H); LRMS (FAB+) m/z (rel. intensity) 229.1 (95), 309.0 (100, [M+H]⁺). Found: C, 54.1; H, 3.22; C₁₄H₁₀BrFO₂ requires C, 54.39; H, 3.26%.

### 4-[(3-Benzoyloxy-4-fluorobenzyl)(4-cyanophenyl)amino]-4H-[1,2,4]triazole

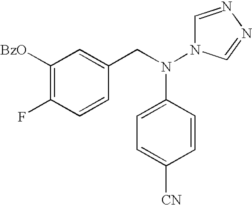

The title compound was prepared by adapting the method for 4-[(3-benzyloxybenzyl)(4-cyanophenyl)amino]-4H-[1,2,4]triazole using NaH (60% dispersion in oil, 0.20 g, 5.0 mmol), 4-[(4-cyanophenyl)amino]-4H-[1,2,4]triazole (0.926 g, 5.0 mmol) and 3-benzoyloxy-4-fluorobenzyl bromide (1.55 g, 5.0 mmol) in anhydrous DMF (20 mL) to give 4-[(3-benzoyloxy-4-fluorobenzyl)(4-cyanophenyl)amino]-4H-[1,2,4]triazole as a white solid (1.16 g, 56%) after chromatography [SiO₂, EtOAc (100%)]; δ_{H} (400 MHz, CDCl₃) 4.91 (s, 2H), 6.67 (AA′BB′, 2H), 7.07 (ddd, J=8.2, 4.3, 2.0, 1H), 7.18 (dd, J=9.4, 8.6, 1H), 7.24 (dd, J=7.0, 2.3, 1H), 7.49-7.56 (m, 2H), 7.58 (AA′BB′, 2H), 7.62-7.70 (m, 1H), 8.15-8.20 (m, 2H), 8.21 (s, 2H); LRMS (FAB+) m/z (rel. intensity) 414.2 (100, [M+H]⁺).

### 4-[(4-Fluoro-3-hydroxybenzyl)(4-cyanophenyl)amino]-4H-[1,2,4]triazole (STX488)

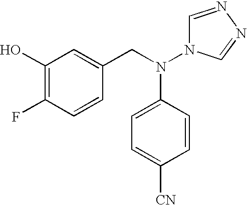

To a solution of 4-[(3-benzoyloxy-4-fluorobenzyl)(4-cyanophenyl)amino]-4H-[1,2,4]triazole (0.958 g, 2.32 mmol) in MeOH (10 mL) was added NaOH (0.25 g, 6.25 mmol), and the solution heated to reflux for 5 minutes and then concentrated in vacuo. Water (10 mL) was added and the milky suspension was neutralised (pH 7-8) with 2N HCl. The white precipitate was filtered off, washed with a small amount of water (5 mL) and dried under high vacuum to give STX488 as a white solid (0.476 g, 66%); δ_{H} (400 MHz, DMSO-d₆) 4.95 (s, 2H), 6.70 (ddd, J=11.4, 8.4, 2.4, 1H), 6.75 (AA′BB′, 2H), 6.84 (dd, J=8.4, 2.4, 1H), 7.06 (dd, J=11.3, 8.4, 1H), 7.76 (AA′BB′, 2H), 8.75 (s, 2H), 9.90 (s, 1H—exchanges with D₂O); LRMS (FAB+) m/z (rel. intensity) 310.1 (100, [M+H]⁺).

### Sulfamic acid 5-{[(4-cyano-phenyl)-[1,2,4]triazol-4-yl-amino]-methyl}-2-fluoro-phenyl ester (CAB03149/STX1122)

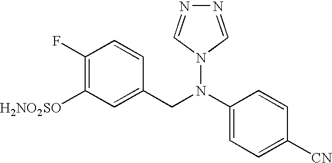

Sulphamoyl chloride solution in toluene (3 ml, 0.7 M, 2.1 mmol) was concentrated under reduced pressure (30° C. water bath temperature) to ca. 0.5 ml volume. The residue was cooled to 0° C. (ice bath) and N,N-dimethyl acetamide (5 ml) was added. 4-[(4-Fluoro-3-hydroxybenzyl)-[1,2,4]triazol-4-yl-amino]-benzonitrile (STX488, 155 mg, 0.50 mmol) was added to the colourless solution and the mixture was stirred for 4 hours at room temperature. Ethyl acetate (50 ml) and water (30 mL) were added to the solution, the organic layer was separated, washed with water (2×30 ml) and brine (1×20 ml), dried over sodium sulphate and concentrated under reduced pressure. The residue was dissolved in acetone (5 ml) and precipitated by addition of diethyl ether. The precipitate (STX1122) was filtered off and dried under high vacuum. Yield: 157 mg (83%) white powder. ¹H-NMR (400 MHz, DMSO-d₆) δ 5.07(s, 2H), 6.74 (d, J=8.9 Hz, 2H), 7.20-7.26 (m, 1H), 7.33 (dd, J=10.1, 8.6 Hz, 1H), 7.38 (dd, J=7.4, 2.3 Hz, 1H), 7.75 (d, J=8.9 Hz, 2H), 8.25 (s, 2H, —NH₂), 8.77 (s, 2H); MS (FAB+): m/z 389.0 (100%, [C₁₆H₁₃FN₆O₃S₂+H]⁺); HRMS (FAB+) for C₁₆H₁₄FN₆O₃S: 389.08321; found, 389.08300; HPLC t_{R}=1.763 min (100%); Anal. (C₁₆H₁₃FN₆O₃S) C, H, N; Calculated: N, 21.64%; C, 49.48%; H, 3.37% Result: N, 21.1%; C, 49.3%; H, 3.39%.

### 3-Benzoyloxy-4-chlorotoluene

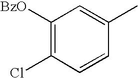

The title compound was prepared by adapting the method for 3-benzoyloxy-4-fluorotoluene using 4-chloro-3-hydroxytoluene (3.92 g, 27.5 mmol), benzoyl chloride (3.51 g, 25 mmol) and NEt₃ (5 mL) in CH₂Cl₂ (100 mL) to give 3-benzoyloxy-4-chlorotoluene as a white solid (5.82 g, 94%), which was used without further purification. δ_{H} (400 MHz, CDCl₃) 2.38 (s, 3H), 7.05 (d, J=8.2, 1H), 7.11 (s, 1H), 7.36 (d, J=8.2, 1H), 7.51-7.56 (m, 2H), 7.64-7.70 (m, 1H), 8.22-8.28 (m, 2H); δ_{C} (100 MHz, CDCl₃) 21.41, 123.97, 124.58, 128.07, 128.85, 129.15, 130.07, 130.55, 134.01, 138.38, 146.98, 164.51.

### 3-Benzoyloxy-4-chlorobenzyl bromide

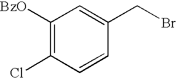

The title compound was prepared by adapting the method for 3-benzoyloxy-4-fluorobenzyl bromide using 3-benzoyloxy-4-chlorotoluene (2.47 g, 10.0 mmol), NBS (1.96 g, 11.0 mmol) and benzoyl peroxide (10 mg) in carbon tetrachloride (25 mL). The residue was purified by flash column chromatography [SiO₂, EtOAc/n-hexane (1:40)] to give 3-benzoyloxy-4-chlorobenzyl bromide as a colourless oil (2.012 g, 62%) which solidified on standing and was used without further purification; δ_{H} (400 MHz, CDCl₃) 4.48 (s, 2H), 7.27 (dd, J=8.2, 2.0, 1H), 7.36 (d, J=2.0, 1H), 7.46 (d, J=8.2, 1H), 7.50-7.58 (m, 2H), 7.64-7.71 (m, 1H), 8.22-8.28 (m, 2H). LRMS (FAB+) m/z (rel. intensity) 325.0 (81, [M+H]⁺), 327.0 (100, [M+H]⁺);

### 4-[(3-Benzoyloxy-4-chlorobenzyl)(4-cyanophenyl)amino]-4H-[1,2,4]triazole

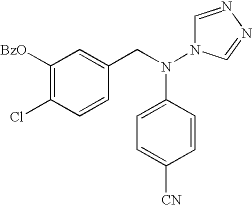

The title compound was prepared by adapting the method for 4-[(3-benzyloxybenzyl)(4-cyanophenyl)amino]-4H-[1,2,4]triazole using NaH (60% dispersion in oil, 0.20 g, 5.0 mmol), 4-[(4-cyanophenyl)amino]-4H-[1,2,4]triazole (0.926 g, 5.0 mmol) and 3-benzoyloxy-4-chlorobenzyl bromide (1.63 g, 5.0 mmol) in anhydrous DMF (20 mL) to give 4-[(3-benzoyloxy-4-chlorobenzyl)(4-cyanophenyl)amino]-4H-[1,2,4]triazole as a white solid (1.773 g, 82%) after chromatography [SiO₂, EtOAc (100%)]; δ_{H} (400 MHz, CDCl₃) 4.93 (s, 2H), 6.66 (AA′BB′, 2H), 7.06 (dd, J=8.2, 2.0, 1H), 7.27 (d, J=2.0, 1H), 7.45 (d, J=8.2, 1H), 7.47-7.58 (m, 4H), 7.60-7.69 (m, 1H), 8.16-8.21 (m, 2H), 8.24 (s, 2H); LRMS (FAB+) m/z (rel. intensity) 430.1 (100, [M+H]⁺).

### 4-[(4-Chloro-3-hydroxybenzyl)(4-cyanophenyl)amino]-4H-[1,2,4]triazole (STX483)

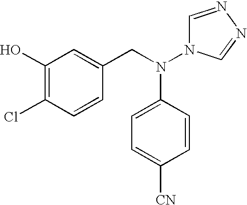

A solution of 4-[(3-benzoyloxy-4-chlorobenzyl)(4-cyanophenyl)amino]-4H-[1,2,4]triazole (1.13 g, 2.63 mmol) and NaOMe (500 mg) in MeOH (20 mL) and water (5 mL) was heated to reflux for 30 minutes. After cooling to room temperature most of the solvent was removed in vacuo and concentrated sodium bicarbonate solution (20 mL) and EtOAc (50 mL) were added. The organic layer was separated, dried (Na₂SO₄) and the solvent was removed under reduced pressure. The resulting white powder was refluxed in EtOAc (10 mL, product did not dissolve completely). The product was filtered off and dried in vacuo to give STX483 as a white powder (0.412 g, 48%); δ_{H} (400 MHz, DMSO-d6) 4.97 (s, 2H), 6.71-6.75 (m, 3H, AA′BB′ and ArH), 6.84 (d, J=2.0, 1H), 7.25 (d, J=8.2, 1H), 7.75 (AA′BB′, 2H), 8.76 (s, 2H), 10.21 (s, 1H-exchanges with D₂O); LRMS (FAB+) m/z (rel. intensity) 326.1 (100, [M+H]⁺).

### 4-[(4-Chloro-3-O-sulfamoylbenzyl)(4-cyanophenyl)amino]-4H-[1,2,4]triazole (STX559)

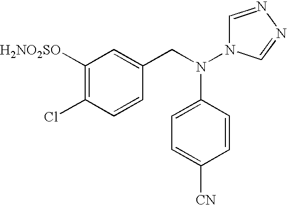

The title compound was sulfamoylated adapting the method for STX334 using STX483 (0.2 g, 0.614 mmol) and sulfamoyl chloride (0.7 M solution toluene, 3 mL, 2.1 mmol) in anhydrous DMA (5 mL) to give STX559 as a white powder (0.136 g, 55%) after precipitation from acetone/Et₂O; δ_{H} (400 MHz, DMSO-d₆) 5.11 (s, 2H), 6.75 (AA′BB′, 2H), 7.25 (dd, J=8.2, 2.0, 1H), 7.46 (d, J=2.0, 1H), 7.54 (d, J=8.2, 1H), 7.77 (AA′BB′, 2H), 8.32 (s, 2H—exchanges with D₂O), 8.82 (s, 2H); LRMS (FAB+) m/z (rel. intensity) 87.0 (100), 404.9 (40, [M+H]⁺); HRMS (FAB+) Found 405.05338; C₁₆H₁₄N₆O₃SCl requires 405.053663

### 4-Bromo-3-hydroxybenzoic acid

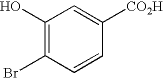

The title compound was prepared according to the method reported by Buehler et al.^{[Buehler 1946]} using 3-hydroxybenzoic acid (18.2 g, 132.0 mmol) and Br₂ (6.76 mL, 132.0 mmol) in glacial AcOH (250 mL) to give 4-bromo-3-hydroxybenzoic acid as a colourless crystalline solid (3.43 g, 12%) after recrystallisation from Et₂O/n-hexane; mp. 225-226° C. (lit.^{[Beyer, 1921]} 227-228° C.); δ_{H} (270 MHz, DMSO-d₆) 7.28 (dd, J=8.2, 2.0, 1H), 7.51 (d, J=1.7, 1H), 7.60 (d, J=8.3, 1H), 10.66 (bs, 1H,—exchanges with D₂O, ArOH) and 13.04 (bs, 1H—exchanges with D₂O, ArCOOH); LRMS (FAB+) m/z (rel. intensity) 218 (94, [C₇H₅⁸¹BrO₃+H]), 216 (100, [C₇H₅⁷⁹BrO₃+H]); LC-MS (APCI−): t_{R}=1.38 min, m/z 216.78 (M−H); HPLC: t_{R}=1.35 min (100%); HRMS Found 218.94656; C₇H₆⁸¹BrO₃ requires 218.94798.
Buehler et al., J. Am. Chem. Soc. 1946, 68, 574-577.

### Methyl 4-bromo-3-hydroxybenzoate

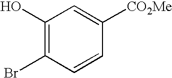

The title compound was prepared according to the method reported by Faltis et al.^{[Faltis, 1941]} using 4-bromo-3-hydroxybenzoic acid (5.7 g, 26.26 mmol) and concentrated sulphuric acid (98% solution, 1.4 mL, 26.26 mmol) in anhydrous MeOH (150 mL) to give methyl 4-bromo-3-hydroxybenzoate as a colourless crystalline powder (5.4 g, 89%) after recrystallisation from MeOH; mp. 126-127° C., lit.^{[Faltis 1941]} (hexane) 124-125° C.; δ_{H} (270 MHz, CDCl₃) 3.90 (s, 3H), 5.75 (bs, 1H—exchanges with D₂O), 7.46 (dd, J=8.4, 2.0, 1H), 7.53 (d, J=8.2, 1H), 7.67 (d, J=1.7, 1H); LRMS (FAB+) m/z (rel. intensity) 232.0 (94, [C₈H₇⁸¹BrO₃+H]), 230.8 (100, [C₈H₇⁷⁹BrO₃+H]); LC-MS (APCI−): t_{R}=3.72 min, m/z 230.96 (M−H); HPLC: t_{R}=2.04 min (99%); HRMS (FAB+) Found 232.96325; C₈H₈O₃⁸¹Br requires 232.96363.
Faltis et al., Chem. Ber., 1941, 74, 79, 84

### Methyl 4-bromo-3-(tetrahydropyran-2-yloxy)benzoate

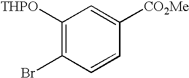

The title compound was prepared by adapting the method reported by Kita et al.^{[Kita, 1994]} To a solution of methyl 4-bromo-3-hydroxybenzoate (4.0 g, 17.31 mmol) in anhydrous CH₂Cl₂ (30 mL) was added 3,4-dihydro-2H-pyran (3.95 mL, 43.28 mmol) and p-toluenesulfonic acid (0.01 g, 0.17 mmol) at 0° C. The mixture was stirred at this temperature for 90 min, warmed to room temperature and quenched with saturated NaHCO₃ solution (50 mL). The organic layer was separated, dried (Na₂SO₄) and concentrated in vacuo. The pale yellow residue was purified by flash column chromatography [SiO₂, Et₂O/n-hexane (4:6)] affording a colourless oil. The oil solidified on standing at room temperature to give methyl 4-bromo-3-(tetrahydropyran-2-yloxy)benzoate as colourless needles (4.12 g, 76%); δ_{H} (270 MHz, CDCl₃) 1.59-1.77 (m, 3H), 1.86-2.13 (m, 3H), 3.6-3.64 (m, 1H), 3.8-3.85 (m, 1H), 3.88 (s, 3H), 5.6-5.62 (m, 1H), 7.52 (dd, J=8.2, 1.7, 1H), 7.59 (d, J=8.4, 1H), 7.76 (d, J=2.0, 1H); LRMS (FAB+) m/z (rel. intensity) 316.9 (30, [C₁₃H₁₅⁸¹BrO₄+H]), 314.9 (47, [C₁₃H₁₅⁷⁹BrO₄+H]); LC-MS (APCI+): t_{R}=1.48 min, m/z 334.13 (C₁₃H₁₅ 8BrO₄+H₃O⁺), 332.11 (C₁₃H₁₅⁷⁹BrO₄+H₃O⁺); HPLC: t_{R}=3.21 min (98%); HRMS (FAB+) Found 317.02290; C₁₃H₁₆⁸¹BrO₄ requires 317.02115.
Kita et al., Chem. Pharm. Bull., 1994, 42 (1), 147-150.

### 4-Bromo-3-(tetrahydropyran-2-yloxy)benzyl alcohol

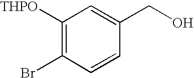

To a suspension of LiAlH₄ (0.72 g, 19.04 mmol) in anhydrous Et₂O (90 mL) was slowly added a solution of methyl 4-bromo-3-(tetrahydropyran-2-yloxy)benzoate (4.0 g, 12.69 mmol) in anhydrous Et₂O (10 mL). The mixture was stirred at room temperature for 2 h and then cautiously quenched with Na₂SO₄.10H₂O (until gas evolution ceases). The solids were filtered off and washed with Et₂O (100 mL). The combined organic fractions were then dried (Na₂SO₄) and concentrated in vacuo. The pale yellow residue was purified by flash column chromatography [SiO₂, Et₂O/n-hexane (7:3)] to give 4-bromo-3-(tetrahydropyran-2-yloxy)benzyl alcohol as a colourless oil (3.21 g, 88%). δ_{H} (270 MHz, CDCl₃) 1.54-1.76 (m, 3H), 1.82-2.12 (m, 3H), 3.54-3.64 (m, 1H), 3.8-3.94 (m, 1H), 4.61 (d, J=4.9, 2H), 5.52 (m, 1H), 6.85 (dd, J=7.9, 1.5, 1H), 7.13 (d, J=2.0, 1H), 7.49 (d, J=7.9, 1H), Nb. ArCH₂OH too broad to be observed; LC-MS (APCI+): t_{R}=3.84 min, m/z 306.11 (C₁₂H₁₅⁸¹BrO₄+H₃O⁺), 304.13 (C₁₂H₁₅⁷⁹BrO₄+H₃O⁺); HPLC: t_{R}=2.34 min (96%); HRMS (FAB+) Found 289.02646; C₁₂H₁₆O₃⁸¹Br requires 289.02624.

### 4-Bromo-3-(tetrahydropyran-2-yloxy)benzyl bromide

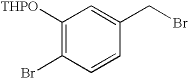

The title compound was prepared adapting the method reported by Wada et al.^{Wada, 1979]} with the following modifications. To a solution of 4-bromo-3-(tetrahydropyran-2-yloxy)benzyl alcohol (3.0 g, 10.45 mmol), carbon tetrabromide (6.93 g, 20.90 mmol) and anhydrous pyridine (0.85 mL, 10.45 mmol) in anhydrous Et₂O (50 mL) at 0° C. was added dropwise a solution of triphenylphosphine (5.48 g, 20.90 mmol) in anhydrous Et₂O (10 mL). The mixture was stirred at 0° C. for 1 h and then at room temperature overnight. The solvent was removed in vacuo and the residue suspended in n-hexane and filtered. The filtered solid was washed with n-hexane and the combined filtrates concentrated in vacuo to ca. 10 mL. The residue was purified by flash column chromatography [SiO₂, EtOAc/n-hexane (5:95)] to give 4-bromo-3-(tetrahydropyran-2-yloxy)benzyl bromide as a pale yellow oil (2.82 g, 77%) which slowly darkened on exposure to light and air; δ_{H} (270 MHz, CDCl₃) 1.58-2.12 (m, 6H), 3.58-3.65 (m, 1H), 3.83-3.92 (m, 1H), 4.41 (d, J=12.6, 2H), 5.53 (m, 1H), 6.89 (dd, J=7.9, 2.0, 1H), 7.16 (d, J=2.2, 1H), 7.48 (d, J=8.2, 1H); LC-MS (APCI−): t_{R}=4.71 min, m/z 266.95 (48, C₁₂H₁₄ 8Br₂O₄—C₅H₈O—H), 264.93 (100, C₁₂H₁₄Br₂O₄—C₅H₈O—H), 262.92 (50, C₁₂H₁₄⁷⁹Br₂O₄—C₅H₈O—H); HPLC: t_{R}=3.91 min (94%)
Wada et al., J. Chem. Soc. (Perkin Trans. 1). 1979, 323-327.

### 4-[(3-(Tetrahydropyran-2-yloxy)-4-bromobenzyl)(4-cyanophenyl)amino]-4H-[1,2,4]triazole

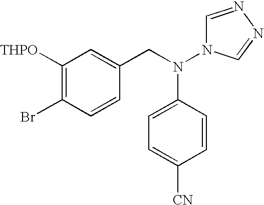

The title compound was prepared by adapting the method for 4-[(3-benzyloxybenzyl)(4-cyanophenyl)amino]-4H-[1,2,4]triazole using the following modifications. To a stirred suspension of NaH (60% dispersion in oil, 0.27 g, 6.78 mmol) in anhydrous DMF (10 mL) was added a solution of 4-[(4-cyanophenyl)amino]-4H-[1,2,4]triazole (1.26 g, 6.78 mmol) in anhydrous DMF (5 mL) and the mixture stirred at r.t. for 0.5 h. A solution of 4-bromo-3-(tetrahydropyran-2-yloxy)benzyl bromide (2.61 g, 7.46 mmol) in anhydrous DMF (5 mL) was then added and the mixture heated at 80-90° C. for 3 h. The mixture was cooled, diluted with EtOAc (50 mL), washed with water (4×100 mL), brine (100 mL) and dried (Na₂SO₄). Concentration in vacuo and subsequent purification by flash column chromatography [SiO₂, EtOAc (100%)] gave 4-[(3-(tetrahydropyran-2-yloxy)-4-bromobenzyl)(4-cyanophenyl)amino]-4H-[1,2,4]triazole as a yellow oil (3.05 g, 99%); δ_{H} (270 MHz, CDCl₃) 1.61-1.99 (m, 6H), 3.55-3.59 (m, 1H), 3.73 (m, 1H), 4.75 (d, J=14.6, 1H), 4.90 (d, J=14.6, 1H), 5.41 (m, 1H), 6.64 (AA′BB′, 2H), 6.69 (dd, J=8.2, 2.2, 1H), 7.00 (d, J=2.2, 1H), 7.49 (d, J=7.9, 1H), 7.57 (AA′BB′, 2H), 8.15 (s, 2H); LRMS (FAB+) m/z (rel. intensity) 456.1 (54, [C₂₁H₂₀N₅O₂⁸¹Br+H]), 454.1 (54, [C₂₁H₂₀N₅O₂⁷⁹Br+H]); LC-MS (APCI+): t_{R}=2.0 min, m/z 456.1 (94, [C₂₁H₂₀N₅O₂⁸¹Br+H]), 454.1 (100, [C₂₁H₂₀N₅O₂⁷⁹Br+H]); HPLC: t_{R}=2.21 min (94%); HRMS (FAB+): Found 454.08627 C₂₁H₂₁N₅O₂⁷⁹Br requires 454.08786.

### 4-[(4-Bromo-3-hydroxybenzyl)(4-cyanophenyl)amino]-4H-[1,2,4]triazole (STX1216)

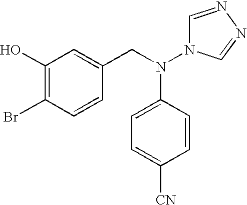

The title compound was prepared by adapting the method reported by Tafi et al.^{[Tafi,2002]} with the following modifications. To a solution of 4-[(3-(tetrahydropyran-2-yloxy)-4-bromobenzyl)(4-cyanophenyl)amino]-4H-[1,2,4]triazole (3.05 g, 6.71 mmol) in MeOH (60 mL) was added a catalytic amount of p-toluenesulfonic acid at 0° C. and the mixture was stirred (allowing to slowly warm to room temperature) overnight. The solvent was removed in vacuo and the residue dissolved in EtOAc (100 mL). The organic fraction was washed with Na₂CO₃ solution (1M aqueous solution, 3×50 mL), brine (3×50 mL) and dried (Na₂SO₄). The solvent was removed in vacuo and the pale yellow residue recrystallised from EtOH to give STX1216 as an off-white powder (1.48 g, 60%); δ_{H} (400 MHz, DMSO-d₆) 5.00 (s, 2H), 6.72 (dd, J=8.3, 2.1, 1H), 6.76 (AA′BB′, 2H), 6.86 (d, J=1.8, 1H), 7.43 (d, J=8.0, 1H), 7.78 (AA′BB′, 2H), 8.81 (s, 2H), 10.32 (s, 1H-exchanges with D₂O); δ_{C} (100 MHz, DMSO-d₆) 57.08 (CH₂), 103.32, 109.42, 114.07 (2×CH), 116.66 (CH), 119.51 (CH), 120.90 (CH), 133.54 (2×CH), 134.45 (2×CH), 136.14, 143.90, 151.82, 154.60; LRMS (FAB+) m/z (rel. intensity) 372.1 (94, [C₁₆H₁₂N₅O⁸¹Br+H], 370.1 (100, [C₁₆H₁₂N₅O⁷⁹Br+H]); LC-MS (ES−) t_{R}=1.98 min, m/z 368.1 (C₁₆H₁₂N₅O⁷⁹Br—H), 370.1 (C₁₆H₁₂N₅O⁸¹Br—H); HPLC t_{R}=1.77 min (100%); HRMS (FAB+) Found 372.02789; C₁₆H₁₃N₅O⁸¹Br requires 372.02830. Found: C, 51.6; H, 3.4; N, 18.9; C₁₆H₁₂N₅OBr requires C, 51.9; H, 3.3; N, 18.9%.
Tafi et al., J. Med. Chem., 2002, 45 (13), 2720-2732.

### 4-[(4-Bromo-3-O-sulfamoylbenzyl)(4-cyanophenyl)amino]-4H-[1,2,4]triazole (STX1217)

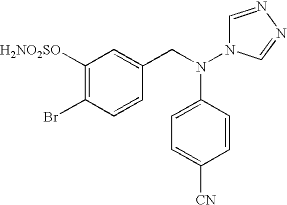

The title compound was sulfamoylated adapting the method for STX334 using STX1216 (0.5 g, 1.35 mmol) and sulfamoyl chloride (0.69 M solution toluene, 19.6 mL, 13.5 mmol) in anhydrous DMA (5 mL) to give STX1217 as an off-white solid (0.39 g, 64%) after chromatography [SiO₂, EtOAc (100%)]; δ_{H} (400 MHz, DMSO-d₆) 5.11 (s, 2H), 6.77 (AA′BB′, 2H), 7.21 (dd, J=8.1, 1.8), 7.49 (d, J=2.1, 1H), 7.70 (d, J=8.1.1H), 7.80 (AA′BB′, 2H), 8.34 (br s, 2H—exchanges with D₂O), 8.85 (s, 2H); δ_{C} (400 MHz, DMSO-d₆) 56.82 (CH₂), 103.52, 114.13 (2×CH), 115.90, 123.59 (CH), 128.18 (CH), 134.30 (CH), 134.49 (2×CH), 136.83, 143.81 (2×CH), 147.94, 151.71; LRMS (FAB+) m/z (rel. intensity) 451.9 (100, [C₁₆H₁₃N₆SO₃⁸¹Br+H]), 449.9 (98, [C₁₆H₁₃N₆SO₃⁷⁹Br+H]); LC-MS (APCI+) t_{R}=3.01 min, m/z 449 (100, [C₁₆H₁₃N₆SO₃⁷⁹Br+H]), 451 (83, [C₁₆H₁₃N₆SO₃⁸¹Br+H]); HPLC t_{R}=1.67 min (100%); Found: C, 43.0; H, 3.0; N, 18.5; C₁₆H₁₃N₆SO₃Br requires C, 42.8; H, 2.9; N, 18.7%.

### 4-[(3-Benzyloxy-4-methoxybenzyl)(4-cyanophenyl)amino]-4H-[1,2,4]triazole

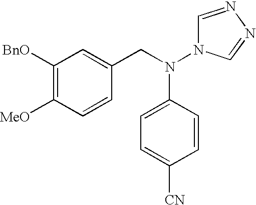

The title compound was prepared by adapting the method for 4-[(3-benzyloxybenzyl)(4-cyanophenyl)amino]-4H-[1,2,4]triazole using NaH (60% dispersion in oil, 0.22 g, 5.4 mmol), 4-[(4-cyanophenyl)amino]-4H-[1,2,4]triazole (1.0 g, 5.68 mmol) and 4-methoxy-3-benzyloxybenzyl bromide^{[Meyers, 1989]} (1.66 g, 5.4 mmol) in anhydrous DMF (5 mL) to give 4-[(3-benzyloxy-4-methoxybenzyl)(4-cyanophenyl)amino]-4H-[1,2,4]triazole as an off-white solid (0.97 g, 44%) after recrystallisation; mp. 138-141° C.; δ_{H} (400 MHz, CDCl₃) 3.89 (s, 3H), 4.72 (s, 2H), 5.12 (s, 2H), 6.59 (AA′BB′, 2H), 6.62 (d, J=2, 1H), 6.64 (dd, J=7.8, 2, 1H), 6.79 (d, J=7.8, 1H), 7.28-7.34 (m, 5H), 7.57 (AA′BB′, 2H), 7.74 (s, 2H); LRMS (FAB+) m/z (rel. intensity) 412 (100, [M+H]), 343 (51), 227 (72%); HPLC: t_{R}=2.21 min (94%); HRMS (FAB+): Found 412.1774 C₂₄H₂₂N₅O₂ requires 412.1776.

Meyer et al., Heterocycles. 1989, 28 (1), 295-301.

### 4-[(3-Hydroxy-4-methoxybenzyl)(4-cyanophenyl)amino]-4H-[1,2,4]triazole (STX363)

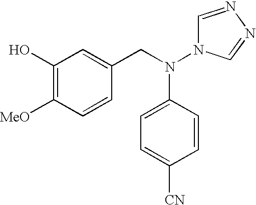

The title compound was hydrogenated (over 16 h) by adapting the method for STX333 using 4-[(3-benzyloxy-4-methoxybenzyl)(4-cyanophenyl)amino]-4H-[1,2,4]triazole (0.411 g, 0.999 mmol) and Pd—C (10% by wt., 0.042 g) in THF/MeOH (1:1) (20 mL) to give STX363 as a colourless powder (0.164 g, 51%) after recrystallisation; δ_{H} (400 MHz, DMSO-d₆) 3.71 (s, 3H); 4.88 (s, 2H), 6.62 (dd, J=8.2, 1.95, 1H), 6.68 (d, J=1.95, 1H), 6.74 (AA′BB′, 2H), 6.80 (d, J=8.2, 1H), 7.74 (AA′BB′, 2H), 8.70 (s, 2H), 9.00 (s, 1H—exchanges with D₂O); LRMS (FAB+) m/z (rel. intensity) 322 (100, [M+H]), 253 (61%); HPLC t_{R}=2.03 min (99% purity); HRMS (FAB+) Found 322.1304; C₁₇H₁₆N₅O₂ requires 322.1304.

### 4-[(4-Methoxy-3-O-sulfamoylbenzyl)(4-cyanophenyl)amino]-4H-[1,2,4]triazole (STX661)

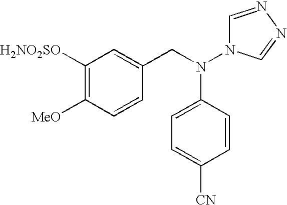

The title compound was sulfamoylated adapting the method for STX334 using STX363 (0.1 g, 0.31 mmol) and sulfamoyl chloride (0.69 M solution toluene, 2.71 mL, x.xx mmol) in anhydrous DMA (2 mL) to give STX661 as a colourless solid (0.04 g, 32%) after precipitation from EtOAc solution by addition of n-hexane; δ_{H} (400 MHz, DMSO-d₆) 3.77 (s, 3H), 5.00 (s, 2H), 6.77 (AA′BB′, 2H), 7.06 (d, J=8.6, 1H), 7.11 (dd, J=8.6, 2.3, 1H), 7.27 (d, J=2.3, 1H), 7.77 (AA′BB′, 2H), 7.94 (s, 2H—exchanges with D₂O), 8.75 (s, 2H); LC-MS (APCI+) t_{R}=4.70 min (M+H=401); HPLC t_{R}=2.04 min (98% purity).

### 4-[(2-Bromo-ethyl)-[1,2,4]triazol-4-yl-amino]-benzonitrile (CAB03031)

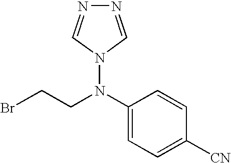

Sodium hydride (240 mg, 6.0 mmol) was added to a solution of 4-([1,2,4]triazol-4-ylamino)-benzonitrile (926 mg, 5.0 mmol) in DMSO (25 ml) at r.t. The mixture was stirred for 1 hour at this temperature and 1,2-dibromethane (5 ml) was added. The reaction mixture was stirred overnight and ethyl acetate (100 ml) was added. The mixture was transferred into a separation funnel and extracted with water (twice 100 ml) and brine (20 ml). The organic layer was dried over sodium sulphate and concentrated under reduced pressure (water bath temperature <30° C.). The resulting orange oil was mixed with diethyl ether (100 ml) and filtered through a layer of silica (ca. 5 cm). The silica was washed with more diethyl ether (ca. 100 ml) to remove the excess of 1,2-dibromoethane; the crude product was washed from the silica with acetone (120 ml). The acetone solution was concentrated under reduced pressure and the residue was purified by column-chromatography (eluent: ethyl acetate) to give the title compound as a white solid. Yield: 628 mg (43%). ¹H-NMR (400 MHz, d6-DMSO) δ=3.61 (t, J=6.2 Hz, 2H), 4.30 (t, J=6.4 Hz, 2H), 6.64 (d, J=9.0 Hz), 7.74 (d, 9.0 Hz, 2H), 8.97 (s, 2H). LRMS (FAB+):292.0 (100, [M+H]⁺)

### 4-{[2-(3-Hydroxy-phenylsulfanyl)-ethyl]-[1,2,4]triazol-4-yl-amino}-benzonitrile (CAB02149, STX512):

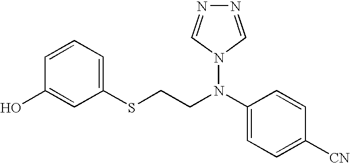

A mixture of 4-[(2-Bromo-ethyl)-[1,2,4]triazol-4-yl-amino]-benzonitrile (CAB02148=CAB03031, 146 mg, 0.50 mmol), 3-(tert-butyl-dimethylsiloxy)-thiophenol (240 mg, 1.0 mmol) and potassium carbonate (276 mg, 2.0 mmol) in DMF (10 ml) was stirred for 48 hours at room temperature. The mixture was transferred into a separation funnel and ethyl acetate (50 ml) and water (50 ml) were added. The organic layer was separated, washed with brine (30 ml), dried over sodium sulphate and concentrated under reduced pressure. The residue was crystallised from methanol. Yield: 93 mg (55%) colourless crystals (STX512). ¹H-NMR (400 MHz, d6-DMSO) δ=3.14 (t, J=7.0 Hz, 2H), 4.02 (t, J=7.0 Hz, 2H), 6.53 (d, J=9.0 Hz, 2H), 6.62 (ddd, J=7.8, 2.0, 0.8 Hz, 1H), 6.70-6.72 (m, 2H), 7.10 (dd, J=7.8, 7.8, 1H), 7.70 (d, J=9.0 Hz, 2H), 8.94 (s, 2H), 9.60 (s, 1H, —OH). ¹³C-NMR (100 MHz, d6-DMSO) δ=29.53, 52.46, 102.38, 112.86, 113.57, 115.36, 119.00, 130.11, 133.94, 135.82, 143.50, 150.71, 157.90, 169.59. LRMS (FAB+): 338.2 (100, [M+H]⁺). C₁₇H₁₅N₅OS (337.4) Calculated: C, 60.52%; H, 4.48%; N, 20.76%, Found: C, 60.6%; H, 4.53%; N, 20.8%.

### Sulfamic acid 3-{2-[(4-cyano-phenyl)-[1, 2, 4]triazol-4-yl-amino]-ethylsulfanyl}-phenyl ester (CAB03131, STX1014)

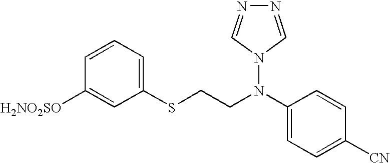

Sulphamoyl chloride solution in toluene (1.5 ml, 0.7 M, 1.05 mmol) was concentrated under reduced pressure (30° C. water bath temperature) to ca. 0.5 ml volume. The residue was cooled to 0° C. (ice bath) and N,N-dimethyl acetamide (5 ml) was added. 4-{[2-(3-Hydroxy-phenylsulfanyl)-ethyl]-[1,2,4]triazol-4-yl-amino}-benzonitrile (CAB02149, 75 mg, 0.22 mmol) was added to the colourless solution and the mixture was stirred for 18 hours at room temperature. Ethyl acetate (50 ml) and water (50 mL) were added to the solution, the organic layer was separated, washed with water (2×30 ml) and brine (1×20 ml), dried over sodium sulphate and concentrated under reduced pressure. The residue was dissolved in a small amount of acetone and precipitated by addition of hexane. The precipitate (STX1014) was filtered off and dried under high vacuum. Yield: 79 mg (85%) light yellow powder. ¹H-NMR (400 MHz, DMSO-d₆) δ 3.23 (t, J=7.0 Hz, 2H), 4.07 (t, J=7.0 Hz, 2H), 6.54 (d, J=8.9 Hz, 2H), 7.08-7.12 (m, 1H), 7.20-7.26 (m, 1H), 7.39 (dd, J=9.2, 9.2 Hz, 1H), 7.69 (d, J=8.9 Hz, 2H), 8.03 (s, 2H, —NH₂), 8.94 (s, 2H); MS (FAB+): m/z 85.1.1 (28%), 417.0 (100%, [C₁₇H₁₆N₆O₃S₂+H]⁺); HRMS (FAB+) for C₁₇H₁₇N₆O₃S₂: 417.08036; Found, 417.08067; HPLC t_{R}=1.53 min (99.7%).

### tert-Butyl-(3-chloromethylsulfanyl-phenoxy)-dimethyl-silane

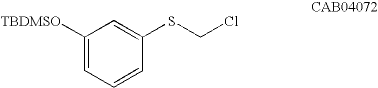

DBU (914 mg, 6.0 mmol) was added to a solution of 3-(tert-Butyl-dimethyl-silanyloxy)-benzenethiol (1.00 g, 4.16 mmol) in bromo-chloro methane (5 ml) at room temperature. Stirring was continued for 2 hours and Et₂O (50 ml) and 2M KHSO₄ (30 ml) were added. The organic layer was separated, washed with 2M KHSO₄ (30 ml) and brine (30 ml), dried (Na₂SO₄) and concentrated under reduced pressure. The resulting oil was analysed by ¹H-NMR spectroscopy and was used without further purification. Yield: 1.01 g (84%).

¹H-NMR (270 MHz, DMSO-d₆) δ 0.21 (6H, s, 2×SiCH₃), 0.99 (9H, s, —C(CH₃)₃), 4.97 (2H, s, —CH₂Cl), 6.70-7.30 (m, 4H).

### 4-[(3-Hydroxy-phenylsulfanylmethyl)-[1,2,4]triazol-4-yl-amino]-benzonitrile

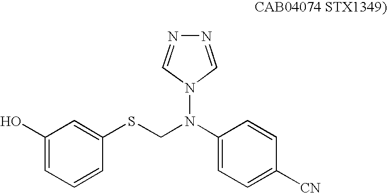

NaH (140 mg, 3.50 mmol, 60% in mineral oil) was added to a solution of 4-([1,2,4]Triazol-4-ylamino)-benzonitrile (648 mg, 3.50 mmol) in DMF. The mixture was stirred for one hour at r.t. and a solution of CAB04072 (1.01 g, 3.50 mmol) in DMF (10 ml) was added. Stirring was continued for 24 hours, EtOAc (75 ml) and water (50 ml) were added, the organic layer was separated, washed with water (3×30 ml) and brine (30 ml), dried (Na₂SO₄) and concentrated under reduced pressure. The residue was purified by flash chromatography (EtOAc, R_{f}: 0.30) to give the title compound as a white solid. Yield: 136 mg (12%).

m.p.: 188-190° C.;

¹H-NMR (270 MHz, DMSO-d₆) δ 5.62 (2H, s), 6.64 (2H, d, J=9.0 Hz), 7.20-7.26 (1H, m), 7.35-7.50 (3H, m), 7.74 (2H, d, 9.0 Hz), 8.81 (2H, s), 9.75 (1H, s, —OH);

MS (FAB+): m/z 324.0 (100%, [C₁₆H₁₄N₅OS]⁺), 255.0 (85%);

HRMS (FAB+) calcd. for C₁₆H₁₄N₅OS:324.09191; found, 324.09173.

### Sulfamic acid 3-{[(4-cyano-phenyl)-[1,2,4]triazol-4-yl-amino]-methylsulfanyl}-phenyl ester

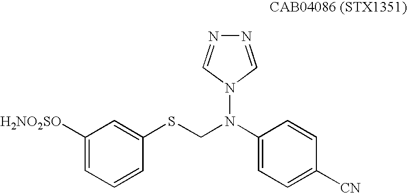

Sulfamoyl chloride solution in toluene (1.5 ml, 0.7 M, 1.05 mmol) was concentrated under reduced pressure. The residue was cooled to 0° C. (icebath) and DMA (5 ml) was added slowly. CAB04074 (97 mg, 0.30 mmol) was added to the colourless solution and the mixture was stirred for 18 h at room temperature. EtOAc (50 ml) and water (50 ml) were added, the organic layer was separated, washed with water (2×30 ml) and brine (20 ml), dried over Na₂SO₄ and concentrated under reduced pressure. The residue was dissolved in a small amount of acetone and precipitated by addition of Et₂O. The precipitate was filtered of and dried under high vacuum. Yield 76 mg (63%).

m.p.: 167-170° C. (dec.);

¹H-NMR (270 MHz, DMSO-d₆) δ 5.64 (2H, s), 6.66 (2H, d, J=9.0 Hz), 7.16-7.20 (1H, m), 7.30-7.44 (3H, m), 7.71 (2H, d, 9.0 Hz), 8.05 (2H, s, —NH₂), 8.77 (2H, s);

MS (FAB+): m/z 403.0 (100%, [C₁₆H₁₅N₆O₃S₂]⁺);

HRMS (FAB+) calcd. for C₁₆H₁₅N₆O₃S₂: 403.06471; found, 403.06623

Biological Data

Compounds were tested for aromatase and steroid sulphatase inhibition in accordance with the above Protocols. Each compound in accordance with the present invention is found to inhibit steroid sulphatase and aromatase.

The following in vitro data were recorded. IC50 values are shown as just numbers (without units). Compounds for which % Inhibition at 10 micromolar was measured this is denoted with a % sign.

| | | JEG3 cells IC₅₀ | JEG3 cells IC₅₀ |
|---|---|---|---|
| | | Aromatase (nM) or % | Sulfatase (nM) or % |
| Compound | Structure | inhibition @ 10 μM | inhibition @ 10 μM |
| STX334 | 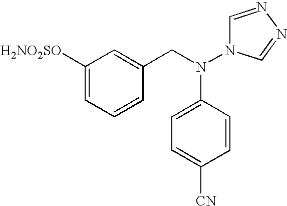 | IC50 39 ± 10.1 | IC50 5133 ± 65.4 |
| STX1122 | 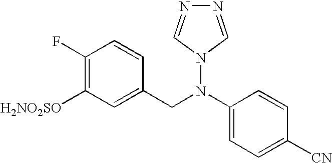 | IC50 0.77 ± 0.03 | IC50 590 |
| STX559 | 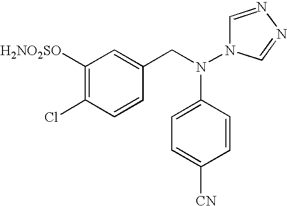 | IC50 0.92 | IC50 >10,000 |
| STX1217 | 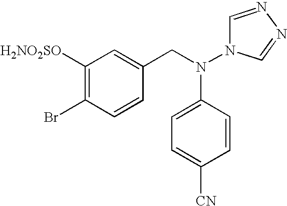 | IC50 3.9 ± 0.9 | IC50 >10,000 |
| STX661 | 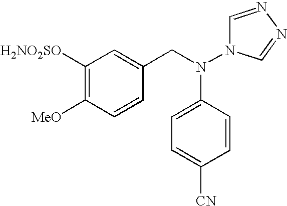 | IC50 12 | 12 ± 3.1% |
| STX1014 | 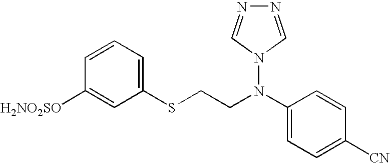 | IC50 1.7 ± 0.5 | IC50 >10,000 |
| STX1351 | 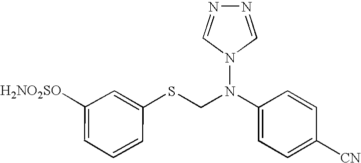 | IC50 3.9 ± 1.8 | IC50 3500 ± 100 |
| STX1001 | 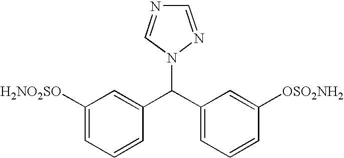 | IC50 99 ± 0.01 | 31.4% |
| STX1007 | 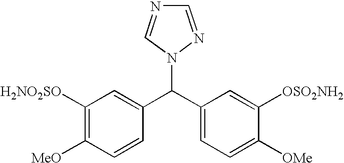 | 40% | 10.9% |

### ADDITIONAL EXAMPLES

The present invention will now be described only by way of additional example with reference to the accompanying drawings in which:—

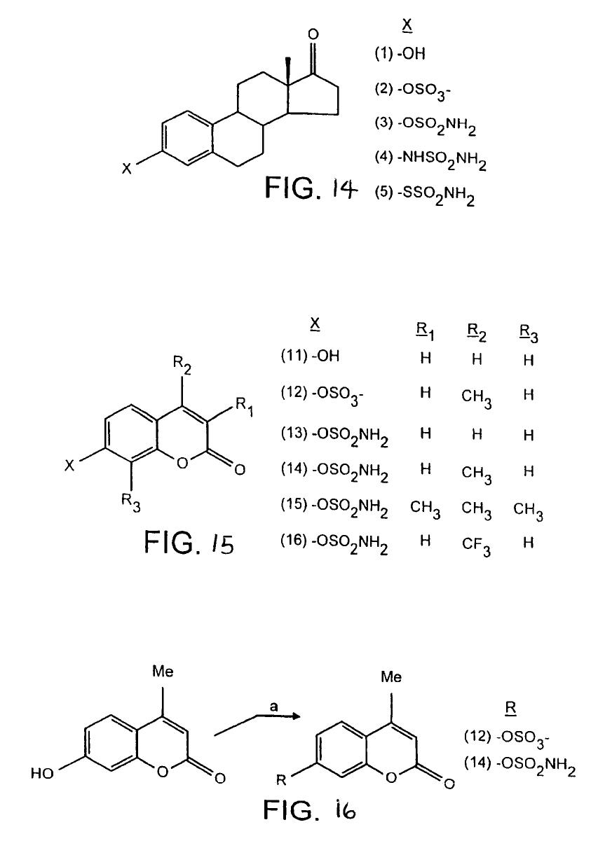

FIG. 14 shows the known structures of oestrone (1), oestrone sulphate (2), EMATE (3) and steroid sulphamates (4-5);

FIG. 15 shows the structures of 7-hydroxycoumarin (11), 7-(sulphoxy)₄-methylcoumarin (12) and coumarin sulphamates (13-16);

FIG. 16 shows the sulphation of 7-hydroxy-4-methylcoumarin; pyridine/SO₃-pyridine complex, NaOH in MeOH (Route a);

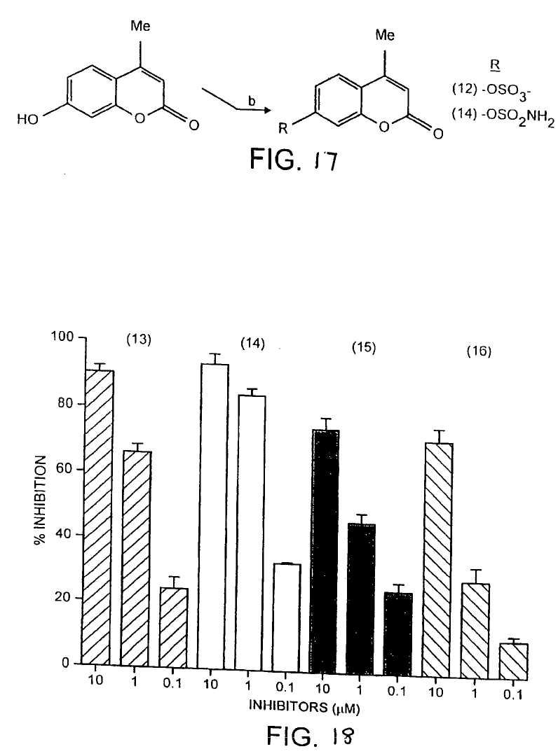

FIG. 17 shows the sulphamoylation of 7-hydroxy-4-methylcoumarin; NaH/DMF, H₂NSO₂Cl in toluene (Route b);

FIG. 18 shows the dose-dependent inhibition of oestrone sulphatase in intact MCF-7 breast cancer cells by coumarin-7-O-sulphamate (13), 4-methylcoumarin-7-O-sulphamate (14), 3,4,8-trimethyl-coumarin-7-O-sulphamate (15) and 4-(trifluoromethyl)coumarin-7-O-sulphamate (16);

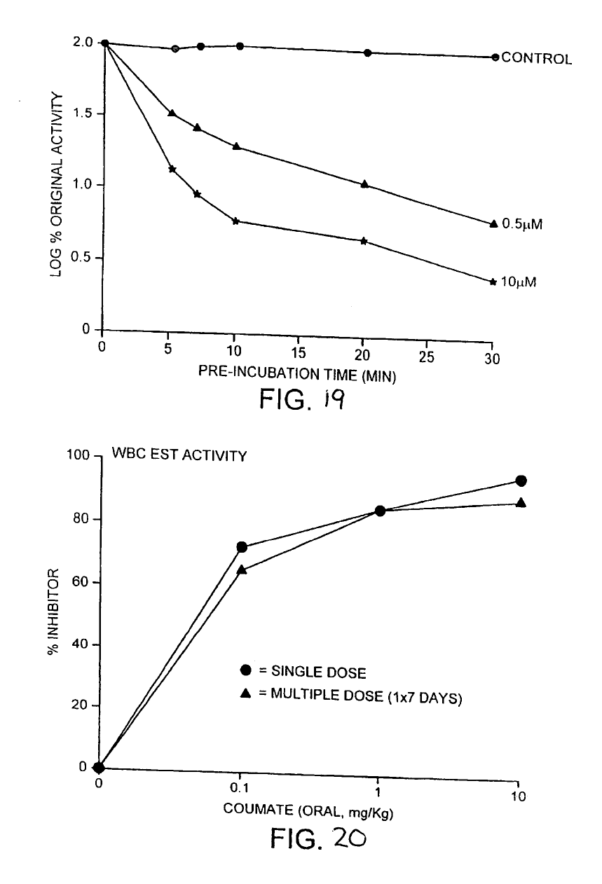

FIG. 19 shows the time-dependent and the concentration-dependent inactivation of oestrone sulphatase by 4-methyl-coumarin-7-O-sulphamate (14);

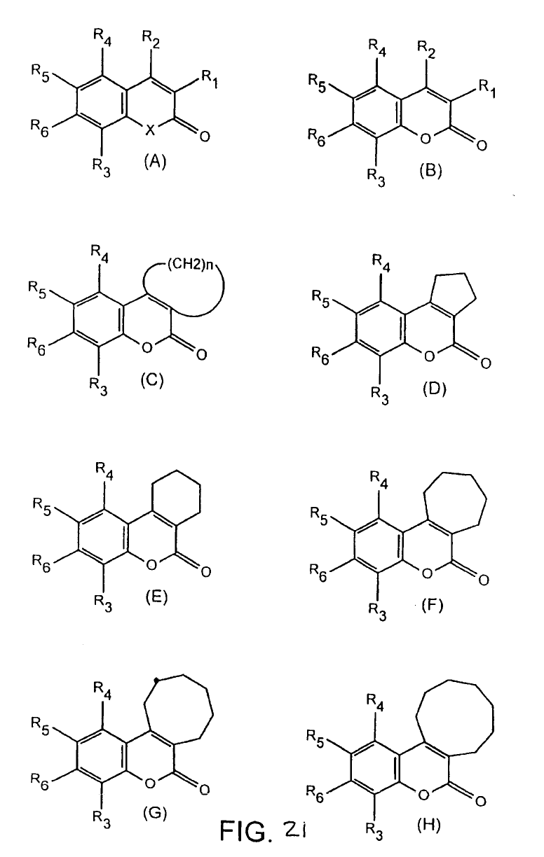

FIG. 20 is a graph;

FIG. 21 presents Formulae (A), (B), (C), (D), (E), (F), (G) and (H).

The compounds of the present invention may be prepared by a process that comprises a Packman synthesis step. Packman synthesis is known in the art.

Sulphamoylation of Coumarins

The general procedure for the sulphamoylation of coumarins was as follows. A solution of an appropriate coumarin in anhydrous DMF (ca. 40 ml per g of coumarin) was treated with sodium hydride [60% dispersion; 1 equiv] at 0° C. under an atmosphere of N₂. After evolution of hydrogen had ceased, sulphamoyl chloride in toluene [ca. 0.68 M, 1.5 equiv] was added and the reaction mixture was poured into water after warming to room temperature overnight and then the crude product was then quenched. The organic fraction in ethyl acetate (150 ml) was washed exhaustively with brine, dried (MgSO₄), filtered and evaporated. The crude product obtained was purified by flash chromatography followed by recrystallisation to give the corresponding sulphamate. All new compounds were fully characterised by spectroscopic and combustion analysis. The synthesis of 4 methylcoumarin-7-O-sulphamate (14) is shown in FIG. 17.

Following this general procedure, compounds 13-16 (as shown in FIG. 15)—i.e. coumarin-7-O-sulphamate (13), 4-methylcoumarin-7-O-sulphamate (14), 3,4,8-trimethylcoumarin-7-O-sulphamate (15) and 4-(trifluoromethylcoumarin)-7-O-sulphamate (16)—were prepared. More details on the synthesis of these compounds now follow.

The synthesis of compound 12 (as shown in FIG. 2) is also discussed below.

### Preparation of Coumarin-7-O-sulphamate (13)

Following the above-mentioned general procedure, 7-Hydroxycoumarin (500 mg, 3.082 mmol) gave a crude product (605 mg) which was fractionated on silica (200 g) by gradient elution with chloroform/acetone (8:1, 500 ml; 4:1, 1000 ml and then 2:1, 500 ml). Upon evaporation, the second fraction gave a creamy yellow residue (389 mg, 52.3%) which was recrystallised in ethyl acetate/hexane (1:1) to give (13) as dull white crystals (239 mg).

Analytical Data were as Follows:

M.p. 170.0-171.5° C.; R_{f}s=0.48 (ether), 0.67 (ethyl acetate), 0.19 (chloroform/acetone, 4:1); vmax (KBr) 3360, 3210, 3060, 1720, 1615, 1370, 1125 cm⁻¹; δ_{H} (DMSO-d₆/CDCl₃, ca. 1: 25) 6.415 (1H, d, J_{C-4-H, C-3-H}=9.7 Hz, C-3-H), 7.285 (1H, dd, J_{C-8-H, C-6-H}=2.3 Hz and J_{C-5-H, C-6-H}=8.5 Hz, C-6-H), 7.379 (1H, d, J_{C-6-H, C-8-H}=2.2 Hz, C-8-H), 7.508 (2H, br s, D₂O exchanged, —NH₂), 7.543 (1H, d, J_{C-6-H, C-5-H}=8.4 Hz, C-5-H) and 7.760 (1H, d, J_{C-3-H, C-4-H=}9.7 Hz, C-4-H). MS: m/z (E.I., rel. intensity) 241.0(10), 162.0(97), 134.0(100), 105.0(23). Acc. MS: m/z 241.0068, C₉H₇NO₅S requires 241.0045. Found: C, 44.8; H, 2.89; N, 5.82. C₉H₇NO₅S requires C, 44.81; H, 2.92; N, 5.81%.

### Preparation of 4-Methylcoumarin-7-O-sulphamate (14)

Following the above-mentioned general procedure, 7-Hydroxy-4-methylcoumarin (500 mg, 2.753 mmol) gave a crude product (633 mg) which was fractionated on silica (200 g) by gradient elution with chloroform/acetone (8:1, 500 ml; 4:1, 1000 ml, 2:1, 500 ml and then 1:1, 500 ml). Upon evaporation, the second fraction gave a creamy yellow residue (425 mg, 60.5%) which was recrystallised in acetone/chloroform (3:5) to give (14) as colorless rhombic crystals (281 mg).

Analytical Data were as Follows:

M.p. 165-167° C.; R_{f}s=0.48 (ether), 0.29 (ether/hexane 8:1), 0.26 (chloroform/acetone, 4:1); vmax (KBr) 3320, 3180, 3080, 1700, 1620, 1560, 1380, 1125 cm⁻¹; δ_{H} (acetone-d₆) 2.507 (3H, s, —CH₃), 6.339 (1H, s, C-3-H), 7.299 (2H, m, C-6-Hand C-8-H), 7.390 (2H, br s, D₂O exchanged, —NH₂) and 7.850 (1H, d, J_{C-6-H, C-5-H}=9 Hz, C-5-H). MS: m/z (+ve ion FAB in m-NBA, rel. intensity) 542.2(15), 511.1[45, (2M+H)⁺], 461.2(20), 409.1[60, (M+H+NBA)⁺], 393.3[60, (M+H+NBA-16)⁺], 329.2[10, (M+H+NBA-80)⁺], 256.1 [100, (M+H)⁺]. MS: m/z (−ve ion FAB in m-NBA, rel. intensity) 421.0(20), 407.1[15, (M−H+NBA)⁻], 335.1(14), 254[100, (M−H)⁻], 175.1[32, (M−H−79)⁻], 121.0(17). Found: C, 47.2; H, 3.56; N, 5.51. C₁₀H₉NO₅S requires C, 47.06; H, 3.55; N, 5.49%.

### Preparation of 3,4,8-Trimethylcoumarin-7-O-sulphamate (15)

Following the above-mentioned general procedure, 7-Hydroxy-3,4,8-trimethylcoumarin (1.0 g, 4.896 mmol) gave a crude product (1.33 g) which upon recrystallisation in hot ethyl acetate yielded 238 mg of starting coumarin. The mother liquor was evaporated and the white residue obtained (1.13 g) was fractionated on silica (200 g) with ether. The second fraction was collected, evaporated and the residue obtained (519 mg, 37.4%) was recrystallised in acetone/hexane (1:2) to give (15) as pale yellow crystals (312 mg).

Analytical Data were as Follows:

M.p. 197-202° C.; R_{f}s=0.50 (ether), 0.69 (ethyl acetate); vmax (KBr) 3310, 3040, 1680, 1600 cm⁻¹; δ_{H} (acetone-d₆) 2.176, 2.383 and 2.458 (9H, three s, 3×CH₃), 7.374 (1H, d, J_{C-5-H, C-6-H}=8.8 Hz, C-6-H), 7.390 (2H, br s, D₂O exchanged, —NH₂) and 7.682 (1H, d, J_{C-6-H, C-5-H}=8.8 Hz, C-5-H). MS: m/z (E.I., rel. intensity) 283.1(10), 204.1(45), 176.1(40), 161.1(22), 69.1(56), 57.1(40), 43.1 (100). Acc.

MS: m/z 283.0497, C₁₂H₁₃NO₅S requires 283.0514. Found: C, 50.86; H, 4.63; N, 4.97. C₁₂H₁₃NO₅S requires C, 50.88; H, 4.63; N, 4.94%.

### Preparation of 4-(Trifluoromethyl)coumarin-7-O-sulphamate (16)

Following the above-mentioned general procedure, 7-Hydroxy-4-(trifluoromethyl)-coumarin (0.90 g, 3.911 mmol) gave a crude product (1.20 g) which was fractionated on silica (200 g) with ether/chloroform (1:4). The residue (392 mg) from the third fraction was further purified by fractionating on silica (100 g) with ether. The first fraction then collected gave a residue (295 mg, 24.4%) which upon recrystallised in ethyl acetate/hexane (1:3) gave (16) as white needle-shaped crystals (160 mg).

Analytical Data were as Follows:

M.p. 165-168° C.; R_{f}s=0.67 (ether), 0.24 (ether/chloroform, 1:4); vmax (KBr) 3360, 3240, 3100, 1720, 1620, 1380, 1160 cm⁻¹; δ_{H} (acetone-d₆) 6.995 (1H, s, C-3-H), 7.461 (1H, dd, J_{C-8-H, C-6-H}=2.8 Hz and J_{C-5-H, C-6-H}=8.1 Hz, C-6-H), 7.478 (1H, s, C-8-H), 7.53 (2H, br s, D₂O exchanged, —NH₂) and 7.89 (1H, m, C-5-H). ¹H-NMR spectrum of (16) in DMSO-d₆/CDCl₃ (ca. 1:15) showed partial decompostion to the starting coumarin. MS: m/z (E.I., rel. intensity) 309.0(2.6), 230.0(77), 202.0(100), 183.5(5), 173.0(10), 69.0(33). Acc. MS: m/z 308.9874, C₁₀H₆F₃NO₅S requires 308.9919. Found: C, 38.8; H, 1.85; N, 4.53%. C₁₀H₆F₃NO₅S requires C, 38.84; H, 1.96; N, 4.53%.

### Preparation of 7-(Sulphoxy)-4-Methylcoumarin, sodium salt (12)

To a solution of 7-hydroxy-4-methylcoumarin (1.0 g, 5.676 mmol) in dried pyridine (20 ml) under an atmosphere of N₂ [FIG. 16] was added sulphur trioxide-pyridine complex (1.8 g, 11.35 mmol, 2 equiv.) and the reaction mixture was stirred overnight. After removal of pyridine, methanol (20 ml) was added to the creamy syrup obtained and the resulting light yellow solution was basified (pH˜8) by dropwise addition of sodium hydroxide in methanol (1 M, ca. 18 ml). The bright yellow suspension formed was filtered and the precipitated washed with more methanol. The filtrate was then concentrated to 30-40 ml and ether (total 120 ml) was added in portions until precipitation completed. The light beige precipitate was collected (711 mg) and 582 mg of which was recrystallised in methanol/ether (1:1) to give (12) as light creamy yellow crystals (335 mg).

Analytical Data were as Follows:

M.p. 172-175° C. (dec.); R_{f}s=0.51 (methanol/ethyl acetate, 1:3), 0.67 (methanol/ether, 1: 3); vmax (KBr) 3500 (br), 3080, 1680, 1610, 1560, 1300, 1260, 1050 cm⁻¹; δ_{H} (DMSO-d₆) 2.407 (3H, s, —CH₃), 6.269 (1H, s, C-3-H), 7.20 (2H, m, C-6-Hand C-8-H), and 7.695 (1H, d, J_{C-6-H, C-5-H}=8.8 Hz, C-5-H). MS: m/z (+ve ion FAB in m-NBA, rel. intensity) 176(100, NBA+Na⁺). MS: m/z (−ve ion FAB in m-NBA, rel. intensity) 175.1 (14, M-Na⁺-SO₃), 255.0 (100, M-Na⁺), 408.0 (8, M-Na⁺+NBA), 431.0 (15, M+153), 444.0(20), 533.0(15). 230.0(77), 202.0(100), 183.5(5), 173.0(10), 69.0(33). Acc. MS: m/z (−ve ion FAB in glycerol, rel. intensity) 254.9982(25), C₁₀H₇O₆S requires 254.9963. Found: C, 40.3; H, 2.92. C₁₀H₇O₆NaS.H₂O requires C, 40.55; H, 3.06%. HPLC [Spherisorb ODS5, 25×4.6 mm; Mobile phase: MeOH/H₂O (70:30), Flow rate: 1 ml/min; λₘₐₓ: 316 nm]: t_{R}=1.5 min, c.f. 7-hydroxy-4-methylcoumarin, 3.6 min.

Other Data were as Follows:

Compound 12 is stable in bases such as sodium hydroxide in methanol but not in acidic conditions. In addition, incomplete basification of the reaction mixture with sodium hydroxide in methanol (<3 equivalents) leads to decomposition of (12). Two equivalents of sodium hydroxide are required for consuming excess sulphur trioxide-pyridine complex to yield the neutral sodium sulphate. Insufficient amount of sodium hydroxide will therefore lead to the formation of sodium hydrogen sulphate which is acidic. Compound 12 appears labile to high temperature as one experiment has shown complete decomposition to 7-hydroxy-4-methylcoumarin after heating (12) as solid at 90° C. for 4 h.

In Vitro Tests

The above-mentioned coumarin sulphamates were tested for their ability to inhibit E1-STS activity using intact MCF-7 breast cancer cells or placental microsomes (100,000 g fraction) essentially as previously described.

To examine whether compound (12) could act as a substrate for E1-STS, 100 μg of the compound was incubated for 1 hour with placental microsomes in the absence or presence of EMATE (10 μM). The unconjugated coumarin formed at the end of the incubation was extracted with diethyl ether. After evaporation of solvent, the residue was examined by TLC using ethyl acetate/methanol (80:20) as eluent, in which the coumarin sulphate (12) and 7-hydroxy-4-methylcoumarin had R_{f} values of 0.79 and 0.95 respectively. Only unconjugated 7-hydroxy-4-methylcoumarin was detected after incubation of compound (12) with placental microsomes. The inclusion of EMATE in the reaction mixture reduced the hydrolysis of compound (12) by E1-STS, indicating that the coumarin sulphate is indeed a substrate for the sulphatase.

The dose-dependent inhibition of oestrone sulphatase in intact MCF-7 breast cancer cells by coumarin-7-O-sulphamate (13), 4-methylcoumarin-7-O-sulphamate (14), 3,4,8-trimethyl-coumarin-7-O-sulphamate (15) and 4-(trifluoromethyl)coumarin-7-O-sulphamate (16) can be seen from FIG. 18. Assays were performed essentially as previously described.(7, 8)

Monolayers of intact MCF-7 cells in 25 cm³ flasks were incubated for 20 h at 37° C. with [³H]oestrone sulphate (2 nM) and coumarin sulphamates at 0.1-10 μM. Oestrone sulphatase activity was determined by measuring the total amount of ³H-labeled oestrone and oestradiol formed. Sulphatase activity in untreated cells was 100-200 fmol/20 h/10⁶ cells. Each point represents the mean s.d. of triplicate measurements.

The free parent coumarins of all coumarin sulphamates prepared showed little or no E1-STS inhibitory activity when tested up to 10 μM. However, in contrast, all four coumarin sulphamates (compounds 13-16) inhibited oestrone sulphatase inhibitory activity in a dose-dependent manner (FIG. 18) and the inhibition at 10 μM ranged from 71.5% for compound 16 to 93.3% for compound 14. The IC₅₀ for inhibition of E1-STS by compound 14, the most effective inhibitor, measured using intact MCF-7 cells was 380 nM.

The time- and concentration-dependent inactivation of oestrone sulphatase by 4-methyl-coumarin-7-O-sulphamate (14) can be seen from FIG. 19. Placental microsomes (200 μg) were preincubated with (14) (control, ●; 0.5 μM, Δ and 10 μM, [symbol] ) for 0-30 min at 37° C. followed by incubation with dextran-charcoal for 10 min at 4° C. Dextran-charcoal was sedimented by centrifugation and portions of the supernatants were then incubated with [³H]oestrone sulphate (20 μM) for 1 h at 37° C. to assess remaining sulphatase activity. Duplicate experiments were run at each concentration, but assays for residual activity were taken at different times in each experiment.

As with EMATE, compound 14 inhibited E1-STS activity in a time- and concentration-dependent manner in a biphasic fashion (FIG. 19), indicating a similar mechanism of action (potential chemical modification of two active site residues). At 10 μM, compound 14 reduced the original E1-STS activity by 95% after preincubating the enzyme with the inhibitor for 20 min.

Additional experiments revealed that compound 14 inhibited placental microsomal DHA-STS activity by 93.6% at the same concentration.

In Vivo Tests

In order to examine if compound 14 possessed oestrogenic activity and also to test its ability to inhibit E1-STS in vivo, it was administered to rats (1 mg/kg subcutaneously, in propylene glycol for 5 days) 14 days after ovariectomy had been performed.

Administration of compound 14 did not result in any significant increase in the uterine weight in these rats (data not shown), showing that compound 14 showed reduced oestrogenic agonist properties. The E1-STS activity in the uteri obtained from these animals was inhibited by 89.4% compared with the activity in untreated animals.

Preliminary data also demonstrate potent oral activity in rats for compound 14, similar to that observed for EMATE.

In addition to these in vivo results, another series of rats (each weighing approximately 200 g) received 4-methyl coumarin-7-O-sulphamate (compound 14) orally in propylene glycol either as a single dose (SD) or daily for seven days (Multiple Dose, MD).

Inhibition of sulphatase activity was assessed in white blood cells (wbcs) that were collected after a SD or MD. Sulphatase activity was assayed using labelled oestrone sulphate as the substrate and measuring the release of oestrone.

The results are shown in FIG. 20 and in the Table below:

% Inhibition

| Dose mg/kg | SD | MD |
|---|---|---|
| 0.1 | 72 | 65 |
| 1.0 | 85 | 85 |
| 10.0 | 96 | 89 |

Similar results were found with liver cells.

Compound 14 therefore demonstrates potent oral activity.

The invention will now be further described by the following numbered paragraphs:

1. A sulphamate compound suitable for use as an inhibitor of both oestrone sulphatase activity and aromatase activity.

2. A compound according to paragraph 1 wherein the compound comprises a first ring structure and a sulphamoyl group, which first ring structure may be substituted and/or unsaturated.

3. A compound according to paragraph 2 wherein the first ring structure is a phenolic ring structure, which phenolic ring may be substituted.

4. A compound according to any one of paragraphs 1 to 3 wherein the compound further comprises a second ring structure, which second ring structure may be substituted and/or unsaturated.

5. A compound according to paragraph 4 wherein the second ring structure is a phenolic ring structure, which phenolic ring may be substituted.

6. A compound according to any one of paragraphs 1 to 5 wherein the compound further comprises a third ring structure which is intermediate the first ring structure and the second ring structure, which third ring structure may be substituted and/or unsaturated.

7. A compound according to paragraph 6 wherein the compound has the general formula I; wherein A represents the first ring structure, B represents the third ring structure, D represents the second ring structure, C is an optional double bond, E is a link joining the second ring structure to the third ring structure, X represents a suitable first group, and Y represents a suitable second group; wherein any one of ring structures A, B and D is a phenolic ring; and wherein any one of ring structures A, B and D has bound thereto a sulphamate group.

8. A compound according to paragraph 7 wherein the compound has the general formula II wherein F represents a phenolic ring structure (the first ring structure), J represents the third ring structure, I represents a phenolic ring structure (the second ring structure), G is an optional double bond, H is a link joining the second ring structure to the third ring structure, and Y represents a suitable second group; wherein any one of ring structures F, J and I has bound thereto a sulphamate group.

9. A compound having the general formula II wherein F represents a phenolic ring structure (a first ring structure), J represents a third ring structure, I represents a phenolic ring structure (a second ring structure), G is an optional double bond, H is a link joining the second ring structure to the third ring structure, and Y represents a suitable second group; wherein any one of ring structures F, J and I has bound thereto a sulphamate group.

10. A compound according to any one of paragraphs 7 to 9 wherein the first ring structure and the second ring structure are substituted.

11. A compound according to any one of paragraphs 7 to 10 wherein any one of ring structures A or F and D or I has bound thereto a sulphamate group.

12. A compound according to any one of paragraphs 7 to 11 wherein the third ring structure is a heterogeneous ring structure.

13. A compound according to any one of paragraphs 7 to 12 wherein X is C═O.

14. A compound according to any one of paragraphs 7 to 13 wherein Y is O.

15. A compound according to any one of the preceding paragraphs wherein the compound of the present invention is a sulphamate of any one of a flavone, an isoflavone or a flavanone.

16. A compound according to paragraph 15 wherein the compound is any one of: a compound of the general formula IV, a compound of the general formula V or a compound of the general formula VI; wherein R₁-R₁₂ are independently selected from H, OH, a halogen, an amine, an amide, a sulphonamine, a sulphonamide, any other sulphur containing group, a saturated or unsaturated C₁₋₁₀ alkyl, an aryl group, a saturated or unsaturated C₁₋₁₀ ether, a saturated or unsaturated C₁₋₁₀ ester, a phosphorous containing group; and wherein at least one of R₁-R₁₂ is a sulphamate group.

17. A compound according to any one of the preceding paragraphs wherein the sulphamate group has the general formula OSO₂NR₁₃R₁₄ wherein R₁₃ and R₁₄ are independently selected from H, OH, a halogen, a saturated or unsaturated C₁₋₁₀ alkyl, an aryl group, a saturated or unsaturated C₁₋₁₀ ether, a saturated or unsaturated C₁₋₁₀ ester.

18. A compound according to any one of the preceding paragraphs wherein the compound is any one of a compound of the general formula IV, a compound of the general formula V or a compound of the general formula VI; wherein R₁-R₁₂ are independently selected from H, OH, OSO₂NR₁₃R₁₄, O—CH₃; wherein at least one of R—R₁₂ is OSO₂NR₁₃R₁₄, and wherein R₁₃ and R₁₄ are defined in paragraph 17.

19. A compound according to paragraph 16 or paragraph 17 wherein R₁₃ and R₁₄ are H.

20. A compound according to any one of the preceding paragraphs wherein the compound is a sulphamate of any one of the flavone of formula VII, the isoflavone of formula VIII or the flavanone of formula IX.

21. A compound according to any one of the preceding paragraphs wherein the compound is the sulphamate of any one of formula VII, formula VIII or formula IX.

22. A compound according to any one of the preceding paragraphs wherein the compound is a sulphamate of any one of a flavone, an isoflavone or a flavanone; and wherein the sulphamoyl group is on the C4′ atom of the flavone, isoflavone or flavanone.

23. A compound according to any one of the preceding paragraphs wherein the compound is a flavanoid sulphamate.

24. A compound according to any one of the preceding paragraphs for use as a pharmaceutical.

25. A compound according to any one of paragraphs 1 to 23 for inhibiting oestrone sulphatase activity and aromatase activity.

26. A pharmaceutical composition comprising a compound according to any one of paragraphs 1 to 23; and a pharmaceutically acceptable carrier, excipient or diluent.

27. Use of a compound according to any one of paragraphs 1 to 23 in the manufacture of a pharmaceutical for inhibiting oestrone sulphatase activity and aromatase activity.

28. A process for preparing a compound according to any one of paragraphs 1 to 23, the process comprising sulphating a flavone, isoflavone or a flavanone.

29. A process for preparing a compound according to any one of paragraphs 1 to 23, the process comprising sulphamaylating a flavone, isoflavone or a flavanone.

30. A compound substantially as described herein.

31. A process of preparing a compound substantially as described herein.

All publications and patents and patent applications mentioned in the above specification are herein incorporated by reference. Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

### REFERENCES

(1) Santner, S. J.; Feil, P. D.; Santen, R. J. In situ oestrogen production via the oestrone sulphatase pathway in breast tumors: relative importance vs. the aromatase pathway. J. Clin. Endocrinol. Metab. 1984, 59, 29-33.
(2) Yamamoto, T.; Kitawaki, J.; Urabe, M.; Honjo, H.; Tamura, T.; Noguchi, T.; Okada, H.; Sasaki, H.; Tada, A.; Terashima, Y.; Nakamura, J.; Yoshihama, M. Oestrogen productivity of endometrium and endometrial cancer tissue—influence of aromatase on proliferation of endometrial cancer cells. J. Steroid Biochem. Mol. Biol. 1993, 44, 463-468.
(3) Santen, R. J.; Santner, S. J.; Davis, B.; Veldhuis, J.; Samojilik, E.; Ruby, E. Aminogluthethimide inhibits extraglandular oestrogen production in post-menopausal women with breast carcinoma. J. Clin. Endocrinol. Metab. 1978, 47, 1257-1265.
(4) Reed, M. J.; Lai, L. C.; Owen, A. M.; Singh, A.; Coldham, N. G.; Purohit, A.; Ghilchik, M. W.; Shaikh, N. A.; James, V. H. T. Effect of treatment with 4-hydroxy-androstenedione on the peripheral conversion of androstenedione to oestrone and in vitro tumour aromatase activity in postmenopausal women with breast cancer. Cancer Res. 1990, 50, 193-196.
(5) Ruder, H. J.; Loriaux, D. L.; Lipsett, M. B. Oestrone sulphate: production rate and metabolism in man. J. Clin. Invest. 1972, 51, 1020-1023.
(6) James, V. H. T.; McNeill, J. M.; Lai, L. C.; Newton, C. J.; Ghilchik, M. W.; Reed, M. J. Aromatase activity in normal breast and breast tumor tissues: in vivo and in vitro studies. Steroids 1987, 50, 269-279.
(7) Howarth, N. M.; Purohit, A.; Reed, M. J.; Potter, B. V. L. Oestrone sulphamates: potent inhibitors of oestrone sulphatase with therapeutic potential. J. Med. Chem. 1994, 37, 219-221.
(8) Purohit, A.; Williams, G. J.; Howarth, N. M.; Potter, B. V. L.; Reed, M. J. Inactivation of steroid sulphatase by an active site-directed inhibitor, oestrone-3-O-sulphamate. Biochemistry 1995, 34, 11508-11514.
(9) Purohit, A.; Dauvois, S.; Parker, M. G.; Potter, B. V. L.; Williams, G. J.; Reed, M. J. The hydrolysis of oestrone sulphate and dehydroepiandrosterone sulphate by human steroid sulphatase expressed in transfected COS-1 cells. J. Steroid Biochem. Mol. Biol. 1994, 50, 101-104.
(10) Dauvois, S.; Labrie, F. Androstenedione and androst-5-ene-3β,17β-diol stimulate DMBA-induced rat mammary tumours—role of aromatase. Breast Cancer Res. Treat. 1989, 13, 61-69.
(11) Purohit, A.; Williams, G. J.; Roberts, C. J.; Potter, B. V. L.; Reed, M. J. In vivo inhibition of oestrone sulphatase and dehydroepiandrosterone sulphatase by oestrone-3-O-sulphamate. Int. J. Cancer 1995, 63, 106-111.
(12) Woo, L. W. L.; Lightowler, M.; Purohit, A.; Reed, M. J.; Potter, B. V. L. Heteroatom-substituted analogues of the active-site directed inhibitor oestra-1,3,5(10)-trien-17-one-3-sulphamate inhibit oestrone sulphatase by a different mechanism. J. Steroid Biochem. Mol. Biol. 1996 (in press).
(13) Elger, W.; Schwarz, S.; Hedden, A.; Reddersen, G.; Schneider, B. Sulphamates of various oestrogens—prodrugs with increased systemic and reduced hepatic oestrogenicity at oral application. J. Steroid Biochem. Mol. Biol. 1995, 55, 395-403.
(14) Li, P. K; Rhodes, M. E.; Jagannathan, S; Johnson, D. A. Memory enhancement mediated by the steroid sulphatase inhibitor oestrone 3-O-sulphamate. J. Endocrinol. 1995, 144, Abstr. P155.
(15) Daynes, R. A.; Araneo, B. A.; Dowell, T. A.; Huang, K.; Dudley, D. Regulation of murine lymphokine production in vivo. 3. The lymphoid tissue micro-environment exerts regulatory influences over T-helper cell function. J. Exp. Med. 1990, 171, 979-996.
(16) Rook, G. A. W.; Hernandez-Pando, R.; Lightman, S. Hormones, peripherally activated prohormones and regulation of the TH1/TH2 balance. Immunol. Today 1994, 15, 301-303.
(21) James & Reed (1980) Prog Cancer Res Ther 14: 471-487.
(22) James et al (1987) Steroids 50: 269-279.
(23) Reed et al (1994) Drugs for the Future 19: 673-680.
(24) Santner et al (1984) J Clin Endocr Metab 59: 29-33.
(25) Yamamoto et al (1993) J Steroid Biochem Molec Biol 44: 463-468.
(26) Poulin & Labrie (1986) Cancer Res 46: 4933-4937.
(27) Dauvois & Labrie (1989) Breast Cancer Res Treat 13: 61-69.
(28) Purohit et al (1994) J Steroid Biochem Mol Biol 50: 101-104.
(29) Howarth et al (1994) J Med Chem 37: 219-221.
(30) Purohit et al (1995) Biochemistry 34: 11508-11514.
(31) Purohit et al (1995) Int J Cancer 62: 106-111.
(32) Svenstrup et al (1994) Eur J Cancer 30A: 1254-1258.
(33) Johnston et al (1994) Cancer Res 54: 5875-5881.
(34) Dowsett et al (1994) Eur J Cancer 30A: 1453-1458.
(35) Kellis et al (1986) Biochem Pharmacol 35: 2887-2891.
(36) Campbell & Kurzer (1993) J Steroid Biochem Molec Biol 46: 381-388.
(37) Newton et al (1986) J Steroid Biochem 24: 1033-1039.
(38) Shields-Botella et al (2003) J Steroid Biochem Molec Biol 84: 327-335.

1. A sulphamate compound that is an inhibitor of both oestrone sulphatase activity and aromatase activity.

2. A compound according to claim 1 wherein the compound comprises a first ring structure and a sulphamoyl group, which first ring structure may be substituted and/or unsaturated.

3. A compound according to claim 2 wherein the first ring structure is a phenolic ring structure, which phenolic ring may be substituted.

4. A compound according to claim 2 wherein the compound further comprises a second ring structure, which second ring structure may be substituted and/or unsaturated.

5. A compound according to claim 4 wherein the second ring structure is a phenolic ring structure, which phenolic ring may be substituted.

6. A compound according to claim 4 wherein the compound further comprises a third ring structure which is intermediate the first ring structure and the second ring structure, which third ring structure may be substituted and/or unsaturated.

7. A compound according to claim 6 wherein the compound has the general formula I; 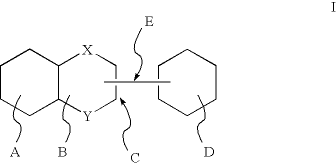
wherein A represents the first ring structure, B represents the third ring structure, D represents the second ring structure, C is an optional double bond, E is a link joining the second ring structure to the third ring structure, X represents a suitable first group, and Y represents a suitable second group; wherein any one of ring structures A, B and D is a phenolic ring; and wherein any one of ring structures A, B and D has bound thereto a sulphamate group.

8. A compound according to claim 7 wherein the compound has the general formula II 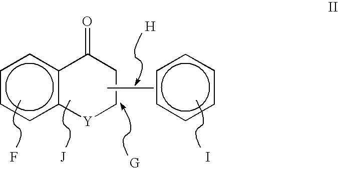
wherein F represents a phenolic ring structure (the first ring structure), J represents the third ring structure, I represents a phenolic ring structure (the second ring structure), G is an optional double bond, H is a link joining the second ring structure to the third ring structure, and Y represents a suitable second group; wherein any one of ring structures F, J and I has bound thereto a sulphamate group.

9. A compound having the general formula II wherein F represents a phenolic ring structure (a first ring structure), J represents a third ring structure, I represents a phenolic ring structure (a second ring structure), G is an optional double bond, H is a link joining the second ring structure to the third ring structure, and Y represents a suitable second group; wherein any one of ring structures F, J and I has bound thereto a sulphamate group.

10. A compound according to claim 7 wherein the first ring structure and the second ring structure are substituted.

11. A compound according to claim 7 wherein any one of ring structures A or F and D or I has bound thereto a sulphamate group.

12. A compound according to claim 7 wherein the third ring structure is a heterogeneous ring structure.

13. A compound according to claim 7 wherein X is C═O.

14. A compound according to claim 7 wherein Y is O.

15. A compound according to claim 1 wherein the compound of the present invention is a sulphamate of any one of a flavone, an isoflavone or a flavanone.

16. A compound according to claim 15 wherein the compound is any one of:
a compound of the general formula IV, 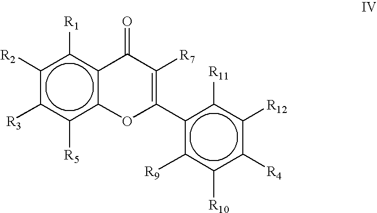
a compound of the general formula V or 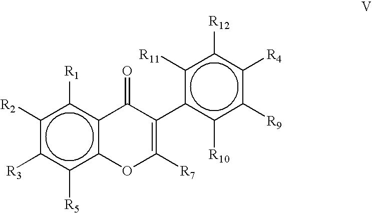
a compound of the general formula VI; 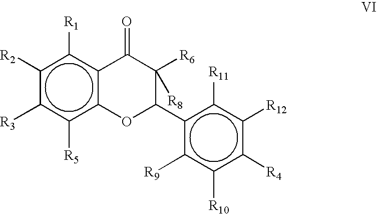
wherein R₁-R₁₂ are independently selected from H, OH, a halogen, an amine, an amide, a sulphonamine, a sulphonamide, any other sulphur containing group, a saturated or unsaturated C₁₋₁₀ alkyl, an aryl group, a saturated or unsaturated C₁₋₁₀ ether, a saturated or unsaturated C₁₋₁₀ ester, a phosphorous containing group; and wherein at least one of R₁-R₁₂ is a sulphamate group.

17. A compound according to claim 7 wherein the sulphamate group has the general formula OSO₂NR₁₃R₁₄ wherein R₁₃ and R₁₄ are independently selected from H, OH, a halogen, a saturated or unsaturated C₁₋₁₀ alkyl, an aryl group, a saturated or unsaturated C₁₋₁₀ ether, a saturated or unsaturated C₁₋₁₀ ester.

18. A compound according to claim 1 wherein the compound is any one of:
a compound of the general formula IV, 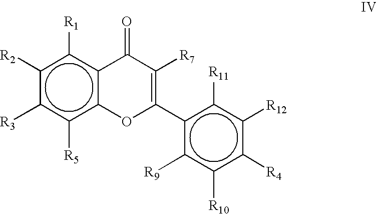
a compound of the general formula V or 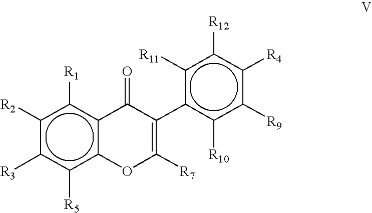
a compound of the general formula VI; 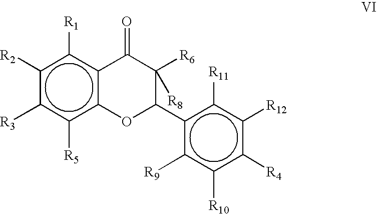
wherein R₁-R₁₂ are independently selected from H, OH, OSO₂NR₁₃R₁₄, O—CH₃; wherein at least one of R₁-R₁₂ is OSO₂NR₁₃R₁₄, and wherein R₁₃ and R₁₄ are independently selected from H, OH, a halogen, a saturated or unsaturated C₁₋₁₀ alkyl, an aryl group, a saturated or unsaturated C₁₋₁₀ ether, a saturated or unsaturated C₁₋₁₀ ester.

19. A compound according to claim 17 wherein R₁₃ and R₁₄ are H.

20. A compound according to claim 18 wherein R₁₃ and R₁₄ are H.

21. A compound according to claim 1 wherein the compound is a sulphamate of any one of the flavone of formula VII, the isoflavone of formula VIII or the flavanone of formula IX.

22. A compound according to claim 1 wherein the compound is the sulphamate of any one of formula VII, formula VIII or formula IX.

23. A compound according to claim 1 wherein the compound is a sulphamate of any one of a flavone, an isoflavone or a flavanone; and wherein the sulphamoyl group is on the C4′ atom of the flavone, isoflavone or flavanone.

24. A compound according to claim 1 wherein the compound is a flavanoid sulphamate.

25. A compound according to claim 1 for use as a pharmaceutical.

26. A pharmaceutical composition comprising a compound according to claim 1 and a pharmaceutically acceptable carrier, excipient or diluent.

27. A process for preparing a compound according to claim 1, the process comprising sulphating a flavone, isoflavone or a flavanone.

28. A process for preparing a compound according to claim 1, the process comprising sulphamaylating a flavone, isoflavone or a flavanone.
